(19) United States
(12) Patent Application Publication
(10) Pub. No.: US 2024/0139319 A1
(43) Pub. Date: May 2, 2024

(54) GENE SILENCING

(71) Applicant: EPSILEN BIO S.R.L, Milan (IT)

(72) Inventors: Angelo Leone Lombardo, Milan (IT); Alice Reschigna, Milan (IT); Tania Baccega, Milan (IT)

(73) Assignee: EPSILEN BIO S.R.L, Milan (IT)

(21) Appl. No.: 18/274,752

(22) PCT Filed: Feb. 1, 2022
(86) PCT No.: PCT/EP2022/052372

§ 371 Date: Jul. 28, 2023

(30) Foreign Application Priority Data
Feb. 1, 2021 (EP) 21154639.5

(51) Int. Cl. A61K 39/00; A61K 31/7088; A61K 38/46; C12N 9/22; C12N 15/11; C12N 15/90

(52) U.S. Cl. CPC A61K 39/4631; A61K 31/7088; A61K 38/465; A61K 39/4611; A61K 39/4632; C12N 9/22; C12N 15/11; C12N 15/907; C12N 2310/20; C12N 2800/80

(57) ABSTRACT

An engineered transcriptional modulator (ETM) comprising: (a) at least one epigenetic effector domain; operably linked to (b) an endonuclease.

### FIELD OF THE INVENTION

The present invention relates to engineered transcriptional modulators (ETM), for example engineered transcriptional repressors (ETRs), for gene editing and epigenetic modification. More specifically, the present invention relates to ETMs (e.g., ETRs) for use in multiplexing methods for modifying the expression of at least two target genes, wherein the expression of a first target gene is modified by gene editing and the expression of second target gene is modified by epigenetic modification, including during gene therapy applications.

### BACKGROUND TO THE INVENTION

Adoptive immunotherapy using engineered T cells has emerged as a powerful approach to treat cancer. These cells can be prepared from the patient's own blood (autologous) or derived from a different donor (allogeneic) and are redirected against cancer cells by ectopic expression of a transgenic T Cell Receptor (TCR) or a Chimeric Antigen Receptor (CAR) recognizing tumour-related antigens. TCRs and CARs may be introduced into ex vivo expanded T cells by different means, including lentiviral and retroviral vectors. These vectors, however, tend to integrate semi-randomly in the genome of T cells, posing safety concerns related to transcriptional deregulation of tumour-promoting genes. To avoid this risk, genome editing with artificial nucleases, such as CRISPR/Cas9, has been used to drive insertion of the CAR sequence into the endogenous TCR locus (J. Eyquem et al., Nature 2017 Mar. 2; 543(7643):113-117), an approach that also enhances T-cell potency.

Genome editing has been further used to improve efficiency and reduce toxicity of T cell therapy via the knockout of additional key genes. In this regard, the most common targets are the TCR genes (encoded by TRAC and TRBC, with the latter present in two copies in cis on the same chromosome), the β-2 microglobulin (B2M) gene, and the programmed cell death 1 (PDCD1, also referred to as PD1) gene. Inactivation of TRAC and B2M is believed to reduce graft-versus-host reactions, whereas inactivation of PDCD1 is used to desensitize transplanted T cells to immune dampening signals originating from the cancer cells/microenvironment.

While promising, these multiplexing gene editing approaches (i.e., disruption of multiple genes per cell) come with two related issues:
(i) Induction of multiple DNA breaks per cell may over-activate cellular DNA damage responses, ultimately leading to apoptosis or poor performance/fitness of the transplanted cells. In this regard, triple editing has been posed as the upper limit for multiplexing, above which significant cell toxicity can be observed.
(ii) Chromosomal translocations may occur between or among multiple DNA breaks (including on- and off-target sites of the nucleases and spontaneous breaks, the latter occurring at a relatively high rate in cultured T cells), further jeopardizing safety of the approach. Clinical and preclinical studies of multiplexing in CAR-T cell products have reported alarming levels of genomic translocations (up to 5%), even when dual-gene editing approaches were used (L. Poirot et al., Cancer Res. 2015 Sep. 15; 75(18):3853-64; W. Qasim et al., Sci Transl Med; 2017 Jan. 25; 9(374); E. Stadtaumer et al., Science 2020 Feb. 28; 367(6481)).

Targeted epigenetic modification (such as epi-silencing) may represent a safer alternative to gene editing approaches for multiplexing in T cells. Epi-silencing exploits epigenetics, rather than DNA breaks, to inactivate its intended target gene, for example through DNA methylation at CpG sites (A. Amabile et al., Cell. 2016 Sep. 22; 167(1):219-232).

Epi-silencing may be achieved by the transient delivery of Engineered Transcriptional Repressors (ETRs), proteins comprising, for example, a catalytically disabled Cas9 (dCas9) or a transcription activator-like effector (TALE) or a Zinc-finger protein (ZFP) fused to epigenetic domains from naturally occurring epigenetic effector proteins (such as KRAB, DNMT3L and DNMT3A). The application of ETRs in silencing individual as well as multi-copy genes in cell lines and in primary T lymphocytes was reported by A. Amabile supra and T. Mlambo et al., Nucleic Acids Res. 2018 May 18; 46(9):4456-4468. However, the activity of ETRs appears to preferably occur at genes that possess a CpG island (CGI), thus excluding several potentially relevant targets (e.g., TCR genes and PD1 amongst others).

Accordingly, there remains a need for the development of technologies capable of modifying multiple genes within the same cell. Technologies which reduce the number of multiple DNA breaks per cell, compared to multiplexing gene editing strategies, may be a safer approach and may avoid cellular DNA damage responses and undesired chromosomal translocations.

### SUMMARY OF THE INVENTION

The present invention relates to the development of a combined gene and epigenetic editing strategy to modify multiple genes within the same cell. In particular, it exploits an engineered transcriptional modulator (ETM), for example an engineered transcriptional repressor (ETR), which comprises an epigenetic effector domain operably linked to an endonuclease (such as a catalytically active Cas9) and guide ribonucleic acids (gRNAs) of different lengths to promote permanent epigenetic editing (e.g., silencing) of one or more genes and genetic editing (e.g., inactivation) of another gene.

This orthogonal approach overcomes the genotoxic risks associated with the use of nuclease-mediated genome editing technologies to inactivate multiple genes per cell. Advantageously, the present invention enables targeting of genes that may be more challenging to achieve with targeted epigenetic modification, enabling targeting of both genes having a CpG island (CGI) and genes which do not have a CGI in one multiplexing strategy.

Thus, the present invention provides a combined strategy of gene editing coupled to epigenetic modification, such as epigenetic silencing. This combination will:
(i) reduce the burden of genomic translocations compared to multiplexing gene editing methods. The target selected for gene editing will typically lack a CGI. This gene may be also used as a target site for insertion of exogenous expression cassettes encoding, for example, tumour restricted TCRs or CARs introduced with homologous recombination; and
(ii) utilise epigenetics to modify, e.g., silence, one or more CGI-containing genes.
(iii) allow the use of the same construct (an ETM) to achieve silencing in two different modalities, thus reducing the amount of gene editor-encoding RNA that needs to be added to the cell for correct silencing. An advantage of the present invention is to reduce the number of constructs required for multiplex modification, thus improving efficiency and decreasing manufacturing costs.

Suitably, gene editing may be limited to one gene (which lacks CGI) and at least one gene (such as at least two, or at least three or more genes) comprising a CGI may be modified epigenetically.

Overall, development of such a combined strategy will result in safer and more efficient T cell products for adoptive immunotherapy of cancer.

In one aspect, the present invention provides an engineered transcriptional modulator (ETM) comprising: a) at least one epigenetic effector domain; operably linked to b) an endonuclease.

In certain embodiments, the ETM is an engineered transcriptional repressor (ETR). In some embodiments, the ETM is an engineered transcriptional activator (ETA).

In some embodiments, the ETM (e.g., ETR) comprises one, two or three epigenetic effector domains. In some embodiments, the ETM (e.g., ETR) comprises one epigenetic effector domain. In some embodiments, the ETM (e.g., ETR) comprises two epigenetic effector domains. In some embodiments, the ETM (e.g., ETR) comprises three epigenetic effector domains.

In some embodiments, the at least one epigenetic effector domain comprises a Kruppel-associated box (KRAB) domain, a DNA methyltransferase (DNMT) domain, a DNMT-like domain, and/or a histone methyltransferase (HMT) domain. In some embodiments, the epigenetic effector domain is a transcriptional repressor domain (e.g., a Kruppel-associated box (KRAB) domain).

In some embodiments, the at least one epigenetic effector domain is selected from the group consisting of: DNMT1, DNMT3A, DNMT3B, DNMT3L and SETDB1.

In some embodiments, the ETM (e.g., ETR) comprises a first epigenetic effector domain comprising a KRAB domain and a second epigenetic effector domain comprising a DNMT domain. In some embodiments, the ETM (e.g., ETR) comprises a first epigenetic effector domain comprising a KRAB domain and a second epigenetic effector domain comprising a DNMT-like domain. In some embodiments, the ETM (e.g., ETR) comprises a first epigenetic effector domain comprising a KRAB domain, a second epigenetic effector domain comprising a DNMT domain, and a third epigenetic effector domain comprising a DNMT-like domain. In certain embodiments, the ETM may comprise as epigenetic effector domains KRAB and DNMT3A; KRAB and DNMT3L; or KRAB, DNMT3A, and DNMT3L. In some embodiments, the ETM (e.g., ETR) comprises a transcriptional repressor domain (e.g., a Kruppel-associated box (KRAB) domain) and a DNMT3L domain. In some embodiments, the ETM (e.g., ETR) comprises a transcriptional repressor domain (e.g., a Kruppel-associated box (KRAB) domain), a DNMT3A domain and a DNMT3L domain.

In some embodiments, the endonuclease comprises an RNA binding domain.

In some embodiments, the endonuclease is a Clustered Regularly Interspaced Short Palindromic Repeats (CRISPR)/Cas system.

In some embodiments, the endonuclease is a Cas endonuclease.

In certain embodiments, the endonuclease is a Cas9 endonuclease. In certain embodiments, the endonuclease is a SpCas9 endonuclease

In some embodiments, the ETM (e.g., ETR) comprises or consists of a Cas9-KRAB, Cas9-DNMT3A or Cas9-DNMT3L fusion protein, which can be used together.

In some embodiments, the ETM (e.g., ETR) is a bi- or tri-partite fusion protein.

In another aspect, the present invention provides a gRNA which comprises a spacer sequence which comprises or consists of the sequence of any one of SEQ ID NOs: 23-46, 562-1076, 2778-4478, or 4553-4565 or a homologue or fragment thereof. In another aspect, the present invention provides a gRNA which comprises a spacer sequence which comprises or consists of the sequence of any one of SEQ ID NOs: 23-46, 562-1076, 2778-4478, and 4553-4565 or a homologue or fragment thereof.

In another aspect, the spacer sequence consists of a fragment of any one of SEQ ID NOs: 23-46, 562-1076 or 2778-4478, such as a 10, 11, 12, 13, 14, 15, 16, 17, 18, 19 or 20 nucleotide fragment of any one of SEQ ID NOs: 23-46, 562-1076, 2778-4478 or 4553-4565. In another aspect, the spacer sequence consists of a fragment of any one of SEQ ID NOs: 23-46, 562-1076, 2778-4478, and 4553-4565, such as a 10, 11, 12, 13, 14, 15, 16, 17, 18, 19 or 20 nucleotide fragment of any one of SEQ ID NOs: 23-46, 562-1076, 2778-4478, and 4553-4565. The fragment may be a truncation of the sequence from the 5′ end.

In another aspect, the spacer sequence consists of a fragment of any one of SEQ ID NOs: 23-46, 562-1076 or 2778-4478, such as at least 10, at least 11, at least 12, at least 13, at least 14, at least 15, at least 16, at least 17, at least 18, at least 19 or at least 20 continuous nucleotides of any one of SEQ ID NOs: 23-46, 562-1076 or 2778-4478. In another aspect, the spacer sequence consists of a fragment of any one of SEQ ID NOs: 23-46, 562-1076, 2778-4478, and 4553-4565, such as at least 10, at least 11, at least 12, at least 13, at least 14, at least 15, at least 16, at least 17, at least 18, at least 19 or at least 20 continuous nucleotides of any one of SEQ ID NOs: 23-46, 562-1076, 2778-4478, and 4553-4565.

In another aspect, the present invention provides a combination (e.g., a system) comprising an ETM (e.g., ETR) according to the present invention, and at least one gRNA. The gRNA(s) may target the ETM (e.g., ETR) to one or more target gene(s). In another aspect, the present invention provides a combination (e.g., a system) comprising an ETM (e.g., ETR) according to the present invention, or polynucleotide(s) encoding therefor, and at least one gRNA, or polynucleotides coding therefor. The combination may comprise one or more ETMs (e.g., ETRs) according to the present invention, such as one, two or three ETMs (e.g., ETRs), or polynucleotides encoding therefor.

In some embodiments, each ETM is a fusion protein comprising a catalytically active CRISPR/Cas endonuclease domain.

In another aspect, the present invention provides a combination for modifying transcription, expression and/or activity of one or more (e.g. two or more) gene in a cell, the combination comprising: (A) one or more fusion proteins each comprising a catalytically active CRISPR/Cas endonuclease domain, wherein the one or more fusion proteins collectively comprise a transcriptional repressor domain and a DNMT3L domain, or polynucleotide(s) encoding the one or more fusion proteins; (B) one or more guide RNAs (gRNAs) having a spacer sequence with a length that allows epigenetic editing and not gene editing of a first gene in the cell, wherein the first gene comprises a CpG island (CGI), or polynucleotide(s) coding for the one or more gRNAs; and (C) one or more gRNAs having a spacer sequence with a length that allows gene editing of a second gene in the cell, or polynucleotide(s) coding for the one or more gRNAs.

In some embodiments, at least one epigenetic effector domain is a transcriptional repressor domain (e.g. a Krüppel-associated box (KRAB) domain), and/or at least one epigenetic effector domain is a DNMT3L domain. In some embodiments, at least one epigenetic effector domain is a transcriptional repressor domain (e.g. a Kruppel-associated box (KRAB) domain), at least one epigenetic effector domain is a DNMT3A domain, and/or at least one epigenetic effector domain is a DNMT3L domain.

In some embodiments, the one or more ETMs collectively comprise a transcriptional repressor domain (e.g. a Kruppel-associated box (KRAB) domain) and a DNMT3L domain. In some embodiments, the one or more ETMs collectively comprise a transcriptional repressor domain (e.g. a Kruppel-associated box (KRAB) domain), a DNMT3A domain and a DNMT3L domain.

In some embodiments, the spacer sequence is less than or equal to 16 nucleotides in length. In some embodiments, the spacer sequence is 11 to 16 nucleotides in length, such as 12 to 16, 13 to 16, 14 to 16 or 15 to 16 nucleotides in length.

In some embodiments, the spacer sequence is 17 or more nucleotides in length, such as 18 or more, 19 or more, or 20 or more nucleotides in length. In some embodiments, the spacer sequence is 17 to 30 nucleotides in length, such as 18 to 30, 19 to 30 or 20 to 30 nucleotides in length. In some embodiments, the spacer sequence is 17 to 25 nucleotides in length, such as 18 to 25, 19 to 25 or 20 to 25 nucleotides in length. In some embodiments, the spacer sequence is 17 to 20 nucleotides in length, such as 18 to 20 or 19 to 20 nucleotides in length.

In some embodiments, the spacer sequence is less than or equal to 17 nucleotides in length. In some embodiments, the spacer sequence is 11 to 17 nucleotides in length, such as 12 to 17, 13 to 17, 14 to 17, 15 to 17, 16 to 17, 12 to 16, 13 to 16, 14 to 16, or 15 nucleotides in length. In some embodiments, the one or more gRNAs in (B) has a spacer sequence of less than or equal to 17 nucleotides. In some embodiments, the one or more gRNAs in (B) has a spacer sequence of 11 to 17 nucleotides, such as 12 to 17, 13 to 17, 14 to 17, 15 to 17, 16 to 17, 12 to 16, 13 to 16, 14 to 16, or 15 nucleotides.

In some embodiments, the spacer sequence is 18 or more nucleotides in length, such as 19 or more, or 20 or more nucleotides in length. In some embodiments, the spacer sequence is 18 to 30 nucleotides in length, such as 19 to 30 or 20 to 30 nucleotides in length. In some embodiments, the spacer sequence is 18 to 25 nucleotides in length, such as 19 to 25 or 20 to 25 nucleotides in length. In some embodiments, the spacer sequence is 18 to 21 nucleotides in length, such as 19 to 21 or 20 to 21 nucleotides in length. In some embodiments, the spacer sequence is 18 to 20 nucleotides in length, such as 19 to 20 nucleotides in length. In some embodiments, the one or more gRNAs in (C) has a spacer sequence of 18 or more nucleotides, such as 19 or more, or 20 or more nucleotides. In some embodiments, the one or more gRNAs in (C) has a spacer sequence of 18 to 30 nucleotides, such as 19 to 30 or 20 to 30 nucleotides. In some embodiments, the one or more gRNAs in (C) has a spacer sequence of 18 to 25 nucleotides, such as 19 to 25 or 20 to 25 nucleotides. In some embodiments, the one or more gRNAs in (C) has a spacer sequence of 18 to 21 nucleotides, such as 19 to 21 or 20 to 21 nucleotides. In some embodiments, the one or more gRNAs in (C) has a spacer sequence of 18 to 20 nucleotides, such as 19 to 20 nucleotides.

In certain embodiments, the combination comprises at least two gRNAs. Suitably, the combination may comprise two gRNAs. Suitably, the combination may comprise three, four, five, six, seven or eight gRNAs.

The at least two gRNAs may target the ETM (e.g., ETR) to different target genes. For example, a first gRNA may target the ETM (e.g., ETR) to a first target gene and a second gRNA may target the ETM (e.g., ETR) to a second target gene. A third gRNA may, for example, target the ETM (e.g., ETR) to a third target gene. Additional gRNAs may target the ETM (e.g., ETR) to additional target genes.

In some embodiments, one target gene may be targeted with two or more gRNAs. For example, it may be beneficial to target the same gene with several gRNAs for optimal epigenetic modification e.g., epigenetic silencing. A second target gene may be targeted with another gRNA.

In particular embodiments, the at least two gRNAs comprise spacer sequences of different lengths.

In some embodiments, at least one gRNA (e.g., one, two, three or more gRNAs) may have a spacer sequence with a length that allows epigenetic editing of a target gene by the ETM and/or at least one gRNA may have a spacer sequence with a length that allows gene editing of a target gene by the ETM.

In some embodiments, a first gRNA may have a spacer sequence with a length that allows epigenetic editing of a first target gene by the ETM and a second gRNA may have a spacer sequence with a length that allows gene editing of a second target gene by the ETM.

In some embodiments, at least one gRNA (e.g., one, two, three or more gRNAs) may have a spacer sequence with a length that allows epigenetic editing and not gene editing of a target gene by the ETM and/or at least one gRNA may have a spacer sequence with a length that allows gene editing of another target gene by the ETM.

In some embodiments, a first gRNA may have a spacer sequence with a length that allows epigenetic editing and not gene editing of a first target gene by the ETM and a second gRNA may have a spacer sequence with a length that allows gene editing of a second target gene by the ETM.

Suitably, at least one gRNA(s) may comprise a spacer sequence which is 15, 16, 17, 18, 19 or 20 nucleotides in length.

Suitably, one of the at least two gRNAs may comprise a spacer sequence which is less than or equal to 17 (e.g., less than or equal to 16) nucleotides in length.

In some embodiments, the combination comprises:
(a) a first gRNA comprises a spacer sequence which is less than or equal to 16 nucleotides in length, such as less than or equal to 15, less than or equal to 14, less than or equal to 13 or less than or equal to 12 nucleotides in length; and/or
(b) a second gRNA comprises a spacer sequence which is 17 or more nucleotides in length, such as 18 or more, 19 or more, or 20 or more nucleotides in length.

In some embodiments, the combination comprises:
(a) a first gRNA comprises a spacer sequence which is 11 to 16 nucleotides in length, such as 12 to 16, 13 to 16, 14 to 16 or 15 to 16 nucleotides in length; and/or
(b) a second gRNA comprises a spacer sequence which is 17 to 30 nucleotides in length, such as 18 to 30, 19 to 30, 20 to 30, 17 to 25, 18 to 25, 19 to 25, 20 to 25, 17 to 20, 18 to 20 or 19 to 20 nucleotides in length.

In some embodiments, the combination comprises:
(a) a first gRNA comprises a spacer sequence which is less than or equal to 17 nucleotides in length, such as less than or equal to 16, less than or equal to 15, less than or equal to 14, less than or equal to 13, less than or equal to 12 nucleotides, or equal to 11 nucleotides in length; and/or
(b) a second gRNA comprises a spacer sequence which is 18 or more nucleotides in length, such as 19 or more, or 20 or more nucleotides in length.

In some embodiments, the combination comprises:
(a) a first gRNA comprises a spacer sequence which is 11 to 17 nucleotides in length, such as 12 to 17 (e.g., 12 or 16), 13 to 17 (e.g., 13 to 16), 14 to 17 (e.g., 14 to 16), 15 to 17 (e.g., 16), or 17 nucleotides in length; and/or
(b) a second gRNA comprises a spacer sequence which is 18 to 30 nucleotides in length, such as 19 to 30, 20 to 30, 18 to 25, 19 to 25, 20 to 25, 18 to 20, or 19 to 20 nucleotides in length.

In some embodiments, the one or more guide RNAs (gRNAs) having a spacer sequence with a length that allows epigenetic editing and not gene editing of a first gene in the cell has a spacer sequence of:
(a) less than or equal to 17 nucleotides (e.g., less than or equal to 16 nucleotides), such as less than or equal to 15, less than or equal to 14, less than or equal to 13, less than or equal to 12 nucleotides, or equal to 11 nucleotides; or
(b) 11 to 17 nucleotides (e.g., 11 to 16 nucleotides), such as 12 to 17 (e.g., 12 or 16), 13 to 17 (e.g., 13 to 16), 14 to 17 (e.g., 14 to 16), 15 to 17 (e.g., 16), or 17 nucleotides.

In some embodiments, the one or more gRNAs having a spacer sequence with a length that allows gene editing of a second gene in the cell has a spacer sequence of:
(a) 17 or more nucleotides (e.g., 18 or more nucleotides), such as 19 or more, or 20 or more nucleotides; or
(b) 17 to 30 nucleotides, such as 18 to 30, 19 to 30, 20 to 30, 18 to 25, 19 to 25, 20 to 25, 18 to 20, or 19 to 20 nucleotides, optionally 18 to 25 nucleotides (e.g., 18 to 21 nucleotides).

In some embodiments, the at least one target gene is selected from: genes without CpG Islands (CGI), such as: TRAC; TRBC; PDCD1; TIM-3; TIGIT; LAG3; CTLA4; AAVS1 and CCR5; and/or genes having CGI, such as: B2M; TET2; TGFBR2; A2AR; CISH; PTPN11; PTPN6; PTPA; PTPN2; JUNB; TOX; TOX2; NR4A1; NR4A2; NR4A3; MAP4K1; REL; IRF4; DGKA; PIK3CD; HLA-A; USP16; DCK; and FAS. For example, the target genes may comprise one or more of B2M, TRAC, TET2, and TGFBR2. In some embodiments, the target genes may comprise, e.g., B2M and TRAC. In some embodiments, the target genes may comprise, e.g., B2M, TRAC, TET2, and TGFBR2. In some embodiments, the target genes may comprise a combination of B2M, TET2, and TRAC; a combination of B2M, TET2, and TGFBR2; a combination of B2M, TGFBR2 and TRAC; or a combination of TET2, TGFBR2, and TRAC.

In some embodiments, the first gene is selected from B2M, TET2, TGFBR2, A2AR, CISH, PTPN11, PTPN6, PTPA, PTPN2, JUNB, TOX, TOX2, NR4A1, NR4A2, NR4A3, MAP4K1, REL, IRF4, DGKA, PIK3CD, HLA-A, USP16, DCK, and FAS; and/or the second gene is selected from TRAC, TRBC, PDCD1, TIM-3, TIGIT, LAG3, CTLA4, AAVS1, and CCR5.

In some embodiments, the second gene is a TRAC gene, optionally wherein the one or more gRNAs targeting the TRAC gene comprise a spacer having the sequence of one of SEQ ID NOs: 562-611, optionally SEQ ID NO: 604.

In some embodiments, the first gene is a B2M gene, optionally wherein the one or more gRNAs targeting the B2M gene each comprise a spacer having the sequence of one of SEQ ID NOs: 28-33 and 39-44; or the sequence of one of SEQ ID NOs: 2778-2878 with a 3 to 9 nucleotide truncation at the 5′ end, optionally one of SEQ ID NOs: 2778, 2780, 2801, and 2863 with a 3 to 9 nucleotide truncation at the 5′ end, selected from SEQ ID NOs: 4486-4492, 4497-4503, 4508-4514, and 4519-4525.

In some embodiments, the first gene is a TGFBR2 gene, optionally wherein the one or more gRNAs targeting the TGFBR2 gene each comprise a spacer having the sequence of one of SEQ ID NOs: 2929-2978 and 4553-4559 with a 3 to 9 nucleotide truncation at the 5′ end.

In some embodiments, the first gene is a TET2 gene, optionally wherein the one or more gRNAs targeting the TET2 gene each comprise a spacer having the sequence of one of SEQ ID NOs: 4429-4478 and 4560-4565 with a 3 to 9 nucleotide truncation at the 5′ end.

In some embodiments, the combination is for modifying transcription, expression and/or activity of one or more (e.g. two or more) gene in a cell, wherein the cell is a mammalian cell, optionally a human cell, optionally wherein the cell is a human immune cell or human T cell.

In some embodiments, the combination, further comprises a donor DNA comprising 5′ and 3′ arms that are homologous to sequences in the second gene.

In some embodiments, the combination further comprises an agent:
i) which promotes the survival, proliferation and/or activity of a cell, such as a cell which comprises the combination or a cell which does not comprise the combination; and/or
ii) which is detrimental to the survival, proliferation, activity, chemoresistance and/or chemotaxis of a cell, such as a cell which comprises the combination or a cell which does not comprise the combination; and/or
iii) which enables selection of a cell, such as a cell which comprises the combination or a cell which does not comprise the combination. In some embodiments, the agent is a CAR or transgenic TCR. In some embodiments, the agent is FIX.

In another aspect the invention provides a combination for regulating one or more gene in a human cell, optionally an immune cell or a T cell, the combination comprising:
one or more (e.g. one to three) fusion proteins each comprising a catalytically inactive Cas9, optionally SpCas9, endonuclease domain, wherein the one or more (e.g. one to three) fusion proteins collectively comprise a transcriptional repressor and a DNMT3L domain, or polynucleotide(s) encoding the one ore more (e.g. one to three) fusion proteins, wherein the gene comprises a CpG island (CGI) and is
(i) a B2M gene and the combination further comprises two or more gRNAs each comprising a spacer having the sequence of one of SEQ ID NOs: 2778-2878 optionally with a 1 to 9 nucleotide truncation at the 5′ end, or comprises polynucleotide(s) coding for the gRNAs;
(ii) a TGFBR2 gene and the combination further comprises a gRNA that comprises a spacer having the sequence of any one of SEQ ID NOs: 2929-2978 and 4553-4559 optionally with a 1 to 9 nucleotide truncation at the 5′ end, or comprises polynucleotide(s) coding for the gRNA; or
(iii) a TET2 gene and the combination further comprises a gRNA that comprises a spacer having the sequence of any one of SEQ ID NOs: 4429-4478 and 4560-4565 optionally with a 1 to 9 nucleotide truncation at the 5′ end, or comprises polynucleotide(s) coding for the gRNA.

In some embodiments, the combination comprises at least one gRNA according to the present invention. In some embodiments, the combination comprises one or more gRNAs comprising one or more gRNA sequences shown in Table 8. In some embodiments, the present disclosure provides a combination for regulating a gene comprising one or more gRNAs comprising one or more gRNA sequences shown in Table 8.

In some embodiments, the gene comprising a CGI is a B2M gene and the gRNAs targeting it are two or three gRNAs each independently comprising a spacer having the sequence of: C8 (SEQ ID NO: 35), F4 (SEQ ID NO: 24), H8 (SEQ ID NO: 2780), H10 (SEQ ID NO: 2863), H11 (SEQ ID NO: 2778), or H12 (SEQ ID NO: 2801), optionally with a 1 to 9, optionally 3 to 9, nucleotide truncation at the 5′ end.

In some embodiments, the B2M-targeting gRNAs comprise a gRNA comprising a spacer having the sequence of F4 optionally with a 1 to 9, optionally 3 to 9, nucleotide truncation at the 5′ end, a gRNA comprising a spacer having the sequence of H8 optionally with a 1 to 9, optionally 3 to 9, nucleotide truncation at the 5′ end, and a gRNA comprising a spacer having the sequence of H10 optionally with a 1 to 9, optionally 3 to 9, nucleotide truncation at the 5′ end.

In some embodiments, the B2M-targeting gRNAs comprise a gRNA comprising a spacer having the sequence of C8 optionally with a 1 to 9, optionally 3 to 9, nucleotide truncation at the 5′ end, a gRNA comprising a spacer having the sequence of H8 optionally with a 1 to 9, optionally 3 to 9, nucleotide truncation at the 5′ end, and a gRNA comprising a spacer having the sequence of H10 optionally with a 1 to 9, optionally 3 to 9, nucleotide truncation at the 5′ end.

In some embodiments, the B2M-targeting gRNAs comprise a gRNA comprising a spacer having the sequence of F4 optionally with a 1 to 9, optionally 3 to 9, nucleotide truncation at the 5′ end, and a gRNA comprising a spacer having the sequence of H8 optionally with a 1 to 9, optionally 3 to 9, nucleotide truncation at the 5′ end.

In some embodiments, the B2M-targeting gRNAs comprise a gRNA comprising a spacer having the sequence of F4 optionally with a 1 to 9, optionally 3 to 9, nucleotide truncation at the 5′ end, and a gRNA comprising a spacer having the sequence of H10 optionally with a 1 to 9, optionally 3 to 9, nucleotide truncation at the 5′ end.

In some embodiments, the B2M-targeting gRNAs comprise a gRNA comprising a spacer having the sequence of H8 optionally with a 1 to 9, optionally 3 to 9, nucleotide truncation at the 5′ end, and a gRNA comprising a spacer having the sequence of H10 optionally with a 1 to 9, optionally 3 to 9, nucleotide truncation at the 5′ end.

In some embodiments, the gene comprising a CGI is a TGFBR2 gene and the combination comprises one or more gRNAs targeting it, or coding sequences of the one or more gRNAs, the one or more gRNAs each independently comprising a spacer having the sequence of
TG1 (SEQ ID NO: 4553),
TG2 (SEQ ID NO: 4554),
TG3 (SEQ ID NO: 4555),
TG4 (SEQ ID NO: 4556),
TG5 (SEQ ID NO: 4557),
TG6 (SEQ ID NO: 2940),
TG7 (SEQ ID NO: 2937),
TG8 (SEQ ID NO: 2930),
TG9 (SEQ ID NO: 2955),
TG10 (SEQ ID NO: 4558),
TG11 (SEQ ID NO: 2957),
TG12 (SEQ ID NO: 2929),
TG13 (SEQ ID NO: 4559),
TG14 (SEQ ID NO: 2945),
TG15 (SEQ ID NO: 2931),
TG16 (SEQ ID NO: 2942),
TG17 (SEQ ID NO: 2939),
TG18 (SEQ ID NO: 2935),
TG19 (SEQ ID NO: 2938), or
TG20 (SEQ ID NO: 2932),
   optionally with a 1 to 9, optionally 3 to 9, nucleotide truncation at the 5′ end.

In some embodiments, the TGFBR2-targeting gRNAs comprise
(i) a gRNA comprising a spacer having the sequence of TG7 optionally with a 1 to 9, optionally 3 to 9, nucleotide truncation at the 5′ end, and
   a gRNA comprising a spacer having the sequence of TG8 optionally with a 1 to 9, optionally 3 to 9, nucleotide truncation at the 5′ end; or
(ii) a gRNA comprising a spacer having the sequence of TG19 optionally with a 1 to 9, optionally 3 to 9, nucleotide truncation at the 5′ end, and
   a gRNA comprising a spacer having the sequence of TG20 optionally with a 1 to 9, optionally 3 to 9, nucleotide truncation at the 5′ end.

In some embodiments, the gene comprising a CGI is a TET2 gene and the combination comprises one or more gRNAs targeting it, or coding sequences of the one or more gRNAs, the one or more gRNAs each independently comprising a spacer having the sequence of
TE1 (SEQ ID NO: 4560),
TE2 (SEQ ID NO: 4561),
TE3 (SEQ ID NO: 4562),
TE4 (SEQ ID NO: 4563),
TE5 (SEQ ID NO: 4443),
TE6 (SEQ ID NO: 4434),
TE7 (SEQ ID NO: 4466),
TE8 (SEQ ID NO: 4438),
TE9 (SEQ ID NO: 4429),
TE10 (SEQ ID NO: 4469),
TE11 (SEQ ID NO: 4564),
TE12 (SEQ ID NO: 4449),
TE13 (SEQ ID NO: 4433),
TE14 (SEQ ID NO: 4442),
TE15 (SEQ ID NO: 4430),
TE16 (SEQ ID NO: 4431),
TE17 (SEQ ID NO: 4474),
TE18 (SEQ ID NO: 4432),
TE19 (SEQ ID NO: 4565), or
TE20 (SEQ ID NO: 4478),
   optionally with a 1 to 9, optionally 3 to 9, nucleotide truncation at the 5′ end.

In some embodiments, the TET2-targeting gRNAs comprise
(i) a gRNA comprising a spacer having the sequence of TE13 optionally with a 1 to 9, optionally 3 to 9, nucleotide truncation at the 5′ end, and
   a gRNA comprising a spacer having the sequence of TE14 optionally with a 1 to 9, optionally 3 to 9, nucleotide truncation at the 5′ end; or
(ii) a gRNA comprising a spacer having the sequence of TE19 optionally with a 1 to 9, optionally 3 to 9, nucleotide truncation at the 5′ end, and
   a gRNA comprising a spacer having the sequence of TE20 optionally with a 1 to 9, optionally 3 to 9, nucleotide truncation at the 5′ end.

In some embodiments, the ETM(s) (e.g., one or more fusion proteins) collectively further comprise a DNMT1, DNMT3A, DNMT3B, or SETDB1 domain, optionally DNMT3A.

In some embodiments, the combination comprises: (i) a first fusion protein comprising a transcriptional repressor domain and a Cas endonuclease domain, and a second fusion protein comprising a DNMT3L domain and a Cas endonuclease domain, or (ii) a fusion protein comprising, optionally from N-terminus to C-terminus, a transcriptional repressor domain, a Cas endonuclease domain, and a DNMT3L domain.

In some embodiments, the combination comprises (i) a first fusion protein comprising a transcriptional repressor domain and a Cas endonuclease domain, a second fusion protein comprising a DNMT3L domain and a Cas endonuclease domain, and a third fusion protein comprising a DNMT3A domain and a Cas endonuclease domain, or (ii) a fusion protein comprising a transcriptional repressor domain, a Cas endonuclease domain, a DNMT3L domain, and a DNMT3A domain.

In some embodiments, the epigenetic effector domain (e.g. transcriptional repressor domain) is a Kruppel-associated box (KRAB) domain, optionally derived from human Kox1 or ZIM3.

In some embodiments, the combination comprises a fusion protein comprising, optionally from N terminus to C terminus, a KRAB domain derived from ZIM3, a catalytically active Cas9 domain, and a DNMT3L domain, optionally comprising an amino acid sequence of SEQ ID NO: 4482.

In some embodiments, the combination further comprises gRNAs for targeting one or more additional genes in the cell, optionally wherein the combination comprises gRNAs targeting the following genes, or comprises polynucleotides coding for the gRNAs: (i) B2M and TRAC, (ii) B2M, TRAC, and TGFBR2, (iii) B2M, TRAC, and TET2, (iv) B2M, TGFBR2, and TET2, or (v) B2M, TGFBR2, TET2, and TRAC.

In some embodiments, the gRNA(s) are chemically modified, optionally wherein the chemically modified gRNA(s) comprise phosphorothioate internucleoside linkages at the 5′ and/or 3′ ends, and/or 2′-O-methyl nucleotides.

In a further aspect, the present invention provides a polynucleotide encoding at least one ETM (e.g., ETR) according to the present invention.

In another aspect, the present invention provides a nucleic acid construct comprising a nucleic acid sequence encoding at least one ETM (e.g., ETR) according to the present invention.

In some embodiments, the nucleic acid construct further comprises a nucleic acid sequence:
i) which promotes the survival, proliferation and/or activity of a cell, such as a cell which expresses said nucleic acid construct or a cell which does not express said nucleic acid construct; and/or
ii) which is detrimental to the survival, proliferation, activity, chemoresistance and/or chemotaxis of a cell, such as a cell which expresses said nucleic acid construct or a cell which does not express said nucleic acid construct; and/or
iii) which enables selection of a cell, such as a cell which comprises the nucleic acid construct or a cell which does not comprise the construct.

In one aspect, the present invention provides a vector comprising a polynucleotide according to the present invention or a nucleic acid construct according to the present invention.

In another aspect, the present invention provides a kit of polynucleotides comprising:
a) at least one polynucleotide encoding at least one ETM (e.g., ETR) according to the present invention; and
b) a polynucleotide providing at least one gRNA disclosed herein; and optionally,
c) a further polynucleotide comprising a nucleic acid sequence which encodes an agent:
   i) which promotes the survival, proliferation and/or activity of a cell, such as a cell which comprises the polynucleotides or a cell which does not comprise the polynucleotides; and/or
   ii) which is detrimental to the survival, proliferation, activity, chemoresistance and/or chemotaxis of a cell, such as a cell which comprises said polynucleotides or a cell which does not comprise said polynucleotides; and/or
   iii) which enables selection of a cell, such as a cell which comprises the polynucleotides or a cell which does not comprise the polynucleotides.

In another aspect, the present invention provides a cell (such as an engineered cell) comprising an ETM (e.g., ETR) according to the present invention, at least one gRNA according to the present invention, a combination according to the present invention, a polynucleotide according to the present invention, a nucleic acid construct according to the present invention, a vector according to the present invention or a kit of polynucleotides according to the present invention. In another aspect, the invention provides a progeny of the cell.

In another aspect, the invention provides a cell obtained by the use or method of the invention, or a progeny thereof.

In some embodiments, the cell is a human T cell, optionally engineered to express a recombinant antigen receptor, optionally selected from a recombinant T cell receptor (TCR) or a chimeric antigen receptor (CAR).

In a further aspect, the present invention provides a composition comprising an ETM (e.g., ETR) according to the present invention, at least one gRNA according to the present invention, a combination according to the present invention, a polynucleotide according to the present invention, a nucleic acid construct according to the present invention, a vector according to the present invention, a kit of polynucleotides according to the present invention or a cell according to the present invention.

In another aspect, the present invention provides a pharmaceutical composition comprising an ETM (e.g., ETR) according to the present invention, at least one gRNA according to the present invention, a combination according to the present invention, a polynucleotide according to the present invention, a nucleic acid construct according to the present invention, a vector according to the present invention, a kit of polynucleotides according to the present invention or a cell according to the present invention.

In a further aspect, the present invention provides the use of an ETM (e.g., ETR) according to the present invention, at least one gRNA according to the present invention, a combination according to the present invention, a polynucleotide according to the present invention, a nucleic acid construct according to the present invention, a vector according to the present invention, a kit of polynucleotides according to the present invention or a cell according to the present invention for modifying the transcription, expression and/or activity at least one target gene. The use may, for example, be in vitro or ex vivo use.

In another aspect, the present invention provides a method of modifying the transcription, expression and/or activity of at least one target gene in a cell comprising the step of administering an ETM (e.g., ETR) according to the present invention, at least one gRNA according to the present invention, a combination according to the present invention, a polynucleotide according to the present invention, a nucleic acid construct according to the present invention, a vector according to the present invention or a kit of polynucleotides according to the present invention to a cell. The cell may be, for example, a T cell.

In some embodiments, the modifying the transcription, expression and/or activity is repressing transcription, expression and/or activity, e.g., silencing.

In some embodiments, the method comprises repressing the transcription and/or expression of at least two different target genes in a cell.

In some embodiments, the method comprises silencing at least two different target genes in a cell.

Suitably, transcription and/or expression of at least one of the at least two target genes may be epigenetically repressed (e.g., silenced) and at least one of the at least two target genes may be repressed (e.g., silenced) by gene editing, wherein at least one ETM (e.g., ETR) and at least two gRNAs are administered to said cell simultaneously, sequentially, or separately.

In one aspect, an ETM (e.g., ETR) according to the present invention, at least one gRNA according to the present invention, a combination according to the present invention, a polynucleotide according to the present invention, a nucleic acid construct according to the present invention, a vector according to the present invention, a kit of polynucleotides according to the present invention, a cell according to the present invention or a pharmaceutical composition according to the present invention may be for use in therapy.

In another aspect the invention provides use of an ETM (e.g., ETR) according to the present invention, at least one gRNA according to the present invention, a combination according to the present invention, a polynucleotide according to the present invention, a nucleic acid construct according to the present invention, a vector according to the present invention, a kit of polynucleotides according to the present invention, a cell according to the present invention or a pharmaceutical composition according to the present invention in the manufacture of medicament for treating a human in need thereof.

Suitably, at least one ETM (e.g., ETR) and at least two gRNAs may be administered to a subject simultaneously, sequentially, or separately.

In another aspect, the present invention provides a method for treating and/or preventing a disease, which comprises the step of administering an ETM (e.g., ETR) according to the present invention, at least one gRNA according to the present invention, a combination according to the present invention, a polynucleotide according to the present invention, a nucleic acid construct according to the present invention, a vector according to the present invention, a kit of polynucleotides according to the present invention, a cell according to the present invention or a pharmaceutical composition according to the present invention to a subject in need thereof.

Suitably, at least one ETM (e.g., ETR) and at least two gRNAs may be administered to a subject simultaneously, sequentially, or separately.

In one aspect, the present invention provides a method of gene therapy which comprises the steps:
(i) isolation of a cell containing sample;
(ii) introduction of an ETM (e.g. ETR) according to the present invention, at least one gRNA according to the present invention, a polynucleotide according the present invention, a nucleic acid construct according to the present invention, a vector according to the present invention or a kit of polynucleotides according to the present invention to the cell(s); and
(iii) administering the cell(s) from step (ii) to a subject.

The polynucleotide, nucleic acid construct or vector may, for example, be introduced by transduction or transfection.

In some embodiments, the cell is autologous. In some embodiments, the cell is allogeneic.

It is understood that an ETM (e.g., ETR) according to the present invention, at least one gRNA according to the present invention, a combination according to the present invention, a polynucleotide according to the present invention, a nucleic acid construct according to the present invention, a vector according to the present invention, a kit of polynucleotides according to the present invention, a cell according to the present invention or a pharmaceutical composition according to the present invention may be used in a method of treatment described herein, may be for use in a treatment described herein, or may be used in the manufacture of a medicament for a treatment described herein.

Other features, objects, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### DESCRIPTION OF THE DRAWINGS

FIG. 1 shows (A) the sequence (SEQ ID NOs: 21 and 22 for the sense and antisense strands, respectively) within the B2M gene which may be targeted by Cas9 or dCas9-ETRs and which is targeted in the Examples herein (the protospacer adjacent motif (PAM) sequence is underlined), and (B) the sequences of spacers which may be used in gRNAs to target B2M and which are used in the Examples herein (SEQ ID NOs: 23-34, in order of appearance).

FIG. 2 shows a histogram illustrating the percentage of mutated B2M alleles in cells transfected with Cas9 and the indicated gRNAs. Data are represented as % of non-homologous end-joining (NHEJ) at B2M (n=3; mean±s.d.). UT: untreated.

FIG. 3 shows a histogram illustrating the percentage of tdTomato-negative cells 44 days upon transfection with the triple combination of dCas9-based ETRs and the indicated gRNAs (n=3; mean±s.d.).

FIG. 4 shows histograms illustrating the percentage of tdTomato-negative cells 20 days upon transfection with the triple combination of dCas9-based ETRs (left panel) or Cas9 (right panel) and the indicated gRNAs H8, C8, and H10, which were either full length or truncated as indicated. The full-length sequences of H8, C8, and H10 are SEQ ID NOs: 2780, 35, and 2863, respectively. The truncated versions (19, 18, 17, 16, 15, 14, 13, 12, 11, or 10 nucleotide versions) are truncated at the 5′ end of the full-length sequence by 1, 2, 3, 4, 5, 6, 7, 8, 9, or 10 nucleotides, respectively.

FIG. 5 shows a histogram illustrating the percentage of mutated TRAC alleles in cells transfected with Cas9 and the TRAC gRNA. Data are represented as % of NHEJ at TRAC (n=3; mean±s.d.).

FIG. 6 shows a histogram illustrating the percentage of B2M-negative cells (B2M⁻ cells) 25 days after transfection with the indicated Cas9 constructs (i.e., Cas9, dCas9-ETRs, Cas9-ETRs (namely ETM)) and gRNA combinations (n=3; mean±s.d.).

FIG. 7 shows representative flow cytometry dot plots analyses of the cells treated with the 16 nt B2M gRNA and either Cas9-ETRs (namely ETM) or dCas9-ETRs. Analysis was performed at day 25 post-treatment.

FIG. 8 shows time-course flow cytometric analysis of cells treated as indicated. Data are shown as % of B2M-negative cells normalized to Untreated (UT) cells. Analysis was performed at day 25 post-treatment (n=3; mean±s.d.).

FIG. 9 shows a histogram illustrating the percentage of gene editing at the B2M or TRAC gene for the indicated treatment conditions (n=3; mean±s.d.).

FIG. 10 shows polymerase chain reaction (PCR) analysis of the indicated treatment conditions for reciprocal chromosomal translocations between the B2M and the TRAC locus. Top: it shows a schematic diagram of the PCR strategy indicating the primers used (arrows) for the analysis. Bottom: it shows a picture of the agarose-stained gel loaded with the PCR products from the indicated treatment conditions (each in triplicate). Translocations were detected only in samples treated with Cas9 or Cas9-ETR (namely ETM) in combination with the 20 nt B2M gRNA. MW: molecular weight.

FIG. 11 is a diagram of the B2M gene showing the CpG island (CGI) and the distribution of gRNAs H8 (SEQ ID NO: 2780), C8 (SEQ ID NO: 35), F4 (SEQ ID NO: 2878), H10 (SEQ ID NO: 2863), H11 (SEQ ID NO: 2778), and H12 (SEQ ID NO: 2801).

FIG. 12 shows the percentage of B2M silencing by the triple combination of dCas9-based ETRs at days 12 and 25 post-treatment with the indicated gRNAs, either alone (first row of each table) or in combinations (second and third row of each table). Data are shown as heatmap.

FIG. 13 shows representative flow cytometry analyses of T cells treated with the indicated gRNA combinations (namely C8+F4, C8+H8 or H8+F4) and the triple combination of dCas9-based ETRs at days 12 and 25 post-treatment. The fold increase in terms of efficiency of B2M epi-silencing between the C8+F4 and H8+F4 conditions is indicated.

FIG. 14 shows a time-course flow cytometry analysis of T cells treated with the triple combination of dCas9-based ETRs and the indicated gRNAs combinations. Data are shown as % of B2M-negative cells. UT: untreated T cells. Vertical dashed red lines indicate the days at which T cells were restimulated.

FIG. 15 shows a histogram illustrating the fold change in the percentage of B2M negative T cells between day 25 and 12 post-treatment, calculated based on the data shown in FIG. 14. Data are represented as fold decrease in B2M negative cells.

FIG. 16 shows a time-course flow cytometry analysis of T cells treated with the triple combination of dCas9-based ETRs and the indicated gRNAs combinations. Data are shown as % of B2M-negative cells. UT: untreated T cells. Vertical dashed red lines indicate the days at which T cells were restimulated.

FIG. 17 shows a histogram illustrating the fold change in the percentage of B2M negative T cells between day 25 and 12 post-treatment, calculated based on the data shown in FIG. 16. Data are represented as fold decrease in B2M negative cells.

FIG. 18A shows a time-course flow cytometry analysis of T cells treated with the indicated ETR combinations and the gRNA combination C8+F4. Data are shown as % of B2M-negative cells. UT: untreated T cells. K+3A+3L: standard triple ETR combination; K: KRAB-based ETR alone: 3A+3L: double ETR combination containing DNMT3A and DNMT3L; K+3A: double ETR combination containing KRAB and DNMT3A; K+3L: double ETR combination containing KRAB and DNMT3L; triple Vertical dashed red lines indicate the days at which T cells were restimulated.

FIG. 18B shows representative flow cytometry analyses of T cells from FIG. 18A and treated with the indicated ETR combinations and the gRNA combination C8+F4. K+3A+3L: standard triple ETR combination; K: KRAB-based ETR alone: 3A+3L: double ETR combination containing DNMT3A and DNMT3L; K+3A: double ETR combination containing KRAB and DNMT3A; K+3L: double ETR combination containing KRAB and DNMT3L.

FIG. 19 shows a time-course flow cytometry analysis of T cells treated with the double ETR combination containing KRAB and DNMT3L, plus the indicated gRNAs combinations. Data are shown as % of B2M-negative cells. UT: untreated T cells. Vertical dashed red lines indicate the days at which T cells were restimulated.

FIG. 20A shows on the left a schematic of the ZIM3:dCas9:3L fusion ETR and on the right a time-course flow cytometry analysis of T cells co-treated with ether the double ETR combination containing DNMT3A and DNMT3L or ZIM3:dCas9:3L, plus the indicated gRNAs combinations. Data are shown as % of B2M-negative cells. UT: untreated T cells. Vertical dashed red lines indicate the days at which T cells were restimulated.

FIG. 20B shows representative flow cytometry analyses of T cells from FIG. 20A and treated with the indicated ETRs and gRNA combinations. Indicated is also the fold change increase in the efficiency of epi-silencing between sample treated with the double ETR combination and the ETR fusion.

FIG. 21 shows representative flow cytometry analyses of T cells treated with decreasing doses (in micrograms) of the mRNA encoding for ZIM3:dCas9:3L fusion ETR and the indicated gRNA combination.

FIG. 22 shows representative flow cytometry analyses of T cells treated or not with Cas9, a gRNA against TRAC (see FIG. 5) and transduced with an AAV6 for targeted integration into TRAC of the NY-ESO engineered TCR. Upper left quadrant shows wild-type, un-edited cells. Bottom left quadrant shows cells with genetically disrupted TCR. Upper right quadrant shows T cells with targeted integration of the NY-ESO TCR.

FIG. 23 shows on the left a schematic representation of the double ETM combination containing the catalytically active Cas9 and the KRAB and DNMT3L effectors, while, on the right, it shows representative flow cytometry analyses of T cells treated with these ETMs and the indicated truncated gRNA against B2M, plus the full-length gRNA against TRAC and the AAV6 for targeted integration of the NY-ESO TCR into TRAC. The flow cytometry dot plot on the left reports the expression levels of B2M. The flow cytometry dot plot on the middle reports the expression levels of the endogenous TCR and the targeted NY-ESO. The flow cytometry dot plot on the right reports the expression level of NY-ESO and B2M. SSCH: side scatter height.

FIG. 24 shows on the left a schematic representation of the ETM containing the catalytically active Cas9 and the ZIM3 and DNMT3L effectors (namely ZIM3:Cas9:3L), while, on the right, it shows representative flow cytometry analyses of T cells treated with this ETM and the indicated truncated gRNA against B2M, plus the full-length gRNA against TRAC and the AAV6 for targeted integration of the NY-ESO TCR into TRAC. The flow cytometry dot plot on the top left reports the expression levels of B2M. The flow cytometry dot plot on the top middle reports the expression levels of the endogenous TCR and the targeted NY-ESO. The flow cytometry dot plot on the bottom reports the expression level of NY-ESO and B2M. The flow cytometry dot plot on the top right shows, within the NY-ESO positive cells, the expression levels of B2M. The flow cytometry dot plot on the bottom right shows, within the endogenous TCR negative cells, the expression levels of B2M.

FIG. 25 shows a polymerase chain reaction (PCR) analysis of the indicated treatment conditions for reciprocal chromosomal translocations between the B2M and TRAC. Top: it shows a schematic diagram of the PCR strategy indicating the primers used (arrows) for the analysis. Bottom: it shows a picture of the agarose-stained gel loaded with the PCR products from the indicated treatment conditions. Expected position of the B2M-TRAC translocation band is shown by the asterisks. MW: molecular weight. Translocations were detected only in samples treated with the ETM in combination with the 20 nt gRNAs for B2M and TRAC.

FIG. 26 shows schematics of the TGFBR2 (top) and TET2 (bottom) genes, in which are indicated the relative positions of each gRNA and their pairing (P). The CpG Island (CGI) of each gene are also indicated.

FIG. 27 shows the percentages of TGFBR2 epi-silencing for the indicated combinations of gRNA pairs. Percentages are reported in the boxes. Unlabeled boxes indicate combinations that were already present in the matrix. np: not performed.

FIG. 28 shows the percentages of TET epi-silencing for the indicated combinations of gRNA pairs. Percentages are reported in the boxes. Unlabeled boxes indicate combinations that were already present in the matrix. Negative data indicate upregulation of TET2. np: not performed.

FIG. 29 shows histograms illustrating the percentages of epi-silencing of TGFBR2 (left) and TET2 (right) in T cells treated with the triple ETR combination and the indicated pairs (P) of gRNAs, either alone or in combination. The pairs used in these studies correspond to those described in FIGS. 27 and 28.

FIG. 30 shows a histogram illustrating the percentage of epigenetic silencing of the indicated genes as measured by ddPCR.

FIG. 31 shows on the left representative flow cytometry analyses for B2M (left plot) and TRAC (right plot) expression by T cells treated as indicated and on the right a histogram illustrating the percentage of epigenetic silencing of TGFBR2.

FIG. 32 shows on the left representative flow cytometry analyses for B2M (left plot) and TRAC (right plot) expression by T cells treated as indicated and on the right a histogram illustrating the percentage of epigenetic silencing of TGFBR2.

FIG. 33 shows polymerase chain reaction (PCR) analyses of the indicated treatment conditions for reciprocal chromosomal translocations among B2M, TGFBR2 and TRAC. Top: a schematic diagram of the PCR strategy for two hypothetical genes (X and Y), where arrows indicate the primers used for analysis. Bottom: pictures of the agarose-stained gels loaded with the PCR products from the indicated treatment conditions. Expected positions of translocations bands are indicated by the asterisks. MW: molecular weight. Translocations were detected only in samples treated with the ETM in combination with the 20 nt gRNAs for B2M, TGFBR2 and TRAC.

FIG. 34 shows on the left representative flow cytometry analyses for B2M (left plot) and TRAC (right plot) expression by T cells treated as indicated and on the right a histogram illustrating the percentage of epigenetic silencing of TET2.

FIG. 35 shows on the left representative flow cytometry analyses for B2M (left plot) and TRAC (right plot) expression by T cells treated as indicated and on the right a histogram illustrating the percentage of epigenetic silencing of TET2.

FIG. 36 shows polymerase chain reaction (PCR) analyses of the indicated treatment conditions for reciprocal chromosomal translocations among B2M, TET2 and TRAC. Top: a schematic diagram of the PCR strategy for two hypothetical genes (X and Y), where arrows indicate the primers used for analysis. Bottom: pictures of the agarose-stained gels loaded with the PCR products from the indicated treatment conditions. Expected positions of translocations bands are indicated by the asterisks. MW: molecular weight. Translocations were detected only in samples treated with the ETM in combination with the 20 nt gRNAs for B2M, TET2 and TRAC.

FIG. 37 shows on the left representative flow cytometry analyses for B2M (left plot) and TRAC (right plot) expression by T cells treated as indicated and on the right a histogram illustrating the percentage of epigenetic silencing of TGFBR2 and TET2.

FIG. 38 shows on the left representative flow cytometry analyses for B2M (left plot) and TRAC (right plot) expression by T cells treated as indicated and on the right a histogram illustrating the percentage of epigenetic silencing of TGFBR2 and TET2.

FIG. 39 shows polymerase chain reaction (PCR) analyses of the indicated treatment conditions for reciprocal chromosomal translocations among B2M, TGFBR2, TET2 and TRAC. Top: a schematic diagram of the PCR strategy for two hypothetical genes (X and Y), where arrows indicate the primers used for analysis. Bottom: pictures of the agarose-stained gels loaded with the PCR products from the indicated treatment conditions. Expected positions of translocations bands are indicated by the asterisks. MW: molecular weight. Translocations were detected only in samples treated with the ETM in combination with the 20 nt gRNAs for B2M, TGFBR2, TET2 and TRAC.

### DETAILED DESCRIPTION OF THE INVENTION

### Engineered Transcriptional Modulator (ETM)

In one aspect, the present invention provides an engineered transcriptional modulator (ETM), for example an engineered transcriptional repressor (ETR), comprising: a) at least one epigenetic effector domain; operably linked to b) an endonuclease.

The ETMs of the invention may be ETRs. ETRs may repress transcription and/or expression of target gene(s).

The ETMs (e.g., ETRs) of the invention are agents that may enable multiplexing of gene editing and epigenetic editing of different target genes. For example, the ETMs (e.g., ETRs) according to the present invention may enable repression of transcription and/or expression (e.g., silencing) of multiple different target genes, wherein one gene is repressed (e.g., silenced) by genetic editing and at least one gene is repressed (e.g., silenced) by epigenetic repression (e.g., silencing). An advantage of this poly-functional editing system is that there is no reciprocal translocation between the simultaneously edited genes, thus greatly improving the safety of multiplex gene editing. Furthermore, application of such a poly-functional editing approach allows performance of orthogonal edits in one step, without the need for sequential engineering procedures, thus greatly facilitating product manufacturing and reducing associated costs and cell toxicity. The target gene selected for gene editing also may be used as a target site for insertion of exogenous expression cassettes.

The ETMs may be referred to as programmable multi-editors (ProMEs). For example, the design of gRNAs may allow an ETM to be programmed to modify transcription, expression and/or activity of multiple targets in the same cell. The ETMs (e.g., ETRs) may be chimeric or fusion proteins that are comprised of at least one (such as one) endonuclease operably linked to at least one effector domain (e.g., a KRAB domain, a SETDB1 domain, a DNMT3A, DNMT3B or DNMT1 domain or a DNMT3L domain, or homologues thereof; wherein the domains may be full-length proteins or functional fragments thereof and may be referred to herein as “KRAB,” “SETDB1,” “DNMT3A,” “DNMT3B,” “DNMT1,” or “DNMT3L,” respectively). The endonuclease may enable cleavage of specific DNA sequence(s), and may be chosen or engineered to bind to nucleic acid sequence(s) of choice. The epigenetic effector domain may harbour a catalytic activity which enables modification (such as repression) of transcription of a target gene. Alternatively, or additionally, the effector domain may recruit additional agents within a cell to a target gene, which results in the modification (such as repression) of transcription of the target gene. The present invention also envisages ETMs that are engineered transcription activators (ETAs). ETAs may increase transcription and/or expression of target gene(s).

By “operably linked”, it is to be understood that the individual components are linked together in a manner which enables them to carry out their function (e.g., cleavage of DNA, binding to DNA, catalysing a reaction or recruiting additional agents from within a cell) substantially unhindered. For example, an endonuclease may be conjugated to an epigenetic effector domain, for example to form a fusion protein. Methods for conjugating polypeptides are known in the art, for example through the provision of a linker amino acid sequence connecting the polypeptides (e.g., a linker comprising glycine and/or serine residues). Alternative methods of conjugating polypeptides known in the art include chemical and light-induced conjugation methods (e.g., using chemical cross-linking agents). In an example, the endonuclease and epigenetic effector domain (e.g., KRAB domain, DNMT3A, DNMT3B or DNMT1 domain or DNMT3L domain, or homologue thereof) of the ETM form a fusion protein.

In one aspect, the ETM (e.g., ETR) comprises an RNA binding domain. The RNA binding domain may bind to a gRNA which is complementary to a genomic target site. Thus, the RNA binding domain may direct the ETM (e.g., ETR) to a target gene.

In one aspect, the ETM (e.g., ETR) is a fusion protein comprising a) at least one epigenetic effector domain; and b) an endonuclease.

In some aspects, the ETM (e.g., ETR) is a bi-partite fusion protein. For example, the ETM (e.g., ETR) may comprise two effector domains fused to the same endonuclease.

In some aspects, the ETM (e.g., ETR) is a tri-partite fusion. For example, the ETM (e.g., ETR) may comprise three effector domains fused to the same endonuclease.

In some aspects, the ETM (e.g., ETR) may comprise four or five or six or more effector domains fused to the same endonuclease.

Suitably, where the ETM (e.g., ETR) comprises multiple effector domains, the effector domains may be different. Suitably, where the ETM (e.g., ETR) comprises multiple effector domains, the effector domains may be the same.

In one aspect, an ETM (e.g., ETR) according to the present invention comprises or consists of a Cas9-KRAB, Cas9-DNMT3A or Cas9-DNMT3L fusion protein.

Suitably, an ETM (e.g., ETR) according to the present invention may be a fusion protein comprising or consisting of endonuclease, KRAB and DNMT3A domains. Suitably, an ETM (e.g., ETR) according to the present invention may be a fusion protein comprising or consisting of endonuclease, DNMT3L and DNMT3A domains. Suitably, an ETM (e.g., ETR) according to the present invention may be a fusion protein comprising or consisting of endonuclease, DNMT3L and KRAB domains. Suitably, an ETM (e.g., ETR) according to the present invention may be a fusion protein comprising or consisting of endonuclease, DNMT3L, KRAB and DNMT3A domains.

Suitably, an ETM (e.g., ETR) according to the present invention may be a fusion protein comprising or consisting of Cas (e.g., Cas9), KRAB, and DNMT3A domains. Suitably, an ETM (e.g., ETR) according to the present invention may be a fusion protein comprising or consisting of Cas (e.g., Cas9), DNMT3L and DNMT3A domains. Suitably, an ETM (e.g., ETR) according to the present invention may be a fusion protein comprising or consisting of Cas (e.g., Cas9), DNMT3L and KRAB domains. Suitably, an ETM (e.g., ETR) according to the present invention may be a fusion protein comprising or consisting of Cas (e.g., Cas9), DNMT3L, KRAB and DNMT3A domains.

In one aspect, the ETM (e.g., ETR) comprises or consists of an endonuclease-KRAB fusion protein such as a Cas-KRAB, e.g., Cas9-KRAB domain fusion protein.

An exemplary sequence of an ETM according to the present invention comprising a KRAB domain (ETM-KRAB) is set forth below in SEQ ID NO: 18:

(SEQ ID NO: 18)

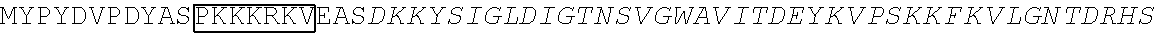

IKKNLIGALLFDSGETAEATRLKRTARRRYTRRKNRICYLQEIFSNEMAKVDDSFFHRLEES
FLVEEDKKHERHPIFGNIVDEVAYHEKYPTIYHLRKKLVDSTDKADLRLIYLALAHMIKFRG
HFLIEGDLNPDNSDVDKLFIQLVQTYNQLFEENPINASGVDAKAILSARLSKSRRLENLIAQ
LPGEKKNGLFGNLIALSLGLTPNFKSNEDLAEDAKLQLSKDTYDDDLDNLLAQIGDQYADLF
LAAKNLSDAILLSDILRVNTEITKAPLSASMIKRYDEHHODLTLLKALVROOLPEKYKEIFF
DQSKNGYAGYIDGGASQEEFYKFIKPILEKMDGTEELLVKLNREDLLRKORTFDNGSIPHQI
HLGELHAILRRQEDFYPFLKDNREKIEKILTFRIPYYVGPLARGNSRFAWMTRKSEETITPW
NFEEVVDKGASAQSFIERMTNFDKNLPNEKVLPKHSLLYEYFTVYNELTKVKYVTEGMRKPA
FLSGEQKKAIVDLLFKTNRKVTVKQLKEDYFKKIECFDSVEISGVEDRFNASLGTYHDLLKI
IKDKDFLDNEENEDILEDIVLTLTLFEDREMIEERLKTYAHLEDDKVMKOLKRRRYTGWGRL
SRKLINGIRDKQSGKTILDFLKSDGFANRNEMQLIHDDSLTFKEDIQKAQVSGQGDSLHEHI
ANLAGSPAIKKGILQTVKVVDELVKVMGRHKPENIVIEMARENQTTQKGQKNSRERMKRIEE
GIKELGSQILKEHPVENTQLQNEKLYLYYLQNGRDMYVDQELDINRLSDYDVDHIVPQSFLK
DDSIDNKVLTRSDKNRGKSDNVPSEEVVKKMKNYWRQLLNAKLITQRKFDNLTKAERGGLSE
LDKAGFIKRQLVETRQITKHVAQILDSRMNTKYDENDKLIREVKVITLKSKLVSDFRKDFQF
YKVREINNYHHAHDAYLNAVVGTALIKKYPKLESEFVYGDYKVYDVRKMIAKSEQEIGKATA
KYFFYSNIMNFFKTEITLANGEIRKRPLIETNGETGEIVWDKGRDFATVRKVLSMPQVNIVK
KTEVQTGGFSKESILPKRNSDKLIARKKDWDPKKYGGFDSPTVAYSVLVVAKVEKGKSKKLK
SVKELLGITIMERSSFEKNPIDFLEAKGYKEVKKDLIIKLPKYSLFELENGRKRMLASAGEL
QKGNELALPSKYVNFLYLASHYEKLKGSPEDNEQKQLFVEQHKHYLDEIIEQISEFSKRVIL
ADANLDKVLSAYNKHRDKPIREQAENIIHLFTLTNLGAPAAFKYFDTTIDRKRYTSTKEVLD

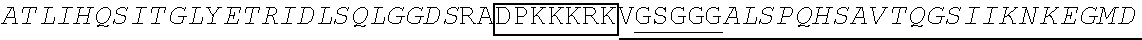

AKSLTAWSRTLVTFKDVFVDFTREEWKLLDTAQQIVYRNVMLENYKNLVSLGYQLTKPDVIL
RLEKGEEPWLVEREIHQETHPDSETAFEIKSSV

In the above sequence, the Cas9 domain is shown in italics, a haemagglutinin (HA) tag is shown in bold, a linker domain is shown in bold and double-underlined, and the KRAB domain is in italics and underlined. Nuclear localization signal (NLS) sequences are boxed.

It will be appreciated that alternatives to the HA tag and glycine-serine linker shown in these exemplary ETMs may be used in ETMs according to the present invention, or they may be absent.

In one aspect, the ETM (e.g., ETR) comprises or consists of an endonuclease-DNMT3A fusion protein such as a Cas-DNMT3A, e.g., a Cas9-DNMT3A domain fusion protein.

An exemplary sequence of an ETM according to the present invention comprising a DNMT3A domain (ETM-D3A) is set forth below in SEQ ID NO: 19:

(SEQ ID NO: 19)

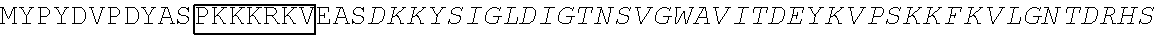

IKKNLIGALLFDSGETAEATRLKRTARRRYTRRKNRICYLQEIFSNEMAKVDDSFFHRLEES
FLVEEDKKHERHPIFGNIVDEVAYHEKYPTIYHLRKKLVDSTDKADLRLIYLALAHMIKFRG
HFLIEGDLNPDNSDVDKLFIOLVQTYNOLFEENPINASGVDAKAILSARLSKSRRLENLIAQ
LPGEKKNGLFGNLIALSLGLTPNFKSNFDLAEDAKLQLSKDTYDDDLDNLLAQIGDQYADLF
LAAKNLSDAILLSDILRVNTEITKAPLSASMIKRYDEHHQDLTLLKALVROQLPEKYKEIFF
DQSKNGYAGYIDGGASQEEFYKFIKPILEKMDGTEELLVKLNREDLLRKQRTFDNGSIPHQI
HLGELHAILRRQEDFYPFLKDNREKIEKILTFRIPYYVGPLARGNSRFAWMTRKSEETITPW
NFEEVVDKGASAQSFIERMTNFDKNLPNEKVLPKHSLLYEYFTVYNELTKVKYVTEGMRKPA
FLSGEQKKAIVDLLFKTNRKVTVKQLKEDYFKKIECFDSVEISGVEDRENASLGTYHDLLKI
IKDKDFLDNEENEDILEDIVLTLTLFEDREMIEERLKTYAHLFDDKVMKOLKRRRYTGWGRL
SRKLINGIRDKQSGKTILDFLKSDGFANRNFMQLIHDDSLTFKEDIQKAQVSGQGDSLHEHI
ANLAGSPAIKKGILQTVKVVDELVKVMGRHKPENIVIEMARENOTTOKGQKNSRERMKRIEE
GIKELGSQILKEHPVENTQLQNEKLYLYYLQNGRDMYVDQELDINRLSDYDVDHIVPQSFLK
DDSIDNKVLTRSDKNRGKSDNVPSEEVVKKMKNYWRQLLNAKLITQRKEDNLTKAERGGLSE
LDKAGFIKRQLVETRQITKHVAQILDSRMNTKYDENDKLIREVKVITLKSKLVSDFRKDFQF
YKVREINNYHHAHDAYLNAVVGTALIKKYPKLESEFVYGDYKVYDVRKMIAKSEQEIGKATA
KYFFYSNIMNFFKTEITLANGEIRKRPLIETNGETGEIVWDKGRDFATVRKVLSMPQVNIVK
KTEVQTGGFSKESILPKRNSDKLIARKKDWDPKKYGGFDSPTVAYSVLVVAKVEKGKSKKLK
SVKELLGITIMERSSFEKNPIDFLEAKGYKEVKKDLIIKLPKYSLFELENGRKRMLASAGEL
QKGNELALPSKYVNFLYLASHYEKLKGSPEDNEQKQLFVEQHKHYLDEIIEQISEFSKRVIL
ADANLDKVLSAYNKHRDKPIREQAENIIHLFTLTNLGAPAAFKYEDTTIDRKRYTSTKEVLD

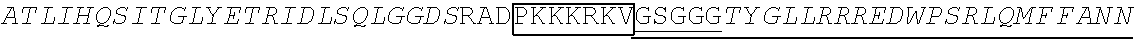

HDQEFDPPKVYPPVPAEKRKPIRVLSLFDGIATGLLVLKDLGIQVDRYIASEVCEDSITVGM
VRHQGKIMYVGDVRSVTQKHIQEWGPFDLVIGGSPCNDLSIVNPARKGLYEGTGRLFFEFYR
LLHDARPKEGDDRPFFWLFENVVAMGVSDKRDISRFLESNPVMIDAKEVSAAHRARYFWGNL
PGMNRPLASTVNDKLELQECLEHGRIAKFSKVRTITTRSNSIKQGKDQHFPVFMNEKEDILW
CTEMERVFGFPVHYTDVSNMSRLARQRLLGRSWSVPVIRHLFAPLKEYFACV

In the above sequence, the Cas9 domain is shown in italics, an HA tag is shown in bold, a linker domain is shown in bold and double-underlined, and the DNMT3A domain is in italics and underlined. NLS sequences are boxed.

In one aspect, the ETM (e.g., ETR) comprises or consists of an endonuclease-DNMT3L fusion protein such as a Cas-DNMT3L, e.g., a Cas9-DNMT3L domain fusion protein.

An exemplary sequence of an ETM according to the present invention comprising a DNMT3L domain (ETM-D3L) is set forth below in SEQ ID NO: 20:

(SEQ ID NO: 20)

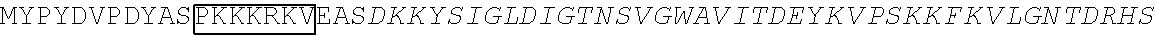

IKKNLIGALLFDSGETAEATRLKRTARRRYTRRKNRICYLQEIFSNEMAKVDDSFFHRLEES
FLVEEDKKHERHPIFGNIVDEVAYHEKYPTIYHLRKKLVDSTDKADLRLIYLALAHMIKFRG
HFLIEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYADLF
LAAKNLSDAILLSDILRVNTEITKAPLSASMIKRYDEHHQDLTLLKALVRQQLPEKYKEIFF
DQSKNGYAGYIDGGASQEEFYKFIKPILEKMDGTEELLVKLNREDLLRKORTFDNGSIPHQI
HLGELHAILRRQEDFYPFLKDNREKIEKILTFRIPYYVGPLARGNSRFAWMTRKSEETITPW
NFEEVVDKGASAQSFIERMTNFDKNLPNEKVLPKHSLLYEYFTVYNELTKVKYVTEGMRKPA
FLSGEQKKAIVDLLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKOLKRRRYTGWGRL
SRKLINGIRDKQSGKTILDFLKSDGFANRNFMQLIHDDSLTFKEDIQKAQVSGQGDSLHEHI
ANLAGSPAIKKGILQTVKVVDELVKVMGRHKPENIVIEMARENQTTQKGQKNSRERMKRIEE
GIKELGSQILKEHPVENTQLQNEKLYLYYLQNGRDMYVDQELDINRLSDYDVDHIVPQSFLK
DDSIDNKVLTRSDKNRGKSDNVPSEEVVKKMKNYWRQLLNAKLITORKFDNLTKAERGGLSE
LDKAGFIKRQLVETRQITKHVAQILDSRMNTKYDENDKLIREVKVITLKSKLVSDERKDFQF
YKVREINNYHHAHDAYLNAVVGTALIKKYPKLESEFVYGDYKVYDVRKMIAKSEQEIGKATA
KYFFYSNIMNFFKTEITLANGEIRKRPLIETNGETGEIVWDKGRDFATVRKVLSMPQVNIVK
KTEVQTGGFSKESILPKRNSDKLIARKKDWDPKKYGGFDSPTVAYSVLVVAKVEKGKSKKLK
SVKELLGITIMERSSFEKNPIDFLEAKGYKEVKKDLIIKLPKYSLFELENGRKRMLASAGEL
QKGNELALPSKYVNFLYLASHYEKLKGSPEDNEQKQLFVEQHKHYLDEIIEQISEFSKRVIL
ADANLDKVLSAYNKHRDKPIREQAENIIHLFTLTNLGAPAAFKYFDTTIDRKRYTSTKEVLD

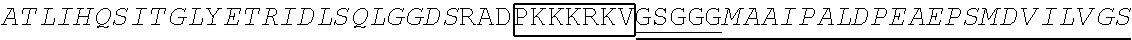

SELSSSVSPGTGRDLIAYEVKANQRNIEDICICCGSLOVHTQHPLFEGGICAPCKDKFLDAL
FLYDDDGYQSYCSICCSGETLLICGNPDCTRCYCFECVDSLVGPGTSGKVHAMSNWVCYLCL
PSSRSGLLQRRRKWRSQLKAFYDRESENPLEMFETVPVWRRQPVRVLSLFEDIKKELTSLGF
LESGSDPGQLKHVVDVTDTVRKDVEEWGPFDLVYGATPPLGHTCDRPPSWYLFQFHRLLQYA
RPKPGSPRPFFWMFVDNLVLNKEDLDVASRFLEMEPVTIPDVHGGSLQNAVRVWSNIPAIRS
RHWALVSEEELSLLAQNKQSSKLAAKWPTKLVKNCFLPLREYFKYFSTELTSSL

The Cas9 domain is shown in italics, an HA tag is shown in bold, a linker domain is shown in bold and double-underlined, and the DNMT3L domain is in italics and underlined. NLS sequences are boxed.

A fusion protein may, for example, comprise an amino acid sequence that has at least 70%, 80%, 90%, 95%, 96%, 97%, 98%, 99% or 100% identity to SEQ ID NO: 18, 19, 20, 4481 or 4482, e.g., wherein the amino acid sequence substantially retains the natural function of the protein represented by SEQ ID NO: 18, 19, 20, 4481 or 4482.

A fusion protein may, for example, be encoded by a polynucleotide comprising a nucleic acid sequence which encodes the protein of SEQ ID NO: 18, 19, 20, 4481 or 4482, or a protein that has at least 70%, 80%, 90%, 95%, 96%, 97%, 98%, 99% or 100% amino acid identity to SEQ ID NO: 18, 19, 20, 4481 or 4482, e.g., wherein the amino acid sequence substantially retains the natural function of the protein represented by SEQ ID NO: 18, 19, 20, 4481 or 4482, respectively. The coding sequence may be codon-optimized for optimal expression in human cells.

### Epigenetic Effector Domains

The term “epigenetic effector domain”, is to be understood as referring to the part of the ETM which provides for the epigenetic effect on a target gene, for example by catalysing a reaction on the DNA or chromatin (e.g., methylation of DNA), or by recruiting an additional agent from within a cell, e.g., resulting in the repression of the transcription of a gene.

“Domain” is to be understood in this context as referring to a part of the ETM that harbours a certain function. The domain may be an individual domain (e.g., a catalytic domain) isolated from a natural protein or it may be an entire, full-length natural protein. Put another way, either the full-length protein or a functional fragment thereof can be used as an effector domain. Therefore, for example, “Kruppel-associated box (KRAB) domain” or “KRAB domain” refers to the part of the ETM that comprises an amino acid sequence with the function of a KRAB domain.

Chromatin remodeling enzymes that are known to be involved in the permanent epigenetic silencing of endogenous retroviruses (ERVs; Feschotte, C. et al. (2012) Nat. Rev. Genet. 13: 283-96; Leung, D. C. et al. (2012) Trends Biochem. Sci. 37: 127-33) may provide suitable effector domains for exploitation in the present invention.

In one aspect, the epigenetic effector domain is capable of repressing transcription and/or expression of at least one target gene. A factor capable of repressing transcription of a gene is also called a transcriptional repressor. In one aspect, the epigenetic effector domain is a repressor domain, e.g., a transcriptional repressor domain.

In one aspect, the epigenetic effector domain initiates chemical modification of chromatin and/or chromatin remodeling.

In one aspect, the epigenetic effector domain initiates DNA modification, such as DNA methylation. In one aspect, the epigenetic effector domain is a DNA methyltransferase and/or is capable of recruiting a DNA methyltransferase.

In one aspect, the epigenetic effector domain initiates histone modification, such as histone methylation or histone acetylation. In one aspect, the epigenetic effector domain is a histone methyltransferase or histone acetyltransferase.

In one aspect, the at least one epigenetic effector domain comprises a Kruppel-associated box (KRAB) domain, a DNA methyltransferase (DNMT) domain, a DNMT-like domain, or a histone methyltransferase (HMT) domain.

In one aspect, the at least one epigenetic effector domain is an antibody or derivative thereof, such as a nanobody, which binds an epigenetic regulator, such as a chromatin regulator which may chemically modify chromatin and/or remodel chromatin.

See, for example, Van et al., Nat Commun. 2021 Jan. 22; 12(1)537, which describes nanobody-mediated control of gene expression and epigenetic memory.

### KRAB

In some aspects, the at least one epigenetic effector domain comprises a KRAB domain. The family of the Kruppel-associated box containing zinc finger proteins (KRAB-ZFP; Huntley, S. et al. (2006) Genome Res. 16: 669-77) plays an important role in the silencing of endogenous retroviruses. These transcription factors bind to specific ERV sequences through their ZFP DNA binding domain, while they recruit the KRAB Associated Protein 1 (KAP1) with their conserved KRAB domain. KAP1 in turn binds a large number of effectors that promote the local formation of repressive chromatin (Iyengar, S. et al. (2011) J. Biol. Chem. 286: 26267-76).

An ETM of the present invention may, for example, comprise a KRAB domain. Various KRAB domains are known in the family of KRAB-ZFP proteins. For example, an ETM of the present invention may comprise the KRAB domain of human zinc finger protein 10 (ZNF10; Szulc, J. et al. (2006) Nat. Methods 3: 109-16):

(SEQ ID NO: 1)
ALSPQHSAVTQGSIIKNKEGMDAKSLTAWSRTLVTFKDVFVDFTREEWKL
LDTAQQIVYRNVMLENYKNLVSLGYQLTKPDVILRLEKGEEPWLVEREIH
QETHPDSETAFEIKSSV

Further examples of suitable KRAB domains for use in the present invention include:

(the KRAB domain of the human ZIM3 protein; SEQ ID NO: 2)
MNNSQGRVTFEDVTVNFTQGEWORLNPEQRNLYRDVMLENYSNLVSVGQGETTKPDV
ILRLEQGKEPWLEEEEVLGSGRAEKNGDIGGQIWKPKDVKESL
(the KRAB domain of the ZNF350 protein; SEQ ID NO: 3)
ITLEDVAVDFTWEEWQLLGAAQKDLYRDVMLENYSNLVAVGYQASKPDALFKLEQGE
QLWTIEDGIHSGACS
(the KRAB domain of the ZNF197 protein; SEQ ID NO: 4)
VMFEEVSVCFTSEEWACLGPIQRALYWDVMLENYGNVTSLEWETMTENEEVTSKPSS
SQRADSHKGTSKRLQG
(the KRAB domain of the RBAK protein; SEQ ID NO: 5)
VSFKDVAVDFTQEEWQQLDPDEKITYRDVMLENYSHLVSVGYDTTKPNVIIKLEQGE
EPWIMGGEFPCQHSP
(the KRAB domain of the ZKSCAN1 protein; SEQ ID NO: 6)
VKIEDMAVSLILEEWGCONLARRNLSRDNRQENYGSAFPQGGENRNENEESTSKAET
SEDSASRGETTGRSQKE
(the KRAB domain of the KRBOX4 protein; SEQ ID NO: 7)
LTFKDVFVDFTLEEWQQLDSAQKNLYRDVMLENYSHLVSVGYLVAKPDVIFRLGPGE
ESWMADGGTPVRTCA
(the KRAB domain of the ZNF274 protein; SEQ ID NO: 8)
VTFEDVTLGFTPEEWGLLDLKOKSLYREVMLENYRNLVSVEHQLSKPDVVSQLEEAE
DFWPVERGIPODTIP

The above KRAB domains are illustrative only. Functional variants thereof are also contemplated herein. For example, the ZIM3 KRAB domain shown in SEQ ID NO: 4481 and 4482 (see Examples 3 and 4 below) may also be used. That ZIM3 KRAB domain has the following sequence:

(SEQ ID NO: 4637)
MGRVTFEDVTVNFTQGEWQRLNPEQRNLYRDVMLENYSNLVSVGQGETTK
PDVILRLEQGKEPWLEEEEVLGSGRAEKNGDIGGQIWKPKDVKESL

### DNMT

In some aspects, the epigenetic effector domain comprises a DNA methyltransferase (DNMT) domain. DNMTs catalyse the transfer of a methyl group to DNA. Examples of DNMTs are DNMT1, DNMT3A and DNMT3B.

An ETM of the present invention may, for example, comprise a domain of human DNA methyltransferase 3A (DNMT3A; Law, J. A. et al. (2010) Nat. Rev. Genet. 11: 204-20), e.g., the catalytic domain. For example, an ETM of the present invention may comprise the sequence:

(the catalytic domain of human DNMT3A; SEQ ID NO: 9)
TYGLLRRREDWPSRLQMFFANNHDQEFDPPKVYPPVPAEKRKPIRVLSLEDGIATGL
LVLKDLGIQVDRYIASEVCEDSITVGMVRHQGKIMYVGDVRSVTQKHIQEWGPFDLV
IGGSPCNDLSIVNPARKGLYEGTGRLFFEFYRLLHDARPKEGDDRPFFWLFENVVAM
GVSDKRDISRFLESNPVMIDAKEVSAAHRARYFWGNLPGMNRPLASTVNDKLELQEC
LEHGRIAKFSKVRTITTRSNSIKQGKDQHFPVEMNEKEDILWCTEMERVFGFPVHYT
DVSNMSRLARQRLLGRSWSVPVIRHLFAPLKEYFACV

DNA methyltransferases 3B and 1 (DNMT3B and DNMT1), similarly to DNMT3A, are also responsible for the deposition and maintenance of DNA methylation, and may also be used in an ETM of the present invention. For example, an ETM of the present invention may comprise any of the sequences:

(the catalytic domain of human DNMT3B; SEQ ID NO: 10)
CHGVLRRRKDWNVRLOAFFTSDTGLEYEAPKLYPAIPAARRRPIRVLSLEDGIATGY
LVLKELGIKVGKYVASEVCEESIAVGTVKHEGNIKYVNDVRNITKKNIEEWGPFDLV
IGGSPCNDLSNVNPARKGLYEGTGRLFFEFYHLLNYSRPKEGDDRPFFWMFENVVAM
KVGDKRDISRFLECNPVMIDAIKVSAAHRARYFWGNLPGMNRPVIASKNDKLELQDC
LEYNRIAKLKKVQTITTKSNSIKQGKNQLFPVVMNGKEDVLWCTELERIFGFPVHYT
DVSNMGRGARQKLLGRSWSVPVIRHLFAPLKDYFACE
(human DNMT3B: SEQ ID NO: 11)
MVAELISEEDLEFMKGDTRHLNGEEDAGGREDSILVNGACSDOSSDSPPILEAIRTP
EIRGRRSSSRLSKREVSSLLSYTQDLTGDGDGEDGDGSDTPVMPKLFRETRTRSESP
AVRTRNNNSVSSRERHRPSPRSTRGRQGRNHVDESPVEFPATRSLRRRATASAGTPW
PSPPSSYLTIDLTDDTEDTHGTPOSSSTPYARLAQDSQQGGMESPQVEADSGDGDSS
EYQDGKEFGIGDLVWGKIKGFSWWPAMVVSWKATSKRQAMSGMRWVQWFGDGKFSEV
SADKLVALGLESQHFNLATENKLVSYRKAMYHALEKARVRAGKTFPSSPGDSLEDQL
KPMLEWAHGGFKPTGIEGLKPNNTQPENKTRRRTADDSATSDYCPAPKRLKINCYNN
GKDRGDEDQSREQMASDVANNKSSLEDGCLSCGRKNPVSFHPLFEGGLCQTCRDREL
ELFYMYDDDGYQSYCTVCCEGRELLLCSNTSCCRCFCVECLEVLVGTGTAAEAKLQE
PWSCYMCLPQRCHGVLRRRKDWNVRLQAFFTSDTGLEYEAPKLYPAIPAARRRPIRV
LSLFDGIATGYLVLKELGIKVGKYVASEVCEESIAVGTVKHEGNIKYVNDVRNITKK
NIEEWGPFDLVIGGSPCNDLSNVNPARKGLYEGTGRLFFEFYHLLNYSRPKEGDDRP
FFWMFENVVAMKVGDKRDISRFLECNPVMIDAIKVSAAHRARYFWGNLPGMNRPVIA
SKNDKLELQDCLEYNRIAKLKKVQTITTKSNSIKQGKNQLFPVVMNGKEDVLWCTEL
ERIFGFPVHYTDVSNMGRGARQKLLGRSWSVPVIRHLFAPLKDYFACE
(the catalytic domain of human DNMT1; SEQ ID NO: 12)
LRTLDVFSGCGGLSEGFHQAGISDTLWAIEMWDPAAQAFRLNNPGSTVFTEDCNILL
KLVMAGETTNSRGQRLPQKGDVEMLCGGPPCQGFSGMNRFNSRTYSKFKNSLVVSFL
SYCDYYRPRFFLLENVRNFVSFKRSMVLKLTLRCLVRMGYQCTFGVLQAGQYGVAQT
RRRAIILAAAPGEKLPLFPEPLHVFAPRACQLSVVVDDKKFVSNITRLSSGPFRTIT
VRDTMSDLPEVRNGASALEISYNGEPQSWFQRQLRGAQYQPILRDHICKDMSALVAA
RMRHIPLAPGSDWRDLPNIEVRLSDGTMARKLRYTHHDRKNGRSSSGALRGVCSCVE
AGKACDPAARQFNTLIPWCLPHTGNRHNHWAGLYGRLEWDGFFSTTVINPEPMGKQG
RVLHPEQHRVVSVRECARSQGFPDTYRLFGNILDKHRQVGNAVPPPLAKAIGLEIKL
CMLAKARESASAKIKEEEAAKD

### DNMT-Like

In some aspects, the epigenetic effector domain may be a DNMT-like domain. A “DNMT-like” domain refers to a protein, or a functional fragment thereof, wherein the protein is a member of a DNMT family but does not possess DNA methylation activity. The DNMT-like protein typically activates or recruits other epigenetic effector domains.

An ETM of the present invention may, for example, comprise DNA (cytosine-5)-methyltransferase 3-like (DNMT3L), a catalytically inactive DNA methyltransferase that activates DNMT3A by binding to its catalytic domain. For example, an ETM of the present invention may comprise the sequence:

(human DNMT3L; SEQ ID NO: 13)
MAAIPALDPEAEPSMDVILVGSSELSSSVSPGTGRDLIAYEVKANQRNIE
DICICCGSLQVHTQHPLFEGGICAPCKDKFLDALFLYDDDGYQSYCSICC
SGETLLICGNPDCTRCYCFECVDSLVGPGTSGKVHAMSNWVCYLCLPSSR
SGLLQRRRKWRSQLKAFYDRESENPLEMFETVPVWRRQPVRVLSLFEDIK
KELTSLGFLESGSDPGQLKHVVDVTDTVRKDVEEWGPFDLVYGATPPLGH
TCDRPPSWYLFQFHRLLQYARPKPGSPRPFFWMFVDNLVLNKEDLDVASR
FLEMEPVTIPDVHGGSLQNAVRVWSNIPAIRSRHWALVSEEELSLLAQNK
QSSKLAAKWPTKLVKNCFLPLREYFKYFSTELTSSL

### HMT

In some aspects, the epigenetic effector domain may be a histone methyltransferase (HMT) domain, e.g., the catalytic domain. HMTs are histone modifying enzymes which catalyse the transfer of methyl groups to lysine and arginine residues of histone proteins.

Lysine-specific HMTs may contain a SET (Su(var)3-9, Enhancer of Zeste, Trithorax) domain or may be non-SET domain containing.

An example of an HMT is SET domain bifurcated 1 (SETDB1).

In early embryonic development, KAP1 is known to recruit SETDB1, a histone methyltransferase that deposits histone H3 lysine-9 di- and tri-methylation (H3K9me2 and H3K9me3, respectively), two histone marks associated with transcriptional repression. Concurrently, KAP1 binds to Heterochromatin Protein 1 alpha (HP1α), which reads H3K9me2 and H3K9me3 and stabilises the KAP1-containing complex. KAP1 can also interact with other well-known epigenetic silencers, such as lysine-specific histone demethylase 1 (LSD1) that inhibits transcription by removing histone H3 lysine-4 methylation, and the nucleosome remodeling and deacetylase complex (NURD), which removes acetyl groups from histones. Finally, the KAP1-containing complex contributes to the recruitment of the de novo DNA methyltransferase 3A (DNMT3A), which methylates cytosines at CpG sites (Jones, P. A. (2012) Nat. Rev. Genet. 13: 484-92). Together, these data suggest a model in which, in the pre-implantation embryo, the KAP1 complex ensures ERV silencing through the concerted action of histone modifying enzymes and DNA methylation. Then, after implantation, the DNA methylation previously targeted by KRAB-ZFPs to the ERVs becomes stable (Reik, W. (2007) Nature 447: 425-32), being inherited throughout mitosis and somatic cell differentiation without the need for continuous expression of ERVs-specific KRAB-ZFPs. Unlike in embryonic stem cells, the KAP1 complex is not able to efficiently induce DNA methylation in somatic cells, being only able to deposit H3K9 methylation. However, this histone mark is not maintained without continuous deposition at the targeted site by the KRAB-ZFPs (Hathaway, N. A. et al. (2012) Cell 149: 1447-60).

In some aspects, at least two epigenetic effector domains may be utilised, one based on, for example, the KRAB domain (e.g., the initiator of the epigenetic cascade occurring at ERVs in embryonic stem cells), and the other based on, for example, DNMT3A (e.g., the final lock of this process). This approach may allow recapitulating on a pre-selected target gene those repressive chromatin states established at ERVs in the pre-implantation embryo and then permanently inherited throughout mammalian development and adult life.

An ETM of the present invention may, for example, comprise a SETDB1 domain. For example, an ETM of the present invention may comprise any of the sequences:

(human SETDB1; SEQ ID NO: 14)
MSSLPGCIGLDAATATVESEEIAELQQAVVEELGISMEELRHFIDEELEKMDCVQQR
KKQLAELETWVIQKESEVAHVDQLEDDASRAVINCESLVKDFYSKLGLQYRDSSSED
ESSRPTEIIEIPDEDDDVLSIDSGDAGSRTPKDOKLREAMAALRKSAQDVQKEMDAV
NKKSSSQDLHKGTLSQMSGELSKDGDLIVSMRILGKKRTKTWHKGTLIAIQTVGPGK
KYKVKFDNKGKSLLSGNHIAYDYHPPADKLYVGSRVVAKYKDGNQVWLYAGIVAETP
NVKNKLRFLIFFDDGYASYVTQSELYPICRPLKKTWEDIEDISCRDFIEEYVTAYPN
RPMVLLKSGQLIKTEWEGTWWKSRVEEVDGSLVRILFLDDKRCEWIYRGSTRLEPMF
SMKTSSASALEKKQGQLRTRPNMGAVRSKGPVVQYTQDLTGTGTQFKPVEPPQPTAP
PAPPFPPAPPLSPQAGDSDLESQLAQSRKQVAKKSTSFRPGSVGSGHSSPTSPALSE
NVSGGKPGINQTYRSPLGSTASAPAPSALPAPPAPPVFHGMLERAPAEPSYRAPMEK
LFYLPHVCSYTCLSRVRPMRNEQYRGKNPLLVPLLYDERRMTARRRVNRKMGFHVIY
KTPCGLCLRTMQEIERYLFETGCDFLFLEMFCLDPYVLVDRKFQPYKPFYYILDITY
GKEDVPLSCVNEIDTTPPPQVAYSKERIPGKGVFINTGPEFLVGCDCKDGCRDKSKC
ACHQLTIQATACTPGGQINPNSGYQYKRLEECLPTGVYECNKRCKCDPNMCTNRLVQ
HGLQVRLQLFKTQNKGWGIRCLDDIAKGSFVCIYAGKILTDDFADKEGLEMGDEYFA
NLDHIESVENFKEGYESDAPCSSDSSGVDLKDQEDGNSGTEDPEESNDDSSDDNFCK
DEDESTSSVWRSYATRRQTRGOKENGLSETTSKDSHPPDLGPPHIPVPPSIPVGGCN
PPSSEETPKNKVASWLSCNSVSEGGFADSDSHSSFKTNEGGEGRAGGSRMEAEKAST
SGLGIKDEGDIKQAKKEDTDDRNKMSVVTESSRNYGYNPSPVKPEGLRRPPSKTSMH
QSRRLMASAQSNPDDVLTLSSSTESEGESGTSRKPTAGQTSATAVDSDDIQTISSGS
EGDDFEDKKNMTGPMKRQVAVKSTRGFALKSTHGIAIKSTNMASVDKGESAPVRKNT
RQFYDGEESCYIIDAKLEGNLGRYLNHSCSPNLFVQNVFVDTHDLRFPWVAFFASKR
IRAGTELTWDYNYEVGSVEGKELLCCCGAIECRGRLL
(the catalytic domain of human SETDB1; SEQ ID NO: 15)
VGCDCKDGCRDKSKCACHOLTIQATACTPGGQINPNSGYQYKRLEECLPTGVYECNK
RCKCDPNMCTNRLVQHGLQVRLQLFKTQNKGWGIRCLDDIAKGSFVCIYAGKILTDD
FADKEGLEMGDEYFANLDHIESVENFKEGYESDAPCSSDSSGVDLKDQEDGNSGTED
PEESNDDSSDDNFCKDEDFSTSSVWRSYATRRQTRGQKENGLSETTSKDSHPPDLGP
PHIPVPPSIPVGGCNPPSSEETPKNKVASWLSCNSVSEGGFADSDSHSSFKTNEGGE
GRAGGSRMEAEKASTSGLGIKDEGDIKQAKKEDTDDRNKMSVVTESSRNYGYNPSPV
KPEGLRRPPSKTSMHQSRRLMASAQSNPDDVLTLSSSTESEGESGTSRKPTAGOTSA
TAVDSDDIQTISSGSEGDDFEDKKNMTGPMKRQVAVKSTRGFALKSTHGIAIKSTNM
ASVDKGESAPVRKNTRQFYDGEESCYIIDAKLEGNLGRYLNHSCSPNLFVQNVFVDT
HDLRFPWVAFFASKRIRAGTELTWDYNYEVGSVEGKELLCCCGAIECRGRLL

The ETM of the present invention may, for example, comprise an amino acid sequence that has at least 70%, 80%, 90%, 95%, 96%, 97%, 98%, 99% or 100% identity to SEQ ID NO: 1, 2, 3, 4, 5, 6, 7, 8, 9, 10, 11, 12, 13, 14 or 15, e.g., wherein the amino acid sequence substantially retains the natural function of the protein represented by SEQ ID NO: 1, 2, 3, 4, 5, 6, 7, 8, 9, 10, 11, 12, 13, 14 or 15, respectively.

The ETM of the present invention may, for example, be encoded by a polynucleotide comprising a nucleic acid sequence which encodes the protein of SEQ ID NOs: 1, 2, 3, 4, 5, 6, 7, 8, 9, 10, 11, 12, 13, 14 or 15, or a protein that has at least 70%, 80%, 90%, 95%, 96%, 97%, 98%, 99% or 100% amino acid identity to SEQ ID NOs: 1, 2, 3, 4, 5, 6, 7, 8, 9, 10, 11, 12, 13, 14 or 15, e.g., wherein the amino acid sequence substantially retains the natural function of the protein represented by SEQ ID NOs: 1, 2, 3, 4, 5, 6, 7, 8, 9, 10, 11, 12, 13, 14 or 15, respectively. The coding sequence may be codon-optimized for optimal expression in human cells.

The ETM of the present invention may, for example, comprise an amino acid sequence that has at least 70%, 80%, 90%, 95%, 96%, 97%, 98%, 99% or 100% identity to SEQ ID NO: 4637, e.g., wherein the amino acid sequence substantially retains the natural function of the protein represented by SEQ ID NO: 4637.

The ETM of the present invention may, for example, be encoded by a polynucleotide comprising a nucleic acid sequence which encodes the protein of SEQ ID NO: 4637, or a protein that has at least 70%, 80%, 90%, 95%, 96%, 97%, 98%, 99% or 100% amino acid identity to SEQ ID NO: 4637, e.g., wherein the amino acid sequence substantially retains the natural function of the protein represented by SEQ ID NO: 4637. The coding sequence may be codon-optimized for optimal expression in human cells.

### Endonuclease

The ETM (e.g., ETR) of the invention may comprise an endonuclease.

The endonuclease may be, for example, site-specific. As used herein, “site-specific endonuclease” may refer to an enzyme which induces site-directed double-strand breaks in DNA. The site-specific endonuclease enables the activity of the ETM (e.g., ETR) to be targeted to specific sites in a polynucleotide, for example the genome of a cell. For example, the endonuclease may be site-specific when used in combination with gRNAs, in other words, the endonuclease is capable of inducing site-directed DNA breaks when used in combination with gRNAs.

In one aspect, the endonuclease has exonuclease activity in addition to endonuclease activity.

The endonuclease may, e.g., bind to binding sites within a target gene or within regulatory sequences for the target gene, for example promoter or enhancer sequences.

The endonuclease may, e.g., bind to binding sites within splicing sites. Splicing variants of a given gene may be regulated by DNA methylation/demethylation at splicing sites. In turn, these modifications may cause exon exclusion/inclusion in the mature transcript. This exclusion/inclusion may have therapeutic relevance, such as in the case of Duchenne Muscular Dystrophy, in which exclusion (by genetic ablation or exon skipping) from the mature mRNA of an exon bearing the most frequent disease-causing mutation has been proposed for therapy (Ousterout, D. G. et al. (2015) Mol. Ther. 23: 523-32; Ousterout, D. G. et al. (2015) Nat. Commun. 6: 6244; Kole, R. et al. (2015) Adv. Drug Deliv. Rev. 87: 104-7; Touznik, A. et al. (2014) Expert Opin. Biol. Ther. 14: 809-19).

A number of suitable endonucleases are known in the art. For example, CRISPR/Cas systems (Sander, J. D. et al. (2014) Nat. Biotechnol. 32: 347-55) may be employed as suitable endonucleases in the ETMs (e.g., ETRs) of the present invention.

“CRISPR/Cas system” refers to a clustered regularly interspaced short palindromic repeats/CRISPR associated nuclease system.

Clustered Regularly Interspaced Short Palindromic Repeats consist of short sequences that originate from viral genomes and have been incorporated into the bacterial genome. CRISPR associated proteins (Cas) process these sequences and cut matching viral DNA sequences. By introducing Cas and specifically constructed CRISPRs into eukaryotic cells, the eukaryotic genome can be cut at any desired position.

The CRISPR/Cas system is an RNA-guided DNA binding system (van der Oost et al. (2014) Nat. Rev. Microbiol. 12: 479-92), wherein the guide RNA (gRNA) may be selected to enable an ETM (e.g., ETR) comprising a Cas domain to be targeted to a specific sequence. Thus, to employ the CRISPR/Cas system as an endonuclease in the present invention, it is to be understood that an epigenetic effector domain may be operably linked to a Cas endonuclease such as a Cas9 endonuclease. The ETM (e.g., ETR) comprising the Cas endonuclease may be delivered to a target cell in combination with one or more gRNAs. The gRNAs are designed to target the ETM (e.g., ETR) to a target gene of interest or a regulatory element (e.g., a promoter, enhancer, or splicing site) of the target gene. Methods for the design of gRNAs are known in the art. Furthermore, fully orthogonal Cas9 proteins, as well as Cas9/gRNA ribonucleoprotein complexes and modifications of the gRNA structure/composition to bind different proteins, have been developed to simultaneously and directionally target different effector domains to desired genomic sites of cells (Esvelt et al. (2013) Nat. Methods 10: 1116-21; Zetsche, B. et al. (2015) Cell pii: S0092-8674(15)01200-3; Zalatan, J. G. et al. (2015) Cell 160: 339-50; Paix, A. et al. (2015) Genetics 201: 47-54), and are suitable for use in the present invention.

In one aspect, the ETM (e.g., ETR) comprises at least one endonuclease derived from type II CRISPR bacterial immune systems. In other words, the ETM (e.g., ETR) may comprise a Type II Cas.

Examples of Cas Type II enzymes include Cas9, Csn2 and Cas4.

Cas9 endonucleases typically comprise RecI, RecII, bridge helix, RuvC, HNH and PAM interacting domains.

The HNH and RuvC domains are nuclease domains. The RecI domain binds gRNA. The bridge helix initiates cleavage upon binding of target DNA. The PAM-interacting domain confers PAM specificity and is responsible for initiating binding to target DNA.

The endonuclease may comprise or consist of a Cas endonuclease. Thus, the endonuclease may have nuclease activity. For example, the endonuclease may be a catalytically active nuclease, bind gRNA, and bind to target DNA.

The endonuclease comprised in an ETM (e.g., ETR) according to the invention is a catalytically active endonuclease. In other words, the ETM (e.g., ETR) is capable of cleaving a target sequence, such as target DNA.

In one aspect, the endonuclease is catalytically active Cas nuclease.

In one aspect, the endonuclease is a modified or a variant endonuclease, such as a modified Cas or modified Cas9 enzyme. For example, it will be appreciated that the enzyme may be modified to recognise a specific PAM site suitable for a target gene. The modified PAM may be different to the PAM naturally recognised by the enzyme.

In one aspect, the ETM (e.g., ETR) according to the present invention does not comprise only catalytically inactive, or catalytically dead (dCas) nuclease. In one aspect, the ETM (e.g., ETR) according to the present invention does not comprise a catalytically inactive, or catalytically dead (dCas) nuclease, such as dCas9.

In one aspect, the endonuclease is a catalytically active Cas9 nuclease.

In one aspect, the endonuclease is a catalytically active Cas9 nuclease from Streptococcus pyogenes (SpCas9).

Methods for determining whether a protein is a catalytically active nuclease are known in the art, for example using gel assays, Kunitz assays, radiolabel assays and fluorescence-based methods. Gel assays may be performed using purified recombinant target DNA as a substrate in an assay buffer. The protein to be tested may be incubated with the substrate, for example incubated at 37° C. for 1 hour. The reaction products can be separated by electrophoresis, for example, on an agarose gel with ethidium bromide to visualize the products of the nuclease reaction. Other methods include, for example, fluorescence real-time quantification of DNA and RNA nuclease activity as reported in Sheppard, E. C., et al. Sci Rep 9, 8853 (2019) and cell free detection of Cas nucleases as reported in J. Cox et al., Chem Sci. 2019 Mar. 7; 10(9): 2653-2662.

For example, an ETM (e.g., ETR) of the present invention may comprise the following catalytically active Cas9 sequence:

(SEQ ID NO: 16)
DKKYSIGLDIGTNSVGWAVITDEYKVPSKKFKVLGNTDRHSIKKNLIGAL
LFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADL
RLIYLALAHMIKERGHFLIEGDLNPDNSDVDKLFIQLVQTYNQLFEENPI
NASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRK
ORTFDNGSIPHQIHLGELHAILRRQEDFYPFLKDNREKIEKILTFRIPYY
VGPLARGNSRFAWMTRKSEETITPWNFEEVVDKGASAQSFIERMTNFDKN
LPNEKVLPKHSLLYEYFTVYNELTKVKYVTEGMRKPAFLSGEQKKAIVDL
LFKTNRKVTVKOLKEDYFKKIECFDSVEISGVEDRFNASLGTYHDLLKII
KDKDELDNEENEDILEDIVLTLTLFEDREMIEERLKTYAHLFDDKVMKOL
KRRRYTGWGRLSRKLINGIRDKOSGKTILDFLKSDGFANRNEMQLIHDDS
LTFKEDIQKAQVSGQGDSLHEHIANLAGSPAIKKGILQTVKVVDELVKVM
GRHKPENIVIEMARENQTTQKGQKNSRERMKRIEEGIKELGSQILKEHPV
ENTQLQNEKLYLYYLONGRDMYVDQELDINRLSDYDVDHIVPQSFLKDDS
IDNKVLTRSDKNRGKSDNVPSEEVVKKMKNYWRQLLNAKLITORKFDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGFDSPTVAYSVLVVAKVEK
GKSKKLKSVKELLGITIMERSSFEKNPIDFLEAKGYKEVKKDLIIKLPKY
SLFELENGRKRMLASAGELQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPAAFKYEDTTIDRKRYTSTKEVLDATLIHQS
ITGLYETRIDLSQLGGDS

The ETM (e.g., ETR) of the present invention may, for example, comprise an amino acid sequence that has at least 70%, 80%, 90%, 95%, 96%, 97%, 98%, 99% or 100% identity to SEQ ID NO: 16, e.g., wherein the amino acid sequence substantially retains the natural function (e.g., endonuclease function) of the protein represented by SEQ ID NO: 16.

The ETM (e.g., ETR) of the present invention may, for example, be encoded by a polynucleotide comprising a nucleic acid sequence which encodes the protein of SEQ ID NO: 16, or a protein that has at least 70%, 80%, 90%, 95%, 96%, 97%, 98%, 99% or 100% amino acid identity to SEQ ID NO: 16, e.g., wherein the amino acid sequence substantially retains the natural function of the protein represented by SEQ ID NO: 16. The coding sequence may be codon-optimized for optimal expression in human cells.

For comparison, the sequence of a catalytically dead Cas9 (dCas9) is:

(catalytically dead Cas9; dCas9; SEQ ID NO: 17)
DKKYSIGLAIGTNSVGWAVITDEYKVPSKKFKVLGNTDRHSIKKNLIGAL
LFDSGETAEATRLKRTARRRYTRRKNRICYLQEIFSNEMAKVDDSFFHRL
EESELVEEDKKHERHPIFGNIVDEVAYHEKYPTIYHLRKKLVDSTDKADL
RLIYLALAHMIKERGHFLIEGDLNPDNSDVDKLFIQLVQTYNQLFEENPI
NASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAIL
LSDILRVNTEITKAPLSASMIKRYDEHHQDLTLLKALVROQLPEKYKEIF
FDQSKNGYAGYIDGGASQEEFYKFIKPILEKMDGTEELLVKLNREDLLRK
ORTFDNGSIPHQIHLGELHAILRRQEDFYPFLKDNREKIEKILTFRIPYY
VGPLARGNSRFAWMTRKSEETITPWNFEEVVDKGASAQSFIERMTNFDKN
LPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKII
KDKDELDNEENEDILEDIVLTLTLFEDREMIEERLKTYAHLFDDKVMKQL
KRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVM
GRHKPENIVIEMARENQTTQKGQKNSRERMKRIEEGIKELGSQILKEHPV
ENTQLQNEKLYLYYLONGRDMYVDQELDINRLSDYDVDAIVPQSFLKDDS
IDNKVLTRSDKNRGKSDNVPSEEVVKKMKNYWRQLLNAKLITORKEDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGFDSPTVAYSVLVVAKVEK
GKSKKLKSVKELLGITIMERSSFEKNPIDFLEAKGYKEVKKDLIIKLPKY
SLFELENGRKRMLASAGELQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPAAFKYFDTTIDRKRYTSTKEVLDATLIHQS
ITGLYETRIDLSQLGGDS

The above sequence contains D9A and H839A substitutions relative to its catalytically active (i.e., live) counterpart (SEQ ID NO: 16). A catalytically dead Cas9 (e.g., the above dCas9) may be used in the ETM for epi-editing of one or more target genes, without simultaneous genetic editing of another gene in a cell. For this use, the ETM (e.g., ETR) may, for example, comprise an amino acid sequence that has at least 70%, 80%, 90%, 95%, 96%, 97%, 98%, or 99% identity to SEQ ID NO: 17, e.g., wherein the amino acid sequence substantially retains the natural function of the protein represented by SEQ ID NO: 16, except for the endonuclease function. The ETM (e.g., ETR) may, for example, be encoded by a polynucleotide comprising a nucleic acid sequence which encodes the protein of SEQ ID NO: 17, or a protein that has at least 70%, 80%, 90%, 95%, 96%, 97%, 98%, 99% or 100% amino acid identity to SEQ ID NO: 17, e.g., wherein the amino acid sequence substantially retains the natural function of the protein represented by SEQ ID NO: 16 but for the endonuclease function. The coding sequence may be codon-optimized for optimal expression in human cells.

gRNA

In one aspect, the present invention provides guide RNAs (gRNAs).

The gRNA targets the ETM (e.g., ETR) to a target gene. The gRNA may, for example, be an RNA sequence which recognises the target DNA region of interest and directs the endonuclease within the ETM (e.g., ETR) to that region.

A gRNA is typically made up of two parts:
a) a spacer sequence (which may also be referred to as a targeting domain, guide sequence, or complementarity region, and which may constitute a CRISPR RNA (crRNA)); and
b) a scaffold sequence (which may also be referred to as a tracrRNA in a CRISPR/Cas system).

The spacer and the scaffold sequences may, for example, be provided as separate molecules, or they may be linked, such as via a linker loop or other sequence or may be fused together.

For example, the gRNA may be constituted by two separate molecules, e.g., the spacer (crRNA) and the scaffold (tracrRNA). The 3′ end of the spacer (crRNA) may be complementary to the 5′ end of the scaffold (tracrRNA), which complementarity may lead to dimerization of the two molecules.

In another example, the spacer (crRNA) and the scaffold (tracrRNA) may be fused, for example via a linker loop. This artificial configuration may also be known as a single guide RNA (sgRNA).

In some aspects, variants of the scaffold (tracrRNA) may be used. For example, the tetraloop and stem loop of the scaffold (tracrRNA) sequence may be modified to include RNA aptamers, which can be bound by specific protein domains. In some aspects, such modified gRNAs can be used to facilitate the recruitment of repressive or activating domains fused to the protein-interacting RNA aptamers.

Exemplary tracrRNA sequences include, without limitation:

(SEQ ID NO: 4566)
5′-GUUUAAGAGCUAUGCUGGAAACAGCAUAGCAAGUUUAAAUAAGGCUA
GUCCGUUAUCAACUUGAAAAAGUGGCACCGAGUCGGUGCUUUUUUU-3′,
and
(SEQ ID NO: 4567)
5′-GUUUUAGAGCUAGAAAUAGCAAGUUAAAAUAAGGCUAGUCCGUUA
UCAACUUGAAAAAGUGGCACCGAGUCGGUGCUUUU-3′

A “spacer” or “spacer sequence” refers to a sequence that may be fully complementary to a target domain (i.e., region) within a target sequence.

The 3′ end of the genomic target sequence generally comprises a protospacer adjacent motif (PAM) sequence. A “PAM” sequence is typically a 2 to 6 base pair DNA sequence immediately following the DNA sequence targeted by the nuclease. The PAM sequence is required for cleavage but is not part of the target of the gRNA sequence. The PAM sequence varies depending on the species of the nuclease. For example, the canonical PAM associated with the Cas9 nuclease of Streptococcus pyogenes is the sequence 5′-NGG-3′ where “N” is any nucleobase. Nuclease enzymes derived from different organisms or which have been engineered may recognise different PAM sequences.

For example, the Cas9 of Francisella novicida recognizes the canonical PAM sequence 5′-NGG-3′, but has been engineered to recognize 5′-YG-3′ (where “Y” is a pyrimidine), thus adding to the range of possible Cas9 targets. The Cas12a (or Cpf1) nuclease of Francisella novicida recognizes the PAM 5′-TTTN-3′ or 5′-YTN-3′.

The nucleotides upstream (towards the 5′ end of the target sequence) of the PAM sequence is the protospacer sequence.

A Cas9 nuclease will typically cleave approximately three bases upstream of the PAM.

It will be appreciated that one may choose a suitable nuclease of a particular context based on PAM specificity and the genomic target.

A “scaffold” or “scaffold sequence” is a sequence necessary for endonuclease binding e.g., Cas binding.

In one aspect, the present invention provides single guide RNAs (sgRNAs). In one aspect, the gRNA according to the present invention is a sgRNA. sgRNAs are single RNA molecules which contain a crRNA sequence fused to the scaffold tracrRNA sequence. In nature, crRNAs and tracrRNAs exist as two separate RNA molecules, but sgRNAs have become a common format for CRISPR gRNAs in research.

In one aspect the gRNA comprises a spacer sequence which is 10 nucleotides in length. In one aspect the gRNA comprises a spacer sequence which is 11 nucleotides in length. In one aspect the gRNA comprises a spacer sequence which is 12 nucleotides in length. In one aspect the gRNA comprises a spacer sequence which is 13 nucleotides in length. In one aspect the gRNA comprises a spacer sequence which is 14 nucleotides in length. In one aspect the gRNA comprises a spacer sequence which is 15 nucleotides in length. In one aspect the gRNA comprises a spacer sequence which is 16 nucleotides in length. In one aspect the gRNA comprises a spacer sequence which is 17 nucleotides in length. In one aspect the gRNA/comprises a spacer sequence which is 18 nucleotides in length. In one aspect the gRNA comprises a spacer sequence which is 19 nucleotides in length. In one aspect the gRNA comprises a spacer sequence which is 20 nucleotides in length. In one aspect the gRNA comprises a spacer sequence which is 21 nucleotides in length.

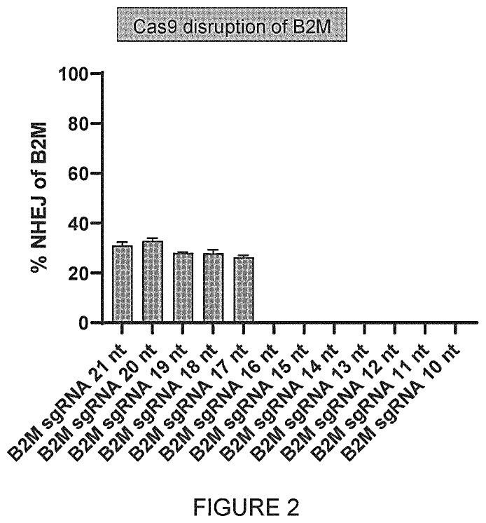

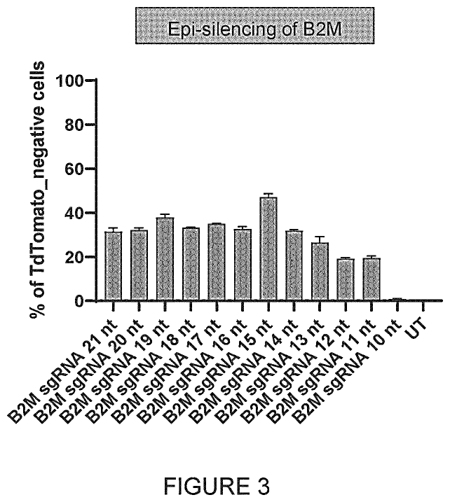

Without wishing to be bound by theory, certain gRNAs (e.g., gRNAs comprising a spacer sequence of around 20 nucleotides in length) may be used to induce gene editing by an ETM (e.g., ETR) whilst gRNAs comprising shorter spacer sequences (e.g., gRNAs comprising spacer sequences of around 16 nucleotides in length) may favour epigenetic editing such as epi-silencing by an ETM (e.g., ETR). See, for example, FIG. 2, which shows that gRNAs comprising spacer sequences of about 18 to 20 nucleotides in length induce NHEJ whilst gRNAs comprising spacer sequences of about 16 nucleotides in length or less do not induce NHEJ. FIG. 3 shows that gRNAs comprising spacer sequences of about 11 to 16 nucleotides in length are capable of inducing epigenetic modification, e.g., epi-silencing of B2M.

In some embodiments, the gRNA comprises a spacer sequence which is less than or equal to 15, 16, or 17 (e.g., less than or equal to 17 or 16) nucleotides in length. In some embodiments, the gRNA comprises a spacer sequence which is 11 to 16 nucleotides in length, such as 12 to 16, 13 to 16, 14 to 16, 15 to 16, 12 to 17, 13 to 17, 14 to 17, 15 to 17, 16, or 17 nucleotides in length.

In some embodiments, the gRNA comprises a spacer sequence which is greater than or equal to 16, 17, or 18 (e.g., greater than or equal to 17 or 18) nucleotides in length, such as 18 or more, 19 or more, or 20 or more nucleotides in length. In some embodiments, the gRNA comprises a spacer sequence which is 17 to 30 nucleotides in length, such as 18 to 30, 19 to 30 or 20 to 30 nucleotides in length. In some embodiments, the gRNA comprises a spacer sequence which is 17 to 25 nucleotides in length, such as 18 to 25, 19 to 25 or 20 to 25 nucleotides in length. In some embodiments, the gRNA comprises a spacer sequence which is 17 to 20 nucleotides in length, such as 18 to 20 or 19 to 20 nucleotides in length.

The ETM according to the present invention may be capable of modifying the transcription, expression and/or activity (e.g., repressing transcription and/or expression) of multiple target genes within the same cell by epigenetic editing and by gene editing.

The present invention enables the selection of gRNAs which promote either gene editing or epigenetic editing of a target. In this manner, it is possible to choose to perform gene editing on gene targets which are not susceptible to epigenetic editing whilst simultaneously epigenetically targeting genes which are susceptible to epigenetic editing in a multiplexing approach.

In one aspect, a gRNA is capable of promoting epigenetic editing of a target. Epigenetic editing may be measured using methods known in the art. For example, as described in Example 2, the level of expression of a reporter gene may be measured as a model of epigenetic editing.

In one aspect, a gRNA is capable of promoting gene editing of a target. Gene editing may be measured using methods known in the art. For example, as described in Example 1, the level of non-homologous end joining may be measured as a model of gene editing.

An exemplary sequence of a genomic target site (i.e., protospacer and PAM) recognised by gRNAs for use in targeting the β2-microglobulin (B2M) gene includes:

5′-AGGGTAGGAGAGACTCACGCTGG-3′ (SEQ ID NO: 22)
|||||||||||||||||||||
3′-TCCCATCCTCTCTGAGTGCGACC-5′ (SEQ ID NO: 21)

The underlined nucleotides are the PAM.

In one aspect, the present invention provides gRNAs which target the β2-microglobulin gene region set forth in SEQ ID NO: 21 or SEQ ID NO: 22 above.

Examples of spacer sequences which may be used in gRNAs targeting the β2-microglobulin gene, and in particular the target site above, include:

(SEQ ID NO: 23)
GAGGGUAGGAGAGACUCACGC-21-nt

AGGGUAGGAGAGACUCACGC-20-nt (SEQ ID NO: 24)—This spacer sequence may be incorporated in a gRNA and may be used for gene editing of B2M when used in combination with an ETM as shown in Example 2.

(SEQ ID NO: 34)
GGGUAGGAGAGACUCACGC-19-nt
(SEQ ID NO: 26)
GGUAGGAGAGACUCACGC-18-nt
(SEQ ID NO: 27)
GUAGGAGAGACUCACGC-17-nt
(SEQ ID NO: 28)
UAGGAGAGACUCACGC-16-nt
(SEQ ID NO: 29)
AGGAGAGACUCACGC-15-nt
(SEQ ID NO: 30)
GGAGAGACUCACGC-14-nt
(SEQ ID NO: 31)
GAGAGACUCACGC-13-nt
(SEQ ID NO: 32)
AGAGACUCACGC-12-nt
(SEQ ID NO: 33)
GAGACUCACGC-11-nt
(SEQ ID NO: 25)
AGACUCACGC-10-nt

In some aspects, the spacer sequence comprises a “G” nucleotide at the 5′ end. This “G” may, for example, not be part of the targeting sequence and may be necessary when the promoter that drives its expression is a U6 promoter.

For example, the “G” at the 5′ end of SEQ ID NO: 23 is used herein to drive expression from a U6 promoter. Thus, it will be understood that if the spacer sequence in SEQ ID NO: 23 is not driven by a U6 promoter, the “G” at the 5′ end may not be necessary.

In some aspects the spacer sequences according to the present invention comprise a “G” nucleotide at the 5′ end.

Examples of a gRNA according to the present invention are:

(SEQ ID NO: 4479)
AGGGUAGGAGAGACUCACGCGUUUAAGAGCUAUGCUGGAAACAGCAUAGC
AAGUUUAAAUAAGGCUAGUCCGUUAUCAACUUGAAAAAGUGGCACCGAGU
CGGUGCUUUUUUU,
and
(SEQ ID NO: 4568)
AGGGUAGGAGAGACUCACGCGUUUUAGAGCUAGAAAUAGCAAGUUAAAAU
AAGGCUAGUCCGUUAUCAACUUGAAAAAGUGGCACCGAGUCGGUGCUUU
U,

which comprise the spacer sequence SEQ ID NO: 24 (underlined above).

Alternative gRNAs for epi-silencing of B2M may be found, e.g., in Amabile et al., supra.

For example, an alternative spacer sequence which may be used in a gRNA according to the present invention is:

(SEQ ID NO: 35)
GAGUAGCGCGAGCACAGCUA-20-nt

Examples of gRNA according to the present invention is:

(SEQ ID NO: 4480)
GAGUAGCGCGAGCACAGCUAGUUUAAGAGCUAUGCUGGAAACAGCAUAGC
AAGUUUAAAUAAGGCUAGUCCGUUAUCAACUUGAAAAAGUGGCACCGAGU
CGGUGCUUUUUUU,
and
(SEQ ID NO: 4569)
GAGUAGCGCGAGCACAGCUAGUUUUAGAGCUAGAAAUAGCAAGUUAAAAU
AAGGCUAGUCCGUUAUCAACUUGAAAAAGUGGCACCGAGUCGGUGCUUU
U.

which comprise the spacer sequence SEQ ID NO: 35 (underlined above).

Truncated spacer sequences based on SEQ ID NO: 35 suitable for use in gRNAs according to the present invention include:

(SEQ ID NO: 36)
AGUAGCGCGAGCACAGCUA-19-nt
(SEQ ID NO: 37)
GUAGCGCGAGCACAGCUA-18-nt
(SEQ ID NO: 38)
UAGCGCGAGCACAGCUA-17-nt
(SEQ ID NO: 39)
AGCGCGAGCACAGCUA-16-nt
(SEQ ID NO: 40)
GCGCGAGCACAGCUA-15-nt
(SEQ ID NO: 41)
CGCGAGCACAGCUA-14-nt
(SEQ ID NO: 42)
GCGAGCACAGCUA-13-nt
(SEQ ID NO: 43)
CGAGCACAGCUA-12-nt
(SEQ ID NO: 44)
GAGCACAGCUA-11-nt
(SEQ ID NO: 45)
AGCACAGCUA-10-nt

Another spacer sequence (H8) which may be used in a gRNA according to the present invention is:

(SEQ ID NO: 2780)
CAUCGGCGCCCUCCGAUCUG-20-nt

Examples of gRNAs having this spacer (underlined) are:

(SEQ ID NO: 4483)
CAUCGGCGCCCUCCGAUCUGGUUUAAGAGCUAUGCUGGAAACAGCAUAGC
AAGUUUAAAUAAGGCUAGUCCGUUAUCAACUUGAAAAAGUGGCACCGAGU
CGGUGCUUUUUUU,
and
(SEQ ID NO: 4570)
CAUCGGCGCCCUCCGAUCUGGUUUUAGAGCUAGAAAUAGCAAGUUAAAAU
AAGGCUAGUCCGUUAUCAACUUGAAAAAGUGGCACCGAGUCGGUGCUUU
U.

Truncated spacer sequences based on SEQ ID NO: 2780 suitable for use in gRNAs according to the present invention include:

(SEQ ID NO: 4484)
AUCGGCGCCCUCCGAUCUG-19-nt
(SEQ ID NO: 4485)
UCGGCGCCCUCCGAUCUG-18-nt
(SEQ ID NO: 4486)
CGGCGCCCUCCGAUCUG-17-nt
(SEQ ID NO: 4487)
GGCGCCCUCCGAUCUG-16-nt
(SEQ ID NO: 4488)
GCGCCCUCCGAUCUG-15-nt
(SEQ ID NO: 4489)
CGCCCUCCGAUCUG-14-nt
(SEQ ID NO: 4490)
GCCCUCCGAUCUG-13-nt
(SEQ ID NO: 4491)
CCCUCCGAUCUG-12-nt
(SEQ ID NO: 4492)
CCUCCGAUCUG-11-nt
(SEQ ID NO: 4493)
CUCCGAUCUG-10-nt

Another spacer sequence (H10) which may be used in a gRNA according to the present invention is:

(SEQ ID NO: 2863)
GCGGGCCACCAAGGAGAACU-20-nt

Examples of gRNAs having this spacer (underlined) are:

(SEQ ID NO: 4494)
GCGGGCCACCAAGGAGAACUGUUUAAGAGCUAUGCUGGAAACAGCAUAG
CAAGUUUAAAUAAGGCUAGUCCGUUAUCAACUUGAAAAAGUGGCACCGA
GUCGGUGCUUUUUUU,
and
(SEQ ID NO: 4571)
GCGGGCCACCAAGGAGAACUGUUUUAGAGCUAGAAAUAGCAAGUUAAAA
UAAGGCUAGUCCGUUAUCAACUUGAAAAAGUGGCACCGAGUCGGUGCUU
UU.

Truncated spacer sequences based on SEQ ID NO: 2863 suitable for use in gRNAs according to the present invention include:

(SEQ ID NO: 4495)
CGGGCCACCAAGGAGAACU-19-nt
(SEQ ID NO: 4496)
GGGCCACCAAGGAGAACU-18-nt
(SEQ ID NO: 4497)
GGCCACCAAGGAGAACU-17-nt
(SEQ ID NO: 4498)
GCCACCAAGGAGAACU-16-nt
(SEQ ID NO: 4499)
CCACCAAGGAGAACU-15-nt
(SEQ ID NO: 4500)
CACCAAGGAGAACU-14-nt
(SEQ ID NO: 4501)
ACCAAGGAGAACU-13-nt
(SEQ ID NO: 4502)
CCAAGGAGAACU-12-nt
(SEQ ID NO: 4503)
CAAGGAGAACU-11-nt
(SEQ ID NO: 4504)
AAGGAGAACU-10-nt

Another spacer sequence (H11) which may be used in a gRNA according to the present invention is:

(SEQ ID NO: 2778)
CGAUAAGCGUCAGAGCGCCG-20-nt

Examples of gRNAs having this spacer (underlined) are:

(SEQ ID NO: 4505)
CGAUAAGCGUCAGAGCGCCGGUUUAAGAGCUAUGCUGGAAACAGCAUAG
CAAGUUUAAAUAAGGCUAGUCCGUUAUCAACUUGAAAAAGUGGCACCGA
GUCGGUGCUUUUUUU,
and
(SEQ ID NO: 4572)
CGAUAAGCGUCAGAGCGCCGGUUUUAGAGCUAGAAAUAGCAAGUUAAAA
UAAGGCUAGUCCGUUAUCAACUUGAAAAAGUGGCACCGAGUCGGUGCUU
UU.

Truncated spacer sequences based on SEQ ID NO: 2778 suitable for use in gRNAs according to the present invention include:

(SEQ ID NO: 4506)
GAUAAGCGUCAGAGCGCCG-19-nt
(SEQ ID NO: 4507)
AUAAGCGUCAGAGCGCCG-18-nt
(SEQ ID NO: 4508)
UAAGCGUCAGAGCGCCG-17-nt
(SEQ ID NO: 4509)
AAGCGUCAGAGCGCCG-16-nt
(SEQ ID NO: 4510)
AGCGUCAGAGCGCCG-15-nt
(SEQ ID NO: 4511)
GCGUCAGAGCGCCG-14-nt
(SEQ ID NO: 4512)
CGUCAGAGCGCCG-13-nt
(SEQ ID NO: 4513)
GUCAGAGCGCCG-12-nt
(SEQ ID NO: 4514)
UCAGAGCGCCG-11-nt
(SEQ ID NO: 4515)
CAGAGCGCCG-10-nt

Another spacer sequence (H12) which may be used in a gRNA according to the present invention is:

(SEQ ID NO: 2801)
GAACGCGUGGAGGGGCGCUU-20-nt

Examples of gRNAs having this spacer (underlined) are:

(SEQ ID NO: 4516)
GAACGCGUGGAGGGGCGCUUGUUUAAGAGCUAUGCUGGAAACAGCAUAGCA
AGUUUAAAUAAGGCUAGUCCGUUAUCAACUUGAAAAAGUGGCACCGAGUCG
GUGCUUUUUUU,
and
(SEQ ID NO: 4573)
GAACGCGUGGAGGGGCGCUUGUUUUAGAGCUAGAAAUAGCAAGUUAAAAUA
AGGCUAGUCCGUUAUCAACUUGAAAAAGUGGCACCGAGUCGGUGCUUUU.

Truncated spacer sequences based on SEQ ID NO: 2801 suitable for use in gRNAs according to the present invention include:

(SEQ ID NO: 4517)
AACGCGUGGAGGGGCGCUU-19-nt
(SEQ ID NO: 4518)
ACGCGUGGAGGGGCGCUU-18-nt
(SEQ ID NO: 4519)
CGCGUGGAGGGGCGCUU-17-nt
(SEQ ID NO: 4520)
GCGUGGAGGGGCGCUU-16-nt
(SEQ ID NO: 4521)
CGUGGAGGGGCGCUU-15-nt
(SEQ ID NO: 4522)
GUGGAGGGGCGCUU-14-nt
(SEQ ID NO: 4523)
UGGAGGGGCGCUU-13-nt
(SEQ ID NO: 4524)
GGAGGGGCGCUU-12-nt
(SEQ ID NO: 4525)
GAGGGGCGCUU-11-nt
(SEQ ID NO: 4526)
AGGGGCGCUU-10-nt

An example of a spacer sequence for use in a gRNA targeting the TRAC gene, includes:

(SEQ ID NO: 46)
AGAGUCUCUCAGCUGGUACA

Examples of gRNAs having this spacer (underlined) are:

(SEQ ID NO: 4574)
AGAGUCUCUCAGCUGGUACAGUUUAAGAGCUAUGCUGGAAACAGCAUAGC
AAGUUUAAAUAAGGCUAGUCCGUUAUCAACUUGAAAAAGUGGCACCGAGU
CGGUGCUUUUUUU,
and
(SEQ ID NO: 4575)
AGAGUCUCUCAGCUGGUACAGUUUUAGAGCUAGAAAUAGCAAGUUAAAAU
AAGGCUAGUCCGUUAUCAACUUGAAAAAGUGGCACCGAGUCGGUGCUUU
U.

The present disclosure also provides variations of the above exemplified gRNAs in which the spacer sequences (those underlined) are truncated by, e.g., 1 to 9 (e.g., 3 to 9) nucleotides at the 5′ end. The present disclosure also provides gRNAs in which the spacers (full-length or truncated versions) described herein are linked to the above-exemplified tracr RNA (the portions of the above gRNAs, e.g., SEQ ID NOs: 4574 and 4575, that are not underlined).

In one aspect, the present invention provides a gRNA which comprises a spacer sequence which comprises or consists of a sequence set forth in any one of SEQ ID NOs: 23-46, 562-1076, 2778-4478, and 4553-4565, or a homologue thereof.

In one aspect, the present invention provides a gRNA which comprises a spacer sequence wherein the spacer sequence comprises or consists of a sequence set forth in any one of SEQ ID NOs: 23-46, 562-1076, 2778-4478, and 4553-4565 having one or more (such as two, or three, or four, or five) conservative substitutions. The spacer sequence comprising one or more conservative substitution(s) retains substantially the same activity as the spacer sequence having a sequence set forth in any one of SEQ ID NOs: 23-46, 562-1076, 2778-4478, and 4553-4565.

In one aspect, the present invention provides a gRNA which comprises a spacer sequence which comprises or consists of a sequence set forth in any one of SEQ ID NOs: 23-46, 562-1076, 2778-4478, and 4553-4565, or a fragment thereof.

Suitably, the spacer sequence may comprise or consist of a sequence set forth in any one of SEQ ID NO: 23-46, 562-1076, 2778-4478, and 4553-4565, and is 21 nucleotides in length or less (such as 20 nucleotides, such as 19 nucleotides, such as 18 nucleotides, such as 17 nucleotides, such as 16 nucleotides, such as 15 nucleotides, such as 14 nucleotides, such as 13 nucleotides, such as 12 nucleotides, such as 11 nucleotides, or such as 10 nucleotides).

In one aspect, the spacer sequence may comprise a sequence set forth in any one of SEQ ID NOs: 23-46, 562-1076, 2778-4478, and 4553-4565, or a fragment thereof that comprises or consists of 21 continuous nucleotides in length or less (such as 20 continuous nucleotides, such as 19 continuous nucleotides, such as 18 continuous nucleotides, such as 17 continuous nucleotides, such as 16 continuous nucleotides, such as 15 continuous nucleotides, such as 14 continuous nucleotides, such as continuous 13 nucleotides, such as 12 continuous nucleotides, such as 11 continuous nucleotides, or such as 10 continuous nucleotides) of SEQ ID NO: 23-46, 562-1076, 2778-4478, and 4553-4565. The fragment may be, e.g., a truncation of SEQ ID NO: 23-46, 562-1076, 2778-4478, and 4553-4565 from the 5′ end (i.e., nucleotides at the 5′ end are removed).

In some aspects, gRNA can be chemically modified. For example, chemical modification may increase the stability of the gRNA once administrated in a target cell as described for example in (Yin et al., Nat Biotechnol. 2017 December; 35(12):1179-1187). Such chemical modifications are known in the literature and can comprise but are not limited to locked nucleic acids (LNA), phosphorothioate modified oligonucleotides, 2′-O-methoxyethyl modified oligonucleotides, and 2′ O-methyl modified oligonucleotides.

In some aspects, the first three nucleosides and the last three nucleosides of a gRNA, regardless of the gRNA's length, are 2′-O-methyl modified nucleosides. In some aspects, the first three internucleoside linkages and the last three internucleoside linkages of a gRNA, regardless of the gRNA's length, are phosphorothioate linkages.

For gRNA sequences having the tracr RNA of Seq ID No: 4567 (which is 80 nucleotides in length), the tracr sequence portion of the full-length gRNA may be modified as follows (with nucleoside 1 being at the 5′ end of the tracr RNA sequence, and nucleoside 80 being at the 3′ end of the tracr RNA sequence):
nucleosides 1-8: unmodified RNA nucleosides,
nucleosides 9-20: 2′-O-Me modified nucleosides,
nucleosides 21-48: unmodified RNA nucleosides, and
nucleosides 49-80: 2′-O-Me modified nucleosides.
   In such a modified tracr sequence, the internucleoside linkages between nucleosides 77 and 78, 78 and 79, and 79 and 80 (i.e., the last three internucleoside linkages) may be phosphorothioate linkages. A spacer RNA may be attached at the 5′ end of this modified tracr sequence to form a full-length gRNA. In this full-length gRNA, the tracr portion of the gRNA sequence is modified as described above, and the spacer portion of the gRNA sequence is modified as follows:
the first three nucleosides of the spacer sequence are 2′-O-Me nucleosides, and
the first three internucleoside linkages are phosphorothioate linkages.
   The general schematic for this full-length gRNA is shown below, wherein lowercase letters represent 2′-O-Me nucleosides, capital letters represent unmodified RNA nucleosides, s represents a phosphorothioate linkage, each X independently represents an A, C, G, or U nucleoside, and each x represents a 2′-O-Me A, C, G, or U nucleoside:

(SEQ ID NO: 4638)
5′-xsxsxs[X₇-X₁₇]GUUUUAGAgcuagaaauagcAAGUUAAAAUAAG
GCUAGUCCGUUAUCAacuugaaaaaguggcaccgagucggugcususus
u-3′

More specifically, for gRNA sequences having full-length spacer RNAs (i.e., 20 nucleotides) and the tracr RNA of Seq ID No: 4567 (which is 80 nucleotides in length, for a gRNA of 100 nucleotides in length), the gRNA may be modified as follows (with nucleoside 1 being at the 5′ end of the oligonucleotide, and nucleotide 100 being at the 3′ end of the oligonucleotide):
nucleosides 1-3: 2′-O-Me modified nucleosides,
nucleosides 4-28: Unmodified RNA nucleosides,
nucleosides 29-40: 2′-O-Me modified nucleosides,
nucleosides 41-68: Unmodified RNA nucleosides, and
nucleosides 79-100: 2′-O-Me modified nucleosides.
   In such a modified gRNA, the internucleoside linkages between nucleosides 1 and 2, 2 and 3, 3 and 4, 97 and 98, 98 and 99, and 99 and 100 (i.e., the first three internucleoside linkages and the last three internucleoside linkages) may be phosphorothioate linkages. The remainder of the internucleoside linkages are phosphate linkages.

Similar modifications may be made to truncated gRNAs (e.g., a gRNA with a spacer that is 11 to 19 nucleotides). For example, the first three and the last three internucleoside linkages of the gRNA may be phosphorothioate linkages, and/or some or all of the nucleotides may be chemically modified, e.g., 2′-O-methyl nucleotides.

For example, the sequence of SEQ ID NO: 4568 can be modified as follows:

5′-asgsgsGUAGGAGAGACUCACGCGUUUUAGAgcuagaaauagcAAGU
UAAAAUAAGGCUAGUCCGUUAUCAacuugaaaaaguggcaccgagucggu
gcusususu-3′ (gRNA ID F4_20)

where:
N: RNA nucleosides; n: 2′-O-methyl nucleosides; s: phosphorothioate backbone modification between two nucleosides.

Another example is the modification of the sequence of SEQ ID NO: 4569

5′-gsasgsUAGCGCGAGCACAGCUAGUUUUAGAgcuagaaauagcAAGU
UAAAAUAAGGCUAGUCCGUUAUCAacuugaaaaaguggcaccgagucggu
gcusususu-3′ (gRNA ID C8_20)

Exemplary full-length modified gRNAs targeting B2M are shown below:

5′-csasusCGGCGCCCUCCGAUCUGGUUUUAGAgcuagaaauagcAAGU
UAAAAUAAGGCUAGUCCGUUAUCAacuugaaaaaguggcaccgagucggu
gcusususu-3′ (modified version of SEQ ID NO: 4570;
gRNA ID H8_20)
5′-gscsgsGGCCACCAAGGAGAACUGUUUUAGAgcuagaaauagcAAGU
UAAAAUAAGGCUAGUCCGUUAUCAacuugaaaaaguggcaccgagucggu
gcusususu-3′ (modified version of SEQ ID NO: 4571;
gRNA ID H10_20)
5′-csgsasUAAGCGUCAGAGCGCCGGUUUUAGAgcuagaaauagcAAGU
UAAAAUAAGGCUAGUCCGUUAUCAacuugaaaaaguggcaccgagucggu
gcusususu-3′ (modified version of SEQ ID NO: 4572;
gRNA ID H11_20)
5′-gsasasCGCGUGGAGGGGCGCUUGUUUUAGAgcuagaaauagcAAGU
UAAAAUAAGGCUAGUCCGUUAUCAacuugaaaaaguggcaccgagucggu
gcusususu-3′ (modified version of SEQ ID NO: 4573;
gRNA ID H12_20)

Exemplary truncated modified gRNAs targeting B2M are shown below:

(SEQ ID NO: 4576; gRNA ID H10_14)
5′-csascsCAAGGAGAACUGUUUUAGAgcuagaaauagcAAGUUAAAAU
AAGGCUAGUCCGUUAUCAacuugaaaaaguggcaccgagucggugcusus
usu-3′
(SEQ ID NO: 4577; gRNA ID H8_15)
5′-gscsgsCCCUCCGAUCUGGUUUUAGAgcuagaaauagcAAGUUAAAA
UAAGGCUAGUCCGUUAUCAacuugaaaaaguggcaccgagucggugcusu
susu-3′
(SEQ ID NO: 4578; gRNA ID C8_16)
5′-asgscsGCGAGCACAGCUAGUUUUAGAgcuagaaauagcAAGUUAAA
AUAAGGCUAGUCCGUUAUCAacuugaaaaaguggcaccgagucggugcus
ususu-3′
(SEQ ID NO: 4579; gRNA ID F4_16)
5′-usasgsGAGAGACUCACGCGUUUUAGAgcuagaaauagcAAGUUAAA
AUAAGGCUAGUCCGUUAUCAacuugaaaaaguggcaccgagucggugcus
ususu-3′

An exemplary full-length modified gRNA targeting TRAC is shown below:

5′-asgsasGUCUCUCAGCUGGUACAGUUUUAGAgcuagaaauagcAAGU
UAAAAUAAGGCUAGUCCGUUAUCAacuugaaaaaguggcaccgagucggu
gcusususu-3′ (modified version of SEQ ID NO: 4575)

Exemplary truncated modified gRNAs targeting TET2 are shown below:

(SEQ ID NO: 4580; gRNA ID sgRNA TE13_15)
5′-cscsgsUGCAGUGGCGCGGUUUUAGAgcuagaaauagcAAGUUAAAA
UAAGGCUAGUCCGUUAUCAacuugaaaaaguggcaccgagucggugcusu
susu-3′
(SEQ ID NO: 4581; gRNA ID sgRNA TE14_15)
5′-csgscsCGGCCUUUGUGCGUUUUAGAgcuagaaauagcAAGUUAAAA
UAAGGCUAGUCCGUUAUCAacuugaaaaaguggcaccgagucggugcusu
susu-3′
(SEQ ID NO: 4582; gRNA ID sgRNA TE19_15)
5′-gscsgsGGGCCGGCGUCUGUUUUAGAgcuagaaauagcAAGUUAAAA
UAAGGCUAGUCCGUUAUCAacuugaaaaaguggcaccgagucggugcusu
susu-3′
SEQ ID NO: 4583; gRNA ID sgRNA TE20_15)
5′-usgsasAUAUUGAUGCGGGUUUUAGAgcuagaaauagcAAGUUAAAA
UAAGGCUAGUCCGUUAUCAacuugaaaaaguggcaccgagucggugcusu
susu-3′

Exemplary truncated modified gRNAs targeting TGFBR2 are shown below:

(SEQ ID NO: 4584; gRNA ID sgRNA TG7_15)
5′-uscscsUCGCCAACAGCUGUUUUAGAgcuagaaauagcAAGUUAAAA
UAAGGCUAGUCCGUUAUCAacuugaaaaaguggcaccgagucggugcusu
susu-3′
(SEQ ID NO: 4585; gRNA ID sgRNA TG8_15)
5′-asgsusCACUCGCGCGCAGUUUUAGAgcuagaaauagcAAGUUAAAA
UAAGGCUAGUCCGUUAUCAacuugaaaaaguggcaccgagucggugcusu
susu-3′
(SEQ ID NO: 4586; gRNA ID sgRNA TG19_15)
5′-ascsusCCCGUAGCUGCAGUUUUAGAgcuagaaauagcAAGUUAAAA
UAAGGCUAGUCCGUUAUCAacuugaaaaaguggcaccgagucggugcusu
susu-3′
(SEQ ID NO: 4587; gRNA ID sgRNA TG20_15)
5′-usgsusUGGCCGCGUUCGGUUUUAGAgcuagaaauagcAAGUUAAAA
UAAGGCUAGUCCGUUAUCAacuugaaaaaguggcaccgagucggugcusu
susu-3′

Exemplary full-length modified gRNAs targeting TET2 are shown below:

(SEQ ID NO: 4588; gRNA ID TE1_20)
5′-gsgsasAUUAGCUCUGUAUCGGUGUUUUAGAgcuagaaauagcAAGUUAAAA
UAAGGCUAGUCCGUUAUCAacuugaaaaaguggcaccgagucggugcusususu-3′
(SEQ ID NO: 4589; gRNA ID TE2_20)
5′-asasasGUAAGGGCUCUUACGAGGUUUUAGAgcuagaaauagcAAGUUAAAA
UAAGGCUAGUCCGUUAUCAacuugaaaaaguggcaccgagucggugcusususu-3′
(SEQ ID NO: 4590; gRNA ID TE3_20)
5′-gsgscsGUCUCACAGAUUGAAAUGUUUUAGAgcuagaaauagcAAGUUAAAA
UAAGGCUAGUCCGUUAUCAacuugaaaaaguggcaccgagucggugcusususu-3′
(SEQ ID NO: 4591; gRNA ID TE4_20)
5′-csgsgsUCAAUUUCCCAGUUUGUGUUUUAGAgcuagaaauagcAAGUUAAAA
UAAGGCUAGUCCGUUAUCAacuugaaaaaguggcaccgagucggugcusususu-3′
(SEQ ID NO: 4592; gRNA ID TE5_20)
5′-asgscsGCUCCCCUGUUUCACCGGUUUUAGAgcuagaaauagcAAGUUAAAA
UAAGGCUAGUCCGUUAUCAacuugaaaaaguggcaccgagucggugcusususu-3′
(SEQ ID NO: 4593; gRNA ID TE6_20)
5′-csgscsGGGCAACGGGAUCUAAAGUUUUAGAgcuagaaauagcAAGUUAAAA
UAAGGCUAGUCCGUUAUCAacuugaaaaaguggcaccgagucggugcusususu-3′
(SEQ ID NO: 4594; gRNA ID TE7_20)
5′-csgscsAAGCGGAGGUGUGGUGCGUUUUAGAgcuagaaauagcAAGUUAAAA
UAAGGCUAGUCCGUUAUCAacuugaaaaaguggcaccgagucggugcusususu-3′
(SEQ ID NO: 4595; gRNA ID TE8_20)
5′-gsusgsCGGGUACACUCCGGAGGGUUUUAGAgcuagaaauagcAAGUUAAAA
UAAGGCUAGUCCGUUAUCAacuugaaaaaguggcaccgagucggugcusususu-3′
(SEQ ID NO: 4596; gRNA ID TE9_20)
5′-usgscsGCGGGACCUCGAAGUGGGUUUUAGAgcuagaaauagcAAGUUAAAA
UAAGGCUAGUCCGUUAUCAacuugaaaaaguggcaccgagucggugcusususu-3′
(SEQ ID NO: 4597; gRNA ID TE10_20)
5′-asgscsAGAGCAAGCGCGAAGGUGUUUUAGAgcuagaaauagcAAGUUAAAA
UAAGGCUAGUCCGUUAUCAacuugaaaaaguggcaccgagucggugcusususu-3′
(SEQ ID NO: 4598; gRNA ID TE11_20)
5′-usgscsAGCCCUCGGGAACCCCGGUUUUAGAgcuagaaauagcAAGUUAAAA
UAAGGCUAGUCCGUUAUCAacuugaaaaaguggcaccgagucggugcusususu-3′
(SEQ ID NO: 4599; gRNA ID TE12_20)
5′-gsusgsGUGCGCCCGGACCAGCGGUUUUAGAgcuagaaauagcAAGUUAAAA
UAAGGCUAGUCCGUUAUCAacuugaaaaaguggcaccgagucggugcusususu-3′
(SEQ ID NO: 4600; gRNA ID TE13_20)
5′-uscsasCGCCGUGCAGUGGCGCGGUUUUAGAgcuagaaauagcAAGUUAAAA
UAAGGCUAGUCCGUUAUCAacuugaaaaaguggcaccgagucggugcusususu-3′
(SEQ ID NO: 4601; gRNA ID TE14_20)
5′-gsgsusGCCGCCGGCCUUUGUGCGUUUUAGAgcuagaaauagcAAGUUAAAA
UAAGGCUAGUCCGUUAUCAacuugaaaaaguggcaccgagucggugcusususu-3′
(SEQ ID NO: 4602; gRNA ID TE15_20)
5′-gscsasCCGGGCGUCCAGCACAAGUUUUAGAgcuagaaauagcAAGUUAAAA
UAAGGCUAGUCCGUUAUCAacuugaaaaaguggcaccgagucggugcusususu-3′
(SEQ ID NO: 4603; gRNA ID TE16_20)
5′-asgsgsGAAUUAGCCCCCCGCACGUUUUAGAgcuagaaauagcAAGUUAAAA
UAAGGCUAGUCCGUUAUCAacuugaaaaaguggcaccgagucggugcusususu-3′
(SEQ ID NO: 4604; gRNA ID TE17_20)
5′-asgsusGGCAGCGGCGAGAGCUUGUUUUAGAgcuagaaauagcAAGUUAAAA
UAAGGCUAGUCCGUUAUCAacuugaaaaaguggcaccgagucggugcusususu-3′
(SEQ ID NO: 4605; gRNA ID TE18_20)
5′-ascsusUGCAUGCGAGCGGGACCGUUUUAGAgcuagaaauagcAAGUUAAAA
UAAGGCUAGUCCGUUAUCAacuugaaaaaguggcaccgagucggugcusususu-3′
(SEQ ID NO: 4606; gRNA ID TE19_20)
5′-ascsusCAGCGGGGCCGGCGUCUGUUUUAGAgcuagaaauagcAAGUUAAAA
UAAGGCUAGUCCGUUAUCAacuugaaaaaguggcaccgagucggugcusususu-3′
(SEQ ID NO: 4607; gRNA ID TE20_20)
5′-cscsusUAUGAAUAUUGAUGCGGGUUUUAGAgcuagaaauagcAAGUUAAAA
UAAGGCUAGUCCGUUAUCAacuugaaaaaguggcaccgagucggugcusususu-3′

Exemplary full-length modified gRNAs targeting TGFBR2 are shown below:

(SEQ ID NO: 4608; gRNA ID TG1_20)
5′-ususcsUUUAGGUCGAAGUCUAGGUUUUAGAgcuagaaauagcAAGUUAAAA
UAAGGCUAGUCCGUUAUCAacuugaaaaaguggcaccgagucggugcusususu-3′
(SEQ ID NO: 4609; gRNA ID TG2_20)
5′-gsusgsCUCGCGACUCAAUAGAUGUUUUAGAgcuagaaauagcAAGUUAAAA
UAAGGCUAGUCCGUUAUCAacuugaaaaaguggcaccgagucggugcusususu-3′
(SEQ ID NO: 4610; gRNA ID TG3_20)
5′-asascsGCAUCUCUAAAGCACCUGUUUUAGAgcuagaaauagcAAGUUAAAA
UAAGGCUAGUCCGUUAUCAacuugaaaaaguggcaccgagucggugcusususu-3′
(SEQ ID NO: 4611; gRNA ID TG4_20)
5′-csusgsAUCUACUAGGGAAAACGGUUUUAGAgcuagaaauagcAAGUUAAAA
UAAGGCUAGUCCGUUAUCAacuugaaaaaguggcaccgagucggugcusususu-3′
(SEQ ID NO: 4612; gRNA ID TG5_20)
5′-ususgsAGUAAAUACUUGGAGCGGUUUUAGAgcuagaaauagcAAGUUAAAA
UAAGGCUAGUCCGUUAUCAacuugaaaaaguggcaccgagucggugcusususu-3′
(SEQ ID NO: 4613; gRNA ID TG6_20)
5′-asgsusCGGCCAAAGCUCUCGGAGUUUUAGAgcuagaaauagcAAGUUAAAA
UAAGGCUAGUCCGUUAUCAacuugaaaaaguggcaccgagucggugcusususu-3′
(SEQ ID NO: 4614; gRNA ID TG7_20)
5′-gsasasACUCCUCGCCAACAGCUGUUUUAGAgcuagaaauagcAAGUUAAAA
UAAGGCUAGUCCGUUAUCAacuugaaaaaguggcaccgagucggugcusususu-3′
(SEQ ID NO: 4615; gRNA ID TG8_20)
5′-gsasgsUGAGUCACUCGCGCGCAGUUUUAGAgcuagaaauagcAAGUUAAAA
UAAGGCUAGUCCGUUAUCAacuugaaaaaguggcaccgagucggugcusususu-3′
(SEQ ID NO: 4616; gRNA ID TG9_20)
5′-csgscsGUGCACCCGCUCGGGACGUUUUAGAgcuagaaauagcAAGUUAAAA
UAAGGCUAGUCCGUUAUCAacuugaaaaaguggcaccgagucggugcusususu-3′
(SEQ ID NO: 4617; gRNA ID TG10_20)
5′-gsgsgsGCCUCCCCGCGCCUCGCGUUUUAGAgcuagaaauagcAAGUUAAAA
UAAGGCUAGUCCGUUAUCAacuugaaaaaguggcaccgagucggugcusususu-3′
(SEQ ID NO: 4618; gRNA ID TG11_20)
5′-usgsgsCGAGCGGGCGCCACAUCGUUUUAGAgcuagaaauagcAAGUUAAAA
UAAGGCUAGUCCGUUAUCAacuugaaaaaguggcaccgagucggugcusususu-3′
(SEQ ID NO: 4619; gRNA ID TG12_20)
5′-uscsgsGUCUAUGACGAGCAGCGGUUUUAGAgcuagaaauagcAAGUUAAAA
UAAGGCUAGUCCGUUAUCAacuugaaaaaguggcaccgagucggugcusususu-3′
(SEQ ID NO: 4620; gRNA ID TG13_20)
5′-cscsusGAGCAGCCCCCGACCCAGUUUUAGAgcuagaaauagcAAGUUAAAA
UAAGGCUAGUCCGUUAUCAacuugaaaaaguggcaccgagucggugcusususu-3′
(SEQ ID NO: 4621; gRNA ID TG14_20)
5′-gsgsasCGAUGUGCAGCGGCCACGUUUUAGAgcuagaaauagcAAGUUAAAA
UAAGGCUAGUCCGUUAUCAacuugaaaaaguggcaccgagucggugcusususu-3′
(SEQ ID NO: 4622; gRNA ID TG15_20)
5′-usgscsUGGCGAUACGCGUCCACGUUUUAGAgcuagaaauagcAAGUUAAAA
UAAGGCUAGUCCGUUAUCAacuugaaaaaguggcaccgagucggugcusususu-3′
(SEQ ID NO: 4623; gRNA ID TG16_20)
5′-asascsGUGCGGUGGGAUCGUGCGUUUUAGAgcuagaaauagcAAGUUAAAA
UAAGGCUAGUCCGUUAUCAacuugaaaaaguggcaccgagucggugcusususu-3′
(SEQ ID NO: 4624; gRNA ID TG17_20)
5′-gsascsUGUCAAGCGCAGCGGAGGUUUUAGAgcuagaaauagcAAGUUAAAA
UAAGGCUAGUCCGUUAUCAacuugaaaaaguggcaccgagucggugcusususu-3′
(SEQ ID NO: 4625; gRNA ID TG18_20)
5′-csususUCCUCGUUUCCGCCCGGGUUUUAGAgcuagaaauagcAAGUUAAAA
UAAGGCUAGUCCGUUAUCAacuugaaaaaguggcaccgagucggugcusususu-3′
(SEQ ID NO: 4626; gRNA ID TG19_20)
5′-gscscsCGACUCCCGUAGCUGCAGUUUUAGAgcuagaaauagcAAGUUAAAA
UAAGGCUAGUCCGUUAUCAacuugaaaaaguggcaccgagucggugcusususu-3′
(SEQ ID NO: 4627; gRNA ID TG20_20)
5′-csgsusUGUGUUGGCCGCGUUCGGUUUUAGAgcuagaaauagcAAGUUAAAA
UAAGGCUAGUCCGUUAUCAacuugaaaaaguggcaccgagucggugcusususu-3′

An exemplary full-length modified gRNA targeting GFP is shown below:

(SEQ ID NO: 4628; gRNA ID GFP1)
5′-csuscsCUCGCCCUUGCUCACCAGUUUUAGAgcuagaaauagcAAGU
UAAAAUAAGGCUAGUCCGUUAUCAacuugaaaaaguggcaccgagucggu
gcusususu-3′

In some aspects, the present invention utilizes two or more gRNAs.

Suitably, the two or more gRNAs may target the ETM (e.g., ETR) to different target genes. Suitably, the two or more gRNAs may comprise spacer sequences of different lengths. For example, the spacer sequences of different lengths may target the endonuclease of the ETM (e.g., ETR) to different target genes.

In some aspects, a two or more gRNAs may target the same target gene. For example, it may be beneficial to target the same gene with two gRNAs for optimal epigenetic modification e.g., epigenetic silencing.

In one aspect, at least one of the at least two gRNAs comprises a spacer sequence which is 18, 19 or 20 nucleotides in length.

In one aspect, at least one of the at least two gRNAs comprises a spacer sequence which is less than or equal to 17 nucleotides in length, such as 16 nucleotides in length, 15 nucleotides in length, such as 14 nucleotides in length, such as less than 13 nucleotides in length, such as 12 nucleotides in length, such as 11 nucleotides in length, or such as 10 nucleotides in length.

### Multiplexing—Modifying Multiple Genes in the Same Cell

The present invention relates to the development of a combined gene editing and epigenetic editing strategy to modify the expression and/or activity of multiple target genes within the same cell. In particular, it may exploit an ETM (e.g., ETR) which comprises an epigenetic effector domain and an endonuclease and gRNAs comprising spacer sequences of different lengths to promote epigenetic editing of one or more genes and genetic editing of another gene.

As used herein “modify the expression and/or activity” refers to increasing or decreasing (e.g., decreasing) the expression and/or activity of a target gene.

In one aspect, transcription and/or expression of a target gene may be repressed.

In one aspect, a target gene may be silenced.

In one aspect, a target gene may be enhanced. In other words, the expression of the target gene may be increased. For example, the expression of an endogenous target gene may be increased.

In another example, an endogenous target (e.g., gene) may be modified (e.g., mutated) by gene editing and the expression of the modified target (e.g., gene) may be increased.

The effect of an ETM or combination of ETMs may be studied by comparing the transcription or expression of the target gene, for example a gene endogenous to a cell, in the presence and absence of the ETM or combination of ETMs. Methods of analysing transcription or expression of a gene are well known in the art.

The effect of an ETM or a combination of ETM and gRNAs may also be studied using a model system wherein the expression of a reporter gene, for example a gene encoding a fluorescent protein, is monitored. Suitable methods for monitoring expression of such reporter genes include flow cytometry, fluorescence-activated cell sorting (FACS) and fluorescence microscopy.

For example, a population of cells may be transfected with a vector which harbours a reporter gene. The vector may be constructed such that the reporter gene is expressed when the vector transfects a cell. Suitable reporter genes include genes encoding fluorescent proteins, for example green, yellow, cherry, cyan or orange fluorescent proteins. In addition, the population of cells may be transfected with vectors encoding the ETMs of interest and/or gRNAs. Subsequently, the number of cells expressing and not-expressing the reporter gene, as well as the level of expression of the reporter gene may be quantified using a suitable technique, such as FACS. The level of reporter gene expression may then be compared in the presence and absence of the ETM and/or gRNAs.

Methods for determining the transcription of a gene, for example the target of an ETM, are known in the art. Suitable methods include reverse transcription PCR and Northern blot-based approaches. In addition to the methods for determining the transcription of a gene, methods for determining the expression of a gene are known in the art. Suitable additional methods include Western blot-based or flow cytometry approaches.

### Target Gene Transcription and Expression

In some aspects, the product (e.g., ETM and/or gRNA) according to the present invention is used in a method which represses transcription and/or expression of at least one target gene. Suitably, the target gene may be an endogenous gene.

In one aspect, the target gene transcription and/or expression is repressed by epigenetic editing. In one aspect, the target gene transcription and/or expression is repressed by gene editing.

In some aspects, the product (e.g., ETM and/or gRNA) according to the present invention is used in a method which represses transcription and/or expression of at least two target genes. Suitably, at least one or both of the target genes may be an endogenous gene.

In one aspect, transcription and/or expression of only one gene is repressed by gene editing.

Following administration of an ETM (e.g., ETR) of the invention (e.g., with suitable gRNA(s)), the level of transcription or expression of the target gene may be reduced by, for example, at least 30%, 40%, 50%, 60%, 70%, 80%, 90%, 95%, 99% or 100% compared to the level of transcription or expression in the absence of the ETM (e.g., ETR).

In some aspects, the product (e.g., ETM and/or gRNA) according to the present invention is used in a method which silences at least one target gene. Suitably, the target gene may be an endogenous gene. Suitably, the target gene may be an exogenous gene, such as a viral gene.

In one aspect, the target gene is silenced by epigenetic editing. In one aspect, the target gene is silenced by gene editing.

In some aspects, the product (e.g., ETM and/or gRNA) according to the present invention is used in a method which silences at least two target genes. Suitably, at least one or both of the target genes may be an endogenous gene.

In one aspect, only one gene is silenced by gene editing.

Without wishing to be bound by theory, restricting gene editing activity to one gene may reduce the potential for undesirable genomic translocations.

By “silencing a target gene”, it is to be understood that the expression of the target gene is reduced to an extent sufficient to achieve a desired effect. The reduced expression may be sufficient to achieve a therapeutically relevant effect, such as the prevention or treatment of a disease. For example, a dysfunctional target gene which gives rise to a disease may be repressed to an extent that there is either no expression of the target gene, or the residual level of expression of the target gene is sufficiently low to ameliorate or prevent the disease state. Furthermore, the reduced expression may allow for purification of the cells harbouring gene silencing.

The reduced expression may be sufficient to enable investigations to be performed into the gene's function by studying cells reduced in or lacking that function.

The repression of the target gene may occur, e.g., following transient delivery or expression of the ETMs (e.g., ETRs) of the present invention to or in a cell (e.g., along with suitable gRNAs).

### Enhancing a Target Gene

By “enhancing a target gene”, it is to be understood that the expression of the target gene is increased to an extent sufficient to achieve a desired effect. The increased expression may be sufficient to achieve a therapeutically relevant effect, such as the prevention or treatment of a disease. For example, a dysfunctional target gene which gives rise to a disease may be enhanced to an extent that there is sufficient expression of the target gene to ameliorate or prevent the disease state. Alternatively, increased expression of the target gene may compensate for the dysfunctional activity of a disease-related gene. Furthermore, increased expression of the target gene may allow for selection of the cells expressing de novo that specific target gene.

Following administration of an ETM of the invention (e.g., with suitable gRNA(s)), the level of transcription or expression of the target gene may be increased by, for example, at least 50%, 60%, 70%, 80%, 90%, 95%, 99%, 100%, 200%, 300%, 400% or 500% compared to the level of transcription or expression in the absence of the ETM.

The enhancement of the target gene may occur, e.g., following transient delivery or expression of the ETMs of the present invention to or in a cell (along with suitable gRNAs).

### Transient Expression

By “transient expression”, it is to be understood that the expression of the ETM (e.g., ETR) is not stable over a prolonged period of time. For example, the polynucleotide encoding the ETM (e.g., ETR) may not integrate into the host genome. More specifically, transient expression may be expression which is substantially lost within 20 weeks following introduction of the polynucleotide encoding the ETM (e.g., ETR) into the cell. For example, expression may be substantially lost within 12, 6, 4, or 2 weeks following introduction of the polynucleotide encoding the ETM (e.g., ETR) into the cell.

Similarly, by “transient delivery”, it is to be understood that the ETM (e.g., ETR) substantially does not remain in the cell (i.e., is substantially lost by the cell) over a prolonged period of time. More specifically, transient delivery may result in the ETM (e.g., ETR) being substantially lost by the cell within 20 weeks following introduction of the ETM (e.g., ETR) into the cell. For example, the ETM (e.g., ETR) may be substantially lost within 12, 6, 4, or 2 weeks following introduction of the ETM (e.g., ETR) into the cell.

In one aspect, the ETM and/or gRNA may be delivered transiently. Transient delivery may result in permanent changes for example; transient delivery of the ETM and/or gRNA may lead to DNA methylation of a repressive regulatory element which in turn may lead to gene activation (e.g., given the stability of this epigenetic modification, permanent gene activation).

The target gene may, for example, be repressed, silenced, or enhanced permanently. By “permanent repression”, “permanent silencing” or “permanent enhancement” of a target gene, it is to be understood that transcription or expression of the target gene is reduced or increased (e.g., reduced or increased by at least 60%, at least 70%, at least 80%, at least 90% or 100%) compared to the level of transcription or expression in the absence of the ETM (e.g., ETR) for at least 2 months, 6 months, 1 year, 2 year or the entire lifetime of the cell/organism. For example, a permanently repressed, silenced, or enhanced target gene may remain repressed, silenced, or enhanced for the remainder of the cell's life.

In one aspect, the ETM and/or gRNA is stably expressed. For example, stable expression may be required to achieve permanent gene activation of some targets. The target gene may, for example, remain repressed, silenced, or enhanced in the progeny of the cell to which the product of the invention has been administered (i.e., the repression, silencing or enhancement of the target gene is inherited by the cell's progeny). For example, the ETM (e.g., ETR) and gRNAs of the invention may be administered to a stem cell (e.g., a haematopoietic stem cell) to repress or silence a target gene in a stem cell and also in the stem cell's progeny, which may include cells that have differentiated from the stem cell.

### Target Gene

The target gene may, for example, give rise to a therapeutic effect when modified, e.g., repressed or silenced.

The products, of the present invention may be used to modify, e.g., repress or silence, genes without CpG islands (CGI). Genes without CGI include: TRAC; TRBC; PDCD1; TIM-3; TIGIT; LAG3; CTLA4; AAVS1 and CCR5.

For example, targeting genes, such as genes without a GI, may:
produce allogenic products (e.g., by targeting TRAC and/or TRABC); alter resistance to an immunosuppressive tumour microenvironment (e.g., by targeting of PDCD1, TIM-3, TIGIT, LAG3 and/or CTLA4); and/or
allow CAR/transgenic TCR integration in a safe site (e.g., by targeting of AAVS1 and/or CCR5).

In one aspect, the present invention provides gRNAs which target a sequence set forth in any one of SEQ ID NOs: 47 to 561.

By way of example, target genes without CGI islands and exemplary gRNAs suitable for targeting said genes are presented in Table 1 below (SEQ: SEQ ID NO).

TABLE 1
Target genes without CGI islands and exemplary gRNAs

| Target gene | Exemplary target regions | | Exemplary gRNA spacer | |
|---|---|---|---|---|
| (no CGI) | (including PAM) | SEQ | sequence | SEQ |
| TRAC | GATTAAACCCGGCCACTTTCAGG | 47 | GAUUAAACCCGGCCACUUUC | 562 |
| | CGTCATGAGCAGATTAAACCCGG | 48 | CGUCAUGAGCAGAUUAAACC | 563 |
| | CTCGACCAGCTTGACATCACAGG | 49 | CUCGACCAGCUUGACAUCAC | 564 |
| | AAGTTCCTGTGATGTCAAGCTGG | 50 | AAGUUCCUGUGAUGUCAAGC | 565 |
| | TTCGGAACCCAATCACTGACAGG | 51 | UUCGGAACCCAAUCACUGAC | 566 |
| | TCAGGGTTCTGGATATCTGTGGG | 52 | UCAGGGUUCUGGAUAUCUGU | 567 |
| | GAGAATCAAAATCGGTGAATAGG | 53 | GAGAAUCAAAAUCGGUGAAU | 568 |
| | CTCTCAGCTGGTACACGGCAGGG | 54 | CUCUCAGCUGGUACACGGCA | 569 |
| | TAAACCCGGCCACTTTCAGGAGG | 55 | UAAACCCGGCCACUUUCAGG | 570 |
| | GGTAAGACAGGGGTCTAGCCTGG | 56 | GGUAAGACAGGGGUCUAGCC | 571 |
| | TGGATTTAGAGTCTCTCAGCTGG | 57 | UGGAUUUAGAGUCUCUCAGC | 572 |
| | GCACCAAAGCTGCCCTTACCTGG | 58 | GCACCAAAGCUGCCCUUACC | 573 |
| | GTAAGACAGGGGTCTAGCCTGGG | 59 | GUAAGACAGGGGUCUAGCCU | 574 |
| | ACCCGGCCACTTTCAGGAGGAGG | 60 | ACCCGGCCACUUUCAGGAGG | 575 |
| | TCTCTCAGCTGGTACACGGCAGG | 61 | UCUCUCAGCUGGUACACGGC | 576 |
| | GTCGAGAAAAGCTTTGAAACAGG | 62 | GUCGAGAAAAGCUUUGAAAC | 577 |
| | ACACGGCAGGGTCAGGGTTCTGG | 63 | ACACGGCAGGGUCAGGGUUC | 578 |
| | CTGGATATCTGTGGGACAAGAGG | 64 | CUGGAUAUCUGUGGGACAAG | 579 |
| | CCGAATCCTCCTCCTGAAAGTGG | 65 | CCGAAUCCUCCUCCUGAAAG | 580 |
| | AAAGTCAGATTTGTTGCTCCAGG | 66 | AAAGUCAGAUUUGUUGCUCC | 581 |
| | GCTGGTACACGGCAGGGTCAGGG | 67 | GCUGGUACACGGCAGGGUCA | 582 |
| | TGTGCTAGACATGAGGTCTATGG | 68 | UGUGCUAGACAUGAGGUCUA | 583 |
| | ACAAAACTGTGCTAGACATGAGG | 69 | ACAAAACUGUGCUAGACAUG | 584 |
| | CTGACAGGTTTTGAAAGTTTAGG | 70 | CUGACAGGUUUUGAAAGUUU | 585 |
| | ATCCTCCTCCTGAAAGTGGCCGG | 71 | AUCCUCCUCCUGAAAGUGGC | 586 |
| | TAGGCAGACAGACTTGTCACTGG | 72 | UAGGCAGACAGACUUGUCAC | 587 |
| | AGCTTTGAAACAGGTAAGACAGG | 73 | AGCUUUGAAACAGGUAAGAC | 588 |
| | AAGCTGCCCTTACCTGGGCTGGG | 74 | AAGCUGCCCUUACCUGGGCU | 589 |
| | TTCAAAACCTGTCAGTGATTGGG | 75 | UUCAAAACCUGUCAGUGAUU | 590 |
| | TCAAGGCCCCTCACCTCAGCTGG | 76 | UCAAGGCCCCUCACCUCAGC | 591 |
| | GCTTTGAAACAGGTAAGACAGGG | 77 | GCUUUGAAACAGGUAAGACA | 592 |
| | GTCAGGGTTCTGGATATCTGTGG | 78 | GUCAGGGUUCUGGAUAUCUG | 593 |
| | CTTCAAGAGCAACAGTGCTGTGG | 79 | CUUCAAGAGCAACAGUGCUG | 594 |
| | AAAGCTGCCCTTACCTGGGCTGG | 80 | AAAGCUGCCCUUACCUGGGC | 595 |
| | ATCTGTGGGACAAGAGGATCAGG | 81 | AUCUGUGGGACAAGAGGAUC | 596 |
| | TTAATCTGCTCATGACGCTGCGG | 82 | UUAAUCUGCUCAUGACGCUG | 597 |
| | AGCCCAGGTAAGGGCAGCTTTGG | 83 | AGCCCAGGUAAGGGCAGCUU | 598 |
| | CTGCGGCTGTGGTCCAGCTGAGG | 84 | CUGCGGCUGUGGUCCAGCUG | 599 |
| | TGCTCATGACGCTGCGGCTGTGG | 85 | UGCUCAUGACGCUGCGGCUG | 600 |
| | CATCACAGGAACTTTCTAAAAGG | 86 | CAUCACAGGAACUUUCUAAA | 601 |
| | TCTGTGGGACAAGAGGATCAGGG | 87 | UCUGUGGGACAAGAGGAUCA | 602 |
| | TTCGTATCTGTAAAACCAAGAGG | 88 | UUCGUAUCUGUAAAACCAAG | 603 |
| | AGAGTCTCTCAGCTGGTACACGG | 89 | AGAGUCUCUCAGCUGGUACA | 604 |
| | AGGTGAGGGGCCTTGAAGCTGGG | 90 | AGGUGAGGGGCCUUGAAGCU | 605 |
| | TTCTTCCCCAGCCCAGGTAAGGG | 91 | UUCUUCCCCAGCCCAGGUAA | 606 |
| | CACCAAAGCTGCCCTTACCTGGG | 92 | CACCAAAGCUGCCCUUACCU | 607 |
| | GAGGTGAGGGGCCTTGAAGCTGG | 93 | GAGGUGAGGGGCCUUGAAGC | 608 |
| | TCCTCCTCCTGAAAGTGGCCGGG | 94 | UCCUCCUCCUGAAAGUGGCC | 609 |
| | AGCTGGTACACGGCAGGGTCAGG | 95 | AGCUGGUACACGGCAGGGUC | 610 |
| | AACAAATGTGTCACAAAGTAAGG | 96 | AACAAAUGUGUCACAAAGUA | 611 |
| TRBC1 | AGGAAGGGCTTACTTACCCGAGG | 97 | AGGAAGGGCUUACUUACCCG | 612 |
| (ENST00000633705) | TCAAACACAGCGACCTCGGGTGG | 98 | UCAAACACAGCGACCUCGGG | 613 |
| | CGGGTGGGAACACCTTGTTCAGG | 99 | CGGGUGGGAACACCUUGUUC | 614 |
| | GTAGGACACTGTTGGCACGGAGG | 100 | GUAGGACACUGUUGGCACGG | 615 |
| | CACCCAGATCGTCAGCGCCGAGG | 101 | CACCCAGAUCGUCAGCGCCG | 616 |
| | ATCGTCAGCGCCGAGGCCTGGGG | 102 | AUCGUCAGCGCCGAGGCCUG | 617 |
| | AGTCCAGTTCTACGGGCTCTCGG | 103 | AGUCCAGUUCUACGGGCUCU | 618 |
| | GACGGGTTTGGCCCTATCCTGGG | 104 | GACGGGUUUGGCCCUAUCCU | 619 |
| | TGACGGGTTTGGCCCTATCCTGG | 105 | UGACGGGUUUGGCCCUAUCC | 620 |
| | GAACAAGGTGTTCCCACCCGAGG | 106 | GAACAAGGUGUUCCCACCCG | 621 |
| | TCTCCGAGAGCCCGTAGAACTGG | 107 | UCUCCGAGAGCCCGUAGAAC | 622 |
| | GGCTCTCGGAGAATGACGAGTGG | 108 | GGCUCUCGGAGAAUGACGAG | 623 |
| | AGACAGGACCCCTTGCTGGTAGG | 109 | AGACAGGACCCCUUGCUGGU | 624 |
| | GGCGCTGACGATCTGGGTGACGG | 110 | GGCGCUGACGAUCUGGGUGA | 625 |
| | CAAACACAGCGACCTCGGGTGGG | 111 | CAAACACAGCGACCUCGGGU | 626 |
| | TGACAGCGGAAGTGGTTGCGGGG | 112 | UGACAGCGGAAGUGGUUGCG | 627 |
| | TGACGAGTGGACCCAGGATAGGG | 113 | UGACGAGUGGACCCAGGAUA | 628 |
| | CGCCCTTGTGTTGATGGCCATGG | 114 | CGCCCUUGUGUUGAUGGCCA | 629 |
| | ATGACGAGTGGACCCAGGATAGG | 115 | AUGACGAGUGGACCCAGGAU | 630 |
| | CTTTCCAGAGGACCTGAACAAGG | 116 | CUUUCCAGAGGACCUGAACA | 631 |
| | GGCCTCGGCGCTGACGATCTGGG | 117 | GGCCUCGGCGCUGACGAUCU | 632 |
| | CGCTGTCAAGTCCAGTTCTACGG | 118 | CGCUGUCAAGUCCAGUUCUA | 633 |
| | GGTCAGCGCCCTTGTGTTGATGG | 119 | GGUCAGCGCCCUUGUGUUGA | 634 |
| | AACACCTTGTTCAGGTCCTCTGG | 120 | AACACCUUGUUCAGGUCCUC | 635 |
| | TTGACAGCGGAAGTGGTTGCGGG | 121 | UUGACAGCGGAAGUGGUUGC | 636 |
| | GACGATCTGGGTGACGGGTTTGG | 122 | GACGAUCUGGGUGACGGGUU | 637 |
| | GACCAGCACAGCATACAGGGTGG | 123 | GACCAGCACAGCAUACAGGG | 638 |
| | TCCCTAGCAGGATCTCATAGAGG | 124 | UCCCUAGCAGGAUCUCAUAG | 639 |
| | TGTTGATGGCCATGGTAAGCAGG | 125 | UGUUGAUGGCCAUGGUAAGC | 640 |
| | CTGGTAGGACACTGTTGGCACGG | 126 | CUGGUAGGACACUGUUGGCA | 641 |
| | AGGCCTCGGCGCTGACGATCTGG | 127 | AGGCCUCGGCGCUGACGAUC | 642 |
| | CGTAGAACTGGACTTGACAGCGG | 128 | CGUAGAACUGGACUUGACAG | 643 |
| | CCAACAGTGTCCTACCAGCAAGG | 129 | CCAACAGUGUCCUACCAGCA | 644 |
| | TGAGGGTCTCGGCCACCTTCTGG | 130 | UGAGGGUCUCGGCCACCUUC | 645 |
| | GTATCTGGAGTCATTGAGGGCGG | 131 | GUAUCUGGAGUCAUUGAGGG | 646 |
| | TATCTGGAGTCATTGAGGGCGGG | 132 | UAUCUGGAGUCAUUGAGGGC | 647 |
| | GGCTCAAACACAGCGACCTCGGG | 133 | GGCUCAAACACAGCGACCUC | 648 |
| | GGCCACCCTGTATGCTGTGCTGG | 134 | GGCCACCCUGUAUGCUGUGC | 649 |
| | GCGGCTGCTCAGGCAGTATCTGG | 135 | GCGGCUGCUCAGGCAGUAUC | 650 |
| | TAGCAGGATCTCATAGAGGATGG | 136 | UAGCAGGAUCUCAUAGAGGA | 651 |
| | CTTGTTCAGGTCCTCTGGAAAGG | 137 | CUUGUUCAGGUCCUCUGGAA | 652 |
| | GTTGCGGGGGTTCTGCCAGAAGG | 138 | GUUGCGGGGGUUCUGCCAGA | 653 |
| | TCAGACTGTGGCTTTACCTCGGG | 139 | UCAGACUGUGGCUUUACCUC | 654 |
| | CTTGACAGCGGAAGTGGTTGCGG | 140 | CUUGACAGCGGAAGUGGUUG | 655 |
| | GCTGTCAAGTCCAGTTCTACGGG | 141 | GCUGUCAAGUCCAGUUCUAC | 656 |
| | CAGCTCAGCTCCACGTGGTCAGG | 142 | CAGCUCAGCUCCACGUGGUC | 657 |
| | CAACAGTGTCCTACCAGCAAGGG | 143 | CAACAGUGUCCUACCAGCAA | 658 |
| | AGATCGTCAGCGCCGAGGCCTGG | 144 | AGAUCGUCAGCGCCGAGGCC | 659 |
| | GATCGTCAGCGCCGAGGCCTGGG | 145 | GAUCGUCAGCGCCGAGGCCU | 660 |
| | AACAGTGTCCTACCAGCAAGGGG | 146 | AACAGUGUCCUACCAGCAAG | 661 |
| TRBC2 | GACCAGCACGGCATACAAGGTGG | 147 | GACCAGCACGGCAUACAAGG | 662 |
| (ENST00000466254) | CACCCAGATCGTCAGCGCCGAGG | 148 | CACCCAGAUCGUCAGCGCCG | 663 |
| | ATCGTCAGCGCCGAGGCCTGGGG | 149 | AUCGUCAGCGCCGAGGCCUG | 664 |
| | AGTCCAGTTCTACGGGCTCTCGG | 150 | AGUCCAGUUCUACGGGCUCU | 665 |
| | ACTGACCAGCACGGCATACAAGG | 151 | ACUGACCAGCACGGCAUACA | 666 |
| | AGGAGAGACTCACTTACCGGAGG | 152 | AGGAGAGACUCACUUACCGG | 667 |
| | TCTCCGAGAGCCCGTAGAACTGG | 153 | UCUCCGAGAGCCCGUAGAAC | 668 |
| | GGCTCTCGGAGAATGACGAGTGG | 154 | GGCUCUCGGAGAAUGACGAG | 669 |
| | GGCCACCTTGTATGCCGTGCTGG | 155 | GGCCACCUUGUAUGCCGUGC | 670 |
| | TACCATGGCCATCAGCACGAGGG | 156 | UACCAUGGCCAUCAGCACGA | 671 |
| | TCAACAGAGTCTTACCAGCAAGG | 157 | UCAACAGAGUCUUACCAGCA | 672 |
| | TGACAGCGGAAGTGGTTGCGGGG | 158 | UGACAGCGGAAGUGGUUGCG | 673 |
| | CTATGAGATCTTGCTAGGGAAGG | 159 | CUAUGAGAUCUUGCUAGGGA | 674 |
| | TGACGAGTGGACCCAGGATAGGG | 160 | UGACGAGUGGACCCAGGAUA | 675 |
| | ATGACGAGTGGACCCAGGATAGG | 161 | AUGACGAGUGGACCCAGGAU | 676 |
| | GACAGGTTTGGCCCTATCCTGGG | 162 | GACAGGUUUGGCCCUAUCCU | 677 |
| | TGACAGGTTTGGCCCTATCCTGG | 163 | UGACAGGUUUGGCCCUAUCC | 678 |
| | GGCCTCGGCGCTGACGATCTGGG | 164 | GGCCUCGGCGCUGACGAUCU | 679 |
| | CGCTGTCAAGTCCAGTTCTACGG | 165 | CGCUGUCAAGUCCAGUUCUA | 680 |
| | GGCTCAAACACAGCGACCTTGGG | 166 | GGCUCAAACACAGCGACCUU | 681 |
| | TTGACAGCGGAAGTGGTTGCGGG | 167 | UUGACAGCGGAAGUGGUUGC | 682 |
| | TGGGTGGGAACACGTTTTTCAGG | 168 | UGGGUGGGAACACGUUUUUC | 683 |
| | TTACCATGGCCATCAGCACGAGG | 169 | UUACCAUGGCCAUCAGCACG | 684 |
| | CAAACACAGCGACCTTGGGTGGG | 170 | CAAACACAGCGACCUUGGGU | 685 |
| | TCAAACACAGCGACCTTGGGTGG | 171 | UCAAACACAGCGACCUUGGG | 686 |
| | ATGGTTTTGGAGCTAGCCTCTGG | 172 | AUGGUUUUGGAGCUAGCCUC | 687 |
| | CAACAGAGTCTTACCAGCAAGGG | 173 | CAACAGAGUCUUACCAGCAA | 688 |
| | AGGCCTCGGCGCTGACGATCTGG | 174 | AGGCCUCGGCGCUGACGAUC | 689 |
| | CGTAGAACTGGACTTGACAGCGG | 175 | CGUAGAACUGGACUUGACAG | 690 |
| | CACGAGGGCACTGACCAGCACGG | 176 | CACGAGGGCACUGACCAGCA | 691 |
| | TCGTGCTGATGGCCATGGTAAGG | 177 | UCGUGCUGAUGGCCAUGGUA | 692 |
| | AACAGAGTCTTACCAGCAAGGGG | 178 | AACAGAGUCUUACCAGCAAG | 693 |
| | TGAGGGTCTCGGCCACCTTCTGG | 179 | UGAGGGUCUCGGCCACCUUC | 694 |
| | TCCCTAGCAAGATCTCATAGAGG | 180 | UCCCUAGCAAGAUCUCAUAG | 695 |
| | GTATCTGGAGTCATTGAGGGCGG | 181 | GUAUCUGGAGUCAUUGAGGG | 696 |
| | TATCTGGAGTCATTGAGGGCGGG | 182 | UAUCUGGAGUCAUUGAGGGC | 697 |
| | CCGACCACGTGGAGCTGAGCTGG | 183 | CCGACCACGUGGAGCUGAGC | 698 |
| | AGGCTTCTACCCCGACCACGTGG | 184 | AGGCUUCUACCCCGACCACG | 699 |
| | GCGGCTGCTCAGGCAGTATCTGG | 185 | GCGGCUGCUCAGGCAGUAUC | 700 |
| | GAAAAACGTGTTCCCACCCAAGG | 186 | GAAAAACGUGUUCCCACCCA | 701 |
| | CAAGATCTCATAGAGGATGGTGG | 187 | CAAGAUCUCAUAGAGGAUGG | 702 |
| | TCCTCTATGAGATCTTGCTAGGG | 188 | UCCUCUAUGAGAUCUUGCUA | 703 |
| | GTTGCGGGGGTTCTGCCAGAAGG | 189 | GUUGCGGGGGUUCUGCCAGA | 704 |
| | AACACGTTTTTCAGGTCCTCTGG | 190 | AACACGUUUUUCAGGUCCUC | 705 |
| | CTTGACAGCGGAAGTGGTTGCGG | 191 | CUUGACAGCGGAAGUGGUUG | 706 |
| | ATCCTCTATGAGATCTTGCTAGG | 192 | AUCCUCUAUGAGAUCUUGCU | 707 |
| | GCTGTCAAGTCCAGTTCTACGGG | 193 | GCUGUCAAGUCCAGUUCUAC | 708 |
| | CAGCTCAGCTCCACGTGGTCGGG | 194 | CAGCUCAGCUCCACGUGGUC | 709 |
| | AGATCGTCAGCGCCGAGGCCTGG | 195 | AGAUCGUCAGCGCCGAGGCC | 710 |
| | GATCGTCAGCGCCGAGGCCTGGG | 196 | GAUCGUCAGCGCCGAGGCCU | 711 |
| PDCD1 | ACCGCCCAGACGACTGGCCAGGG | 197 | ACCGCCCAGACGACUGGCCA | 712 |
| | TGACGTTACCTCGTGCGGCCCGG | 198 | UGACGUUACCUCGUGCGGCC | 713 |
| | ATGTGGAAGTCACGCCCGTTGGG | 199 | AUGUGGAAGUCACGCCCGUU | 714 |
| | TGGGATGACGTTACCTCGTGCGG | 200 | UGGGAUGACGUUACCUCGUG | 715 |
| | GTCTGGGCGGTGCTACAACTGGG | 201 | GUCUGGGCGGUGCUACAACU | 716 |
| | GACGTTACCTCGTGCGGCCCGGG | 202 | GACGUUACCUCGUGCGGCCC | 717 |
| | CGTCTGGGCGGTGCTACAACTGG | 203 | CGUCUGGGCGGUGCUACAAC | 718 |
| | GCGTGACTTCCACATGAGCGTGG | 204 | GCGUGACUUCCACAUGAGCG | 719 |
| | CGACTGGCCAGGGCGCCTGTGGG | 205 | CGACUGGCCAGGGCGCCUGU | 720 |
| | TGTAGCACCGCCCAGACGACTGG | 206 | UGUAGCACCGCCCAGACGAC | 721 |
| | CACGAAGCTCTCCGATGTGTTGG | 207 | CACGAAGCUCUCCGAUGUGU | 722 |
| | TGACACGGAAGCGGCAGTCCTGG | 208 | UGACACGGAAGCGGCAGUCC | 723 |
| | TCAGTGGCTGGGCACTCCGAGGG | 209 | UCAGUGGCUGGGCACUCCGA | 724 |
| | CGGAGAGCTTCGTGCTAAACTGG | 210 | CGGAGAGCUUCGUGCUAAAC | 725 |
| | AGGTGCCGCTGTCATTGCGCCGG | 211 | AGGUGCCGCUGUCAUUGCGC | 726 |
| | AGCTTGTCCGTCTGGTTGCTGGG | 212 | AGCUUGUCCGUCUGGUUGCU | 727 |
| | CACCTACCTAAGAACCATCCTGG | 213 | CACCUACCUAAGAACCAUCC | 728 |
| | CGCCCACGACACCAACCACCAGG | 214 | CGCCCACGACACCAACCACC | 729 |
| | ATTGTCTTTCCTAGCGGAATGGG | 215 | AUUGUCUUUCCUAGCGGAAU | 730 |
| | GTGGCATACTCCGTCTGCTCAGG | 216 | GUGGCAUACUCCGUCUGCUC | 731 |
| | CCCCTTCGGTCACCACGAGCAGG | 217 | CCCCUUCGGUCACCACGAGC | 732 |
| | AGGCGCCCTGGCCAGTCGTCTGG | 218 | AGGCGCCCUGGCCAGUCGUC | 733 |
| | AGCCGGCCAGTTCCAAACCCTGG | 219 | AGCCGGCCAGUUCCAAACCC | 734 |
| | ACTTCCACATGAGCGTGGTCAGG | 220 | ACUUCCACAUGAGCGUGGUC | 735 |
| | CGTTGGGCAGTTGTGTGACACGG | 221 | CGUUGGGCAGUUGUGUGACA | 736 |
| | CCCTTCGGTCACCACGAGCAGGG | 222 | CCCUUCGGUCACCACGAGCA | 737 |
| | ATCTGCTCCCGGGCCGCACGAGG | 223 | AUCUGCUCCCGGGCCGCACG | 738 |
| | ACCCTGGTGGTTGGTGTCGTGGG | 224 | ACCCUGGUGGUUGGUGUCGU | 739 |
| | CACCGCCCAGACGACTGGCCAGG | 225 | CACCGCCCAGACGACUGGCC | 740 |
| | GGGCGGTGCTACAACTGGGCTGG | 226 | GGGCGGUGCUACAACUGGGC | 741 |
| | CAGCTTGTCCGTCTGGTTGCTGG | 227 | CAGCUUGUCCGUCUGGUUGC | 742 |
| | CATGTGGAAGTCACGCCCGTTGG | 228 | CAUGUGGAAGUCACGCCCGU | 743 |
| | CGTGTCACACAACTGCCCAACGG | 229 | CGUGUCACACAACUGCCCAA | 744 |
| | AGGGCCCGGCGCAATGACAGCGG | 230 | AGGGCCCGGCGCAAUGACAG | 745 |
| | GGTGACAGGTGCGGCCTCGGAGG | 231 | GGUGACAGGUGCGGCCUCGG | 746 |
| | GTGTCACACAACTGCCCAACGGG | 232 | GUGUCACACAACUGCCCAAC | 747 |
| | AGGGTTTGGAACTGGCCGGCTGG | 233 | AGGGUUUGGAACUGGCCGGC | 748 |
| | TGGCGGCCAGGATGGTTCTTAGG | 234 | UGGCGGCCAGGAUGGUUCUU | 749 |
| | CGACACCAACCACCAGGGTTTGG | 235 | CGACACCAACCACCAGGGUU | 750 |
| | AGGCGGCCAGCTTGTCCGTCTGG | 236 | AGGCGGCCAGCUUGUCCGUC | 751 |
| | CTACAACTGGGCTGGCGGCCAGG | 237 | CUACAACUGGGCUGGCGGCC | 752 |
| | GCTCTCTTTGATCTGCGCCTTGG | 238 | GCUCUCUUUGAUCUGCGCCU | 753 |
| | CTCTCTTTGATCTGCGCCTTGGG | 239 | CUCUCUUUGAUCUGCGCCUU | 754 |
| | TCGGTCACCACGAGCAGGGCTGG | 240 | UCGGUCACCACGAGCAGGGC | 755 |
| | TCCGCTAGGAAAGACAATGGTGG | 241 | UCCGCUAGGAAAGACAAUGG | 756 |
| | GATGAGGTGCCCATTCCGCTAGG | 242 | GAUGAGGUGCCCAUUCCGCU | 757 |
| | ACCTCATCCCCCGCCCGCAGGGG | 243 | ACCUCAUCCCCCGCCCGCAG | 758 |
| | GATCTGCGCCTTGGGGGCCAGGG | 244 | GAUCUGCGCCUUGGGGGCCA | 759 |
| | GGTGCCGCTGTCATTGCGCCGGG | 245 | GGUGCCGCUGUCAUUGCGCC | 760 |
| | AGGATGGTTCTTAGGTAGGTGGG | 246 | AGGAUGGUUCUUAGGUAGGU | 761 |
| TIM- | ATAGGCATCTACATCGGAGCAGG | 247 | AUAGGCAUCUACAUCGGAGC | 762 |
| 3/HAVCR2 | TCTCTCTGCCGAGTCGGTGCAGG | 248 | UCUCUCUGCCGAGUCGGUGC | 763 |
| | ATGAGAATACCCTAGTAAGGGGG | 249 | AUGAGAAUACCCUAGUAAGG | 764 |
| | CGACAACCCAAAGGTTGTGAGGG | 250 | CGACAACCCAAAGGUUGUGA | 765 |
| | CCGTAACTCATTGGCCAATGTGG | 251 | CCGUAACUCAUUGGCCAAUG | 766 |
| | TATGAGAATACCCTAGTAAGGGG | 252 | UAUGAGAAUACCCUAGUAAG | 767 |
| | TGAGTTACGGGACTCTAGATTGG | 253 | UGAGUUACGGGACUCUAGAU | 768 |
| | TCTAGAGTCCCGTAACTCATTGG | 254 | UCUAGAGUCCCGUAACUCAU | 769 |
| | GCCAATGACTTACGGGACTCTGG | 255 | GCCAAUGACUUACGGGACUC | 770 |
| | GACGGGCACGAGGTTCCCTGGGG | 256 | GACGGGCACGAGGUUCCCUG | 771 |
| | AGACGGGCACGAGGTTCCCTGGG | 257 | AGACGGGCACGAGGUUCCCU | 772 |
| | TCTGGAGCAACCATCAGAATAGG | 258 | UCUGGAGCAACCAUCAGAAU | 773 |
| | CAGACGGGCACGAGGTTCCCTGG | 259 | CAGACGGGCACGAGGUUCCC | 774 |
| | CTGGTTTGATGACCAACTTCAGG | 260 | CUGGUUUGAUGACCAACUUC | 775 |
| | GGCCCAGGTAACTATGCATGGGG | 261 | GGCCCAGGUAACUAUGCAUG | 776 |
| | ATTGCAAAGCGACAACCCAAAGG | 262 | AUUGCAAAGCGACAACCCAA | 777 |
| | TGGTCATCAAACCAGGTGAGTGG | 263 | UGGUCAUCAAACCAGGUGAG | 778 |
| | CTTACAAGTAAGTCTCGGCATGG | 264 | CUUACAAGUAAGUCUCGGCA | 779 |
| | CTAAATGGGGATTTCCGCAAAGG | 265 | CUAAAUGGGGAUUUCCGCAA | 780 |
| | CATGCAAATGTCCACTCACCTGG | 266 | CAUGCAAAUGUCCACUCACC | 781 |
| | GCTATGAGAATACCCTAGTAAGG | 267 | GCUAUGAGAAUACCCUAGUA | 782 |
| | CTCTCTGCCGAGTCGGTGCAGGG | 268 | CUCUCUGCCGAGUCGGUGCA | 783 |
| | GAACCTCGTGCCCGTCTGCTGGG | 269 | GAACCUCGUGCCCGUCUGCU | 784 |
| | GTGAAGTCTCTCTGCCGAGTCGG | 270 | GUGAAGUCUCUCUGCCGAGU | 785 |
| | TCCAGAGTCCCGTAAGTCATTGG | 271 | UCCAGAGUCCCGUAAGUCAU | 786 |
| | AATGTGACTCTAGCAGACAGTGG | 272 | AAUGUGACUCUAGCAGACAG | 787 |
| | TAGGCATCTACATCGGAGCAGGG | 273 | UAGGCAUCUACAUCGGAGCA | 788 |
| | GTTGTTTCTGACATTAGCCAAGG | 274 | GUUGUUUCUGACAUUAGCCA | 789 |
| | TGCTGCCGGATCCAAATCCCAGG | 275 | UGCUGCCGGAUCCAAAUCCC | 790 |
| | GCCAATGTGGATATTTGCTATGG | 276 | GCCAAUGUGGAUAUUUGCUA | 791 |
| | CTAGATTGGCCAATGACTTACGG | 277 | CUAGAUUGGCCAAUGACUUA | 792 |
| | CTGCCCCATGCATAGTTACCTGG | 278 | CUGCCCCAUGCAUAGUUACC | 793 |
| | TGTGTTTGAATGTGGCAACGTGG | 279 | UGUGUUUGAAUGUGGCAACG | 794 |
| | AGAAGTGGAATACAGAGCGGAGG | 280 | AGAAGUGGAAUACAGAGCGG | 795 |
| | TGGCCCAGGTAACTATGCATGGG | 281 | UGGCCCAGGUAACUAUGCAU | 796 |
| | TAGATTGGCCAATGACTTACGGG | 282 | UAGAUUGGCCAAUGACUUAC | 797 |
| | GGAACCTCGTGCCCGTCTGCTGG | 283 | GGAACCUCGUGCCCGUCUGC | 798 |
| | ACGTTGCCACATTCAAACACAGG | 284 | ACGUUGCCACAUUCAAACAC | 799 |
| | TGCCCCAGCAGACGGGCACGAGG | 285 | UGCCCCAGCAGACGGGCACG | 800 |
| | ATGGCCCAGGTAACTATGCATGG | 286 | AUGGCCCAGGUAACUAUGCA | 801 |
| | AGGTCACCCCTGCACCGACTCGG | 287 | AGGUCACCCCUGCACCGACU | 802 |
| | AATGTGGCAACGTGGTGCTCAGG | 288 | AAUGUGGCAACGUGGUGCUC | 803 |
| | GAGTCACATTCTCTATGGTCAGG | 289 | GAGUCACAUUCUCUAUGGUC | 804 |
| | ATCCCCATTTAGCCAGTATCTGG | 290 | AUCCCCAUUUAGCCAGUAUC | 805 |
| | CATCCAGATACTGGCTAAATGGG | 291 | CAUCCAGAUACUGGCUAAAU | 806 |
| | ATGTGACTCTAGCAGACAGTGGG | 292 | AUGUGACUCUAGCAGACAGU | 807 |
| | GATGTAGATGCCTATTCTGATGG | 293 | GAUGUAGAUGCCUAUUCUGA | 808 |
| | CTTACTGTTAGATTTATATCAGG | 294 | CUUACUGUUAGAUUUAUAUC | 809 |
| | ATCAGAATAGGCATCTACATCGG | 295 | AUCAGAAUAGGCAUCUACAU | 810 |
| | ATTATTGCTATGTCAGCAGCAGG | 296 | AUUAUUGCUAUGUCAGCAGC | 811 |
| TIGIT | GTACTCCCCTGTATCGTTCACGG | 297 | GUACUCCCCUGUAUCGUUCA | 812 |
| | TATCGTTCACGGTCAGCGACTGG | 298 | UAUCGUUCACGGUCAGCGAC | 813 |
| | TCGCTGACCGTGAACGATACAGG | 299 | UCGCUGACCGUGAACGAUAC | 814 |
| | TGGGGCCACTCGATCCTTGAAGG | 300 | UGGGGCCACUCGAUCCUUGA | 815 |
| | CGTTCACGGTCAGCGACTGGAGG | 301 | CGUUCACGGUCAGCGACUGG | 816 |
| | ACCCTGATGGGACGTACACTGGG | 302 | ACCCUGAUGGGACGUACACU | 817 |
| | GCGGCCATGGCTCCAAGCAATGG | 303 | GCGGCCAUGGCUCCAAGCAA | 818 |
| | CGCTGACCGTGAACGATACAGGG | 304 | CGCUGACCGUGAACGAUACA | 819 |
| | TCCCAGTGTACGTCCCATCAGGG | 305 | UCCCAGUGUACGUCCCAUCA | 820 |
| | CCCATCCTTCAAGGATCGAGTGG | 306 | CCCAUCCUUCAAGGAUCGAG | 821 |
| | CGCGTTGACTAGAAAGGTAATGG | 307 | CGCGUUGACUAGAAAGGUAA | 822 |
| | CTCCCAGTGTACGTCCCATCAGG | 308 | CUCCCAGUGUACGUCCCAUC | 823 |
| | AGTGTACGTCCCATCAGGGTAGG | 309 | AGUGUACGUCCCAUCAGGGU | 824 |
| | TTCAAGGATCGAGTGGCCCCAGG | 310 | UUCAAGGAUCGAGUGGCCCC | 825 |
| | GAAAGCTCAGGTATTCCTGCTGG | 311 | GAAAGCUCAGGUAUUCCUGC | 826 |
| | GGTGGTCGCGTTGACTAGAAAGG | 312 | GGUGGUCGCGUUGACUAGAA | 827 |
| | GACCACCAGCGTCGCGGCCATGG | 313 | GACCACCAGCGUCGCGGCCA | 828 |
| | GCCACTCGATCCTTGAAGGATGG | 314 | GCCACUCGAUCCUUGAAGGA | 829 |
| | GCAGATGACCACCAGCGTCGCGG | 315 | GCAGAUGACCACCAGCGUCG | 830 |
| | GTTCACGGTCAGCGACTGGAGGG | 316 | GUUCACGGUCAGCGACUGGA | 831 |
| | CAGGCACAATAGAAACAACGGGG | 317 | CAGGCACAAUAGAAACAACG | 832 |
| | TGGAGCCATGGCCGCGACGCTGG | 318 | UGGAGCCAUGGCCGCGACGC | 833 |
| | CACAAGTGACCCAGGTCAACTGG | 319 | CACAAGUGACCCAGGUCAAC | 834 |
| | TAGCAACCAGAGGCATCTTCTGG | 320 | UAGCAACCAGAGGCAUCUUC | 835 |
| | GCTGACCGTGAACGATACAGGGG | 321 | GCUGACCGUGAACGAUACAG | 836 |
| | GACCTGGGTCACTTGTGCCGTGG | 322 | GACCUGGGUCACUUGUGCCG | 837 |
| | TACCCTGATGGGACGTACACTGG | 323 | UACCCUGAUGGGACGUACAC | 838 |
| | GACTAGAAAGGTAATGGCTCCGG | 324 | GACUAGAAAGGUAAUGGCUC | 839 |
| | TCTATCACACCTACCCTGATGGG | 325 | UCUAUCACACCUACCCUGAU | 840 |
| | AGGTTCCAGATTCCATTGCTTGG | 326 | AGGUUCCAGAUUCCAUUGCU | 841 |
| | ATTGAAGTAGTCATGCAGCTCGG | 327 | AUUGAAGUAGUCAUGCAGCU | 842 |
| | CACCACGGCACAAGTGACCCAGG | 328 | CACCACGGCACAAGUGACCC | 843 |
| | TTTGTAATGCTGACTTGGGGTGG | 329 | UUUGUAAUGCUGACUUGGGG | 844 |
| | TCAGGCCTTACCTGAGGCGAGGG | 330 | UCAGGCCUUACCUGAGGCGA | 845 |
| | GATTCCATTGCTTGGAGCCATGG | 331 | GAUUCCAUUGCUUGGAGCCA | 846 |
| | CTGCACAGCAGTCATCGTGGTGG | 332 | CUGCACAGCAGUCAUCGUGG | 847 |
| | GATCGAGTGGCCCCAGGTCCCGG | 333 | GAUCGAGUGGCCCCAGGUCC | 848 |
| | AGCCATGGCCGCGACGCTGGTGG | 334 | AGCCAUGGCCGCGACGCUGG | 849 |
| | ATCTATCACACCTACCCTGATGG | 335 | AUCUAUCACACCUACCCUGA | 850 |
| | AGAGACTGGTTAGCAACCAGAGG | 336 | AGAGACUGGUUAGCAACCAG | 851 |
| | ACAAGTGACCCAGGTCAACTGGG | 337 | ACAAGUGACCCAGGUCAACU | 852 |
| | CGGTCAGCGACTGGAGGGTGAGG | 338 | CGGUCAGCGACUGGAGGGUG | 853 |
| | GTACACTGGGAGAATCTTCCTGG | 339 | GUACACUGGGAGAAUCUUCC | 854 |
| | ATTCTGTGGAAGGTGACCTCAGG | 340 | AUUCUGUGGAAGGUGACCUC | 855 |
| | TACCCAGGCTTCTGTAACTCAGG | 341 | UACCCAGGCUUCUGUAACUC | 856 |
| | CCATTTGTAATGCTGACTTGGGG | 342 | CCAUUUGUAAUGCUGACUUG | 857 |
| | CAGGCCTTACCTGAGGCGAGGGG | 343 | CAGGCCUUACCUGAGGCGAG | 858 |
| | GTCCAGCTGATTTTCTCCTGAGG | 344 | GUCCAGCUGAUUUUCUCCUG | 859 |
| | CACTCGATCCTTGAAGGATGGGG | 345 | CACUCGAUCCUUGAAGGAUG | 860 |
| | GCCATTTGTAATGCTGACTTGGG | 346 | GCCAUUUGUAAUGCUGACUU | 861 |
| LAG3 | GTGCATTGGTTCCGGAACCGGGG | 347 | GUGCAUUGGUUCCGGAACCG | 862 |
| | CGACTTTACCCTTCGACTAGAGG | 348 | CGACUUUACCCUUCGACUAG | 863 |
| | TCGACTAGAGGATGTGAGCCAGG | 349 | UCGACUAGAGGAUGUGAGCC | 864 |
| | GCTTTCCGCTAAGTGGTGATGGG | 350 | GCUUUCCGCUAAGUGGUGAU | 865 |
| | CGCTACACGGTGCTGAGCGTGGG | 351 | CGCUACACGGUGCUGAGCGU | 866 |
| | GCGTACACTGTCAAGGGAGTTGG | 352 | GCGUACACUGUCAAGGGAGU | 867 |
| | AGCGCGGGGACTTCTCGCTATGG | 353 | AGCGCGGGGACUUCUCGCUA | 868 |
| | GCTCCAGCGTACACTGTCAAGGG | 354 | GCUCCAGCGUACACUGUCAA | 869 |
| | GCTCACATCCTCTAGTCGAAGGG | 355 | GCUCACAUCCUCUAGUCGAA | 870 |
| | GGCTCACATCCTCTAGTCGAAGG | 356 | GGCUCACAUCCUCUAGUCGA | 871 |
| | CGCCCCACATACTCGAGGCCTGG | 357 | CGCCCCACAUACUCGAGGCC | 872 |
| | CTGTGCATTGGTTCCGGAACCGG | 358 | CUGUGCAUUGGUUCCGGAAC | 873 |
| | TTGGTTCCGGAACCGGGGCCAGG | 359 | UUGGUUCCGGAACCGGGGCC | 874 |
| | CGCTCATCCAGCTGGACGCGGGG | 360 | CGCUCAUCCAGCUGGACGCG | 875 |
| | TTCCGCTAAGTGGTGATGGGGGG | 361 | UUCCGCUAAGUGGUGAUGGG | 876 |
| | CAGGCCTCGAGTATGTGGGGCGG | 362 | CAGGCCUCGAGUAUGUGGGG | 877 |
| | GCAAGGGATTCACCCTCCGCAGG | 363 | GCAAGGGAUUCACCCUCCGC | 878 |
| | AGCTTTCCGCTAAGTGGTGATGG | 364 | AGCUUUCCGCUAAGUGGUGA | 879 |
| | CGCTCAGCACCGTGTAGCGGCGG | 365 | CGCUCAGCACCGUGUAGCGG | 880 |
| | CGTACACTGTCAAGGGAGTTGGG | 366 | CGUACACUGUCAAGGGAGUU | 881 |
| | ACCGTGTAGCGGCGGGGCCTGGG | 367 | ACCGUGUAGCGGCGGGGCCU | 882 |
| | GCCGGCCGCGCTCATCCAGCTGG | 368 | GCCGGCCGCGCUCAUCCAGC | 883 |
| | CGTCCCGCCCCACATACTCGAGG | 369 | CGUCCCGCCCCACAUACUCG | 884 |
| | ACTCCCTTGACAGTGTACGCTGG | 370 | ACUCCCUUGACAGUGUACGC | 885 |
| | CGCCGGCGAGTACCGCGCCGCGG | 371 | CGCCGGCGAGUACCGCGCCG | 886 |
| | GTTCCGGAACCAATGCACAGAGG | 372 | GUUCCGGAACCAAUGCACAG | 887 |
| | CGCGTCCAGCTGGATGAGCGCGG | 373 | CGCGUCCAGCUGGAUGAGCG | 888 |
| | GTACGCTGGAGCAGGTTCCAGGG | 374 | GUACGCUGGAGCAGGUUCCA | 889 |
| | TCTAAGGCAGAAAATCGTCTTGG | 375 | UCUAAGGCAGAAAAUCGUCU | 890 |
| | AAGCGTTCTTGTCCAGATACTGG | 376 | AAGCGUUCUUGUCCAGAUAC | 891 |
| | GCGAGAAGTCCCCGCGCTGCCGG | 377 | GCGAGAAGUCCCCGCGCUGC | 892 |
| | CACCGCGGCGCGGTACTCGCCGG | 378 | CACCGCGGCGCGGUACUCGC | 893 |
| | TCCATAGGTGCCCAACGCTCTGG | 379 | UCCAUAGGUGCCCAACGCUC | 894 |
| | CACCGTGTAGCGGCGGGGCCTGG | 380 | CACCGUGUAGCGGCGGGGCC | 895 |
| | GGGTGGCTCCAGGTAAAACGGGG | 381 | GGGUGGCUCCAGGUAAAACG | 896 |
| | GACGTTGAAGCCATCTCTGTAGG | 382 | GACGUUGAAGCCAUCUCUGU | 897 |
| | GATGGGGGGACTCCCGGACAGGG | 383 | GAUGGGGGGACUCCCGGACA | 898 |
| | AGTATGTGGGGGGGGACGATGGG | 384 | AGUAUGUGGGGGGGGACGAU | 899 |
| | CCAGGTAAAACGGGGATGGCGGG | 385 | CCAGGUAAAACGGGGAUGGC | 900 |
| | GGGCCAGGCCTCGAGTATGTGGG | 386 | GGGCCAGGCCUCGAGUAUGU | 901 |
| | GGTAAAACGGGGATGGCGGGAGG | 387 | GGUAAAACGGGGAUGGCGGG | 902 |
| | ACCGCGCCGCGGTGCACCTCAGG | 388 | ACCGCGCCGCGGUGCACCUC | 903 |
| | ACTCGCCGGCGTCCGCGCGCCGG | 389 | ACUCGCCGGCGUCCGCGCGC | 904 |
| | GATCTCTCAGAGCCTCCGACTGG | 390 | GAUCUCUCAGAGCCUCCGAC | 905 |
| | GCGGTCCCTGAGGTGCACCGCGG | 391 | GCGGUCCCUGAGGUGCACCG | 906 |
| | GTCCCCCCATCACCACTTAGCGG | 392 | GUCCCCCCAUCACCACUUAG | 907 |
| | AGAGGAAGCTTTCCGCTAAGTGG | 393 | AGAGGAAGCUUUCCGCUAAG | 908 |
| | TGCTCCAGCGTACACTGTCAAGG | 394 | UGCUCCAGCGUACACUGUCA | 909 |
| | TTGACAGTGTACGCTGGAGCAGG | 395 | UUGACAGUGUACGCUGGAGC | 910 |
| | AGGCCTCGAGTATGTGGGGGGGG | 396 | AGGCCUCGAGUAUGUGGGGC | 911 |
| CTLA4 | ACACCGCTCCCATAAAGCCATGG | 397 | ACACCGCUCCCAUAAAGCCA | 912 |
| | GTGCGGCAACCTACATGATGGGG | 398 | GUGCGGCAACCUACAUGAUG | 913 |
| | TACCCACCGCCATACTACCTGGG | 399 | UACCCACCGCCAUACUACCU | 914 |
| | CCGCCATACTACCTGGGCATAGG | 400 | CCGCCAUACUACCUGGGCAU | 915 |
| | GTACCCACCGCCATACTACCTGG | 401 | GUACCCACCGCCAUACUACC | 916 |
| | GGGTTCCGTTGCCTATGCCCAGG | 402 | GGGUUCCGUUGCCUAUGCCC | 917 |
| | CATAGACCCCTGTTGTAAGAGGG | 403 | CAUAGACCCCUGUUGUAAGA | 918 |
| | TGCCCAGGTAGTATGGCGGTGGG | 404 | UGCCCAGGUAGUAUGGCGGU | 919 |
| | AGGTCCGGGTGACAGTGCTTCGG | 405 | AGGUCCGGGUGACAGUGCUU | 920 |
| | TGAACCTGGCTACCAGGACCTGG | 406 | UGAACCUGGCUACCAGGACC | 921 |
| | TTGCCTATGCCCAGGTAGTATGG | 407 | UUGCCUAUGCCCAGGUAGUA | 922 |
| | CTGTGCGGCAACCTACATGATGG | 408 | CUGUGCGGCAACCUACAUGA | 923 |
| | TGTGCGGCAACCTACATGATGGG | 409 | UGUGCGGCAACCUACAUGAU | 924 |
| | CCGGGTGACAGTGCTTCGGCAGG | 410 | CCGGGUGACAGUGCUUCGGC | 925 |
| | ACATAGACCCCTGTTGTAAGAGG | 411 | ACAUAGACCCCUGUUGUAAG | 926 |
| | CCTTGGATTTCAGCGGCACAAGG | 412 | CCUUGGAUUUCAGCGGCACA | 927 |
| | GTTCACTTGATTTCCACTGGAGG | 413 | GUUCACUUGAUUUCCACUGG | 928 |
| | GGCCACGTGCATTGCTAGCATGG | 414 | GGCCACGUGCAUUGCUAGCA | 929 |
| | TACTACCTGGGCATAGGCAACGG | 415 | UACUACCUGGGCAUAGGCAA | 930 |
| | GCTCACCAATTACATAAATCTGG | 416 | GCUCACCAAUUACAUAAAUC | 931 |
| | ACTGGAGGTGCCCGTGCAGATGG | 417 | ACUGGAGGUGCCCGUGCAGA | 932 |
| | TTCCATGCTAGCAATGCACGTGG | 418 | UUCCAUGCUAGCAAUGCACG | 933 |
| | AAGGCAAGCCATGGCTTTATGGG | 419 | AAGGCAAGCCAUGGCUUUAU | 934 |
| | CAAGGCAAGCCATGGCTTTATGG | 420 | CAAGGCAAGCCAUGGCUUUA | 935 |
| | ATCTGCACGGGCACCTCCAGTGG | 421 | AUCUGCACGGGCACCUCCAG | 936 |
| | CACTGTCACCCGGACCTCAGTGG | 422 | CACUGUCACCCGGACCUCAG | 937 |
| | CCTCACTATCCAAGGACTGAGGG | 423 | CCUCACUAUCCAAGGACUGA | 938 |
| | CTAGATGATTCCATCTGCACGGG | 424 | CUAGAUGAUUCCAUCUGCAC | 939 |
| | GCTTCGGCAGGCTGACAGCCAGG | 425 | GCUUCGGCAGGCUGACAGCC | 940 |
| | CACGGGACTCTACATCTGCAAGG | 426 | CACGGGACUCUACAUCUGCA | 941 |
| | ATGCCCAGGTAGTATGGCGGTGG | 427 | AUGCCCAGGUAGUAUGGCGG | 942 |
| | AAGAAGCCCTCTTACAACAGGGG | 428 | AAGAAGCCCUCUUACAACAG | 943 |
| | GCAAAGGTGAGTGAGACTTTTGG | 429 | GCAAAGGUGAGUGAGACUUU | 944 |
| | GGGACTCTACATCTGCAAGGTGG | 430 | GGGACUCUACAUCUGCAAGG | 945 |
| | ACCTCACTATCCAAGGACTGAGG | 431 | ACCUCACUAUCCAAGGACUG | 946 |
| | CAAGTGAACCTCACTATCCAAGG | 432 | CAAGUGAACCUCACUAUCCA | 947 |
| | GCTGGCGATGCCTCGGCTGCTGG | 433 | GCUGGCGAUGCCUCGGCUGC | 948 |
| | CTCACCAATTACATAAATCTGGG | 434 | CUCACCAAUUACAUAAAUCU | 949 |
| | GGAACCCAGATTTATGTAATTGG | 435 | GGAACCCAGAUUUAUGUAAU | 950 |
| | CCTAGATGATTCCATCTGCACGG | 436 | CCUAGAUGAUUCCAUCUGCA | 951 |
| | AAAGAAGCCCTCTTACAACAGGG | 437 | AAAGAAGCCCUCUUACAACA | 952 |
| | GAGGTTCACTTGATTTCCACTGG | 438 | GAGGUUCACUUGAUUUCCAC | 953 |
| | CGGACCTCAGTGGCTTTGCCTGG | 439 | CGGACCUCAGUGGCUUUGCC | 954 |
| | TGTCCATGGCCCTCAGTCCTTGG | 440 | UGUCCAUGGCCCUCAGUCCU | 955 |
| | ACACAAAGCTGGCGATGCCTCGG | 441 | ACACAAAGCUGGCGAUGCCU | 956 |
| | AAGCCATGGCTTTATGGGAGCGG | 442 | AAGCCAUGGCUUUAUGGGAG | 957 |
| | CTCAGCTGAACCTGGCTACCAGG | 443 | CUCAGCUGAACCUGGCUACC | 958 |
| | GATGTAGAGTCCCGTGTCCATGG | 444 | GAUGUAGAGUCCCGUGUCCA | 959 |
| | AAAAGAAGCCCTCTTACAACAGG | 445 | AAAAGAAGCCCUCUUACAAC | 960 |
| | GCACGTGGCCCAGCCTGCTGTGG | 446 | GCACGUGGCCCAGCCUGCUG | 961 |
| AAVS1 | GCTGCTCTGACGCGGCCGTCTGG | 447 | GCUGCUCUGACGCGGCCGUC | 962 |
| | TATAAGGTGGTCCCAGCTCGGGG | 448 | UAUAAGGUGGUCCCAGCUCG | 963 |
| | GACGCAAGGGAGACATCCGTCGG | 449 | GACGCAAGGGAGACAUCCGU | 964 |
| | AGGGAGACATCCGTCGGAGAAGG | 450 | AGGGAGACAUCCGUCGGAGA | 965 |
| | CTTAGGATGGCCTTCTCCGACGG | 451 | CUUAGGAUGGCCUUCUCCGA | 966 |
| | CTGGTGCGTTTCACTGATCCTGG | 452 | CUGGUGCGUUUCACUGAUCC | 967 |
| | CAGGTAAAACTGACGCACGGAGG | 453 | CAGGUAAAACUGACGCACGG | 968 |
| | GATCAGTGAAACGCACCAGACGG | 454 | GAUCAGUGAAACGCACCAGA | 969 |
| | GTCACCAATCCTGTCCCTAGTGG | 455 | GUCACCAAUCCUGUCCCUAG | 970 |
| | GAGAGGTGACCCGAATCCACAGG | 456 | GAGAGGUGACCCGAAUCCAC | 971 |
| | CCTCTAAGGTTTGCTTACGATGG | 457 | CCUCUAAGGUUUGCUUACGA | 972 |
| | TAAGGAATCTGCCTAACAGGAGG | 458 | UAAGGAAUCUGCCUAACAGG | 973 |
| | ATTCCCAGGGCCGGTTAATGTGG | 459 | AUUCCCAGGGCCGGUUAAUG | 974 |
| | CCCAAAGTACCCCGTCTCCCTGG | 460 | CCCAAAGUACCCCGUCUCCC | 975 |
| | ATATAAGGTGGTCCCAGCTCGGG | 461 | AUAUAAGGUGGUCCCAGCUC | 976 |
| | TAACCGGCCCTGGGAATATAAGG | 462 | UAACCGGCCCUGGGAAUAUA | 977 |
| | CTGCATCATCACCGTTTTTCTGG | 463 | CUGCAUCAUCACCGUUUUUC | 978 |
| | TAAGAAACGAGAGATGGCACAGG | 464 | UAAGAAACGAGAGAUGGCAC | 979 |
| | AGAGCTAGCACAGACTAGAGAGG | 465 | AGAGCUAGCACAGACUAGAG | 980 |
| | GGCTACTGGCCTTATCTCACAGG | 466 | GGCUACUGGCCUUAUCUCAC | 981 |
| | ACCCCGTTCTCCTGTGGATTCGG | 467 | ACCCCGUUCUCCUGUGGAUU | 982 |
| | CGGAGGAACAATATAAATTGGGG | 468 | CGGAGGAACAAUAUAAAUUG | 983 |
| | ACAGTGGGGCCACTAGGGACAGG | 469 | ACAGUGGGGCCACUAGGGAC | 984 |
| | CGGCCGCGTCAGAGCAGCTCAGG | 470 | CGGCCGCGUCAGAGCAGCUC | 985 |
| | ACGGAGGAACAATATAAATTGGG | 471 | ACGGAGGAACAAUAUAAAUU | 986 |
| | GGGACCACCTTATATTCCCAGGG | 472 | GGGACCACCUUAUAUUCCCA | 987 |
| | TGGGACCACCTTATATTCCCAGG | 473 | UGGGACCACCUUAUAUUCCC | 988 |
| | CCATCTCTCGTTTCTTAGGATGG | 474 | CCAUCUCUCGUUUCUUAGGA | 989 |
| | TAAGCAAACCTTAGAGGTTCTGG | 475 | UAAGCAAACCUUAGAGGUUC | 990 |
| | CGTCAGAGCAGCTCAGGTTCTGG | 476 | CGUCAGAGCAGCUCAGGUUC | 991 |
| | GACCCGAATCCACAGGAGAACGG | 477 | GACCCGAAUCCACAGGAGAA | 992 |
| | AGAGCCACATTAACCGGCCCTGG | 478 | AGAGCCACAUUAACCGGCCC | 993 |
| | TCACAGGTAAAACTGACGCACGG | 479 | UCACAGGUAAAACUGACGCA | 994 |
| | TTCTGGGAGAGGGTAGCGCAGGG | 480 | UUCUGGGAGAGGGUAGCGCA | 995 |
| | GGATCCTGTGTCCCCGAGCTGGG | 481 | GGAUCCUGUGUCCCCGAGCU | 996 |
| | TGGGGGTTAGACCCAATATCAGG | 482 | UGGGGGUUAGACCCAAUAUC | 997 |
| | GTCCCTAGTGGCCCCACTGTGGG | 483 | GUCCCUAGUGGCCCCACUGU | 998 |
| | TGTTAGGCAGATTCCTTATCTGG | 484 | UGUUAGGCAGAUUCCUUAUC | 999 |
| | AAACCTTAGAGGTTCTGGCAAGG | 485 | AAACCUUAGAGGUUCUGGCA | 1000 |
| | CTGGACACCCCGTTCTCCTGTGG | 486 | CUGGACACCCCGUUCUCCUG | 1001 |
| | GGGGGGATGCGTGACCTGCCCGG | 487 | GGGGGGAUGCGUGACCUGCC | 1002 |
| | GGTTAATGTGGCTCTGGTTCTGG | 488 | GGUUAAUGUGGCUCUGGUUC | 1003 |
| | TGATGCAGGCCTACAAGAAGGGG | 489 | UGAUGCAGGCCUACAAGAAG | 1004 |
| | TAGCTGAGCTCTCGGACCCCTGG | 490 | UAGCUGAGCUCUCGGACCCC | 1005 |
| | TGCTTACGATGGAGCCAGAGAGG | 491 | UGCUUACGAUGGAGCCAGAG | 1006 |
| | TGCTGTCCTGAAGTGGACATAGG | 492 | UGCUGUCCUGAAGUGGACAU | 1007 |
| | CTGTCCTGAAGTGGACATAGGGG | 493 | CUGUCCUGAAGUGGACAUAG | 1008 |
| | CAGGGAGACGGGGTACTTTGGGG | 494 | CAGGGAGACGGGGUACUUUG | 1009 |
| | ATGATGCAGGCCTACAAGAAGGG | 495 | AUGAUGCAGGCCUACAAGAA | 1010 |
| | ACCCGAATCCACAGGAGAACGGG | 496 | ACCCGAAUCCACAGGAGAAC | 1011 |
| | GCAAACATGCTGTCCTGAAGTGG | 497 | GCAAACAUGCUGUCCUGAAG | 1012 |
| | GACATAGGGGCCCGGGTTGGAGG | 498 | GACAUAGGGGCCCGGGUUGG | 1013 |
| | TGGGGGTGTGTCACCAGATAAGG | 499 | UGGGGGUGUGUCACCAGAUA | 1014 |
| | TGGCTAAAGCCAGGGAGACGGGG | 500 | UGGCUAAAGCCAGGGAGACG | 1015 |
| | TTGGTCCTGAGTTCTAACTTTGG | 501 | UUGGUCCUGAGUUCUAACUU | 1016 |
| | TCCCTAGTGGCCCCACTGTGGGG | 502 | UCCCUAGUGGCCCCACUGUG | 1017 |
| | CAGAAAAACGGTGATGATGCAGG | 503 | CAGAAAAACGGUGAUGAUGC | 1018 |
| | CTTCCTAGTCTCCTGATATTGGG | 504 | CUUCCUAGUCUCCUGAUAUU | 1019 |
| | CACGGAGGAACAATATAAATTGG | 505 | CACGGAGGAACAAUAUAAAU | 1020 |
| | GAACCTGAGCTGCTCTGACGCGG | 506 | GAACCUGAGCUGCUCUGACG | 1021 |
| | GAGCCACATTAACCGGCCCTGGG | 507 | GAGCCACAUUAACCGGCCCU | 1022 |
| | ACCCCACAGTGGGGCCACTAGGG | 508 | ACCCCACAGUGGGGCCACUA | 1023 |
| | GTCCCGCCTCCCCTTCTTGTAGG | 509 | GUCCCGCCUCCCCUUCUUGU | 1024 |
| | CCCCGTTCTCCTGTGGATTCGGG | 510 | CCCCGUUCUCCUGUGGAUUC | 1025 |
| | CCACCTTATATTCCCAGGGCCGG | 511 | CCACCUUAUAUUCCCAGGGC | 1026 |
| CCR5 | TCAGTTTACACCCGATCCACTGG | 512 | UCAGUUUACACCCGAUCCAC | 1027 |
| | AGTTTACACCCGATCCACTGGGG | 513 | AGUUUACACCCGAUCCACUG | 1028 |
| | TCATCCTCCTGACAATCGATAGG | 514 | UCAUCCUCCUGACAAUCGAU | 1029 |
| | CTTGTGACACGGACTCAAGTGGG | 515 | CUUGUGACACGGACUCAAGU | 1030 |
| | CAGTTTACACCCGATCCACTGGG | 516 | CAGUUUACACCCGAUCCACU | 1031 |
| | ACAATGTGTCAACTCTTGACAGG | 517 | ACAAUGUGUCAACUCUUGAC | 1032 |
| | GGTACCTATCGATTGTCAGGAGG | 518 | GGUACCUAUCGAUUGUCAGG | 1033 |
| | GTAAACTGAGCTTGCTCGCTCGG | 519 | GUAAACUGAGCUUGCUCGCU | 1034 |
| | GACAAGTGTGATCACTTGGGTGG | 520 | GACAAGUGUGAUCACUUGGG | 1035 |
| | TCTGAACTTCTCCCCGACAAAGG | 521 | UCUGAACUUCUCCCCGACAA | 1036 |
| | CCTGACAATCGATAGGTACCTGG | 522 | CCUGACAAUCGAUAGGUACC | 1037 |
| | CTCGCTCGGGAGCCTCTTGCTGG | 523 | CUCGCUCGGGAGCCUCUUGC | 1038 |
| | CAGGTTGGACCAAGCTATGCAGG | 524 | CAGGUUGGACCAAGCUAUGC | 1039 |
| | TGACCATGACAAGCAGCGGCAGG | 525 | UGACCAUGACAAGCAGCGGC | 1040 |
| | CACCCCAAAGGTGACCGTCCTGG | 526 | CACCCCAAAGGUGACCGUCC | 1041 |
| | TAAACTGAGCTTGCTCGCTCGGG | 527 | UAAACUGAGCUUGCUCGCUC | 1042 |
| | TCACTATGCTGCCGCCCAGTGGG | 528 | UCACUAUGCUGCCGCCCAGU | 1043 |
| | AGCGTTTGGCAATGTGCTTTTGG | 529 | AGCGUUUGGCAAUGUGCUUU | 1044 |
| | TTGACAGGGCTCTATTTTATAGG | 530 | UUGACAGGGCUCUAUUUUAU | 1045 |
| | CATCATCTATGCCTTTGTCGGGG | 531 | CAUCAUCUAUGCCUUUGUCG | 1046 |
| | CAATGTGTCAACTCTTGACAGGG | 532 | CAAUGUGUCAACUCUUGACA | 1047 |
| | TTGCAGTAGCTCTAACAGGTTGG | 533 | UUGCAGUAGCUCUAACAGGU | 1048 |
| | GCTGCCGCCCAGTGGGACTTTGG | 534 | GCUGCCGCCCAGUGGGACUU | 1049 |
| | AAGCCAGGACGGTCACCTTTGGG | 535 | AAGCCAGGACGGUCACCUUU | 1050 |
| | TGACACGGACTCAAGTGGGCTGG | 536 | UGACACGGACUCAAGUGGGC | 1051 |
| | CGACAAAGGCATAGATGATGGGG | 537 | CGACAAAGGCAUAGAUGAUG | 1052 |
| | ATAATTGCAGTAGCTCTAACAGG | 538 | AUAAUUGCAGUAGCUCUAAC | 1053 |
| | CAGGACGGTCACCTTTGGGGTGG | 539 | CAGGACGGUCACCUUUGGGG | 1054 |
| | AGCCAGGACGGTCACCTTTGGGG | 540 | AGCCAGGACGGUCACCUUUG | 1055 |
| | CAGAATTGATACTGACTGTATGG | 541 | CAGAAUUGAUACUGACUGUA | 1056 |
| | GACACCGAAGCAGAGTTTTTAGG | 542 | GACACCGAAGCAGAGUUUUU | 1057 |
| | GGTGACAAGTGTGATCACTTGGG | 543 | GGUGACAAGUGUGAUCACUU | 1058 |
| | AACACCAGTGAGTAGAGCGGAGG | 544 | AACACCAGUGAGUAGAGCGG | 1059 |
| | CTCACTATGCTGCCGCCCAGTGG | 545 | CUCACUAUGCUGCCGCCCAG | 1060 |
| | CTGTTCTATTTTCCAGCAAGAGG | 546 | CUGUUCUAUUUUCCAGCAAG | 1061 |
| | TGTCATGGTCATCTGCTACTCGG | 547 | UGUCAUGGUCAUCUGCUACU | 1062 |
| | CCATCATCTATGCCTTTGTCGGG | 548 | CCAUCAUCUAUGCCUUUGUC | 1063 |
| | GTCATGGTCATCTGCTACTCGGG | 549 | GUCAUGGUCAUCUGCUACUC | 1064 |
| | CATACAGTCAGTATCAATTCTGG | 550 | CAUACAGUCAGUAUCAAUUC | 1065 |
| | TTTACCAGATCTCAAAAAGAAGG | 551 | UUUACCAGAUCUCAAAAAGA | 1066 |
| | ACAGCATTTGCAGAAGCGTTTGG | 552 | ACAGCAUUUGCAGAAGCGUU | 1067 |
| | ATATCTGTGGGCTTGTGACACGG | 553 | AUAUCUGUGGGCUUGUGACA | 1068 |
| | AAGTGTGATCACTTGGGTGGTGG | 554 | AAGUGUGAUCACUUGGGUGG | 1069 |
| | TTGTATTTCCAAAGTCCCACTGG | 555 | UUGUAUUUCCAAAGUCCCAC | 1070 |
| | CCCATCATCTATGCCTTTGTCGG | 556 | CCCAUCAUCUAUGCCUUUGU | 1071 |
| | TGTATTTCCAAAGTCCCACTGGG | 557 | UGUAUUUCCAAAGUCCCACU | 1072 |
| | ATGCAGGTGACAGAGACTCTTGG | 558 | AUGCAGGUGACAGAGACUCU | 1073 |
| | TCAGCCTTTTGCAGTTTATCAGG | 559 | UCAGCCUUUUGCAGUUUAUC | 1074 |
| | AAAGATAGTCATCTTGGGGCTGG | 560 | AAAGAUAGUCAUCUUGGGGC | 1075 |
| | AAAGCCAGGACGGTCACCTTTGG | 561 | AAAGCCAGGACGGUCACCUU | 1076 |

The products of the present invention may be used to modify, e.g., repress or silence, genes having CpG islands (CGI). Genes having CGI include: B2M; TET2; TGFBR2; A2AR; CISH; PTPN11; PTPN6; PTPA; PTPN2; JUNB; TOX; TOX2; NR4A1; NR4A2; NR4A3; MAP4K1; REL; IRF4; DGKA; PIK3CD; HLA-A; USP16; DCK and FAS.

For example, targeting genes, such as genes with a CGI, may:
produce allogenic products (e.g., by targeting B2M and/or HLA-A);
alter resistance to an immunosuppressive tumour microenvironment (e.g., by targeting of TGFBR2, A2AR, PTPN11, PTPN6, PTPN2, and/or DGKA);
allow CAR/transgenic TCR integration in a safe site (e.g., by targeting of AAVS1 and/or CCR5);
provide resistance to exhaustion (e.g., by targeting of FAS, CISH, PTPA, PIK3CD, MAP4K1, NR4A1, NR4A2, NR4A3, JUNB, REL, TOX, TOX2, IRF4 and/or TET2); and/or
delay T cell senescence (e.g., by targeting USP16).

Silencing of the TCR genes, PDCD1 and CTLA4 may be used to improve efficacy of cancer immunotherapy approaches.

Silencing of B2M may be used to generate allogeneic HSPCs, T cells or mesenchymal cells to be used for transplantation.

In one aspect, the present invention provides gRNAs which target a sequence set forth in any one of SEQ ID NOs: 1077 to 2777.

By way of example, target genes having CGI islands and exemplary gRNAs suitable for targeting said genes are presented in Table 2 below (SEQ: SEQ ID NO).

TABLE 2
Target genes having CGI islands and exemplary gRNAs

| Target | | | | |
|---|---|---|---|---|
| gene | Exemplary target regions | | Exemplary gRNA spacer | |
| (with CGI) | (including PAM) | SEQ | sequence | SEQ |
| B2M | CGATAAGCGTCAGAGCGCCGAGG | 1077 | CGAUAAGCGUCAGAGCGCCG | 2778 |
| | TTTGGCCTACGGCGACGGGAGGG | 1078 | UUUGGCCUACGGCGACGGGA | 2779 |
| | CATCGGCGCCCTCCGATCTGGGG | 1079 | CAUCGGCGCCCUCCGAUCUG | 2780 |
| | CTTTGGCCTACGGCGACGGGAGG | 1080 | CUUUGGCCUACGGCGACGGG | 2781 |
| | TATAAGTGGAGGCGTCGCGCTGG | 1081 | UAUAAGUGGAGGCGUCGCGC | 2782 |
| | CTCCCGTCGCCGTAGGCCAAAGG | 1082 | CUCCCGUCGCCGUAGGCCAA | 2783 |
| | GACCTTTGGCCTACGGCGACGGG | 1083 | GACCUUUGGCCUACGGCGAC | 2784 |
| | AGACCTTTGGCCTACGGCGACGG | 1084 | AGACCUUUGGCCUACGGCGA | 2785 |
| | CGCTACTTGCCCCTTTCGGCGGG | 1085 | CGCUACUUGCCCCUUUCGGC | 2786 |
| | ACATCGGCGCCCTCCGATCTGGG | 1086 | ACAUCGGCGCCCUCCGAUCU | 2787 |
| | CGCGCGCTACTTGCCCCTTTCGG | 1087 | CGCGCGCUACUUGCCCCUUU | 2788 |
| | TACATCGGCGCCCTCCGATCTGG | 1088 | UACAUCGGCGCCCUCCGAUC | 2789 |
| | GTCCGAGCAGTTAACTGGCTGGG | 1089 | GUCCGAGCAGUUAACUGGCU | 2790 |
| | CACGCGTTTAATATAAGTGGAGG | 1090 | CACGCGUUUAAUAUAAGUGG | 2791 |
| | CGCGACGTTTGTAGAATGCTTGG | 1091 | CGCGACGUUUGUAGAAUGCU | 2792 |
| | GGGCACGCGTTTAATATAAGTGG | 1092 | GGGCACGCGUUUAAUAUAAG | 2793 |
| | GCTACTTGCCCCTTTCGGCGGGG | 1093 | GCUACUUGCCCCUUUCGGCG | 2794 |
| | AAGCGTCAGAGCGCCGAGGTTGG | 1094 | AAGCGUCAGAGCGCCGAGGU | 2795 |
| | GCGCTACTTGCCCCTTTCGGCGG | 1095 | GCGCUACUUGCCCCUUUCGG | 2796 |
| | AAGTGGAGGCGTCGCGCTGGCGG | 1096 | AAGUGGAGGCGUCGCGCUGG | 2797 |
| | GCCTACGGCGACGGGAGGGTCGG | 1097 | GCCUACGGCGACGGGAGGGU | 2798 |
| | GGTCCGAGCAGTTAACTGGCTGG | 1098 | GGUCCGAGCAGUUAACUGGC | 2799 |
| | AGCGTCAGAGCGCCGAGGTTGGG | 1099 | AGCGUCAGAGCGCCGAGGUU | 2800 |
| | GAACGCGTGGAGGGGCGCTTGGG | 1100 | GAACGCGUGGAGGGGCGCUU | 2801 |
| | GGCGCTCATTCTAGGACTTCAGG | 1101 | GGCGCUCAUUCUAGGACUUC | 2802 |
| | TTCGCATGTCCTAGCACCTCTGG | 1102 | UUCGCAUGUCCUAGCACCUC | 2803 |
| | AACCTCAGCGCCGCGCCTTTGGG | 1103 | AACCUCAGCGCCGCGCCUUU | 2804 |
| | CTCCTTGGTGGCCCGCCGTGGGG | 1104 | CUCCUUGGUGGCCCGCCGUG | 2805 |
| | ACTCACGCTGGATAGCCTCCAGG | 1105 | ACUCACGCUGGAUAGCCUCC | 2806 |
| | GGCGCGCACCCCAGATCGGAGGG | 1106 | GGCGCGCACCCCAGAUCGGA | 2807 |
| | TCGCATGTCCTAGCACCTCTGGG | 1107 | UCGCAUGUCCUAGCACCUCU | 2808 |
| | AACGCGTGGAGGGGCGCTTGGGG | 1108 | AACGCGUGGAGGGGCGCUUG | 2809 |
| | TTCTCTTCCGCTCTTTCGCGGGG | 1109 | UUCUCUUCCGCUCUUUCGCG | 2810 |
| | GACGGGTAGGCTCGTCCCAAAGG | 1110 | GACGGGUAGGCUCGUCCCAA | 2811 |
| | CCCGCCGTGGGGCTAGTCCAGGG | 1111 | CCCGCCGUGGGGCUAGUCCA | 2812 |
| | GAGTAGCGCGAGCACAGCTAAGG | 1112 | GAGUAGCGCGAGCACAGCUA | 2813 |
| | GGGGCAAGTAGCGCGCGTCCCGG | 1113 | GGGGCAAGUAGCGCGCGUCC | 2814 |
| | AGCGCCCGGTGTCCCAAGCTGGG | 1114 | AGCGCCCGGUGUCCCAAGCU | 2815 |
| | CCTACGGCGACGGGAGGGTCGGG | 1115 | CCUACGGCGACGGGAGGGUC | 2816 |
| | CAAGCCAGCGACGCAGTGCCAGG | 1116 | CAAGCCAGCGACGCAGUGCC | 2817 |
| | AAACCTCAGCGCCGCGCCTTTGG | 1117 | AAACCUCAGCGCCGCGCCUU | 2818 |
| | TGAACGCGTGGAGGGGCGCTTGG | 1118 | UGAACGCGUGGAGGGGCGCU | 2819 |
| | CTAACCTGGCACTGCGTCGCTGG | 1119 | CUAACCUGGCACUGCGUCGC | 2820 |
| | CGTCAGAGCGCCGAGGTTGGGGG | 1120 | CGUCAGAGCGCCGAGGUUGG | 2821 |
| | GGCCGAGATGTCTCGCTCCGTGG | 1121 | GGCCGAGAUGUCUCGCUCCG | 2822 |
| | GCTAGGACATGCGAACTTAGCGG | 1122 | GCUAGGACAUGCGAACUUAG | 2823 |
| | CGCTGAGGTTTGTGAACGCGTGG | 1123 | CGCUGAGGUUUGUGAACGCG | 2824 |
| | GGGCGCGCACCCCAGATCGGAGG | 1124 | GGGCGCGCACCCCAGAUCGG | 2825 |
| | AAACGCGTGCCCAGCCAATCAGG | 1125 | AAACGCGUGCCCAGCCAAUC | 2826 |
| | TGCAGGTCCGAGCAGTTAACTGG | 1126 | UGCAGGUCCGAGCAGUUAAC | 2827 |
| | GGACACCGGGCGCTCATTCTAGG | 1127 | GGACACCGGGCGCUCAUUCU | 2828 |
| | CCGCTCTTTCGCGGGGCCTCTGG | 1128 | CCGCUCUUUCGCGGGGCCUC | 2829 |
| | GTAGGCTCGTCCCAAAGGCGCGG | 1129 | GUAGGCUCGUCCCAAAGGCG | 2830 |
| | TCCGAGCAGTTAACTGGCTGGGG | 1130 | UCCGAGCAGUUAACUGGCUG | 2831 |
| | TAGTCCAGGGCTGGATCTCGGGG | 1131 | UAGUCCAGGGCUGGAUCUCG | 2832 |
| | CTAGGACATGCGAACTTAGCGGG | 1132 | CUAGGACAUGCGAACUUAGC | 2833 |
| | AGTGGAGGCGTCGCGCTGGCGGG | 1133 | AGUGGAGGCGUCGCGCUGGC | 2834 |
| | TCTATGTGGGGCCACACCGTGGG | 1134 | UCUAUGUGGGGCCACACCGU | 2835 |
| | CTATGTGGGGCCACACCGTGGGG | 1135 | CUAUGUGGGGCCACACCGUG | 2836 |
| | GCGTCAGAGCGCCGAGGTTGGGG | 1136 | GCGUCAGAGCGCCGAGGUUG | 2837 |
| | GCGCCCGGTGTCCCAAGCTGGGG | 1137 | GCGCCCGGUGUCCCAAGCUG | 2838 |
| | CGCAGCAGACAGGCTTACCCGGG | 1138 | CGCAGCAGACAGGCUUACCC | 2839 |
| | CAATCAGGACAAGGCCCGCAGGG | 1139 | CAAUCAGGACAAGGCCCGCA | 2840 |
| | GAGTCTCGTGATGTTTAAGAAGG | 1140 | GAGUCUCGUGAUGUUUAAGA | 2841 |
| | TGGATCTCGGGGAAGCGGCGGGG | 1141 | UGGAUCUCGGGGAAGCGGCG | 2842 |
| | CATCACGAGACTCTAAGAAAAGG | 1142 | CAUCACGAGACUCUAAGAAA | 2843 |
| | GGGCAAGTAGCGCGCGTCCCGGG | 1143 | GGGCAAGUAGCGCGCGUCCC | 2844 |
| | GAGGGTCGGGACAAAGTTTAGGG | 1144 | GAGGGUCGGGACAAAGUUUA | 2845 |
| | GCCCCTTTCGGCGGGGAGCAGGG | 1145 | GCCCCUUUCGGCGGGGAGCA | 2846 |
| | TCTCCTTGGTGGCCCGCCGTGGG | 1146 | UCUCCUUGGUGGCCCGCCGU | 2847 |
| | TCCCCTGCTCCCCGCCGAAAGGG | 1147 | UCCCCUGCUCCCCGCCGAAA | 2848 |
| | GCCCGCCGTGGGGCTAGTCCAGG | 1148 | GCCCGCCGUGGGGCUAGUCC | 2849 |
| | TGCCCCTTTCGGCGGGGAGCAGG | 1149 | UGCCCCUUUCGGCGGGGAGC | 2850 |
| | GAGGTTTGTGAACGCGTGGAGGG | 1150 | GAGGUUUGUGAACGCGUGGA | 2851 |
| | TGGGGTGCGCGCCCCAGCTTGGG | 1151 | UGGGGUGCGCGCCCCAGCUU | 2852 |
| | AGGTTTGTGAACGCGTGGAGGGG | 1152 | AGGUUUGUGAACGCGUGGAG | 2853 |
| | GCCCGAATGCTGTCAGCTTCAGG | 1153 | GCCCGAAUGCUGUCAGCUUC | 2854 |
| | GAGAGCTGTGGACTTCGTCTAGG | 1154 | GAGAGCUGUGGACUUCGUCU | 2855 |
| | CTAGCACCTCTGGGTCTATGTGG | 1155 | CUAGCACCUCUGGGUCUAUG | 2856 |
| | CCGGGTAAGCCTGTCTGCTGCGG | 1156 | CCGGGUAAGCCUGUCUGCUG | 2857 |
| | CGCAGTGCCAGGTTAGAGAGAGG | 1157 | CGCAGUGCCAGGUUAGAGAG | 2858 |
| | TGAGGTTTGTGAACGCGTGGAGG | 1158 | UGAGGUUUGUGAACGCGUGG | 2859 |
| | AGCCCCACGGCGGGCCACCAAGG | 1159 | AGCCCCACGGCGGGCCACCA | 2860 |
| | TGCGTCGCTGGCTTGGAGACAGG | 1160 | UGCGUCGCUGGCUUGGAGAC | 2861 |
| | GGCCACGGAGCGAGACATCTCGG | 1161 | GGCCACGGAGCGAGACAUCU | 2862 |
| | GCGGGCCACCAAGGAGAACTTGG | 1162 | GCGGGCCACCAAGGAGAACU | 2863 |
| | TTCTCCTTGGTGGCCCGCCGTGG | 1163 | UUCUCCUUGGUGGCCCGCCG | 2864 |
| | CCTGCGGGCCTTGTCCTGATTGG | 1164 | CCUGCGGGCCUUGUCCUGAU | 2865 |
| | GCCCCAGCCAGTTAACTGCTCGG | 1165 | GCCCCAGCCAGUUAACUGCU | 2866 |
| | CTGGATCTCGGGGAAGCGGCGGG | 1166 | CUGGAUCUCGGGGAAGCGGC | 2867 |
| | CTCGCGCTACTCTCTCTTTCTGG | 1167 | CUCGCGCUACUCUCUCUUUC | 2868 |
| | CGCGAGCACAGCTAAGGCCACGG | 1168 | CGCGAGCACAGCUAAGGCCA | 2869 |
| | GAAAGTCCCTCTCTCTAACCTGG | 1169 | GAAAGUCCCUCUCUCUAACC | 2870 |
| | GTCCCAAAGGCGCGGCGCTGAGG | 1170 | GUCCCAAAGGCGCGGCGCUG | 2871 |
| | CGGGCCTTGTCCTGATTGGCTGG | 1171 | CGGGCCUUGUCCUGAUUGGC | 2872 |
| | CGGAGCGAGAGAGCACAGCGAGG | 1172 | CGGAGCGAGAGAGCACAGCG | 2873 |
| | GTCTATGTGGGGCCACACCGTGG | 1173 | GUCUAUGUGGGGCCACACCG | 2874 |
| | CGGCTCTGCTTCCCTTAGACTGG | 1174 | CGGCUCUGCUUCCCUUAGAC | 2875 |
| | CTCATTCTAGGACTTCAGGCTGG | 1175 | CUCAUUCUAGGACUUCAGGC | 2876 |
| | CGCGCCCCAGCTTGGGACACCGG | 1176 | CGCGCCCCAGCUUGGGACAC | 2877 |
| | AGGGTAGGAGAGACTCACGCTGG | 1177 | AGGGUAGGAGAGACUCACGC | 2878 |
| HLA-A | GGCGCTTCCTCCGCGGGTACCGG | 1178 | GGCGCUUCCUCCGCGGGUAC | 2879 |
| | GAGTCCCGGTGGGTGCGTGCGGG | 1179 | GAGUCCCGGUGGGUGCGUGC | 2880 |
| | CAGACTGACCGAGTGGACCTGGG | 1180 | CAGACUGACCGAGUGGACCU | 2881 |
| | TACCGGCAGGACGCCTACGACGG | 1181 | UACCGGCAGGACGCCUACGA | 2882 |
| | TGGTACAGGATCTGGAACCCAGG | 1182 | UGGUACAGGAUCUGGAACCC | 2883 |
| | CGTCCTGCCGGTACCCGCGGAGG | 1183 | CGUCCUGCCGGUACCCGCGG | 2884 |
| | AGGCGTCCTGCCGGTACCCGCGG | 1184 | AGGCGUCCUGCCGGUACCCG | 2885 |
| | CTTCCTCCGCGGGTACCGGCAGG | 1185 | CUUCCUCCGCGGGUACCGGC | 2886 |
| | ACAGACTGACCGAGTGGACCTGG | 1186 | ACAGACUGACCGAGUGGACC | 2887 |
| | CCAGTCACAGACTGACCGAGTGG | 1187 | CCAGUCACAGACUGACCGAG | 2888 |
| | TGCCGTCGTAGGCGTCCTGCCGG | 1188 | UGCCGUCGUAGGCGUCCUGC | 2889 |
| | CAGGATGAAGGACCCTACGTAGG | 1189 | CAGGAUGAAGGACCCUACGU | 2890 |
| | GACCAACCCGGGGGGATTTTTGG | 1190 | GACCAACCCGGGGGGAUUUU | 2891 |
| | TACAGGATCTGGAACCCAGGAGG | 1191 | UACAGGAUCUGGAACCCAGG | 2892 |
| | ACGACACTGATTGGCTTCTCTGG | 1192 | ACGACACUGAUUGGCUUCUC | 2893 |
| | GGCCAAAAATCCCCCCGGGTTGG | 1193 | GGCCAAAAAUCCCCCCGGGU | 2894 |
| | GGCCCGTCCGTGGGGGATGAGGG | 1194 | GGCCCGUCCGUGGGGGAUGA | 2895 |
| | TCCTGGCGGGGGCGCAGGACCGG | 1195 | UCCUGGCGGGGGCGCAGGAC | 2896 |
| | GCGACCGCGACGACACTGATTGG | 1196 | GCGACCGCGACGACACUGAU | 2897 |
| | CTACGTAGGGTCCTTCATCCTGG | 1197 | CUACGUAGGGUCCUUCAUCC | 2898 |
| | TTTAGGCCAAAAATCCCCCCGGG | 1198 | UUUAGGCCAAAAAUCCCCCC | 2899 |
| | GAGGGTTCGGGGCGCCATGACGG | 1199 | GAGGGUUCGGGGCGCCAUGA | 2900 |
| | TGAAGGACCCTACGTAGGTTGGG | 1200 | UGAAGGACCCUACGUAGGUU | 2901 |
| | CGCCTCTGCGGGGAGAAGCAAGG | 1201 | CGCCUCUGCGGGGAGAAGCA | 2902 |
| | GCCCGTCCGTGGGGGATGAGGGG | 1202 | GCCCGUCCGUGGGGGAUGAG | 2903 |
| | ACCCCTCATCCCCCACGGACGGG | 1203 | ACCCCUCAUCCCCCACGGAC | 2904 |
| | TCAGGACCCCTCATCCCCCACGG | 1204 | UCAGGACCCCUCAUCCCCCA | 2905 |
| | TGGGCGACCTGGCCCGTCCGTGG | 1205 | UGGGCGACCUGGCCCGUCCG | 2906 |
| | GACCCTACGTAGGTTGGGAGAGG | 1206 | GACCCUACGUAGGUUGGGAG | 2907 |
| | GACGCCGAGGATGGCCGTCATGG | 1207 | GACGCCGAGGAUGGCCGUCA | 2908 |
| | TTCACATCCGTGTCCCGGCCCGG | 1208 | UUCACAUCCGUGUCCCGGCC | 2909 |
| | GACGGCCATCCTCGGCGTCTGGG | 1209 | GACGGCCAUCCUCGGCGUCU | 2910 |
| | ACCCTACGTAGGTTGGGAGAGGG | 1210 | ACCCUACGUAGGUUGGGAGA | 2911 |
| | ACGGCCATCCTCGGCGTCTGGGG | 1211 | ACGGCCAUCCUCGGCGUCUG | 2912 |
| | ATGAAGGACCCTACGTAGGTTGG | 1212 | AUGAAGGACCCUACGUAGGU | 2913 |
| | AAGCAAGGGGCCCTCCTGGCGGG | 1213 | AAGCAAGGGGCCCUCCUGGC | 2914 |
| | TGACGGCCATCCTCGGCGTCTGG | 1214 | UGACGGCCAUCCUCGGCGUC | 2915 |
| | AGGCGCCTGGGCCTCTCCCGGGG | 1215 | AGGCGCCUGGGCCUCUCCCG | 2916 |
| | ATTTCTTCACATCCGTGTCCCGG | 1216 | AUUUCUUCACAUCCGUGUCC | 2917 |
| | TCTCCCGGGGCAAGGGTCTCGGG | 1217 | UCUCCCGGGGCAAGGGUCUC | 2918 |
| | TCCCTCTCCCAACCTACGTAGGG | 1218 | UCCCUCUCCCAACCUACGUA | 2919 |
| | GGGCGACCTGGCCCGTCCGTGGG | 1219 | GGGCGACCUGGCCCGUCCGU | 2920 |
| | GAAGCAAGGGGCCCTCCTGGCGG | 1220 | GAAGCAAGGGGCCCUCCUGG | 2921 |
| | GTCTCGGGGTCCCGCGGCTTCGG | 1221 | GUCUCGGGGUCCCGCGGCUU | 2922 |
| | GCGGAGTTGGGGAATCCCCAAGG | 1222 | GCGGAGUUGGGGAAUCCCCA | 2923 |
| | GCGCCCGCGGCTCCATCCTCTGG | 1223 | GCGCCCGCGGCUCCAUCCUC | 2924 |
| | TCTCGGGGTCCCGCGGCTTCGGG | 1224 | UCUCGGGGUCCCGCGGCUUC | 2925 |
| | GACCCCTCATCCCCCACGGACGG | 1225 | GACCCCUCAUCCCCCACGGA | 2926 |
| | AGCAAGGGGCCCTCCTGGCGGGG | 1226 | AGCAAGGGGCCCUCCUGGCG | 2927 |
| | CATCCTGGATACTCACGACGCGG | 1227 | CAUCCUGGAUACUCACGACG | 2928 |
| TGFBR2 | TCGGTCTATGACGAGCAGCGGGG | 1228 | UCGGUCUAUGACGAGCAGCG | 2929 |
| | GAGTGAGTCACTCGCGCGCACGG | 1229 | GAGUGAGUCACUCGCGCGCA | 2930 |
| | TGCTGGCGATACGCGTCCACAGG | 1230 | UGCUGGCGAUACGCGUCCAC | 2931 |
| | CGTTGTGTTGGCCGCGTTCGAGG | 1231 | CGUUGUGUUGGCCGCGUUCG | 2932 |
| | GTGGGGGCTCGCCTCGAACGCGG | 1232 | GUGGGGGCUCGCCUCGAACG | 2933 |
| | TGGGCACGCGGCATCGCCATGGG | 1233 | UGGGCACGCGGCAUCGCCAU | 2934 |
| | CTTTCCTCGTTTCCGCCCGGGGG | 1234 | CUUUCCUCGUUUCCGCCCGG | 2935 |
| | GCACGCGGCATCGCCATGGGCGG | 1235 | GCACGCGGCAUCGCCAUGGG | 2936 |
| | GAAACTCCTCGCCAACAGCTGGG | 1236 | GAAACUCCUCGCCAACAGCU | 2937 |
| | GCCCGACTCCCGTAGCTGCAGGG | 1237 | GCCCGACUCCCGUAGCUGCA | 2938 |
| | GACTGTCAAGCGCAGCGGAGAGG | 1238 | GACUGUCAAGCGCAGCGGAG | 2939 |
| | AGTCGGCCAAAGCTCTCGGAGGG | 1239 | AGUCGGCCAAAGCUCUCGGA | 2940 |
| | TGGTTATCTGAAGGCGGCCGGGG | 1240 | UGGUUAUCUGAAGGCGGCCG | 2941 |
| | AACGTGCGGTGGGATCGTGCTGG | 1241 | AACGUGCGGUGGGAUCGUGC | 2942 |
| | ACTTTCCTCGTTTCCGCCCGGGG | 1242 | ACUUUCCUCGUUUCCGCCCG | 2943 |
| | TCTCCGCTGCGCTTGACAGTCGG | 1243 | UCUCCGCUGCGCUUGACAGU | 2944 |
| | GGACGATGTGCAGCGGCCACAGG | 1244 | GGACGAUGUGCAGCGGCCAC | 2945 |
| | CTCGGTCTATGACGAGCAGCGGG | 1245 | CUCGGUCUAUGACGAGCAGC | 2946 |
| | GTGGGCACGCGGCATCGCCATGG | 1246 | GUGGGCACGCGGCAUCGCCA | 2947 |
| | GTCGGCCAAAGCTCTCGGAGGGG | 1247 | GUCGGCCAAAGCUCUCGGAG | 2948 |
| | TCACCCGACTTCTGAACGTGCGG | 1248 | UCACCCGACUUCUGAACGUG | 2949 |
| | ACGTTCAGAAGTCGGGTGAGTGG | 1249 | ACGUUCAGAAGUCGGGUGAG | 2950 |
| | GTTCAGTTGCAAGGGGCGCGGGG | 1250 | GUUCAGUUGCAAGGGGCGCG | 2951 |
| | CGGCATCGCCATGGGCGGAGTGG | 1251 | CGGCAUCGCCAUGGGCGGAG | 2952 |
| | GCTCGGTCTATGACGAGCAGCGG | 1252 | GCUCGGUCUAUGACGAGCAG | 2953 |
| | GACAGTCGGGCCCGGCAACCCGG | 1253 | GACAGUCGGGCCCGGCAACC | 2954 |
| | CGCGTGCACCCGCTCGGGACAGG | 1254 | CGCGUGCACCCGCUCGGGAC | 2955 |
| | CTCCGCTGCGCTTGACAGTCGGG | 1255 | CUCCGCUGCGCUUGACAGUC | 2956 |
| | TGGCGAGCGGGCGCCACATCTGG | 1256 | UGGCGAGCGGGCGCCACAUC | 2957 |
| | AACTTCAACTCAGCGCTGCGGGG | 1257 | AACUUCAACUCAGCGCUGCG | 2958 |
| | ACTTCAACTCAGCGCTGCGGGGG | 1258 | ACUUCAACUCAGCGCUGCGG | 2959 |
| | GTCCCGAGCGGGTGCACGCGCGG | 1259 | GUCCCGAGCGGGUGCACGCG | 2960 |
| | AGCCCGACTCCCGTAGCTGCAGG | 1260 | AGCCCGACUCCCGUAGCUGC | 2961 |
| | AACTTTCCTCGTTTCCGCCCGGG | 1261 | AACUUUCCUCGUUUCCGCCC | 2962 |
| | GCCTTTCCTGCTCGCACAAAGGG | 1262 | GCCUUUCCUGCUCGCACAAA | 2963 |
| | GGCCCGACTGTCAAGCGCAGCGG | 1263 | GGCCCGACUGUCAAGCGCAG | 2964 |
| | GTCGGGCTGCGTGAGTGTCGCGG | 1264 | GUCGGGCUGCGUGAGUGUCG | 2965 |
| | TTGGTCCCCTTTGTGCGAGCAGG | 1265 | UUGGUCCCCUUUGUGCGAGC | 2966 |
| | CGCAGCGGACGGCGCCTTCCCGG | 1266 | CGCAGCGGACGGCGCCUUCC | 2967 |
| | CTCGTTTCCGCCCGGGGGCCGGG | 1267 | CUCGUUUCCGCCCGGGGGCC | 2968 |
| | CCTCGTTTCCGCCCGGGGGCCGG | 1268 | CCUCGUUUCCGCCCGGGGGC | 2969 |
| | AGTCCGGCTCCTGTCCCGAGCGG | 1269 | AGUCCGGCUCCUGUCCCGAG | 2970 |
| | GTGGCCGTCTCCAGGAGCTAAGG | 1270 | GUGGCCGUCUCCAGGAGCUA | 2971 |
| | GGCAGCTACGAGAGAGCTAGGGG | 1271 | GGCAGCUACGAGAGAGCUAG | 2972 |
| | TCAAGCGCAGCGGAGAGGCGGGG | 1272 | UCAAGCGCAGCGGAGAGGCG | 2973 |
| | CCCACCGCACGTTCAGAAGTCGG | 1273 | CCCACCGCACGUUCAGAAGU | 2974 |
| | TGGCAGCTACGAGAGAGCTAGGG | 1274 | UGGCAGCUACGAGAGAGCUA | 2975 |
| | GAGCTGGCCTTTTGAACGGGTGG | 1275 | GAGCUGGCCUUUUGAACGGG | 2976 |
| | TGTCAAGCGCAGCGGAGAGGCGG | 1276 | UGUCAAGCGCAGCGGAGAGG | 2977 |
| | CGAGCAGCGGGGTCTGCCATGGG | 1277 | CGAGCAGCGGGGUCUGCCAU | 2978 |
| | TTCTTTAGGTCGAAGTCTAGAGG | 4539 | UUCUUUAGGUCGAAGUCUAG | 4553 |
| | GTGCTCGCGACTCAATAGATTGG | 4540 | GUGCUCGCGACUCAAUAGAU | 4554 |
| | AACGCATCTCTAAAGCACCTAGG | 4541 | AACGCAUCUCUAAAGCACCU | 4555 |
| | CTGATCTACTAGGGAAAACGTGG | 4542 | CUGAUCUACUAGGGAAAACG | 4556 |
| | TTGAGTAAATACTTGGAGCGAGG | 4543 | UUGAGUAAAUACUUGGAGCG | 4557 |
| | GGGGCCTCCCCGCGCCTCGCCGG | 4544 | GGGGCCUCCCCGCGCCUCGC | 4558 |
| | CCTGAGCAGCCCCCGACCCATGG | 4545 | CCUGAGCAGCCCCCGACCCA | 4559 |
| A2AR | AAGGTTCATGCGAGCGCGCGGGG | 1278 | AAGGUUCAUGCGAGCGCGCG | 2979 |
| | GAAGGTTCATGCGAGCGCGCGGG | 1279 | GAAGGUUCAUGCGAGCGCGC | 2980 |
| | ATTTGGCGCAAGGCGGCCCAAGG | 1280 | AUUUGGCGCAAGGCGGCCCA | 2981 |
| | TCCTGGAAGGACGATCCCGCAGG | 1281 | UCCUGGAAGGACGAUCCCGC | 2982 |
| | CGAAGGTTCATGCGAGCGCGCGG | 1282 | CGAAGGUUCAUGCGAGCGCG | 2983 |
| | GTCTGCGGCGCATGGACGGACGG | 1283 | GUCUGCGGCGCAUGGACGGA | 2984 |
| | TCCGTCCCCCGTCGTCTCCTGGG | 1284 | UCCGUCCCCCGUCGUCUCCU | 2985 |
| | TCCGTCCATGCGCCGCAGACCGG | 1285 | UCCGUCCAUGCGCCGCAGAC | 2986 |
| | AACTGCACCGGAAGGCGCGCAGG | 1286 | AACUGCACCGGAAGGCGCGC | 2987 |
| | GTGGCGGCTCTCGAGGGATTTGG | 1287 | GUGGCGGCUCUCGAGGGAUU | 2988 |
| | ACCTGCGGGATCGTCCTTCCAGG | 1288 | ACCUGCGGGAUCGUCCUUCC | 2989 |
| | ATCCGTCCCCCGTCGTCTCCTGG | 1289 | AUCCGUCCCCCGUCGUCUCC | 2990 |
| | CGCGCCTTCCGGTGCAGTTTGGG | 1290 | CGCGCCUUCCGGUGCAGUUU | 2991 |
| | CTCGGTTTCTCCGCGCAGCGGGG | 1291 | CUCGGUUUCUCCGCGCAGCG | 2992 |
| | CTGTCCCAAACTGCACCGGAAGG | 1292 | CUGUCCCAAACUGCACCGGA | 2993 |
| | CGAGCTGTCCCAAACTGCACCGG | 1293 | CGAGCUGUCCCAAACUGCAC | 2994 |
| | GTCGCGGCCTCGTCCTGACAGGG | 1294 | GUCGCGGCCUCGUCCUGACA | 2995 |
| | AGGACTCGGACCCCGCGCCGGGG | 1295 | AGGACUCGGACCCCGCGCCG | 2996 |
| | TCACGTCCCAGGCGCAGTTGCGG | 1296 | UCACGUCCCAGGCGCAGUUG | 2997 |
| | TGTCAGGACGAGGCCGCGACGGG | 1297 | UGUCAGGACGAGGCCGCGAC | 2998 |
| | ACCGGAAGGCGCGCAGGGGTAGG | 1298 | ACCGGAAGGCGCGCAGGGGU | 2999 |
| | CTCTCGAGGGATTTGGCGCAAGG | 1299 | CUCUCGAGGGAUUUGGCGCA | 3000 |
| | TTCATGCGAGCGCGCGGGGCCGG | 1300 | UUCAUGCGAGCGCGCGGGGC | 3001 |
| | TTTCTCCGCGCAGCGGGGCGGGG | 1301 | UUUCUCCGCGCAGCGGGGCG | 3002 |
| | GCCCGGGACGCGCCGAGAAAGGG | 1302 | GCCCGGGACGCGCCGAGAAA | 3003 |
| | TCGAGGGATTTGGCGCAAGGCGG | 1303 | UCGAGGGAUUUGGCGCAAGG | 3004 |
| | CGGCGGGAAAGGAACCCTGAGGG | 1304 | CGGCGGGAAAGGAACCCUGA | 3005 |
| | GCGCCGGGGAACTGGTCTCGGGG | 1305 | GCGCCGGGGAACUGGUCUCG | 3006 |
| | GAGACCAGTTCCCCGGCGCGGGG | 1306 | GAGACCAGUUCCCCGGCGCG | 3007 |
| | AGGATCGCCTGCGGGCCTCGCGG | 1307 | AGGAUCGCCUGCGGGCCUCG | 3008 |
| | CCGAGCGCTGGCGTCTTCCGTGG | 1308 | CCGAGCGCUGGCGUCUUCCG | 3009 |
| | CCGGGACGCGCCGAGAAAGGGGG | 1309 | CCGGGACGCGCCGAGAAAGG | 3010 |
| | AAAGTCTGAGTGCGGGACACAGG | 1310 | AAAGUCUGAGUGCGGGACAC | 3011 |
| | TTTGGCGCAAGGCGGCCCAAGGG | 1311 | UUUGGCGCAAGGCGGCCCAA | 3012 |
| | GGGAATGGTGGCGGCTCTCGAGG | 1312 | GGGAAUGGUGGCGGCUCUCG | 3013 |
| | CACGCCGGCTCCCGCTGTCTCGG | 1313 | CACGCCGGCUCCCGCUGUCU | 3014 |
| | CGTCGCGGCCTCGTCCTGACAGG | 1314 | CGUCGCGGCCUCGUCCUGAC | 3015 |
| | CGCCCGGGACGCGCCGAGAAAGG | 1315 | CGCCCGGGACGCGCCGAGAA | 3016 |
| | GGCCTCGCGGGCCGATGCCTCGG | 1316 | GGCCUCGCGGGCCGAUGCCU | 3017 |
| | TGCAGTTTGGGACAGCTCGGAGG | 1317 | UGCAGUUUGGGACAGCUCGG | 3018 |
| | CTGACCTGCCGCTCGCACGCCGG | 1318 | CUGACCUGCCGCUCGCACGC | 3019 |
| | GCCGGTCTGCGGCGCATGGACGG | 1319 | GCCGGUCUGCGGCGCAUGGA | 3020 |
| | GCGCGCCTTCCGGTGCAGTTTGG | 1320 | GCGCGCCUUCCGGUGCAGUU | 3021 |
| | GCCTCGGTTTCTCCGCGCAGCGG | 1321 | GCCUCGGUUUCUCCGCGCAG | 3022 |
| | GCCGGCTCCCGCTGTCTCGGCGG | 1322 | GCCGGCUCCCGCUGUCUCGG | 3023 |
| | ACCCGAGGCATCGGCCCGCGAGG | 1323 | ACCCGAGGCAUCGGCCCGCG | 3024 |
| | GGGACGGATGCGAGCCCGGGAGG | 1324 | GGGACGGAUGCGAGCCCGGG | 3025 |
| | GGCCCGCAGGCGATCCTGGAAGG | 1325 | GGCCCGCAGGCGAUCCUGGA | 3026 |
| | TAGTTGCCCCGACTGTACCATGG | 1326 | UAGUUGCCCCGACUGUACCA | 3027 |
| | AAGGCGCGCAGGGGTAGGCGGGG | 1327 | AAGGCGCGCAGGGGUAGGCG | 3028 |
| | GAGACGACGGGGGACGGATGGGG | 1328 | GAGACGACGGGGGACGGAUG | 3029 |
| | GCGGCGCCGCAACTGCGCCTGGG | 1329 | GCGGCGCCGCAACUGCGCCU | 3030 |
| | CCTCGGTTTCTCCGCGCAGCGGG | 1330 | CCUCGGUUUCUCCGCGCAGC | 3031 |
| | CGTGCGAGCGGCAGGTCAGCCGG | 1331 | CGUGCGAGCGGCAGGUCAGC | 3032 |
| | CGGGGGATGTGGCGCGGTCCAGG | 1332 | CGGGGGAUGUGGCGCGGUCC | 3033 |
| | CGGCGGGACGGATGCGAGCCCGG | 1333 | CGGCGGGACGGAUGCGAGCC | 3034 |
| | CGATGCCTCGGGTCCCCCTCCGG | 1334 | CGAUGCCUCGGGUCCCCCUC | 3035 |
| | ACTGCACCGGAAGGCGCGCAGGG | 1335 | ACUGCACCGGAAGGCGCGCA | 3036 |
| | GGAGACGACGGGGGACGGATGGG | 1336 | GGAGACGACGGGGGACGGAU | 3037 |
| | GTTTCTCCGCGCAGCGGGGCGGG | 1337 | GUUUCUCCGCGCAGCGGGGC | 3038 |
| | CTTCGCGAGCTCCTCCAGCAGGG | 1338 | CUUCGCGAGCUCCUCCAGCA | 3039 |
| | CTGGCGTCTTCCGTGGACAGTGG | 1339 | CUGGCGUCUUCCGUGGACAG | 3040 |
| | GCGGCGCATGGACGGACGGACGG | 1340 | GCGGCGCAUGGACGGACGGA | 3041 |
| | GCAGAGATACCCGAGCGCCCGGG | 1341 | GCAGAGAUACCCGAGCGCCC | 3042 |
| | CGGGCGGAGACCGGTTCCCCGGG | 1342 | CGGGCGGAGACCGGUUCCCC | 3043 |
| | GGTTTCTCCGCGCAGCGGGGCGG | 1343 | GGUUUCUCCGCGCAGCGGGG | 3044 |
| | TGCGAGCGGCAGGTCAGCCGGGG | 1344 | UGCGAGCGGCAGGUCAGCCG | 3045 |
| | ACCGGTTCCCCGGGAAGGTGAGG | 1345 | ACCGGUUCCCCGGGAAGGUG | 3046 |
| | GGAACTGGTCTCGGGGCGGCGGG | 1346 | GGAACUGGUCUCGGGGCGGC | 3047 |
| | AGACGACGGGGGACGGATGGGGG | 1347 | AGACGACGGGGGACGGAUGG | 3048 |
| | CGAGACCAGTTCCCCGGCGCGGG | 1348 | CGAGACCAGUUCCCCGGCGC | 3049 |
| | TGGGGCCCGGAGCGCTCCAAGGG | 1349 | UGGGGCCCGGAGCGCUCCAA | 3050 |
| | GTCAGCCGGGGTGCTAGGTCTGG | 1350 | GUCAGCCGGGGUGCUAGGUC | 3051 |
| | CCGGGCGGAGACCGGTTCCCCGG | 1351 | CCGGGCGGAGACCGGUUCCC | 3052 |
| | CTGCACCGGAAGGCGCGCAGGGG | 1352 | CUGCACCGGAAGGCGCGCAG | 3053 |
| | GCGTCTTCCGTGGACAGTGGTGG | 1353 | GCGUCUUCCGUGGACAGUGG | 3054 |
| | GGCAGAGATACCCGAGCGCCCGG | 1354 | GGCAGAGAUACCCGAGCGCC | 3055 |
| | CGCCATTCCTACCTCCGCTCCGG | 1355 | CGCCAUUCCUACCUCCGCUC | 3056 |
| | TTCCTCCCATGGTACAGTCGGGG | 1356 | UUCCUCCCAUGGUACAGUCG | 3057 |
| | GGTGCTAGGTCTGGCGTGCGGGG | 1357 | GGUGCUAGGUCUGGCGUGCG | 3058 |
| | GGAATGGTGGCGGCTCTCGAGGG | 1358 | GGAAUGGUGGCGGCUCUCGA | 3059 |
| | CTGTCAGGACGAGGCCGCGACGG | 1359 | CUGUCAGGACGAGGCCGCGA | 3060 |
| | CGGAGCCGCAGGTAGCGGGCGGG | 1360 | CGGAGCCGCAGGUAGCGGGC | 3061 |
| | GCCTACCCCTGCGCGCCTTCCGG | 1361 | GCCUACCCCUGCGCGCCUUC | 3062 |
| | GGCGCAGAGGCGCTTCCTGAGGG | 1362 | GGCGCAGAGGCGCUUCCUGA | 3063 |
| | TGCGCGCCTCGGACTGGCCCCGG | 1363 | UGCGCGCCUCGGACUGGCCC | 3064 |
| | TGCCCCGACTGTACCATGGGAGG | 1364 | UGCCCCGACUGUACCAUGGG | 3065 |
| | GCGCAGAGGCGCTTCCTGAGGGG | 1365 | GCGCAGAGGCGCUUCCUGAG | 3066 |
| | GTCCTTCCAGGATCGCCTGCGGG | 1366 | GUCCUUCCAGGAUCGCCUGC | 3067 |
| | CGCCCCCGGTCCATCCCTGCTGG | 1367 | CGCCCCCGGUCCAUCCCUGC | 3068 |
| | GGGTGCTAGGTCTGGCGTGCGGG | 1368 | GGGUGCUAGGUCUGGCGUGC | 3069 |
| | GGTGGGTGCGCGCCTCGGACTGG | 1369 | GGUGGGUGCGCGCCUCGGAC | 3070 |
| | CGCGGAGAAACCGAGGCCGGAGG | 1370 | CGCGGAGAAACCGAGGCCGG | 3071 |
| | CTGCAGGGGGCGCCCGTGAGCGG | 1371 | CUGCAGGGGGCGCCCGUGAG | 3072 |
| | GCCCGGAGCGCTCCAAGGGGCGG | 1372 | GCCCGGAGCGCUCCAAGGGG | 3073 |
| | AAGTCTGAGTGCGGGACACAGGG | 1373 | AAGUCUGAGUGCGGGACACA | 3074 |
| | CGTCCTTCCAGGATCGCCTGCGG | 1374 | CGUCCUUCCAGGAUCGCCUG | 3075 |
| | GCGGCGGGAAAGGAACCCTGAGG | 1375 | GCGGCGGGAAAGGAACCCUG | 3076 |
| | CGGTGCAGTTTGGGACAGCTCGG | 1376 | CGGUGCAGUUUGGGACAGCU | 3077 |
| | CCAGCGGCCGCCGAGACAGCGGG | 1377 | CCAGCGGCCGCCGAGACAGC | 3078 |
| FAS | CGGTTTACGAGTGACTTGGCTGG | 1378 | CGGUUUACGAGUGACUUGGC | 3079 |
| | AACTTGGCCTGCGCGCGGGTAGG | 1379 | AACUUGGCCUGCGCGCGGGU | 3080 |
| | GACCCGCTCAGTACGGAGTTGGG | 1380 | GACCCGCUCAGUACGGAGUU | 3081 |
| | CTATCCCCGGGACTAAGACGGGG | 1381 | CUAUCCCCGGGACUAAGACG | 3082 |
| | CGAAGCAGTGGTTAAGCCGGAGG | 1382 | CGAAGCAGUGGUUAAGCCGG | 3083 |
| | CCCGTCTTAGTCCCGGGGATAGG | 1383 | CCCGUCUUAGUCCCGGGGAU | 3084 |
| | GGACGCGTGCGGGATTGCGGCGG | 1384 | GGACGCGUGCGGGAUUGCGG | 3085 |
| | TGCCGTTCTTCCGAGCCCTCCGG | 1385 | UGCCGUUCUUCCGAGCCCUC | 3086 |
| | GTTGGTGGACCCGCTCAGTACGG | 1386 | GUUGGUGGACCCGCUCAGUA | 3087 |
| | CCAAAGGTCCGCTCCGGCGCGGG | 1387 | CCAAAGGUCCGCUCCGGCGC | 3088 |
| | ATGCGAAGTGCTGACCCCGCTGG | 1388 | AUGCGAAGUGCUGACCCCGC | 3089 |
| | GCCGGAGCGGACCTTTGGCTTGG | 1389 | GCCGGAGCGGACCUUUGGCU | 3090 |
| | CTCGCGCAAGAGTGACACACAGG | 1390 | CUCGCGCAAGAGUGACACAC | 3091 |
| | CTTACCCCGTCTTAGTCCCGGGG | 1391 | CUUACCCCGUCUUAGUCCCG | 3092 |
| | GAAGCGGTTTACGAGTGACTTGG | 1392 | GAAGCGGUUUACGAGUGACU | 3093 |
| | GGGGTCAGCACTTCGCATCAAGG | 1393 | GGGGUCAGCACUUCGCAUCA | 3094 |
| | ACTGCGCTCCACGTTGAGGTGGG | 1394 | ACUGCGCUCCACGUUGAGGU | 3095 |
| | TGCGAAGTGCTGACCCCGCTGGG | 1395 | UGCGAAGUGCUGACCCCGCU | 3096 |
| | GAGCGGGTCCACCAACCCGCGGG | 1396 | GAGCGGGUCCACCAACCCGC | 3097 |
| | AGACGGGGTAAGCCTCCACCCGG | 1397 | AGACGGGGUAAGCCUCCACC | 3098 |
| | ACCCGCTCAGTACGGAGTTGGGG | 1398 | ACCCGCUCAGUACGGAGUUG | 3099 |
| | GCCAAAGGTCCGCTCCGGCGCGG | 1399 | GCCAAAGGUCCGCUCCGGCG | 3100 |
| | GGACCCGCTCAGTACGGAGTTGG | 1400 | GGACCCGCUCAGUACGGAGU | 3101 |
| | GCCTATCCCCGGGACTAAGACGG | 1401 | GCCUAUCCCCGGGACUAAGA | 3102 |
| | GAGCTCACGAAAAGCCCCGGTGG | 1402 | GAGCUCACGAAAAGCCCCGG | 3103 |
| | TGAGCGGGTCCACCAACCCGCGG | 1403 | UGAGCGGGUCCACCAACCCG | 3104 |
| | GAAAAGCCCCGGTGGTCAGGAGG | 1404 | GAAAAGCCCCGGUGGUCAGG | 3105 |
| | GACGAGCTCACGAAAAGCCCCGG | 1405 | GACGAGCUCACGAAAAGCCC | 3106 |
| | GCGTTGGAGACTGGCTCCCGGGG | 1406 | GCGUUGGAGACUGGCUCCCG | 3107 |
| | AGGTCCGCTCCGGCGCGGGTGGG | 1407 | AGGUCCGCUCCGGCGCGGGU | 3108 |
| | GCTTACCCCGTCTTAGTCCCGGG | 1408 | GCUUACCCCGUCUUAGUCCC | 3109 |
| | CTCCACGTTGAGGTGGGCGTGGG | 1409 | CUCCACGUUGAGGUGGGCGU | 3110 |
| | GGCTTACCCCGTCTTAGTCCCGG | 1410 | GGCUUACCCCGUCUUAGUCC | 3111 |
| | GCGGGACGCGTGCGGGATTGCGG | 1411 | GCGGGACGCGUGCGGGAUUG | 3112 |
| | CACGAAAAGCCCCGGTGGTCAGG | 1412 | CACGAAAAGCCCCGGUGGUC | 3113 |
| | GTCAGGGTTCGTTGCACAAATGG | 1413 | GUCAGGGUUCGUUGCACAAA | 3114 |
| | GACGGGGTAAGCCTCCACCCGGG | 1414 | GACGGGGUAAGCCUCCACCC | 3115 |
| | CGTTGGAGACTGGCTCCCGGGGG | 1415 | CGUUGGAGACUGGCUCCCGG | 3116 |
| | TTCTGGCAGTTCTCAGACGTAGG | 1416 | UUCUGGCAGUUCUCAGACGU | 3117 |
| | GTCCCGGGGCGTTCCTGCAGTGG | 1417 | GUCCCGGGGCGUUCCUGCAG | 3118 |
| | GCCAAGCCAAAGGTCCGCTCCGG | 1418 | GCCAAGCCAAAGGUCCGCUC | 3119 |
| | AAGGTCCGCTCCGGCGCGGGTGG | 1419 | AAGGUCCGCUCCGGCGCGGG | 3120 |
| | GCGCACTCACCCACCCGCGCCGG | 1420 | GCGCACUCACCCACCCGCGC | 3121 |
| | CGGAAGTCTGGGAAGCTTTAGGG | 1421 | CGGAAGUCUGGGAAGCUUUA | 3122 |
| | CACCTCAACGTGGAGCGCAGTGG | 1422 | CACCUCAACGUGGAGCGCAG | 3123 |
| | AACCCGGGCGTTCCCCAGCGAGG | 1423 | AACCCGGGCGUUCCCCAGCG | 3124 |
| | TCAGCAACTTGGCCTGCGCGCGG | 1424 | UCAGCAACUUGGCCUGCGCG | 3125 |
| | GGGAAGCTCTTTCACTTCGGAGG | 1425 | GGGAAGCUCUUUCACUUCGG | 3126 |
| | CAGTGGTCTCCGAGGAGCGCCGG | 1426 | CAGUGGUCUCCGAGGAGCGC | 3127 |
| | GTTCCGCTCCTCTCTCCAACCGG | 1427 | GUUCCGCUCCUCUCUCCAAC | 3128 |
| DCK | CGGGGACCGCAGTCACCCCGTGG | 1428 | CGGGGACCGCAGUCACCCCG | 3129 |
| | GGTTTGACTTTGGCGCGCGGAGG | 1429 | GGUUUGACUUUGGCGCGCGG | 3130 |
| | CTTGCGTCCCACATTTCCGGAGG | 1430 | CUUGCGUCCCACAUUUCCGG | 3131 |
| | GCGCGCCTCACAGAGACCGCAGG | 1431 | GCGCGCCUCACAGAGACCGC | 3132 |
| | CTCCGGAAATGTGGGACGCAAGG | 1432 | CUCCGGAAAUGUGGGACGCA | 3133 |
| | CGGGGTTTGACTTTGGCGCGCGG | 1433 | CGGGGUUUGACUUUGGCGCG | 3134 |
| | GAACATCGGTAAGGAGCCTCCGG | 1434 | GAACAUCGGUAAGGAGCCUC | 3135 |
| | AGCTCACTAGCTGACCCGGCAGG | 1435 | AGCUCACUAGCUGACCCGGC | 3136 |
| | GGAAAACCCGCCTCTCTAGTGGG | 1436 | GGAAAACCCGCCUCUCUAGU | 3137 |
| | GTGGCGGCCCAGAGCTCGTCCGG | 1437 | GUGGCGGCCCAGAGCUCGUC | 3138 |
| | CATCGAAGGGAACATCGGTAAGG | 1438 | CAUCGAAGGGAACAUCGGUA | 3139 |
| | TTTGCGAGTTCCCAACAAAGAGG | 1439 | UUUGCGAGUUCCCAACAAAG | 3140 |
| | TGAGCTCACCGGCCCGCCGGCGG | 1440 | UGAGCUCACCGGCCCGCCGG | 3141 |
| | CAGAGCTCGTCCGGCAAAGAGGG | 1441 | CAGAGCUCGUCCGGCAAAGA | 3142 |
| | TAGTGAGCTCACCGGCCCGCCGG | 1442 | UAGUGAGCUCACCGGCCCGC | 3143 |
| | CTCGTCCGGCAAAGAGGGCTGGG | 1443 | CUCGUCCGGCAAAGAGGGCU | 3144 |
| | ACTAGCTGACCCGGCAGGTCAGG | 1444 | ACUAGCUGACCCGGCAGGUC | 3145 |
| | ATCTCCATCGAAGGGAACATCGG | 1445 | AUCUCCAUCGAAGGGAACAU | 3146 |
| | CACGGGGTGACTGCGGTCCCCGG | 1446 | CACGGGGUGACUGCGGUCCC | 3147 |
| | GAGGCCTTCCGCCACACGCGCGG | 1447 | GAGGCCUUCCGCCACACGCG | 3148 |
| | CTTTGTTGGGAACTCGCAAAGGG | 1448 | CUUUGUUGGGAACUCGCAAA | 3149 |
| | CGGGAAGAGGTTCCGGAGTCGGG | 1449 | CGGGAAGAGGUUCCGGAGUC | 3150 |
| | CTTACCGATGTTCCCTTCGATGG | 1450 | CUUACCGAUGUUCCCUUCGA | 3151 |
| | ACGGGGTGACTGCGGTCCCCGGG | 1451 | ACGGGGUGACUGCGGUCCCC | 3152 |
| | GAACTCGCAAAGGGAAGCGGGGG | 1452 | GAACUCGCAAAGGGAAGCGG | 3153 |
| | GAAGGGTATTAGATTTCTTGAGG | 1453 | GAAGGGUAUUAGAUUUCUUG | 3154 |
| | AGTCAAACCCCGACACCCGCCGG | 1454 | AGUCAAACCCCGACACCCGC | 3155 |
| | AGCTCCAGTGCGCGCACCCGTGG | 1455 | AGCUCCAGUGCGCGCACCCG | 3156 |
| | TCTGGGCCGCCACAAGACTAAGG | 1456 | UCUGGGCCGCCACAAGACUA | 3157 |
| | GCGGGAAGAGGTTCCGGAGTCGG | 1457 | GCGGGAAGAGGUUCCGGAGU | 3158 |
| | TGGAAAACCCGCCTCTCTAGTGG | 1458 | UGGAAAACCCGCCUCUCUAG | 3159 |
| | CGGGTGTCGGGGTTTGACTTTGG | 1459 | CGGGUGUCGGGGUUUGACUU | 3160 |
| | CTCTTTGCCGGACGAGCTCTGGG | 1460 | CUCUUUGCCGGACGAGCUCU | 3161 |
| | CCTCTTTGCCGGACGAGCTCTGG | 1461 | CCUCUUUGCCGGACGAGCUC | 3162 |
| | AAACCCCGACACCCGCCGGCGGG | 1462 | AAACCCCGACACCCGCCGGC | 3163 |
| | TGGCGCCCAGTCTGACCCCGGGG | 1463 | UGGCGCCCAGUCUGACCCCG | 3164 |
| | TTGCGAGTTCCCAACAAAGAGGG | 1464 | UUGCGAGUUCCCAACAAAGA | 3165 |
| | CAAACCCCGACACCCGCCGGCGG | 1465 | CAAACCCCGACACCCGCCGG | 3166 |
| | TCTTTGTTGGGAACTCGCAAAGG | 1466 | UCUUUGUUGGGAACUCGCAA | 3167 |
| | CGATGGAGATTTTCTTGATGCGG | 1467 | CGAUGGAGAUUUUCUUGAUG | 3168 |
| | CGCCTCTCTAGTGGGCCTGTTGG | 1468 | CGCCUCUCUAGUGGGCCUGU | 3169 |
| | GCCCCGGCCTTCACGTGACCTGG | 1469 | GCCCCGGCCUUCACGUGACC | 3170 |
| | GCTCGTCCGGCAAAGAGGGCTGG | 1470 | GCUCGUCCGGCAAAGAGGGC | 3171 |
| | TGCGGTCCCCGGGGTCAGACTGG | 1471 | UGCGGUCCCCGGGGUCAGAC | 3172 |
| | AGCCTTGCGTCCCACATTTCCGG | 1472 | AGCCUUGCGUCCCACAUUUC | 3173 |
| | CTGCAGAGAGATGCGGCGAAGGG | 1473 | CUGCAGAGAGAUGCGGCGAA | 3174 |
| | GCGGTCCCCGGGGTCAGACTGGG | 1474 | GCGGUCCCCGGGGUCAGACU | 3175 |
| | CAGCTAGGGAGCGCGGCTTGAGG | 1475 | CAGCUAGGGAGCGCGGCUUG | 3176 |
| | ACGCCAGGTCACGTGAAGGCCGG | 1476 | ACGCCAGGUCACGUGAAGGC | 3177 |
| | CGCCAGGTCACGTGAAGGCCGGG | 1477 | CGCCAGGUCACGUGAAGGCC | 3178 |
| DGKA | ATCCCTCCGAATGAGCGGGAGGG | 1478 | AUCCCUCCGAAUGAGCGGGA | 3179 |
| | ATCCCTCCCGCTCATTCGGAGGG | 1479 | AUCCCUCCCGCUCAUUCGGA | 3180 |
| | GTATCGAGAAGGGTCTGCGCTGG | 1480 | GUAUCGAGAAGGGUCUGCGC | 3181 |
| | CTATGTCGTCAGGAACGGGGCGG | 1481 | CUAUGUCGUCAGGAACGGGG | 3182 |
| | GCAGCACGAACGCAGCCCGTGGG | 1482 | GCAGCACGAACGCAGCCCGU | 3183 |
| | CTTCGAAGTTCCCAGAGTCGGGG | 1483 | CUUCGAAGUUCCCAGAGUCG | 3184 |
| | CATCCCTCCGAATGAGCGGGAGG | 1484 | CAUCCCUCCGAAUGAGCGGG | 3185 |
| | GAAACGTTACCCACCGGGTTCGG | 1485 | GAAACGUUACCCACCGGGUU | 3186 |
| | ACGGGCTGCGTTCGTGCTGCTGG | 1486 | ACGGGCUGCGUUCGUGCUGC | 3187 |
| | GAACCTCCACAGTGCCGCACGGG | 1487 | GAACCUCCACAGUGCCGCAC | 3188 |
| | GTTCCTGGGACCCGACTCGGAGG | 1488 | GUUCCUGGGACCCGACUCGG | 3189 |
| | CATCCCTCCCGCTCATTCGGAGG | 1489 | CAUCCCUCCCGCUCAUUCGG | 3190 |
| | AACTTCGAAGTTCCCAGAGTCGG | 1490 | AACUUCGAAGUUCCCAGAGU | 3191 |
| | CTCCGAATGAGCGGGAGGGATGG | 1491 | CUCCGAAUGAGCGGGAGGGA | 3192 |
| | ACTTCGAAGTTCCCAGAGTCGGG | 1492 | ACUUCGAAGUUCCCAGAGUC | 3193 |
| | AAACGTTACCCACCGGGTTCGGG | 1493 | AAACGUUACCCACCGGGUUC | 3194 |
| | CTCCCGCTCATTCGGAGGGATGG | 1494 | CUCCCGCUCAUUCGGAGGGA | 3195 |
| | CCCCCATCGGAAAAGGACAGGGG | 1495 | CCCCCAUCGGAAAAGGACAG | 3196 |
| | CAGCTATGTCGTCAGGAACGGGG | 1496 | CAGCUAUGUCGUCAGGAACG | 3197 |
| | GGAACCTCCACAGTGCCGCACGG | 1497 | GGAACCUCCACAGUGCCGCA | 3198 |
| | GTCTGGGAAACGTTACCCACCGG | 1498 | GUCUGGGAAACGUUACCCAC | 3199 |
| | GGGCGGGCAGCTATGTCGTCAGG | 1499 | GGGCGGGCAGCUAUGUCGUC | 3200 |
| | CGCGGTCGCAGCTGAAGCGCCGG | 1500 | CGCGGUCGCAGCUGAAGCGC | 3201 |
| | TTCGAAGTTCCCAGAGTCGGGGG | 1501 | UUCGAAGUUCCCAGAGUCGG | 3202 |
| | TCTGGGAAACGTTACCCACCGGG | 1502 | UCUGGGAAACGUUACCCACC | 3203 |
| | TATCGAGAAGGGTCTGCGCTGGG | 1503 | UAUCGAGAAGGGUCUGCGCU | 3204 |
| | AGCTGGAGCGGGTATCGAGAAGG | 1504 | AGCUGGAGCGGGUAUCGAGA | 3205 |
| | CCTCCACAGTGCCGCACGGGTGG | 1505 | CCUCCACAGUGCCGCACGGG | 3206 |
| | CCGGTTCCTGGGACCCGACTCGG | 1506 | CCGGUUCCUGGGACCCGACU | 3207 |
| | GAACGCAGCCCGTGGGTCCTCGG | 1507 | GAACGCAGCCCGUGGGUCCU | 3208 |
| | TACCCCTGTCCTTTTCCGATGGG | 1508 | UACCCCUGUCCUUUUCCGAU | 3209 |
| | AAGAGGACTTCCCTCCGAGTCGG | 1509 | AAGAGGACUUCCCUCCGAGU | 3210 |
| | TCACCATCCCTCCGAATGAGCGG | 1510 | UCACCAUCCCUCCGAAUGAG | 3211 |
| | TTCCTGGGACCCGACTCGGAGGG | 1511 | UUCCUGGGACCCGACUCGGA | 3212 |
| | ACCGCGAGCCCTCTCAAGCAAGG | 1512 | ACCGCGAGCCCUCUCAAGCA | 3213 |
| | ACCCCTGTCCTTTTCCGATGGGG | 1513 | ACCCCUGUCCUUUUCCGAUG | 3214 |
| | AGGGTCTGCGCTGGGACGCGGGG | 1514 | AGGGUCUGCGCUGGGACGCG | 3215 |
| | ACTCGGAGGGAAGTCCTCTTCGG | 1515 | ACUCGGAGGGAAGUCCUCUU | 3216 |
| | CTCCCCCATCGGAAAAGGACAGG | 1516 | CUCCCCCAUCGGAAAAGGAC | 3217 |
| | AGAACCCTTCTCCACCCGTGCGG | 1517 | AGAACCCUUCUCCACCCGUG | 3218 |
| | GGTCTCTACCTTGCTTGAGAGGG | 1518 | GGUCUCUACCUUGCUUGAGA | 3219 |
| | CAGTGCCGCACGGGTGGAGAAGG | 1519 | CAGUGCCGCACGGGUGGAGA | 3220 |
| | GCAGCTATGTCGTCAGGAACGGG | 1520 | GCAGCUAUGUCGUCAGGAAC | 3221 |
| | CTGTCCTTTTCCGATGGGGGAGG | 1521 | CUGUCCUUUUCCGAUGGGGG | 3222 |
| | TTCCCTCCGAGTCGGGTCCCAGG | 1522 | UUCCCUCCGAGUCGGGUCCC | 3223 |
| | AGTGCCGCACGGGTGGAGAAGGG | 1523 | AGUGCCGCACGGGUGGAGAA | 3224 |
| | CACCATCCCTCCGAATGAGCGGG | 1524 | CACCAUCCCUCCGAAUGAGC | 3225 |
| | CGAGTCGGGTCCCAGGAACCGGG | 1525 | CGAGUCGGGUCCCAGGAACC | 3226 |
| | AGAGGACTTCCCTCCGAGTCGGG | 1526 | AGAGGACUUCCCUCCGAGUC | 3227 |
| | GGGTCTCTACCTTGCTTGAGAGG | 1527 | GGGUCUCUACCUUGCUUGAG | 3228 |
| USP16 | TAACTGCTCCGATCCCACGGGGG | 1528 | UAACUGCUCCGAUCCCACGG | 3229 |
| | CTAACTGCTCCGATCCCACGGGG | 1529 | CUAACUGCUCCGAUCCCACG | 3230 |
| | TCTCGACCCCGTGGACCCAGAGG | 1530 | UCUCGACCCCGUGGACCCAG | 3231 |
| | GTCGCTCTCAATTCGTCACCAGG | 1531 | GUCGCUCUCAAUUCGUCACC | 3232 |
| | CGCAGTACCGGAAAGTAGCCGGG | 1532 | CGCAGUACCGGAAAGUAGCC | 3233 |
| | CTTCCCATAATGCCGCGTTCCGG | 1533 | CUUCCCAUAAUGCCGCGUUC | 3234 |
| | TTCCGGTACTGCGATCTCATTGG | 1534 | UUCCGGUACUGCGAUCUCAU | 3235 |
| | CGCCGGATGTTCGGGTTTAGGGG | 1535 | CGCCGGAUGUUCGGGUUUAG | 3236 |
| | ACTTCCGGAACGCGGCATTATGG | 1536 | ACUUCCGGAACGCGGCAUUA | 3237 |
| | GCGCCGGATGTTCGGGTTTAGGG | 1537 | GCGCCGGAUGUUCGGGUUUA | 3238 |
| | TGGCGGCCTTCTCGACCCCGTGG | 1538 | UGGCGGCCUUCUCGACCCCG | 3239 |
| | CGGAAGTTATTGCTTTCCAGGGG | 1539 | CGGAAGUUAUUGCUUUCCAG | 3240 |
| | GTAGCCGGGTTACGTGCTTAAGG | 1540 | GUAGCCGGGUUACGUGCUUA | 3241 |
| | TCGCAGTACCGGAAAGTAGCCGG | 1541 | UCGCAGUACCGGAAAGUAGC | 3242 |
| | AGCCAATGAGATCGCAGTACCGG | 1542 | AGCCAAUGAGAUCGCAGUAC | 3243 |
| | GCGGCTTGCGCCGGATGTTCGGG | 1543 | GCGGCUUGCGCCGGAUGUUC | 3244 |
| | TGCGCCGGATGTTCGGGTTTAGG | 1544 | UGCGCCGGAUGUUCGGGUUU | 3245 |
| | TGCGGCTTGCGCCGGATGTTCGG | 1545 | UGCGGCUUGCGCCGGAUGUU | 3246 |
| | CACGTAACCCGGCTACTTTCCGG | 1546 | CACGUAACCCGGCUACUUUC | 3247 |
| | GTTATGGGCTCTGTCGCCGTGGG | 1547 | GUUAUGGGCUCUGUCGCCGU | 3248 |
| | GGAGCATTTATATAACTTCGTGG | 1548 | GGAGCAUUUAUAUAACUUCG | 3249 |
| | TTTACTAGCGTCAGAGCCGATGG | 1549 | UUUACUAGCGUCAGAGCCGA | 3250 |
| | ATCCGGCGCAAGCCGCACGCAGG | 1550 | AUCCGGCGCAAGCCGCACGC | 3251 |
| | CTTCCGGAACGCGGCATTATGGG | 1551 | CUUCCGGAACGCGGCAUUAU | 3252 |
| | GGTTACGTGCTTAAGGAGAGCGG | 1552 | GGUUACGUGCUUAAGGAGAG | 3253 |
| | GCTCTCAATTCGTCACCAGGAGG | 1553 | GCUCUCAAUUCGUCACCAGG | 3254 |
| | AAGCAATAACTTCCGGAACGCGG | 1554 | AAGCAAUAACUUCCGGAACG | 3255 |
| | TCAGAGCCGATGGTCCCGGGAGG | 1555 | UCAGAGCCGAUGGUCCCGGG | 3256 |
| | GCCGTGTTCATACGGCTGGTAGG | 1556 | GCCGUGUUCAUACGGCUGGU | 3257 |
| | TCTAACTGCTCCGATCCCACGGG | 1557 | UCUAACUGCUCCGAUCCCAC | 3258 |
| | AGTAAATCTGCGCCTGCGTGCGG | 1558 | AGUAAAUCUGCGCCUGCGUG | 3259 |
| | CCGGAAGTTATTGCTTTCCAGGG | 1559 | CCGGAAGUUAUUGCUUUCCA | 3260 |
| | AACCCCTAAACCCGAACATCCGG | 1560 | AACCCCUAAACCCGAACAUC | 3261 |
| | GTGGCAGGCGCCGAGCAAATGGG | 1561 | GUGGCAGGCGCCGAGCAAAU | 3262 |
| | CGTGGCAGGGACTTCCCTATGGG | 1562 | CGUGGCAGGGACUUCCCUAU | 3263 |
| | TGCAGCCGTGTTCATACGGCTGG | 1563 | UGCAGCCGUGUUCAUACGGC | 3264 |
| | CCTGGCAGTCGTCGCTCGCCTGG | 1564 | CCUGGCAGUCGUCGCUCGCC | 3265 |
| | CTGTCGCCGTGGGTGAGTTCTGG | 1565 | CUGUCGCCGUGGGUGAGUUC | 3266 |
| | AGCGACGACTGCCAGGCAGTGGG | 1566 | AGCGACGACUGCCAGGCAGU | 3267 |
| | TGGTGACGAATTGAGAGCGACGG | 1567 | UGGUGACGAAUUGAGAGCGA | 3268 |
| | GAACGCGGCATTATGGGAAGTGG | 1568 | GAACGCGGCAUUAUGGGAAG | 3269 |
| | TCCGGAAGTTATTGCTTTCCAGG | 1569 | UCCGGAAGUUAUUGCUUUCC | 3270 |
| | ATCGGAGCAGTTAGAAGGGGAGG | 1570 | AUCGGAGCAGUUAGAAGGGG | 3271 |
| | CTGGGTCCACGGGGTCGAGAAGG | 1571 | CUGGGUCCACGGGGUCGAGA | 3272 |
| | ATACGGCTGGTAGGAAAAGCAGG | 1572 | AUACGGCUGGUAGGAAAAGC | 3273 |
| | TTCTAACTGCTCCGATCCCACGG | 1573 | UUCUAACUGCUCCGAUCCCA | 3274 |
| | GCGCCGAGCAAATGGGTGGGTGG | 1574 | GCGCCGAGCAAAUGGGUGGG | 3275 |
| | GCGTCAGAGCCGATGGTCCCGGG | 1575 | GCGUCAGAGCCGAUGGUCCC | 3276 |
| | GTGGGATCGGAGCAGTTAGAAGG | 1576 | GUGGGAUCGGAGCAGUUAGA | 3277 |
| | CGCCTGCGTGCGGCTTGCGCCGG | 1577 | CGCCUGCGUGCGGCUUGCGC | 3278 |
| PTPN11 | ACGGGTCGGTGGCGTAGACGCGG | 1578 | ACGGGUCGGUGGCGUAGACG | 3279 |
| | ACGGGGCTAACCGAACGCGGCGG | 1579 | ACGGGGCUAACCGAACGCGG | 3280 |
| | CGTCGCGAGCGGTGACATCACGG | 1580 | CGUCGCGAGCGGUGACAUCA | 3281 |
| | TCGGTTAGCCCCGTCCGGAAGGG | 1581 | UCGGUUAGCCCCGUCCGGAA | 3282 |
| | CGGTTAGCCCCGTCCGGAAGGGG | 1582 | CGGUUAGCCCCGUCCGGAAG | 3283 |
| | AACCGAACGCGGCGGTGGCCGGG | 1583 | AACCGAACGCGGCGGUGGCC | 3284 |
| | TAACCGAACGCGGCGGTGGCCGG | 1584 | UAACCGAACGCGGCGGUGGC | 3285 |
| | TCGCGAGCGGTGACATCACGGGG | 1585 | UCGCGAGCGGUGACAUCACG | 3286 |
| | TAGAGCCGCCGAGGGAACCACGG | 1586 | UAGAGCCGCCGAGGGAACCA | 3287 |
| | AACATGACATCGCGGAGGTGAGG | 1587 | AACAUGACAUCGCGGAGGUG | 3288 |
| | GGTTAGCCCCGTCCGGAAGGGGG | 1588 | GGUUAGCCCCGUCCGGAAGG | 3289 |
| | TCGCTCGGTCCTCCGCTGACGGG | 1589 | UCGCUCGGUCCUCCGCUGAC | 3290 |
| | GGGCTAACCGAACGCGGCGGTGG | 1590 | GGGCUAACCGAACGCGGCGG | 3291 |
| | CGCGTTCGGTTAGCCCCGTCCGG | 1591 | CGCGUUCGGUUAGCCCCGUC | 3292 |
| | CGAAATAACCCTGCTCACTTGGG | 1592 | CGAAAUAACCCUGCUCACUU | 3293 |
| | ACACGAGAGGGGAGTTGCGCGGG | 1593 | ACACGAGAGGGGAGUUGCGC | 3294 |
| | TTCGGTTAGCCCCGTCCGGAAGG | 1594 | UUCGGUUAGCCCCGUCCGGA | 3295 |
| | CGCGAGCGGTGACATCACGGGGG | 1595 | CGCGAGCGGUGACAUCACGG | 3296 |
| | GACACGAGAGGGGAGTTGCGCGG | 1596 | GACACGAGAGGGGAGUUGCG | 3297 |
| | TCTACGCCACCGACCCGTCCGGG | 1597 | UCUACGCCACCGACCCGUCC | 3298 |
| | CGGTGACATCACGGGGGCGACGG | 1598 | CGGUGACAUCACGGGGGCGA | 3299 |
| | GTCTACGCCACCGACCCGTCCGG | 1599 | GUCUACGCCACCGACCCGUC | 3300 |
| | GGAGGAACATGACATCGCGGAGG | 1600 | GGAGGAACAUGACAUCGCGG | 3301 |
| | GGCACCCGTGGTTCCCTCGGCGG | 1601 | GGCACCCGUGGUUCCCUCGG | 3302 |
| | AGCAAGGAGCGGGTCCGTCGCGG | 1602 | AGCAAGGAGCGGGUCCGUCG | 3303 |
| | CGGACGGGGCTAACCGAACGCGG | 1603 | CGGACGGGGCUAACCGAACG | 3304 |
| | CACGAGAGGGGAGTTGCGCGGGG | 1604 | CACGAGAGGGGAGUUGCGCG | 3305 |
| | ATGAGTGGAGCGGCGATTTGTGG | 1605 | AUGAGUGGAGCGGCGAUUUG | 3306 |
| | GACCGAGCGACGGCCGGGAATGG | 1606 | GACCGAGCGACGGCCGGGAA | 3307 |
| | GTCGCTCGGTCCTCCGCTGACGG | 1607 | GUCGCUCGGUCCUCCGCUGA | 3308 |
| | GTCCTCCGCTGACGGGAAGCAGG | 1608 | GUCCUCCGCUGACGGGAAGC | 3309 |
| | GTTATTTCGGAATCACCATGAGG | 1609 | GUUAUUUCGGAAUCACCAUG | 3310 |
| | TTCCTGCTTCCCGTCAGCGGAGG | 1610 | UUCCUGCUUCCCGUCAGCGG | 3311 |
| | AGAGCCGCCGAGGGAACCACGGG | 1611 | AGAGCCGCCGAGGGAACCAC | 3312 |
| | GGGTCCGTCGCGGAGCCGGAGGG | 1612 | GGGUCCGUCGCGGAGCCGGA | 3313 |
| | ATGTGGCAGCGGGCCCGGACGGG | 1613 | AUGUGGCAGCGGGCCCGGAC | 3314 |
| | AATCGATGTGGCAGCGGGCCCGG | 1614 | AAUCGAUGUGGCAGCGGGCC | 3315 |
| | TGCCATTCCCGGCCGTCGCTCGG | 1615 | UGCCAUUCCCGGCCGUCGCU | 3316 |
| | CCGAAATAACCCTGCTCACTTGG | 1616 | CCGAAAUAACCCUGCUCACU | 3317 |
| | TCCTGGAAACCGCGGCCGCCAGG | 1617 | UCCUGGAAACCGCGGCCGCC | 3318 |
| | TTCGGGCTCCCGCCCCGGGTCGG | 1618 | UUCGGGCUCCCGCCCCGGGU | 3319 |
| | TTCTCATGAGGCAATGGGTCAGG | 1619 | UUCUCAUGAGGCAAUGGGUC | 3320 |
| | GAGCGGGTCCGTCGCGGAGCCGG | 1620 | GAGCGGGUCCGUCGCGGAGC | 3321 |
| | GCGGGAGGAACATGACATCGCGG | 1621 | GCGGGAGGAACAUGACAUCG | 3322 |
| | CCCGATGTGACCGAGCCCAGCGG | 1622 | CCCGAUGUGACCGAGCCCAG | 3323 |
| | TCCGCTGGGCTCGGTCACATCGG | 1623 | UCCGCUGGGCUCGGUCACAU | 3324 |
| | CGGAGGACCGAGCGACGGCCGGG | 1624 | CGGAGGACCGAGCGACGGCC | 3325 |
| | GAAATGAATGGGGACCCGAGGGG | 1625 | GAAAUGAAUGGGGACCCGAG | 3326 |
| | GCTGCCGCAGCCGGAACTCGGGG | 1626 | GCUGCCGCAGCCGGAACUCG | 3327 |
| | TGACATCACGGGGGCGACGGCGG | 1627 | UGACAUCACGGGGGCGACGG | 3328 |
| PTPN6 | GGGTACCGTCCTTCTAAGTGGGG | 1628 | GGGUACCGUCCUUCUAAGUG | 3329 |
| | CTACTGTACAAAACGCAACTCGG | 1629 | CUACUGUACAAAACGCAACU | 3330 |
| | GTCCGCCTCGACCCAACCGGCGG | 1630 | GUCCGCCUCGACCCAACCGG | 3331 |
| | AATCGTCCTAGTCAAGGCATAGG | 1631 | AAUCGUCCUAGUCAAGGCAU | 3332 |
| | CTACGTCCGCCGGGAAAATGGGG | 1632 | CUACGUCCGCCGGGAAAAUG | 3333 |
| | CCGGGTACCGTCCTTCTAAGTGG | 1633 | CCGGGUACCGUCCUUCUAAG | 3334 |
| | CTGTTCTCCGACGCCTACCCGGG | 1634 | CUGUUCUCCGACGCCUACCC | 3335 |
| | CAGCGCTCAAGGCCGCCGGTTGG | 1635 | CAGCGCUCAAGGCCGCCGGU | 3336 |
| | TGGGTCGAGGCGGACGCCATAGG | 1636 | UGGGUCGAGGCGGACGCCAU | 3337 |
| | AGCGCTCAAGGCCGCCGGTTGGG | 1637 | AGCGCUCAAGGCCGCCGGUU | 3338 |
| | GAAACAGCATCCGGCGCAGCCGG | 1638 | GAAACAGCAUCCGGCGCAGC | 3339 |
| | TAATTCCTTGCGCTCTCCGCTGG | 1639 | UAAUUCCUUGCGCUCUCCGC | 3340 |
| | ACGCCTGGATCACCTCCGCGAGG | 1640 | ACGCCUGGAUCACCUCCGCG | 3341 |
| | GATAACGCCTGCAACGACATGGG | 1641 | GAUAACGCCUGCAACGACAU | 3342 |
| | GGATAACGCCTGCAACGACATGG | 1642 | GGAUAACGCCUGCAACGACA | 3343 |
| | CAAGGCCGCCGGTTGGGTCGAGG | 1643 | CAAGGCCGCCGGUUGGGUCG | 3344 |
| | GGGTCGAGGCGGACGCCATAGGG | 1644 | GGGUCGAGGCGGACGCCAUA | 3345 |
| | CGGGTACCGTCCTTCTAAGTGGG | 1645 | CGGGUACCGUCCUUCUAAGU | 3346 |
| | GCTACGTCCGCCGGGAAAATGGG | 1646 | GCUACGUCCGCCGGGAAAAU | 3347 |
| | GCGCGCAGTGGTCCTCGCGGAGG | 1647 | GCGCGCAGUGGUCCUCGCGG | 3348 |
| | GGTGAGAATCGTCCTAGTCAAGG | 1648 | GGUGAGAAUCGUCCUAGUCA | 3349 |
| | GCCGCCGTGTGGCGAGAAAGGGG | 1649 | GCCGCCGUGUGGCGAGAAAG | 3350 |
| | AATTCCTTGCGCTCTCCGCTGGG | 1650 | AAUUCCUUGCGCUCUCCGCU | 3351 |
| | TTTTCCCGGCGGACGTAGCCAGG | 1651 | UUUUCCCGGCGGACGUAGCC | 3352 |
| | CGGAGAGCGCAAGGAATTAGTGG | 1652 | CGGAGAGCGCAAGGAAUUAG | 3353 |
| | CATCTTACCCATGTCGTTGCAGG | 1653 | CAUCUUACCCAUGUCGUUGC | 3354 |
| | TCTCCGGGGCGGAGAACGCCTGG | 1654 | UCUCCGGGGCGGAGAACGCC | 3355 |
| | TTTCCGCTCCCAGGGGCGTTGGG | 1655 | UUUCCGCUCCCAGGGGCGUU | 3356 |
| | CATGCGCACTGCATTCTCCGGGG | 1656 | CAUGCGCACUGCAUUCUCCG | 3357 |
| | AACTCGGACGCACAAGCTCAGGG | 1657 | AACUCGGACGCACAAGCUCA | 3358 |
| | TGAAGCTCTAGGTTCAGCGGAGG | 1658 | UGAAGCUCUAGGUUCAGCGG | 3359 |
| | GGCGTCGGAGAACAGACAGCGGG | 1659 | GGCGUCGGAGAACAGACAGC | 3360 |
| | AGTTGCGTTTTGTACAGTAGAGG | 1660 | AGUUGCGUUUUGUACAGUAG | 3361 |
| | GTCCGCCGGGAAAATGGGGTAGG | 1661 | GUCCGCCGGGAAAAUGGGGU | 3362 |
| | CTAGAGCTTCAGACGCCCTATGG | 1662 | CUAGAGCUUCAGACGCCCUA | 3363 |
| | ACAAACCTGGCTACGTCCGCCGG | 1663 | ACAAACCUGGCUACGUCCGC | 3364 |
| | TCCGCCGGGAAAATGGGGTAGGG | 1664 | UCCGCCGGGAAAAUGGGGUA | 3365 |
| | TTGGGAACGGTTGTAGGACGTGG | 1665 | UUGGGAACGGUUGUAGGACG | 3366 |
| | GAATGCAGTGCGCATGGACGAGG | 1666 | GAAUGCAGUGCGCAUGGACG | 3367 |
| | GGCGCTTGCCCCCAAGACTTGGG | 1667 | GGCGCUUGCCCCCAAGACUU | 3368 |
| | TCTGTTCTCCGACGCCTACCCGG | 1668 | UCUGUUCUCCGACGCCUACC | 3369 |
| | CAACTCGGACGCACAAGCTCAGG | 1669 | CAACUCGGACGCACAAGCUC | 3370 |
| | GGCTACGTCCGCCGGGAAAATGG | 1670 | GGCUACGUCCGCCGGGAAAA | 3371 |
| | TGCCGCCGTGTGGCGAGAAAGGG | 1671 | UGCCGCCGUGUGGCGAGAAA | 3372 |
| | AGATTGTGGCTGCCGCCGTGTGG | 1672 | AGAUUGUGGCUGCCGCCGUG | 3373 |
| | TCCCCTTTCTCGCCACACGGCGG | 1673 | UCCCCUUUCUCGCCACACGG | 3374 |
| | AAGCGCCCCACTTAGAAGGACGG | 1674 | AAGCGCCCCACUUAGAAGGA | 3375 |
| | TTGAGCGCTGAGCAAGCAAAGGG | 1675 | UUGAGCGCUGAGCAAGCAAA | 3376 |
| | GGCCGCCGGTTGGGTCGAGGCGG | 1676 | GGCCGCCGGUUGGGUCGAGG | 3377 |
| | AATTAGTGGATTGAGGCTGTAGG | 1677 | AAUUAGUGGAUUGAGGCUGU | 3378 |
| PTPA | GCGACTGCCACGATTGTGCGGGG | 1678 | GCGACUGCCACGAUUGUGCG | 3379 |
| | CGTTCCCGGACGCAACCGCACGG | 1679 | CGUUCCCGGACGCAACCGCA | 3380 |
| | TCTCGGTTTTCGGTTATAGCCGG | 1680 | UCUCGGUUUUCGGUUAUAGC | 3381 |
| | CGGACTGCGTGTCCGCGGACGGG | 1681 | CGGACUGCGUGUCCGCGGAC | 3382 |
| | CGTGCGGTTGCGTCCGGGAACGG | 1682 | CGUGCGGUUGCGUCCGGGAA | 3383 |
| | GCGGACTGCGTGTCCGCGGACGG | 1683 | GCGGACUGCGUGUCCGCGGA | 3384 |
| | GCCAACGGCCGCCAAGCGCTAGG | 1684 | GCCAACGGCCGCCAAGCGCU | 3385 |
| | GCCTATTAACGGCCGGCGCGCGG | 1685 | GCCUAUUAACGGCCGGCGCG | 3386 |
| | CGCGACTGCCACGATTGTGCGGG | 1686 | CGCGACUGCCACGAUUGUGC | 3387 |
| | CAGTCGCGGCGCCCGACGTTCGG | 1687 | CAGUCGCGGCGCCCGACGUU | 3388 |
| | CCGTGAGCGGTCCTAGCGCTTGG | 1688 | CCGUGAGCGGUCCUAGCGCU | 3389 |
| | AGTCGCGGCGCCCGACGTTCGGG | 1689 | AGUCGCGGCGCCCGACGUUC | 3390 |
| | CATGGCGGCCGTCTTCGCTGTGG | 1690 | CAUGGCGGCCGUCUUCGCUG | 3391 |
| | CTTGACGCCCCGCACAATCGTGG | 1691 | CUUGACGCCCCGCACAAUCG | 3392 |
| | GGGATCCGCCGCACTCACCACGG | 1692 | GGGAUCCGCCGCACUCACCA | 3393 |
| | ACATCTTCGCTGCCCGTCCGCGG | 1693 | ACAUCUUCGCUGCCCGUCCG | 3394 |
| | CGGAGCGGACTGCGTGTCCGCGG | 1694 | CGGAGCGGACUGCGUGUCCG | 3395 |
| | ACGGCCGCCATGTCGGTGCGGGG | 1695 | ACGGCCGCCAUGUCGGUGCG | 3396 |
| | GCTCACGGCCGCCCGAACGTCGG | 1696 | GCUCACGGCCGCCCGAACGU | 3397 |
| | TATTAACGGCCGGCGCGCGGCGG | 1697 | UAUUAACGGCCGGCGCGCGG | 3398 |
| | AGCCTACTGCGACCCGCTACCGG | 1698 | AGCCUACUGCGACCCGCUAC | 3399 |
| | GCCTACTGCGACCCGCTACCGGG | 1699 | GCCUACUGCGACCCGCUACC | 3400 |
| | GCGGGCCGTGCGGTTGCGTCCGG | 1700 | GCGGGCCGUGCGGUUGCGUC | 3401 |
| | ATTGTGCGGGGCGTCAAGTTTGG | 1701 | AUUGUGCGGGGCGUCAAGUU | 3402 |
| | GCCGCGCGCCGGCCGTTAATAGG | 1702 | GCCGCGCGCCGGCCGUUAAU | 3403 |
| | GCGACGGCCATGTCAGTGCGGGG | 1703 | GCGACGGCCAUGUCAGUGCG | 3404 |
| | AAACACAACCATGTTGACCGGGG | 1704 | AAACACAACCAUGUUGACCG | 3405 |
| | GGGACTGCAAGCATCCGGGTCGG | 1705 | GGGACUGCAAGCAUCCGGGU | 3406 |
| | CTGGAGGCCGGGTCGAACAGCGG | 1706 | CUGGAGGCCGGGUCGAACAG | 3407 |
| | GGAGCAAGCCTATTAACGGCCGG | 1707 | GGAGCAAGCCUAUUAACGGC | 3408 |
| | CCGCACAATCGTGGCAGTCGCGG | 1708 | CCGCACAAUCGUGGCAGUCG | 3409 |
| | TGAGCGGTCCTAGCGCTTGGCGG | 1709 | UGAGCGGUCCUAGCGCUUGG | 3410 |
| | ATGTCGGTGCGGGGCGCTCAGGG | 1710 | AUGUCGGUGCGGGGCGCUCA | 3411 |
| | GCCCGGTAGCGGGTCGCAGTAGG | 1711 | GCCCGGUAGCGGGUCGCAGU | 3412 |
| | TGCACCTTTCCAACTCCGTCTGG | 1712 | UGCACCUUUCCAACUCCGUC | 3413 |
| | TTGTGCGGGGCGTCAAGTTTGGG | 1713 | UUGUGCGGGGCGUCAAGUUU | 3414 |
| | TCCTAGCGCTTGGCGGCCGTTGG | 1714 | UCCUAGCGCUUGGCGGCCGU | 3415 |
| | CGGACTTTGCCCGGTGTGTGGGG | 1715 | CGGACUUUGCCCGGUGUGUG | 3416 |
| | AGACGGCCGCCATGTCGGTGCGG | 1716 | AGACGGCCGCCAUGUCGGUG | 3417 |
| | GGTTTTCGGTTATAGCCGGCCGG | 1717 | GGUUUUCGGUUAUAGCCGGC | 3418 |
| | GGGCGAGAGTCATGACACGGAGG | 1718 | GGGCGAGAGUCAUGACACGG | 3419 |
| | GCATTCAGTTCCAACGACCCAGG | 1719 | GCAUUCAGUUCCAACGACCC | 3420 |
| | GGAACGGAGACCGCGTCCTGCGG | 1720 | GGAACGGAGACCGCGUCCUG | 3421 |
| | ACTCAGCGACCTTGGCCCGAAGG | 1721 | ACUCAGCGACCUUGGCCCGA | 3422 |
| | GCGGGATTAAACCCACGTCCTGG | 1722 | GCGGGAUUAAACCCACGUCC | 3423 |
| | GGTAGCGGGTCGCAGTAGGCTGG | 1723 | GGUAGCGGGUCGCAGUAGGC | 3424 |
| | AGATGTTAGCCTTCGCTGCCAGG | 1724 | AGAUGUUAGCCUUCGCUGCC | 3425 |
| | CGGGCCGTGCGGTTGCGTCCGGG | 1725 | CGGGCCGUGCGGUUGCGUCC | 3426 |
| | CGCGGACTTTGCCCGGTGTGTGG | 1726 | CGCGGACUUUGCCCGGUGUG | 3427 |
| | GAGTCATGACACGGAGGAACTGG | 1727 | GAGUCAUGACACGGAGGAAC | 3428 |
| PTPN2 | CTCTTCGAACTCCCGCTCGATGG | 1728 | CUCUUCGAACUCCCGCUCGA | 3429 |
| | ACGGCCGACAGGGCTTGGCGTGG | 1729 | ACGGCCGACAGGGCUUGGCG | 3430 |
| | TTCGAACTCCCGCTCGATGGTGG | 1730 | UUCGAACUCCCGCUCGAUGG | 3431 |
| | CGTGCCGCGCGCAGGGACCACGG | 1731 | CGUGCCGCGCGCAGGGACCA | 3432 |
| | GCGTGCCGGCGACTTCTCAGGGG | 1732 | GCGUGCCGGCGACUUCUCAG | 3433 |
| | GATGCGCCACCAGCGTTGCGCGG | 1733 | GAUGCGCCACCAGCGUUGCG | 3434 |
| | AGCGAGCTTCGCCTCGCAGAGGG | 1734 | AGCGAGCUUCGCCUCGCAGA | 3435 |
| | GAGGTCGGCGACTGCCGCGTGGG | 1735 | GAGGUCGGCGACUGCCGCGU | 3436 |
| | GCACGATCCGGGGAGAGCGCTGG | 1736 | GCACGAUCCGGGGAGAGCGC | 3437 |
| | AGCGTGCCGGCGACTTCTCAGGG | 1737 | AGCGUGCCGGCGACUUCUCA | 3438 |
| | GTACTTTCCCCACGGCCGACAGG | 1738 | GUACUUUCCCCACGGCCGAC | 3439 |
| | ACGAGTCCGGGTCTCGGAGGAGG | 1739 | ACGAGUCCGGGUCUCGGAGG | 3440 |
| | TAGCGCGGGGTTACTGGAATGGG | 1740 | UAGCGCGGGGUUACUGGAAU | 3441 |
| | CGGACGTCAGCGCGCAGACTCGG | 1741 | CGGACGUCAGCGCGCAGACU | 3442 |
| | AGCGTTGCGCGGCCCGGGTCTGG | 1742 | AGCGUUGCGCGGCCCGGGUC | 3443 |
| | GCGTTGCGCGGCCCGGGTCTGGG | 1743 | GCGUUGCGCGGCCCGGGUCU | 3444 |
| | CGTGGGTAGCGCGGGGTTACTGG | 1744 | CGUGGGUAGCGCGGGGUUAC | 3445 |
| | TGGTGAGTCGCGGACCCACGCGG | 1745 | UGGUGAGUCGCGGACCCACG | 3446 |
| | CCGCGACTCACCAAGTACAGCGG | 1746 | CCGCGACUCACCAAGUACAG | 3447 |
| | TCGGAAGACGCAAGCCCAAGGGG | 1747 | UCGGAAGACGCAAGCCCAAG | 3448 |
| | CGCCCCGAGCGAGAGGCTAGAGG | 1748 | CGCCCCGAGCGAGAGGCUAG | 3449 |
| | GTCGGAAGACGCAAGCCCAAGGG | 1749 | GUCGGAAGACGCAAGCCCAA | 3450 |
| | TTCATTTGTGACACCCGTCTGGG | 1750 | UUCAUUUGUGACACCCGUCU | 3451 |
| | CTCAGGCCCCGCACGATCCGGGG | 1751 | CUCAGGCCCCGCACGAUCCG | 3452 |
| | AGCGCTCTCCCCGGATCGTGCGG | 1752 | AGCGCUCUCCCCGGAUCGUG | 3453 |
| | ACGGCGAAGCTGCGGCCCGGGGG | 1753 | ACGGCGAAGCUGCGGCCCGG | 3454 |
| | AGACCCGGGCCGCGCAACGCTGG | 1754 | AGACCCGGGCCGCGCAACGC | 3455 |
| | TCGGCCGTGGGGAAAGTACCTGG | 1755 | UCGGCCGUGGGGAAAGUACC | 3456 |
| | CGAGCGGGAGTTCGAAGAGTTGG | 1756 | CGAGCGGGAGUUCGAAGAGU | 3457 |
| | CGCCAAGCCCTGTCGGCCGTGGG | 1757 | CGCCAAGCCCUGUCGGCCGU | 3458 |
| | TCGCCTCTAGCCTCTCGCTCGGG | 1758 | UCGCCUCUAGCCUCUCGCUC | 3459 |
| | AGCAAGAGAGCGGTCAGCGCAGG | 1759 | AGCAAGAGAGCGGUCAGCGC | 3460 |
| | ACGCCAAGCCCTGTCGGCCGTGG | 1760 | ACGCCAAGCCCUGUCGGCCG | 3461 |
| | TCGTTCCGGGAAGGTTCTATGGG | 1761 | UCGUUCCGGGAAGGUUCUAU | 3462 |
| | AGCGCAAGCGCAGTTAGTTCTGG | 1762 | AGCGCAAGCGCAGUUAGUUC | 3463 |
| | CACGAGGTGAGCCGCCCCTTGGG | 1763 | CACGAGGUGAGCCGCCCCUU | 3464 |
| | ATTCATTTGTGACACCCGTCTGG | 1764 | AUUCAUUUGUGACACCCGUC | 3465 |
| | GCCGACTTCGCGCCGCGCTCGGG | 1765 | GCCGACUUCGCGCCGCGCUC | 3466 |
| | CTGGCGGAGCCGCGGTGGTTGGG | 1766 | CUGGCGGAGCCGCGGUGGUU | 3467 |
| | CGGTGGTCCGTGGGTAGCGCGGG | 1767 | CGGUGGUCCGUGGGUAGCGC | 3468 |
| | GTCTTCCGACAAGAGAGAGGCGG | 1768 | GUCUUCCGACAAGAGAGAGG | 3469 |
| | GCGCAGTTAGTTCTGGAGGGCGG | 1769 | GCGCAGUUAGUUCUGGAGGG | 3470 |
| | CAGTAACCCCGCGCTACCCACGG | 1770 | CAGUAACCCCGCGCUACCCA | 3471 |
| | GCAAGCGCAGTTAGTTCTGGAGG | 1771 | GCAAGCGCAGUUAGUUCUGG | 3472 |
| | CGCCTCTAGCCTCTCGCTCGGGG | 1772 | CGCCUCUAGCCUCUCGCUCG | 3473 |
| | TGGTCCCTGCGCGCGGCACGAGG | 1773 | UGGUCCCUGCGCGCGGCACG | 3474 |
| | TACTTTCCCCACGGCCGACAGGG | 1774 | UACUUUCCCCACGGCCGACA | 3475 |
| | CGTTCCGGGAAGGTTCTATGGGG | 1775 | CGUUCCGGGAAGGUUCUAUG | 3476 |
| | TGTCGGAAGACGCAAGCCCAAGG | 1776 | UGUCGGAAGACGCAAGCCCA | 3477 |
| | GAGCGAGCTTCGCCTCGCAGAGG | 1777 | GAGCGAGCUUCGCCUCGCAG | 3478 |
| CISH | TCGCGATTGGTCAGCTCGCGGGG | 1778 | UCGCGAUUGGUCAGCUCGCG | 3479 |
| | ACCAATCGCGACGCTGAAGGTGG | 1779 | ACCAAUCGCGACGCUGAAGG | 3480 |
| | CGTCGCGATTGGTCAGCTCGCGG | 1780 | CGUCGCGAUUGGUCAGCUCG | 3481 |
| | CTGACCAATCGCGACGCTGAAGG | 1781 | CUGACCAAUCGCGACGCUGA | 3482 |
| | GTCGCGATTGGTCAGCTCGCGGG | 1782 | GUCGCGAUUGGUCAGCUCGC | 3483 |
| | CAACGACGCAGAATGCCAGAAGG | 1783 | CAACGACGCAGAAUGCCAGA | 3484 |
| | AGGGCCCTCTTATCTCGCGGTGG | 1784 | AGGGCCCUCUUAUCUCGCGG | 3485 |
| | CGGCTAAAGGAGGAACTCACAGG | 1785 | CGGCUAAAGGAGGAACUCAC | 3486 |
| | AATAGCAGCGCGTGGACCCGGGG | 1786 | AAUAGCAGCGCGUGGACCCG | 3487 |
| | TTATCTCGCGGTGGAACTCGTGG | 1787 | UUAUCUCGCGGUGGAACUCG | 3488 |
| | TCCACCTTCAGCGTCGCGATTGG | 1788 | UCCACCUUCAGCGUCGCGAU | 3489 |
| | GTGGCGCGGACCGCCTGCGAGGG | 1789 | GUGGCGCGGACCGCCUGCGA | 3490 |
| | TCGCCGCTGCCGCGGGGACATGG | 1790 | UCGCCGCUGCCGCGGGGACA | 3491 |
| | CCAATAGCAGCGCGTGGACCCGG | 1791 | CCAAUAGCAGCGCGUGGACC | 3492 |
| | AGTTCCACCGCGAGATAAGAGGG | 1792 | AGUUCCACCGCGAGAUAAGA | 3493 |
| | TCTGCGTTCAGGGGTAAGCGCGG | 1793 | UCUGCGUUCAGGGGUAAGCG | 3494 |
| | CCGGTTTCCCAATCCACAGTGGG | 1794 | CCGGUUUCCCAAUCCACAGU | 3495 |
| | GTTCTCCCGTGCGCCCCTCGTGG | 1795 | GUUCUCCCGUGCGCCCCUCG | 3496 |
| | TCGCGGTGGAACTCGTGGCAGGG | 1796 | UCGCGGUGGAACUCGUGGCA | 3497 |
| | GAGTTCCACCGCGAGATAAGAGG | 1797 | GAGUUCCACCGCGAGAUAAG | 3498 |
| | TAGAACCGCGGGCTGAGCGGTGG | 1798 | UAGAACCGCGGGCUGAGCGG | 3499 |
| | GGACCATGTCCCCGCGGCAGCGG | 1799 | GGACCAUGUCCCCGCGGCAG | 3500 |
| | AGTGGCGCGGACCGCCTGCGAGG | 1800 | AGUGGCGCGGACCGCCUGCG | 3501 |
| | GCGCGGAGCGCGTGCTGGGTAGG | 1801 | GCGCGGAGCGCGUGCUGGGU | 3502 |
| | CGTGTTGGGACGGCCGCTCCTGG | 1802 | CGUGUUGGGACGGCCGCUCC | 3503 |
| | CTTCTGGCATTCTGCGTCGTTGG | 1803 | CUUCUGGCAUUCUGCGUCGU | 3504 |
| | ACCGGGGGCTGGCCGGCTAAAGG | 1804 | ACCGGGGGCUGGCCGGCUAA | 3505 |
| | AGAACCGCGGGCTGAGCGGTGGG | 1805 | AGAACCGCGGGCUGAGCGGU | 3506 |
| | CGCAGAGGACCATGTCCCCGCGG | 1806 | CGCAGAGGACCAUGUCCCCG | 3507 |
| | GCAGCGTCTTCCTAGAACCGCGG | 1807 | GCAGCGUCUUCCUAGAACCG | 3508 |
| | GGAGCGGCCGTCCCAACACGGGG | 1808 | GGAGCGGCCGUCCCAACACG | 3509 |
| | GACCGCCGGCTTGACCTCAGTGG | 1809 | GACCGCCGGCUUGACCUCAG | 3510 |
| | GGAGGGCCAATAGCAGCGCGTGG | 1810 | GGAGGGCCAAUAGCAGCGCG | 3511 |
| | TCTGGCATTCTGCGTCGTTGGGG | 1811 | UCUGGCAUUCUGCGUCGUUG | 3512 |
| | ACGCCGACAGACCTCCTTGGAGG | 1812 | ACGCCGACAGACCUCCUUGG | 3513 |
| | AGGAGCGGCCGTCCCAACACGGG | 1813 | AGGAGCGGCCGUCCCAACAC | 3514 |
| | ACTGAGCGCAGACGGACCTCAGG | 1814 | ACUGAGCGCAGACGGACCUC | 3515 |
| | GCGACTCCGGAGTGGGGACTCGG | 1815 | GCGACUCCGGAGUGGGGACU | 3516 |
| | CTTTCCAGGAAAACGGGGCGGGG | 1816 | CUUUCCAGGAAAACGGGGCG | 3517 |
| | GTGTGCAAGCGCCCCGTGTTGGG | 1817 | GUGUGCAAGCGCCCCGUGUU | 3518 |
| | CTGTGTGTCGGGTGTCGGATTGG | 1818 | CUGUGUGUCGGGUGUCGGAU | 3519 |
| | CCCGCGCCCAGATTGCCTTCTGG | 1819 | CCCGCGCCCAGAUUGCCUUC | 3520 |
| | TTCTGGCATTCTGCGTCGTTGGG | 1820 | UUCUGGCAUUCUGCGUCGUU | 3521 |
| | TCGTGCTAGCTGCCGGGCATTGG | 1821 | UCGUGCUAGCUGCCGGGCAU | 3522 |
| | CGTCTGCGCTCAGTCACCTCTGG | 1822 | CGUCUGCGCUCAGUCACCUC | 3523 |
| | CACACGCCGACAGACCTCCTTGG | 1823 | CACACGCCGACAGACCUCCU | 3524 |
| | AGACCGGGTCGGGGAAGTTAAGG | 1824 | AGACCGGGUCGGGGAAGUUA | 3525 |
| | ATTGGCCCTCCCCGACCGCTCGG | 1825 | AUUGGCCCUCCCCGACCGCU | 3526 |
| | CAGGAGCGGCCGTCCCAACACGG | 1826 | CAGGAGCGGCCGUCCCAACA | 3527 |
| | CCCTCGTGGTGGCCGGGAAGGGG | 1827 | CCCUCGUGGUGGCCGGGAAG | 3528 |
| PI3KCD.1 | GGTCCCGAAAAGTGCGCTGTGGG | 1828 | GGUCCCGAAAAGUGCGCUGU | 3529 |
| | AGGTCCCGAAAAGTGCGCTGTGG | 1829 | AGGUCCCGAAAAGUGCGCUG | 3530 |
| | GATCGCCGCTGGCTGCGTCAGGG | 1830 | GAUCGCCGCUGGCUGCGUCA | 3531 |
| | CAGTTCGCCTACCGCTAGAGGGG | 1831 | CAGUUCGCCUACCGCUAGAG | 3532 |
| | AGCAAACGCGGCGAGCAACGCGG | 1832 | AGCAAACGCGGCGAGCAACG | 3533 |
| | CGGTTTTGCCGGCGTAACCCCGG | 1833 | CGGUUUUGCCGGCGUAACCC | 3534 |
| | TTGCCGGCGTAACCCCGGCTCGG | 1834 | UUGCCGGCGUAACCCCGGCU | 3535 |
| | GGACGGTAAGCGATCGCCGCTGG | 1835 | GGACGGUAAGCGAUCGCCGC | 3536 |
| | GCGGCGATCGCTTACCGTCCCGG | 1836 | GCGGCGAUCGCUUACCGUCC | 3537 |
| | CCTCTAGCGGTAGGCGAACTGGG | 1837 | CCUCUAGCGGUAGGCGAACU | 3538 |
| | CGATCGCCGCTGGCTGCGTCAGG | 1838 | CGAUCGCCGCUGGCUGCGUC | 3539 |
| | CCCTCTAGCGGTAGGCGAACTGG | 1839 | CCCUCUAGCGGUAGGCGAAC | 3540 |
| | AGGTAGGGGCGAGATTTCCGGGG | 1840 | AGGUAGGGGCGAGAUUUCCG | 3541 |
| | GATGATGCCCCTCTAGCGGTAGG | 1841 | GAUGAUGCCCCUCUAGCGGU | 3542 |
| | CCGGATCTGCGGCCGAGCCGGGG | 1842 | CCGGAUCUGCGGCCGAGCCG | 3543 |
| | CCGCTCCGAGCGCTGACTAGAGG | 1843 | CCGCUCCGAGCGCUGACUAG | 3544 |
| | CCGAAAAGTGCGCTGTGGGTGGG | 1844 | CCGAAAAGUGCGCUGUGGGU | 3545 |
| | GGCGAGATTTCCGGGGTCGCGGG | 1845 | GGCGAGAUUUCCGGGGUCGC | 3546 |
| | CCCGAAAAGTGCGCTGTGGGTGG | 1846 | CCCGAAAAGUGCGCUGUGGG | 3547 |
| | AGGACCCGGCTCGCTAGACTCGG | 1847 | AGGACCCGGCUCGCUAGACU | 3548 |
| | GGAACTGGGACGACCTTTCGTGG | 1848 | GGAACUGGGACGACCUUUCG | 3549 |
| | GCAAACGCGGCGAGCAACGCGGG | 1849 | GCAAACGCGGCGAGCAACGC | 3550 |
| | GGTCCTCGCGTGGCACCCTTGGG | 1850 | GGUCCUCGCGUGGCACCCUU | 3551 |
| | CCCCCGTGGGCCCGCCGAGAGGG | 1851 | CCCCCGUGGGCCCGCCGAGA | 3552 |
| | CTCTAGTCAGCGCTCGGAGCGGG | 1852 | CUCUAGUCAGCGCUCGGAGC | 3553 |
| | CAGTTTGCGGATGGAGCGCGGGG | 1853 | CAGUUUGCGGAUGGAGCGCG | 3554 |
| | GAACTGGGACGACCTTTCGTGGG | 1854 | GAACUGGGACGACCUUUCGU | 3555 |
| | TGGGCGCGAGTGAGCCTCGAGGG | 1855 | UGGGCGCGAGUGAGCCUCGA | 3556 |
| | AACGCGGCGAGCAACGCGGGAGG | 1856 | AACGCGGCGAGCAACGCGGG | 3557 |
| | TAGACTCGGGGAGGCGCCCAGGG | 1857 | UAGACUCGGGGAGGCGCCCA | 3558 |
| | TCGCGCCTCAGCCGGCGCACCGG | 1858 | UCGCGCCUCAGCCGGCGCAC | 3559 |
| | CGCCTCAGCCGGCGCACCGGAGG | 1859 | CGCCUCAGCCGGCGCACCGG | 3560 |
| | GGACCCGGCTCGCTAGACTCGGG | 1860 | GGACCCGGCUCGCUAGACUC | 3561 |
| | CCAGTTTGCGGATGGAGCGCGGG | 1861 | CCAGUUUGCGGAUGGAGCGC | 3562 |
| | CTCTAGCGGTAGGCGAACTGGGG | 1862 | CUCUAGCGGUAGGCGAACUG | 3563 |
| | TAGGACTTCTCAGGAATCGGCGG | 1863 | UAGGACUUCUCAGGAAUCGG | 3564 |
| | CGAGATCAGCTCCGGATCTGCGG | 1864 | CGAGAUCAGCUCCGGAUCUG | 3565 |
| | GACCCGGCTCGCTAGACTCGGGG | 1865 | GACCCGGCUCGCUAGACUCG | 3566 |
| | TGGACCCCGCTGCCGTACAGAGG | 1866 | UGGACCCCGCUGCCGUACAG | 3567 |
| | CGCGAGTGAGCCTCGAGGGAGGG | 1867 | CGCGAGUGAGCCUCGAGGGA | 3568 |
| | GACGACCTTTCGTGGGCACCAGG | 1868 | GACGACCUUUCGUGGGCACC | 3569 |
| | GAGGGCTGCGCACAGTTCGCCGG | 1869 | GAGGGCUGCGCACAGUUCGC | 3570 |
| | AGTCTAGCGAGCCGGGTCCTGGG | 1870 | AGUCUAGCGAGCCGGGUCCU | 3571 |
| | CGAGCGCTGACTAGAGGACCAGG | 1871 | CGAGCGCUGACUAGAGGACC | 3572 |
| | TCCGGATCTGCGGCCGAGCCGGG | 1872 | UCCGGAUCUGCGGCCGAGCC | 3573 |
| | CGCGTTTGCTGCAGCGGCGCAGG | 1873 | CGCGUUUGCUGCAGCGGCGC | 3574 |
| | ACCGTCCCGGCGCAGCTGGCAGG | 1874 | ACCGUCCCGGCGCAGCUGGC | 3575 |
| | GCTCGCCGCGTTTGCTGCAGCGG | 1875 | GCUCGCCGCGUUUGCUGCAG | 3576 |
| | GTCCGGAAATGCAAAGCTGGGGG | 1876 | GUCCGGAAAUGCAAAGCUGG | 3577 |
| | GATCCCAAGGGTGCCACGCGAGG | 1877 | GAUCCCAAGGGUGCCACGCG | 3578 |
| | GTACCGGGTGTCGCTGCCGGGGG | 1878 | GUACCGGGUGUCGCUGCCGG | 3579 |
| | CTTGCCTGCACCTCGCGCGGCGG | 1879 | CUUGCCUGCACCUCGCGCGG | 3580 |
| | GGCAGGCTGTTTACTTGTCGGGG | 1880 | GGCAGGCUGUUUACUUGUCG | 3581 |
| | ACTTGTCGGGGACCCAGCAGTGG | 1881 | ACUUGUCGGGGACCCAGCAG | 3582 |
| | GAGGCTCCGTCCCGAATAGGGGG | 1882 | GAGGCUCCGUCCCGAAUAGG | 3583 |
| | CGTACCGGGTGTCGCTGCCGGGG | 1883 | CGUACCGGGUGUCGCUGCCG | 3584 |
| | TCCGTCCCGAATAGGGGGCAGGG | 1884 | UCCGUCCCGAAUAGGGGGCA | 3585 |
| | CGTCCCGAATAGGGGGCAGGGGG | 1885 | CGUCCCGAAUAGGGGGCAGG | 3586 |
| | CAGGGGGTTGCGTTCGCGGTGGG | 1886 | CAGGGGGUUGCGUUCGCGGU | 3587 |
| | AGGAGGCTCCGTCCCGAATAGGG | 1887 | AGGAGGCUCCGUCCCGAAUA | 3588 |
| | TTCTCCGCTGCCGCCCTTGATGG | 1888 | UUCUCCGCUGCCGCCCUUGA | 3589 |
| | CGCGGTGGGATTCTCAGCTATGG | 1889 | CGCGGUGGGAUUCUCAGCUA | 3590 |
| | TCCGTACCGGGTGTCGCTGCCGG | 1890 | UCCGUACCGGGUGUCGCUGC | 3591 |
| | CGCACGAGGACGCGCCTGTTCGG | 1891 | CGCACGAGGACGCGCCUGUU | 3592 |
| | AGCGCCCGAGCTCACACGGGCGG | 1892 | AGCGCCCGAGCUCACACGGG | 3593 |
| | CCGCTTGCCTGCACCTCGCGCGG | 1893 | CCGCUUGCCUGCACCUCGCG | 3594 |
| | GGAGGCTCCGTCCCGAATAGGGG | 1894 | GGAGGCUCCGUCCCGAAUAG | 3595 |
| | CGACCCCCGCTGTTCTCGCCCGG | 1895 | CGACCCCCGCUGUUCUCGCC | 3596 |
| | GGGCCTCCGGGCGAGAACAGCGG | 1896 | GGGCCUCCGGGCGAGAACAG | 3597 |
| | AGGACGCGCCTGTTCGGGGCAGG | 1897 | AGGACGCGCCUGUUCGGGGC | 3598 |
| | CGTCCTCGTGCGAAGCCCGCTGG | 1898 | CGUCCUCGUGCGAAGCCCGC | 3599 |
| | TGACCCCCGGGGGGCACAAAAGG | 1899 | UGACCCCCGGGGGGCACAAA | 3600 |
| | TTGCCTGCACCTCGCGCGGCGGG | 1900 | UUGCCUGCACCUCGCGCGGC | 3601 |
| | GCACGAGGACGCGCCTGTTCGGG | 1901 | GCACGAGGACGCGCCUGUUC | 3602 |
| | CCAGCGTGCGCGCGCCGTCGGGG | 1902 | CCAGCGUGCGCGCGCCGUCG | 3603 |
| | GTAAACAGCCTGCCCCGAACAGG | 1903 | GUAAACAGCCUGCCCCGAAC | 3604 |
| | CGGTACGGAGCCCACCTGTGCGG | 1904 | CGGUACGGAGCCCACCUGUG | 3605 |
| | CCCGGCAGCGACACCCGGTACGG | 1905 | CCCGGCAGCGACACCCGGUA | 3606 |
| | GCGGTGGGATTCTCAGCTATGGG | 1906 | GCGGUGGGAUUCUCAGCUAU | 3607 |
| | CCTCCAGCGGGCTTCGCACGAGG | 1907 | CCUCCAGCGGGCUUCGCACG | 3608 |
| | GATCCCATCAAGGGCGGCAGCGG | 1908 | GAUCCCAUCAAGGGCGGCAG | 3609 |
| | TTTCCGCTCCCCGCTTTGCAAGG | 1909 | UUUCCGCUCCCCGCUUUGCA | 3610 |
| | CTCCGTCCCGAATAGGGGGCAGG | 1910 | CUCCGUCCCGAAUAGGGGGC | 3611 |
| | TCTCCGCTGCCGCCCTTGATGGG | 1911 | UCUCCGCUGCCGCCCUUGAU | 3612 |
| | AGCCAGCGTGCGCGCGCCGTCGG | 1912 | AGCCAGCGUGCGCGCGCCGU | 3613 |
| | GCTCAGGGTGCGAACCCCAAGGG | 1913 | GCUCAGGGUGCGAACCCCAA | 3614 |
| | TAGGAGAGGAGAGCGTCGCGCGG | 1914 | UAGGAGAGGAGAGCGUCGCG | 3615 |
| | CGCCCTTGATGGGATCCGTGAGG | 1915 | CGCCCUUGAUGGGAUCCGUG | 3616 |
| | CACCTGTGCGGGCGTCTGCGGGG | 1916 | CACCUGUGCGGGCGUCUGCG | 3617 |
| | TCTGCGGATGCCTTGCAAAGCGG | 1917 | UCUGCGGAUGCCUUGCAAAG | 3618 |
| | GCGCGAGGTGCAGGCAAGCGGGG | 1918 | GCGCGAGGUGCAGGCAAGCG | 3619 |
| | AGGCATCCGCAGAAAGGGCGGGG | 1919 | AGGCAUCCGCAGAAAGGGCG | 3620 |
| | GCCAGCGTGCGCGCGCCGTCGGG | 1920 | GCCAGCGUGCGCGCGCCGUC | 3621 |
| | CTCACGGATCCCATCAAGGGCGG | 1921 | CUCACGGAUCCCAUCAAGGG | 3622 |
| | CCTCCGGGCGAGAACAGCGGGGG | 1922 | CCUCCGGGCGAGAACAGCGG | 3623 |
| | AAGCGCCACCTGCAAAGCAAGGG | 1923 | AAGCGCCACCUGCAAAGCAA | 3624 |
| | GCCTCCGGGCGAGAACAGCGGGG | 1924 | GCCUCCGGGCGAGAACAGCG | 3625 |
| | AGAAGCGCCCGAGCTCACACGGG | 1925 | AGAAGCGCCCGAGCUCACAC | 3626 |
| | GCTTCCTTTTGTGCCCCCCGGGG | 1926 | GCUUCCUUUUGUGCCCCCCG | 3627 |
| | GCGAACGCAACCCCCTGCCTCGG | 1927 | GCGAACGCAACCCCCUGCCU | 3628 |
| MAP4K1 | CACGACCCCCGTTCCCGCGGAGG | 1928 | CACGACCCCCGUUCCCGCGG | 3629 |
| | CGAGATGAGCACCGGTGAGTGGG | 1929 | CGAGAUGAGCACCGGUGAGU | 3630 |
| | ACGGCATCCCCCAAGACTTAGGG | 1930 | ACGGCAUCCCCCAAGACUUA | 3631 |
| | CGCTTAGCCTGAGGCACTACGGG | 1931 | CGCUUAGCCUGAGGCACUAC | 3632 |
| | GGGGGTCGTGACCTCCGAGTGGG | 1932 | GGGGGUCGUGACCUCCGAGU | 3633 |
| | ATGCCACCTTGGCGGCAGACGGG | 1933 | AUGCCACCUUGGCGGCAGAC | 3634 |
| | CGGACAGAGGCGTCGGCAGTGGG | 1934 | CGGACAGAGGCGUCGGCAGU | 3635 |
| | CCCGGTCAGCAGCGCGAACACGG | 1935 | CCCGGUCAGCAGCGCGAACA | 3636 |
| | AGGGCGGGGCTTATCAGATCCGG | 1936 | AGGGCGGGGCUUAUCAGAUC | 3637 |
| | GCGCGCCAAAGCGCACCGTGTGG | 1937 | GCGCGCCAAAGCGCACCGUG | 3638 |
| | AGCAGCGCGAACACGGACAGAGG | 1938 | AGCAGCGCGAACACGGACAG | 3639 |
| | GGTCACGACCCCCGTTCCCGCGG | 1939 | GGUCACGACCCCCGUUCCCG | 3640 |
| | CGGGGCTTATCAGATCCGGAGGG | 1940 | CGGGGCUUAUCAGAUCCGGA | 3641 |
| | CGGGGGTCGTGACCTCCGAGTGG | 1941 | CGGGGGUCGUGACCUCCGAG | 3642 |
| | CTGGTGCCTCCGCGGGAACGGGG | 1942 | CUGGUGCCUCCGCGGGAACG | 3643 |
| | GCGGGGCTTATCAGATCCGGAGG | 1943 | GCGGGGCUUAUCAGAUCCGG | 3644 |
| | TCCGTGTTCGCGCTGCTGACCGG | 1944 | UCCGUGUUCGCGCUGCUGAC | 3645 |
| | GCTTTGGCGCGCTCTCTTGCTGG | 1945 | GCUUUGGCGCGCUCUCUUGC | 3646 |
| | GGCCTGGGACTTCCGAACCAGGG | 1946 | GGCCUGGGACUUCCGAACCA | 3647 |
| | TGGTGCCTCCGCGGGAACGGGGG | 1947 | UGGUGCCUCCGCGGGAACGG | 3648 |
| | GACGGCATCCCCCAAGACTTAGG | 1948 | GACGGCAUCCCCCAAGACUU | 3649 |
| | CTCACGCCGATGCACACAGCGGG | 1949 | CUCACGCCGAUGCACACAGC | 3650 |
| | GGATGCCGTCTAGAAATGTCAGG | 1950 | GGAUGCCGUCUAGAAAUGUC | 3651 |
| | TCGCTTAGCCTGAGGCACTACGG | 1951 | UCGCUUAGCCUGAGGCACUA | 3652 |
| | CGAACCAGGGCCCTAAGTCTTGG | 1952 | CGAACCAGGGCCCUAAGUCU | 3653 |
| | ATGCCGTCTAGAAATGTCAGGGG | 1953 | AUGCCGUCUAGAAAUGUCAG | 3654 |
| | GCATCGGCGTGAGCCCCGGGCGG | 1954 | GCAUCGGCGUGAGCCCCGGG | 3655 |
| | GCGAACACGGACAGAGGCGTCGG | 1955 | GCGAACACGGACAGAGGCGU | 3656 |
| | GGGGCTTATCAGATCCGGAGGGG | 1956 | GGGGCUUAUCAGAUCCGGAG | 3657 |
| | TGGCCTGGGACTTCCGAACCAGG | 1957 | UGGCCUGGGACUUCCGAACC | 3658 |
| | AATGGCAGGTTTTAGTTAACTGG | 1958 | AAUGGCAGGUUUUAGUUAAC | 3659 |
| | TGGAAGCCACACCCACTCGGAGG | 1959 | UGGAAGCCACACCCACUCGG | 3660 |
| | GCTACAAGCCACGCCCCCTGAGG | 1960 | GCUACAAGCCACGCCCCCUG | 3661 |
| | TGGAAGAGCACCGACTTCCCCGG | 1961 | UGGAAGAGCACCGACUUCCC | 3662 |
| | TCGTGACCTCCGAGTGGGTGTGG | 1962 | UCGUGACCUCCGAGUGGGUG | 3663 |
| | CTCTTCCACACGGTGCGCTTTGG | 1963 | CUCUUCCACACGGUGCGCUU | 3664 |
| | AGGCACTACGGGACTGAGAAAGG | 1964 | AGGCACUACGGGACUGAGAA | 3665 |
| | GGCCCTGGTTCGGAAGTCCCAGG | 1965 | GGCCCUGGUUCGGAAGUCCC | 3666 |
| | CCTGGTGCCTCCGCGGGAACGGG | 1966 | CCUGGUGCCUCCGCGGGAAC | 3667 |
| | GCGAGATGAGCACCGGTGAGTGG | 1967 | GCGAGAUGAGCACCGGUGAG | 3668 |
| | GTCTCTTTGAGTGTCTAAGCAGG | 1968 | GUCUCUUUGAGUGUCUAAGC | 3669 |
| | CTAGGGGGTGGTTCAGGACGGGG | 1969 | CUAGGGGGUGGUUCAGGACG | 3670 |
| | GCCTGGTGCCTCCGCGGGAACGG | 1970 | GCCUGGUGCCUCCGCGGGAA | 3671 |
| | GCTTAGACACTCAAAGAGACAGG | 1971 | GCUUAGACACUCAAAGAGAC | 3672 |
| | GATAAGGCCTGGTGCCTCCGCGG | 1972 | GAUAAGGCCUGGUGCCUCCG | 3673 |
| | ACCTTGGCGGCAGACGGGCAGGG | 1973 | ACCUUGGCGGCAGACGGGCA | 3674 |
| | CAAGGTGGCATGCCCCCACATGG | 1974 | CAAGGUGGCAUGCCCCCACA | 3675 |
| | CATGCCACCTTGGCGGCAGACGG | 1975 | CAUGCCACCUUGGCGGCAGA | 3676 |
| | GGCGCGCTCTCTTGCTGGCTGGG | 1976 | GGCGCGCUCUCUUGCUGGCU | 3677 |
| | GATGCCGTCTAGAAATGTCAGGG | 1977 | GAUGCCGUCUAGAAAUGUCA | 3678 |
| NR4A1 | CGCGGGGTTCCATTGACGCAGGG | 1978 | CGCGGGGUUCCAUUGACGCA | 3679 |
| | GGCGGAGGCTACGAAACTTGGGG | 1979 | GGCGGAGGCUACGAAACUUG | 3680 |
| | TAAGCGCTCCGTGACGCACGGGG | 1980 | UAAGCGCUCCGUGACGCACG | 3681 |
| | ACGCGGGGTTCCATTGACGCAGG | 1981 | ACGCGGGGUUCCAUUGACGC | 3682 |
| | AAGAACTTCGGGAGCGCACGCGG | 1982 | AAGAACUUCGGGAGCGCACG | 3683 |
| | TTTGGCCATACAAGGGCGCGGGG | 1983 | UUUGGCCAUACAAGGGCGCG | 3684 |
| | GTTTCGTAGCCTCCGCCACTGGG | 1984 | GUUUCGUAGCCUCCGCCACU | 3685 |
| | ATCCGCGCTCCCTGCGTCAATGG | 1985 | AUCCGCGCUCCCUGCGUCAA | 3686 |
| | TTAAGCGCTCCGTGACGCACGGG | 1986 | UUAAGCGCUCCGUGACGCAC | 3687 |
| | GTGGCGGAGGCTACGAAACTTGG | 1987 | GUGGCGGAGGCUACGAAACU | 3688 |
| | GCGGAGGCTACGAAACTTGGGGG | 1988 | GCGGAGGCUACGAAACUUGG | 3689 |
| | ACAGATGCACGTTCCCCGAAGGG | 1989 | ACAGAUGCACGUUCCCCGAA | 3690 |
| | AACAGATGCACGTTCCCCGAAGG | 1990 | AACAGAUGCACGUUCCCCGA | 3691 |
| | TTGTATGGCCAAAGCTCGACGGG | 1991 | UUGUAUGGCCAAAGCUCGAC | 3692 |
| | CTGCGCGCGTGACGCACGCGGGG | 1992 | CUGCGCGCGUGACGCACGCG | 3693 |
| | CTTAAGCGCTCCGTGACGCACGG | 1993 | CUUAAGCGCUCCGUGACGCA | 3694 |
| | GTCACGCGCGCAGACATTCCAGG | 1994 | GUCACGCGCGCAGACAUUCC | 3695 |
| | TGCGTCACGGAGCGCTTAAGAGG | 1995 | UGCGUCACGGAGCGCUUAAG | 3696 |
| | CGCTCCGTGACGCACGGGGAGGG | 1996 | CGCUCCGUGACGCACGGGGA | 3697 |
| | TGAGACTCGGGGCGCCAGTCCGG | 1997 | UGAGACUCGGGGCGCCAGUC | 3698 |
| | GCGCTGTAGAGACGCGGCCGCGG | 1998 | GCGCUGUAGAGACGCGGCCG | 3699 |
| | TATGGCCAAAGCTCGACGGGCGG | 1999 | UAUGGCCAAAGCUCGACGGG | 3700 |
| | GTCGAGCTTTGGCCATACAAGGG | 2000 | GUCGAGCUUUGGCCAUACAA | 3701 |
| | TCACGGAGCGCTTAAGAGGAGGG | 2001 | UCACGGAGCGCUUAAGAGGA | 3702 |
| | GTCCAGAATAACCAGCGGGAGGG | 2002 | GUCCAGAAUAACCAGCGGGA | 3703 |
| | TGGGACCCGAGTCCGGTGCGGGG | 2003 | UGGGACCCGAGUCCGGUGCG | 3704 |
| | TTGGCCATACAAGGGCGCGGGGG | 2004 | UUGGCCAUACAAGGGCGCGG | 3705 |
| | AAGGAGATGGGTGTACGCGCGGG | 2005 | AAGGAGAUGGGUGUACGCGC | 3706 |
| | GCGCTCCGTGACGCACGGGGAGG | 2006 | GCGCUCCGUGACGCACGGGG | 3707 |
| | CGGGCAATTCGGACACACCCTGG | 2007 | CGGGCAAUUCGGACACACCC | 3708 |
| | CGTCGAGCTTTGGCCATACAAGG | 2008 | CGUCGAGCUUUGGCCAUACA | 3709 |
| | TGGCGGAGGCTACGAAACTTGGG | 2009 | UGGCGGAGGCUACGAAACUU | 3710 |
| | AGTTTCGTAGCCTCCGCCACTGG | 2010 | AGUUUCGUAGCCUCCGCCAC | 3711 |
| | AGGGCTCTAACTGACGTCTCAGG | 2011 | AGGGCUCUAACUGACGUCUC | 3712 |
| | GCAGGCCGCCCGTCGAGCTTTGG | 2012 | GCAGGCCGCCCGUCGAGCUU | 3713 |
| | GCGGGCTGAGGCGGGCAATTCGG | 2013 | GCGGGCUGAGGCGGGCAAUU | 3714 |
| | TCTGCGCGCGTGACGCACGCGGG | 2014 | UCUGCGCGCGUGACGCACGC | 3715 |
| | GTCTGCGCGCGTGACGCACGCGG | 2015 | GUCUGCGCGCGUGACGCACG | 3716 |
| | TAGGCTCCCCGCACCGGACTCGG | 2016 | UAGGCUCCCCGCACCGGACU | 3717 |
| | TTGTAGGGCCGGCATGCAAGAGG | 2017 | UUGUAGGGCCGGCAUGCAAG | 3718 |
| | GCTTTGGCCATACAAGGGCGCGG | 2018 | GCUUUGGCCAUACAAGGGCG | 3719 |
| | GGGCTCTAACTGACGTCTCAGGG | 2019 | GGGCUCUAACUGACGUCUCA | 3720 |
| | CTGTGCACTAGCTGCGCCTAGGG | 2020 | CUGUGCACUAGCUGCGCCUA | 3721 |
| | AGAGTGAGGAGATCCTCATCCGG | 2021 | AGAGUGAGGAGAUCCUCAUC | 3722 |
| | GCTCCGTGACGCACGGGGAGGGG | 2022 | GCUCCGUGACGCACGGGGAG | 3723 |
| | TCGGGGCGCCAGTCCGGGCAGGG | 2023 | UCGGGGCGCCAGUCCGGGCA | 3724 |
| | CGCAGCTAGTGCACAGGACGCGG | 2024 | CGCAGCUAGUGCACAGGACG | 3725 |
| | CGGCCGGGTAGGTTCCCTTCGGG | 2025 | CGGCCGGGUAGGUUCCCUUC | 3726 |
| | CTATTTTTAGCGGGCGCGGCGGG | 2026 | CUAUUUUUAGCGGGCGCGGC | 3727 |
| | CCCGCTGGTTATTCTGGACCTGG | 2027 | CCCGCUGGUUAUUCUGGACC | 3728 |
| NR4A2 | CTCGAAACCGAAGAGCCCACAGG | 2028 | CUCGAAACCGAAGAGCCCAC | 3729 |
| | TCGAGGGCAAACGACCTCTCCGG | 2029 | UCGAGGGCAAACGACCUCUC | 3730 |
| | TAACTATACGACCCATTTGGAGG | 2030 | UAACUAUACGACCCAUUUGG | 3731 |
| | TCGGAAAAGCGGCGCTAACAGGG | 2031 | UCGGAAAAGCGGCGCUAACA | 3732 |
| | CTCGGAAAAGCGGCGCTAACAGG | 2032 | CUCGGAAAAGCGGCGCUAAC | 3733 |
| | AGCCGGGTTGGAGTCGACATGGG | 2033 | AGCCGGGUUGGAGUCGACAU | 3734 |
| | AGTCGACATGGGCCCTGACGAGG | 2034 | AGUCGACAUGGGCCCUGACG | 3735 |
| | AGACTCACCGGGGGCGAAGGGGG | 2035 | AGACUCACCGGGGGCGAAGG | 3736 |
| | CTTTAACTATACGACCCATTTGG | 2036 | CUUUAACUAUACGACCCAUU | 3737 |
| | GTCGACATGGGCCCTGACGAGGG | 2037 | GUCGACAUGGGCCCUGACGA | 3738 |
| | AGCGCCGCTTTTCCGAGCCCAGG | 2038 | AGCGCCGCUUUUCCGAGCCC | 3739 |
| | GGCCCATGTCGACTCCAACCCGG | 2039 | GGCCCAUGUCGACUCCAACC | 3740 |
| | ATGTGGACAAACCGACAGATGGG | 2040 | AUGUGGACAAACCGACAGAU | 3741 |
| | TGTGGGCTCTTCGGTTTCGAGGG | 2041 | UGUGGGCUCUUCGGUUUCGA | 3742 |
| | TCAGACTCACCGGGGGCGAAGGG | 2042 | UCAGACUCACCGGGGGCGAA | 3743 |
| | GTCTGATCAGTGCCCTCGTCAGG | 2043 | GUCUGAUCAGUGCCCUCGUC | 3744 |
| | GCACTGATCAGACTCACCGGGGG | 2044 | GCACUGAUCAGACUCACCGG | 3745 |
| | GACAGTTTAAAAGGCCGGAGAGG | 2045 | GACAGUUUAAAAGGCCGGAG | 3746 |
| | ATCAGACTCACCGGGGGCGAAGG | 2046 | AUCAGACUCACCGGGGGCGA | 3747 |
| | CAACCCGGCTATGACCAGCCTGG | 2047 | CAACCCGGCUAUGACCAGCC | 3748 |
| | GGCACTGATCAGACTCACCGGGG | 2048 | GGCACUGAUCAGACUCACCG | 3749 |
| | CTGAGAGTTAATGACGGATGTGG | 2049 | CUGAGAGUUAAUGACGGAUG | 3750 |
| | TCCAGGGTAAGAAGCTGGCGGGG | 2050 | UCCAGGGUAAGAAGCUGGCG | 3751 |
| | GTTCGCACAGACAGTTTAAAAGG | 2051 | GUUCGCACAGACAGUUUAAA | 3752 |
| | TACCCTGGAATAGTCCAGGCTGG | 2052 | UACCCUGGAAUAGUCCAGGC | 3753 |
| | TGACCAGCCTGGACTATTCCAGG | 2053 | UGACCAGCCUGGACUAUUCC | 3754 |
| | TTAACTCTCAGATTCAACGGGGG | 2054 | UUAACUCUCAGAUUCAACGG | 3755 |
| | ATTAACTCTCAGATTCAACGGGG | 2055 | AUUAACUCUCAGAUUCAACG | 3756 |
| | GTCTGTGCGAACCACTGCAAAGG | 2056 | GUCUGUGCGAACCACUGCAA | 3757 |
| | TGAGAGTTAATGACGGATGTGGG | 2057 | UGAGAGUUAAUGACGGAUGU | 3758 |
| | CAAATGGGTCGTATAGTTAAAGG | 2058 | CAAAUGGGUCGUAUAGUUAA | 3759 |
| | TAGCCGGGTTGGAGTCGACATGG | 2059 | UAGCCGGGUUGGAGUCGACA | 3760 |
| | TCTGATCAGTGCCCTCGTCAGGG | 2060 | UCUGAUCAGUGCCCUCGUCA | 3761 |
| | TTCTTACCCTGGAATAGTCCAGG | 2061 | UUCUUACCCUGGAAUAGUCC | 3762 |
| | CCCCCGCCAGCTTCTTACCCTGG | 2062 | CCCCCGCCAGCUUCUUACCC | 3763 |
| | CAGACTCACCGGGGGCGAAGGGG | 2063 | CAGACUCACCGGGGGCGAAG | 3764 |
| | ACTATTCCAGGGTAAGAAGCTGG | 2064 | ACUAUUCCAGGGUAAGAAGC | 3765 |
| | GACCAGCCTGGACTATTCCAGGG | 2065 | GACCAGCCUGGACUAUUCCA | 3766 |
| | CTGGCGGGGGGGATATCATGTGG | 2066 | CUGGCGGGGGGGAUAUCAUG | 3767 |
| | GAGAGTTAATGACGGATGTGGGG | 2067 | GAGAGUUAAUGACGGAUGUG | 3768 |
| | CAGGCTGGTCATAGCCGGGTTGG | 2068 | CAGGCUGGUCAUAGCCGGGU | 3769 |
| | TTAATGACGGATGTGGGGAGGGG | 2069 | UUAAUGACGGAUGUGGGGAG | 3770 |
| | CGTATAGTTAAAGGAGAGAAGGG | 2070 | CGUAUAGUUAAAGGAGAGAA | 3771 |
| | AGTTAATGACGGATGTGGGGAGG | 2071 | AGUUAAUGACGGAUGUGGGG | 3772 |
| | TTAATGCTTCTAGTCAGTGAAGG | 2072 | UUAAUGCUUCUAGUCAGUGA | 3773 |
| | GAGGGGTCCTGCCCATCTGTCGG | 2073 | GAGGGGUCCUGCCCAUCUGU | 3774 |
| | TCGTATAGTTAAAGGAGAGAAGG | 2074 | UCGUAUAGUUAAAGGAGAGA | 3775 |
| | CTGTGGGCTCTTCGGTTTCGAGG | 2075 | CUGUGGGCUCUUCGGUUUCG | 3776 |
| | TAGTCCAGGCTGGTCATAGCCGG | 2076 | UAGUCCAGGCUGGUCAUAGC | 3777 |
| | CATTAACTCTCAGATTCAACGGG | 2077 | CAUUAACUCUCAGAUUCAAC | 3778 |
| | CTACGCACATGATCGAGCAGAGG | 2078 | CUACGCACAUGAUCGAGCAG | 3779 |
| | GATCCCGGGTCGTCCCACATGGG | 2079 | GAUCCCGGGUCGUCCCACAU | 3780 |
| | CCGGGTCGGCTGAATGCGAGGGG | 2080 | CCGGGUCGGCUGAAUGCGAG | 3781 |
| | TGGACGCGGGCTTGCGAATGGGG | 2081 | UGGACGCGGGCUUGCGAAUG | 3782 |
| | AGTTGCCAGATGCGCTTCGACGG | 2082 | AGUUGCCAGAUGCGCUUCGA | 3783 |
| | GTTGCCAGATGCGCTTCGACGGG | 2083 | GUUGCCAGAUGCGCUUCGAC | 3784 |
| | GGGGCCCGTCGAAGCGCATCTGG | 2084 | GGGGCCCGUCGAAGCGCAUC | 3785 |
| | ATTCGCAAGCCCGCGTCCATGGG | 2085 | AUUCGCAAGCCCGCGUCCAU | 3786 |
| | CATGGACGCGGGCTTGCGAATGG | 2086 | CAUGGACGCGGGCUUGCGAA | 3787 |
| | CATTCGCAAGCCCGCGTCCATGG | 2087 | CAUUCGCAAGCCCGCGUCCA | 3788 |
| | CGGGTCGGCTGAATGCGAGGGGG | 2088 | CGGGUCGGCUGAAUGCGAGG | 3789 |
| | GGGCTTGTAGTAAACCGACCCGG | 2089 | GGGCUUGUAGUAAACCGACC | 3790 |
| | AGATCCCGGGTCGTCCCACATGG | 2090 | AGAUCCCGGGUCGUCCCACA | 3791 |
| | AGCCGGGTCGGCTGAATGCGAGG | 2091 | AGCCGGGUCGGCUGAAUGCG | 3792 |
| | GCCGGGTCGGCTGAATGCGAGGG | 2092 | GCCGGGUCGGCUGAAUGCGA | 3793 |
| | GAGACGCGTGGCCGATCTGCAGG | 2093 | GAGACGCGUGGCCGAUCUGC | 3794 |
| | ATGGACGCGGGCTTGCGAATGGG | 2094 | AUGGACGCGGGCUUGCGAAU | 3795 |
| | GCGTAGTGGCCACGTAGTTCTGG | 2095 | GCGUAGUGGCCACGUAGUUC | 3796 |
| | TTCGGCGGACCCCGGAGAGCTGG | 2096 | UUCGGCGGACCCCGGAGAGC | 3797 |
| | TACGGCGTGCGCACCTGTGAGGG | 2097 | UACGGCGUGCGCACCUGUGA | 3798 |
| | GCGCACGCCGTAGTGTTGGCAGG | 2098 | GCGCACGCCGUAGUGUUGGC | 3799 |
| | AGGTCTGCCCGTCCACCACGTGG | 2099 | AGGUCUGCCCGUCCACCACG | 3800 |
| | CGCATCTGGCAACTAGACACCGG | 2100 | CGCAUCUGGCAACUAGACAC | 3801 |
| | ATCCCGGGTCGTCCCACATGGGG | 2101 | AUCCCGGGUCGUCCCACAUG | 3802 |
| | ACTAGACACCGGGGTGCCAGGGG | 2102 | ACUAGACACCGGGGUGCCAG | 3803 |
| | GTGCCCTCACCGCCGTCGCGGGG | 2103 | GUGCCCUCACCGCCGUCGCG | 3804 |
| | TCGGCGGACCCCGGAGAGCTGGG | 2104 | UCGGCGGACCCCGGAGAGCU | 3805 |
| | CGGACAGCAGTCCTCCATTAAGG | 2105 | CGGACAGCAGUCCUCCAUUA | 3806 |
| | TGTCGAGCAGCTGAGACGCGTGG | 2106 | UGUCGAGCAGCUGAGACGCG | 3807 |
| | TAGTAAACCGACCCGGAGTGCGG | 2107 | UAGUAAACCGACCCGGAGUG | 3808 |
| | CTACGGCGTGCGCACCTGTGAGG | 2108 | CUACGGCGUGCGCACCUGUG | 3809 |
| | TCCGAGGTCCCGGGCACTAGGGG | 2109 | UCCGAGGUCCCGGGCACUAG | 3810 |
| | CCGGCTCCAGCAACTTCGGGCGG | 2110 | CCGGCUCCAGCAACUUCGGG | 3811 |
| | TCGCATTCAGCCGACCCGGCTGG | 2111 | UCGCAUUCAGCCGACCCGGC | 3812 |
| | GGCTCCAGCAACTTCGGGCGGGG | 2112 | GGCUCCAGCAACUUCGGGCG | 3813 |
| | AACTTCGGGCGGGGGCCAGCCGG | 2113 | AACUUCGGGCGGGGGCCAGC | 3814 |
| | AGGTGCGCACGCCGTAGTGTTGG | 2114 | AGGUGCGCACGCCGUAGUGU | 3815 |
| | CTAGACACCGGGGTGCCAGGGGG | 2115 | CUAGACACCGGGGUGCCAGG | 3816 |
| | TAGTGGCCACGTAGTTCTGGTGG | 2116 | UAGUGGCCACGUAGUUCUGG | 3817 |
| | GATGATGCCGCACTCCGGGTCGG | 2117 | GAUGAUGCCGCACUCCGGGU | 3818 |
| | GCGCACAGCCCCTCGTTGGAGGG | 2118 | GCGCACAGCCCCUCGUUGGA | 3819 |
| | GCGTGGCCGATCTGCAGGCCCGG | 2119 | GCGUGGCCGAUCUGCAGGCC | 3820 |
| | ATGCGAGGGGGATGCGACCCTGG | 2120 | AUGCGAGGGGGAUGCGACCC | 3821 |
| | GCTGTCCGGACAGGGGCATTTGG | 2121 | GCUGUCCGGACAGGGGCAUU | 3822 |
| | GCCAGGGGGCGATTGCTTAAAGG | 2122 | GCCAGGGGGCGAUUGCUUAA | 3823 |
| | CGGCATCATCTCCTCAGACTGGG | 2123 | CGGCAUCAUCUCCUCAGACU | 3824 |
| | CCGGGTTCATGGGGACGTGCAGG | 2124 | CCGGGUUCAUGGGGACGUGC | 3825 |
| | CGCGGGCTTGCGAATGGGGTTGG | 2125 | CGCGGGCUUGCGAAUGGGGU | 3826 |
| | AACGCGGCCTGCCAACACTACGG | 2126 | AACGCGGCCUGCCAACACUA | 3827 |
| | CTGCTCGATCATGTGCGTAGTGG | 2127 | CUGCUCGAUCAUGUGCGUAG | 3828 |
| NR4A3 | GTACGGGTGGCTCTCAAGCGCGG | 2128 | GUACGGGUGGCUCUCAAGCG | 3829 |
| | CCACCTCGGCTACGACCCGACGG | 2129 | CCACCUCGGCUACGACCCGA | 3830 |
| | CATAACGCCCCCGCCTGCGGGGG | 2130 | CAUAACGCCCCCGCCUGCGG | 3831 |
| | CGCTTGAGAGCCACCCGTACGGG | 2131 | CGCUUGAGAGCCACCCGUAC | 3832 |
| | CGGCCGTCGGGTCGTAGCCGAGG | 2132 | CGGCCGUCGGGUCGUAGCCG | 3833 |
| | ACCGTGGGGACCGCCTTCATCGG | 2133 | ACCGUGGGGACCGCCUUCAU | 3834 |
| | GACGACGAGCTCCTGCTGGGCGG | 2134 | GACGACGAGCUCCUGCUGGG | 3835 |
| | GTGGGGACCGCCTTCATCGGCGG | 2135 | GUGGGGACCGCCUUCAUCGG | 3836 |
| | TCGGGTCGTAGCCGAGGTGGTGG | 2136 | UCGGGUCGUAGCCGAGGUGG | 3837 |
| | ATAACGCCCCCGCCTGCGGGGGG | 2137 | AUAACGCCCCCGCCUGCGGG | 3838 |
| | GCGCTTGAGAGCCACCCGTACGG | 2138 | GCGCUUGAGAGCCACCCGUA | 3839 |
| | CCCGCAGGCGGGGGCGTTATGGG | 2139 | CCCGCAGGCGGGGGCGUUAU | 3840 |
| | TACGGCGTGCGAACCTGCGAGGG | 2140 | UACGGCGUGCGAACCUGCGA | 3841 |
| | AGGTTCGCACGCCGTAGTGCTGG | 2141 | AGGUUCGCACGCCGUAGUGC | 3842 |
| | CAGGAGCTCGTCGTCTGGCGAGG | 2142 | CAGGAGCUCGUCGUCUGGCG | 3843 |
| | TGGGGACCGCCTTCATCGGCGGG | 2143 | UGGGGACCGCCUUCAUCGGC | 3844 |
| | CCCGGTTTGAGAGCTGTAATCGG | 2144 | CCCGGUUUGAGAGCUGUAAU | 3845 |
| | TCATCGGCGGGTCCAGCAGCGGG | 2145 | UCAUCGGCGGGUCCAGCAGC | 3846 |
| | CTACGGCGTGCGAACCTGCGAGG | 2146 | CUACGGCGUGCGAACCUGCG | 3847 |
| | TCGCACGCCGTAGTGCTGGCAGG | 2147 | UCGCACGCCGUAGUGCUGGC | 3848 |
| | CGGGTGGCTCTCAAGCGCGGCGG | 2148 | CGGGUGGCUCUCAAGCGCGG | 3849 |
| | TTCATCGGCGGGTCCAGCAGCGG | 2149 | UUCAUCGGCGGGUCCAGCAG | 3850 |
| | GCGCCCGGCTGCATCGCACCCGG | 2150 | GCGCCCGGCUGCAUCGCACC | 3851 |
| | TGAGCGCGGCAGCGGCCGTCGGG | 2151 | UGAGCGCGGCAGCGGCCGUC | 3852 |
| | AACGCCGCCTGCCAGCACTACGG | 2152 | AACGCCGCCUGCCAGCACUA | 3853 |
| | GATGAAGGCGGTCCCCACGGTGG | 2153 | GAUGAAGGCGGUCCCCACGG | 3854 |
| | AGGAGCTCGTCGTCTGGCGAGGG | 2154 | AGGAGCUCGUCGUCUGGCGA | 3855 |
| | GCCGATGAAGGCGGTCCCCACGG | 2155 | GCCGAUGAAGGCGGUCCCCA | 3856 |
| | GCGATGCAGCCGGGCGCCGAGGG | 2156 | GCGAUGCAGCCGGGCGCCGA | 3857 |
| | GCTGCTGGACCCGCCGATGAAGG | 2157 | GCUGCUGGACCCGCCGAUGA | 3858 |
| | GCCGATTACAGCTCTCAAACCGG | 2158 | GCCGAUUACAGCUCUCAAAC | 3859 |
| | GGGCACGTGTGCCGTGTGCGGGG | 2159 | GGGCACGUGUGCCGUGUGCG | 3860 |
| | CCACCCGTACGGGCTGCCGCTGG | 2160 | CCACCCGUACGGGCUGCCGC | 3861 |
| | CCCAGCAGGAGCTCGTCGTCTGG | 2161 | CCCAGCAGGAGCUCGUCGUC | 3862 |
| | CAGCAGGCTGGACGCGGTAGGGG | 2162 | CAGCAGGCUGGACGCGGUAG | 3863 |
| | CCCCGCAGGCGGGGGCGTTATGG | 2163 | CCCCGCAGGCGGGGGCGUUA | 3864 |
| | GGCGGCGTTGTCCCCGCACACGG | 2164 | GGCGGCGUUGUCCCCGCACA | 3865 |
| | CACGCCGTAGTGCTGGCAGGCGG | 2165 | CACGCCGUAGUGCUGGCAGG | 3866 |
| | TCCCATAACGCCCCCGCCTGCGG | 2166 | UCCCAUAACGCCCCCGCCUG | 3867 |
| | CCTACCGCGTCCAGCCTGCTGGG | 2167 | CCUACCGCGUCCAGCCUGCU | 3868 |
| | AGGGCACGTGTGCCGTGTGCGGG | 2168 | AGGGCACGUGUGCCGUGUGC | 3869 |
| | TTAGAAGCTCCCTTCAGTGAGGG | 2169 | UUAGAAGCUCCCUUCAGUGA | 3870 |
| | CTCGCCCAGCAGGCTGGACGCGG | 2170 | CUCGCCCAGCAGGCUGGACG | 3871 |
| | GGACTGCTTGAAGTACATGGAGG | 2171 | GGACUGCUUGAAGUACAUGG | 3872 |
| | TGGCCAGCGGCAGCCCGTACGGG | 2172 | UGGCCAGCGGCAGCCCGUAC | 3873 |
| | GGCTGGGACTCTCGCCCAGCAGG | 2173 | GGCUGGGACUCUCGCCCAGC | 3874 |
| | GGCTCTCAAGCGCGGCGGCCTGG | 2174 | GGCUCUCAAGCGCGGCGGCC | 3875 |
| | GGCGGGGGCGTTATGGGACGAGG | 2175 | GGCGGGGGCGUUAUGGGACG | 3876 |
| | TCCTGCTGGGCGGCGACGGCAGG | 2176 | UCCUGCUGGGCGGCGACGGC | 3877 |
| | CTTAGAAGCTCCCTTCAGTGAGG | 2177 | CUUAGAAGCUCCCUUCAGUG | 3878 |
| | CGCACCCAGTAAATGATGCGGGG | 2178 | CGCACCCAGUAAAUGAUGCG | 3879 |
| | CGAGGGGAACTCCTTCGTTGGGG | 2179 | CGAGGGGAACUCCUUCGUUG | 3880 |
| | TCTCCATTCAACGCCGCGCGGGG | 2180 | UCUCCAUUCAACGCCGCGCG | 3881 |
| | AAAAACCTCCGAGGTGCGCGGGG | 2181 | AAAAACCUCCGAGGUGCGCG | 3882 |
| | AGACGTCAATGTGACGCCATGGG | 2182 | AGACGUCAAUGUGACGCCAU | 3883 |
| | GTGATTCAAGCGGACCACATGGG | 2183 | GUGAUUCAAGCGGACCACAU | 3884 |
| | ACGAGCTCCGCCCGAATACGGGG | 2184 | ACGAGCUCCGCCCGAAUACG | 3885 |
| | ATTTCTTTACACGTACGGCGTGG | 2185 | AUUUCUUUACACGUACGGCG | 3886 |
| | GGTCATGCGAGCGCAGCCTGCGG | 2186 | GGUCAUGCGAGCGCAGCCUG | 3887 |
| | CGAGCTCCGCCCGAATACGGGGG | 2187 | CGAGCUCCGCCCGAAUACGG | 3888 |
| | ATCCGGCACTGGACTCGCGATGG | 2188 | AUCCGGCACUGGACUCGCGA | 3889 |
| | TAGGTAACCGGCCGCTTGTGGGG | 2189 | UAGGUAACCGGCCGCUUGUG | 3890 |
| | GCGAACGCTGGGCGCTCGAGGGG | 2190 | GCGAACGCUGGGCGCUCGAG | 3891 |
| | CTGGACTCGCGATGGAATGACGG | 2191 | CUGGACUCGCGAUGGAAUGA | 3892 |
| | AGCTTGCGCTCGATGTAGCGCGG | 2192 | AGCUUGCGCUCGAUGUAGCG | 3893 |
| | GGACGGCGTTAGCGGCTGATGGG | 2193 | GGACGGCGUUAGCGGCUGAU | 3894 |
| | CTATTAGCCGCGAGTTTCGAGGG | 2194 | CUAUUAGCCGCGAGUUUCGA | 3895 |
| | GACGAAGCGGACGGCGTTAGCGG | 2195 | GACGAAGCGGACGGCGUUAG | 3896 |
| | ACCGGCATGTCAGCGACGACAGG | 2196 | ACCGGCAUGUCAGCGACGAC | 3897 |
| | TTTCTTTACACGTACGGCGTGGG | 2197 | UUUCUUUACACGUACGGCGU | 3898 |
| | TTCCATCGCGAGTCCAGTGCCGG | 2198 | UUCCAUCGCGAGUCCAGUGC | 3899 |
| | TGCAGCGGAACCGCTCGCCAGGG | 2199 | UGCAGCGGAACCGCUCGCCA | 3900 |
| | ATGTTACTAAATTCGGCGGTTGG | 2200 | AUGUUACUAAAUUCGGCGGU | 3901 |
| | GCTTTTCGCCTCTTCGAGTGGGG | 2201 | GCUUUUCGCCUCUUCGAGUG | 3902 |
| | ATCAGAACCTACGGGCCGCTGGG | 2202 | AUCAGAACCUACGGGCCGCU | 3903 |
| | CCGACTATATTTGGTTCGGCCGG | 2203 | CCGACUAUAUUUGGUUCGGC | 3904 |
| | TCTATTAGCCGCGAGTTTCGAGG | 2204 | UCUAUUAGCCGCGAGUUUCG | 3905 |
| | GGCCCGGCGGTTCTACCACCCGG | 2205 | GGCCCGGCGGUUCUACCACC | 3906 |
| | AGCTGTCCCGAAATCTGCACTGG | 2206 | AGCUGUCCCGAAAUCUGCAC | 3907 |
| | CTCGAGCGCCCAGCGTTCGCGGG | 2207 | CUCGAGCGCCCAGCGUUCGC | 3908 |
| | GGACTCGGTTCGACCAGGTCTGG | 2208 | GGACUCGGUUCGACCAGGUC | 3909 |
| | CGGACGGCGTTAGCGGCTGATGG | 2209 | CGGACGGCGUUAGCGGCUGA | 3910 |
| | GATTCCGAGCTTACGAAGTCAGG | 2210 | GAUUCCGAGCUUACGAAGUC | 3911 |
| | GCGTTCCTCGGCCAGTCGCACGG | 2211 | GCGUUCCUCGGCCAGUCGCA | 3912 |
| | CGGCCGAACCAAATATAGTCGGG | 2212 | CGGCCGAACCAAAUAUAGUC | 3913 |
| | TTCTCCATTCAACGCCGCGCGGG | 2213 | UUCUCCAUUCAACGCCGCGC | 3914 |
| | TGCGGCCGTGCGACTGGCCGAGG | 2214 | UGCGGCCGUGCGACUGGCCG | 3915 |
| | CTGCCGGGTGGTAGAACCGCCGG | 2215 | CUGCCGGGUGGUAGAACCGC | 3916 |
| | ACTCCGCTTGAAAGGCCCTCAGG | 2216 | ACUCCGCUUGAAAGGCCCUC | 3917 |
| | CGGCTCTCTTCGTCCGGCGCGGG | 2217 | CGGCUCUCUUCGUCCGGCGC | 3918 |
| | AGAGAACGACTCCGCTTGAAAGG | 2218 | AGAGAACGACUCCGCUUGAA | 3919 |
| | TCGTCGCCGGTCACCAGACCTGG | 2219 | UCGUCGCCGGUCACCAGACC | 3920 |
| | AACGAGCTCCGCCCGAATACGGG | 2220 | AACGAGCUCCGCCCGAAUAC | 3921 |
| | TACTAAATTCGGCGGTTGGCCGG | 2221 | UACUAAAUUCGGCGGUUGGC | 3922 |
| | CTAGGTAACCGGCCGCTTGTGGG | 2222 | CUAGGUAACCGGCCGCUUGU | 3923 |
| | GGGCGCTATAGGCCGGAGTTTGG | 2223 | GGGCGCUAUAGGCCGGAGUU | 3924 |
| | TCGAGGGGAACTCCTTCGTTGGG | 2224 | UCGAGGGGAACUCCUUCGUU | 3925 |
| | AGCCGCGAGTTTCGAGGGCCAGG | 2225 | AGCCGCGAGUUUCGAGGGCC | 3926 |
| | CCCTCGAAGACACCGCCCTCTGG | 2226 | CCCUCGAAGACACCGCCCUC | 3927 |
| | GAACGAGCTCCGCCCGAATACGG | 2227 | GAACGAGCUCCGCCCGAAUA | 3928 |
| | ACCCTCGACGACCAGGAAATGGG | 2228 | ACCCUCGACGACCAGGAAAU | 3929 |
| | GGCGCTATAGGCCGGAGTTTGGG | 2229 | GGCGCUAUAGGCCGGAGUUU | 3930 |
| | CCGCACCACCGTGTCTGAATTGG | 2230 | CCGCACCACCGUGUCUGAAU | 3931 |
| | CTCGCACACGCGGAACCGGCTGG | 2231 | CUCGCACACGCGGAACCGGC | 3932 |
| | ATGTGCCCCCGCTAGGCCGCTGG | 2232 | AUGUGCCCCCGCUAGGCCGC | 3933 |
| | TAGCCAGGCCCGACTATATTTGG | 2233 | UAGCCAGGCCCGACUAUAUU | 3934 |
| | ACTGACCCCCCGTATTCGGGCGG | 2234 | ACUGACCCCCCGUAUUCGGG | 3935 |
| | ATAGCGGAGTAGGTTCCCCTCGG | 2235 | AUAGCGGAGUAGGUUCCCCU | 3936 |
| | TGGTCGAACCGAGTCCAAGATGG | 2236 | UGGUCGAACCGAGUCCAAGA | 3937 |
| | GATACCCTTCCCGGACGTCACGG | 2237 | GAUACCCUUCCCGGACGUCA | 3938 |
| | GAGCTCCGCCCGAATACGGGGGG | 2238 | GAGCUCCGCCCGAAUACGGG | 3939 |
| | CGTTGCAAAGTGAGCCCGGGAGG | 2239 | CGUUGCAAAGUGAGCCCGGG | 3940 |
| | ACAAGCCCAGCGGCTCCCGGAGG | 2240 | ACAAGCCCAGCGGCUCCCGG | 3941 |
| | GCGGTTTGTCTAGTCTCCCTCGG | 2241 | GCGGUUUGUCUAGUCUCCCU | 3942 |
| | TCCCGCACACTGACACGTGTGGG | 2242 | UCCCGCACACUGACACGUGU | 3943 |
| | CTCGGCTGGGTCAACTTTCGGGG | 2243 | CUCGGCUGGGUCAACUUUCG | 3944 |
| | TGCACCCGTGATGCAAGTGCAGG | 2244 | UGCACCCGUGAUGCAAGUGC | 3945 |
| | CGGCACGTCATTTATGCCACAGG | 2245 | CGGCACGUCAUUUAUGCCAC | 3946 |
| | TTTTCGCCTCTTCGAGTGGGGGG | 2246 | UUUUCGCCUCUUCGAGUGGG | 3947 |
| | ATCTTGGACTCGGTTCGACCAGG | 2247 | AUCUUGGACUCGGUUCGACC | 3948 |
| | GAGACGTCAATGTGACGCCATGG | 2248 | GAGACGUCAAUGUGACGCCA | 3949 |
| | GACCGGGATTTGTGCTATAGCGG | 2249 | GACCGGGAUUUGUGCUAUAG | 3950 |
| | TGCCGGGTGGTAGAACCGCCGGG | 2250 | UGCCGGGUGGUAGAACCGCC | 3951 |
| | CGATGTAGCGCGGGTAGAAGCGG | 2251 | CGAUGUAGCGCGGGUAGAAG | 3952 |
| | GAACCGCCGGGCCTTCCGCAGGG | 2252 | GAACCGCCGGGCCUUCCGCA | 3953 |
| | CGCGCCGCAGATAGCGGAGTAGG | 2253 | CGCGCCGCAGAUAGCGGAGU | 3954 |
| | AGCCGGTTCCGCGTGTGCGAGGG | 2254 | AGCCGGUUCCGCGUGUGCGA | 3955 |
| | GAACCAAATATAGTCGGGCCTGG | 2255 | GAACCAAAUAUAGUCGGGCC | 3956 |
| | CTTTCTCGTGGGCAGACGAAAGG | 2256 | CUUUCUCGUGGGCAGACGAA | 3957 |
| | GGAACTCCTTCGTTGGGGAGAGG | 2257 | GGAACUCCUUCGUUGGGGAG | 3958 |
| | CGTAAGCTCGGAATCAATTGTGG | 2258 | CGUAAGCUCGGAAUCAAUUG | 3959 |
| | GCAAGAGGGGTGTGAGCGCGCGG | 2259 | GCAAGAGGGGUGUGAGCGCG | 3960 |
| | TATTTCCCGGTCGTGGGAAAAGG | 2260 | UAUUUCCCGGUCGUGGGAAA | 3961 |
| | AGTGCCGGGGATACCCTTCCCGG | 2261 | AGUGCCGGGGAUACCCUUCC | 3962 |
| | TACACCCGATTTACCTCCTAAGG | 2262 | UACACCCGAUUUACCUCCUA | 3963 |
| | CCGCGCACCTCGGAGGTTTTTGG | 2263 | CCGCGCACCUCGGAGGUUUU | 3964 |
| | TCTCGGCTGGGTCAACTTTCGGG | 2264 | UCUCGGCUGGGUCAACUUUC | 3965 |
| | GGTTCCGCGTGTGCGAGGGAGGG | 2265 | GGUUCCGCGUGUGCGAGGGA | 3966 |
| | CGCGCGGCGTTGAATGGAGAAGG | 2266 | CGCGCGGCGUUGAAUGGAGA | 3967 |
| | CCTAGGTAACCGGCCGCTTGTGG | 2267 | CCUAGGUAACCGGCCGCUUG | 3968 |
| | TGCAGATGGTTCCGGGGATAAGG | 2268 | UGCAGAUGGUUCCGGGGAUA | 3969 |
| | GCAACCGAGTCTCTGCACTGCGG | 2269 | GCAACCGAGUCUCUGCACUG | 3970 |
| | CGACAGGCAGACGTGCCTAGTGG | 2270 | CGACAGGCAGACGUGCCUAG | 3971 |
| | GAGTTTGCAGACGCACTCGGAGG | 2271 | GAGUUUGCAGACGCACUCGG | 3972 |
| | TTAGCGGCTGATGGGACGAGCGG | 2272 | UUAGCGGCUGAUGGGACGAG | 3973 |
| | GAAACGCACCATTGTGACCGGGG | 2273 | GAAACGCACCAUUGUGACCG | 3974 |
| | TACGTACTTGAGTGCTGTGGCGG | 2274 | UACGUACUUGAGUGCUGUGG | 3975 |
| | GAAGAATTTCTGTGAGCGCACGG | 2275 | GAAGAAUUUCUGUGAGCGCA | 3976 |
| | GGCGATGTTACTAAATTCGGCGG | 2276 | GGCGAUGUUACUAAAUUCGG | 3977 |
| | ACCCTTCCCGGACGTCACGGAGG | 2277 | ACCCUUCCCGGACGUCACGG | 3978 |
| JUNB | GGGTAAAAGTACTGTCCCGGGGG | 2278 | GGGUAAAAGUACUGUCCCGG | 3979 |
| | TCTGCCCAGTGACGCGACCGCGG | 2279 | UCUGCCCAGUGACGCGACCG | 3980 |
| | GGACAGTACTTTTACCCCCGCGG | 2280 | GGACAGUACUUUUACCCCCG | 3981 |
| | ACTTCCGTGGCTGACTAGCGCGG | 2281 | ACUUCCGUGGCUGACUAGCG | 3982 |
| | GTCCCGTAGGATCCGAGTGACGG | 2282 | GUCCCGUAGGAUCCGAGUGA | 3983 |
| | TAGCGCGGTATAAAGGCGTGTGG | 2283 | UAGCGCGGUAUAAAGGCGUG | 3984 |
| | CTGACAGCCGTTGCTGACGTGGG | 2284 | CUGACAGCCGUUGCUGACGU | 3985 |
| | GACTAAGAGGTTACCATCGAGGG | 2285 | GACUAAGAGGUUACCAUCGA | 3986 |
| | GTACGAGCTCCCGGTCCCGACGG | 2286 | GUACGAGCUCCCGGUCCCGA | 3987 |
| | GCGCTTTGAGACTCCGGTAGGGG | 2287 | GCGCUUUGAGACUCCGGUAG | 3988 |
| | TCGCGCCAGAGAGGGCGACGGGG | 2288 | UCGCGCCAGAGAGGGCGACG | 3989 |
| | ATGCCTGCGCCGAACCGACGAGG | 2289 | AUGCCUGCGCCGAACCGACG | 3990 |
| | TGCGCACTCCAAGTCTCGGCCGG | 2290 | UGCGCACUCCAAGUCUCGGC | 3991 |
| | ATGTGTCCCCCTCGTCGGTTCGG | 2291 | AUGUGUCCCCCUCGUCGGUU | 3992 |
| | CGCCGCCCATATTAGGGCACAGG | 2292 | CGCCGCCCAUAUUAGGGCAC | 3993 |
| | ACTCAAGCCCGCGGGGACATTGG | 2293 | ACUCAAGCCCGCGGGGACAU | 3994 |
| | GTCGCGTCACTGGGCAGAATCGG | 2294 | GUCGCGUCACUGGGCAGAAU | 3995 |
| | TCCCCGCGGGCTTGAGTACCAGG | 2295 | UCCCCGCGGGCUUGAGUACC | 3996 |
| | TGTTCCATTGGCCCGACGGCGGG | 2296 | UGUUCCAUUGGCCCGACGGC | 3997 |
| | ATAGTCGGGTTCCCCGCTTCTGG | 2297 | AUAGUCGGGUUCCCCGCUUC | 3998 |
| | GGTCGCGCGTTCTCGGGGGCTGG | 2298 | GGUCGCGCGUUCUCGGGGGC | 3999 |
| | AACGTGTCCCTGGGCGCTACCGG | 2299 | AACGUGUCCCUGGGCGCUAC | 4000 |
| | CTCCGCTGCGGTGACCGGACTGG | 2300 | CUCCGCUGCGGUGACCGGAC | 4001 |
| | GGCTGACTAGCGCGGTATAAAGG | 2301 | GGCUGACUAGCGCGGUAUAA | 4002 |
| | AGTGACGCGACCGCGGTCTCTGG | 2302 | AGUGACGCGACCGCGGUCUC | 4003 |
| | CGCCGGGTGGCCACCGGCGAAGG | 2303 | CGCCGGGUGGCCACCGGCGA | 4004 |
| | TGACTAAGAGGTTACCATCGAGG | 2304 | UGACUAAGAGGUUACCAUCG | 4005 |
| | ACAGTACTTTTACCCCCGCGGGG | 2305 | ACAGUACUUUUACCCCCGCG | 4006 |
| | GTGCCCTAATATGGGCGGCGGGG | 2306 | GUGCCCUAAUAUGGGCGGCG | 4007 |
| | CCAATCGGAGCGCACTTCCGTGG | 2307 | CCAAUCGGAGCGCACUUCCG | 4008 |
| | GGGGCTTGTAAACGTCGAGGTGG | 2308 | GGGGCUUGUAAACGUCGAGG | 4009 |
| | TCAAGCAATGGTTCCGCCCGCGG | 2309 | UCAAGCAAUGGUUCCGCCCG | 4010 |
| | GTGTTCCATTGGCCCGACGGCGG | 2310 | GUGUUCCAUUGGCCCGACGG | 4011 |
| | TATCGCGCCAGAGAGGGCGACGG | 2311 | UAUCGCGCCAGAGAGGGCGA | 4012 |
| | TGCCTGCGCCGAACCGACGAGGG | 2312 | UGCCUGCGCCGAACCGACGA | 4013 |
| | ACACAGCTACGGGATACGGCCGG | 2313 | ACACAGCUACGGGAUACGGC | 4014 |
| | TATGAGTCGTCGTGGTAGAAGGG | 2314 | UAUGAGUCGUCGUGGUAGAA | 4015 |
| | CGACGACTCATACACAGCTACGG | 2315 | CGACGACUCAUACACAGCUA | 4016 |
| | AAAGGACCTCGGGGTACGCATGG | 2316 | AAAGGACCUCGGGGUACGCA | 4017 |
| | ACGCTCAAGGCCGAGAACGCGGG | 2317 | ACGCUCAAGGCCGAGAACGC | 4018 |
| | GCGGGGCTGTCGAGTACCGCCGG | 2318 | GCGGGGCUGUCGAGUACCGC | 4019 |
| | GACGCTCAAGGCCGAGAACGCGG | 2319 | GACGCUCAAGGCCGAGAACG | 4020 |
| | GAGACCGCGGTCGCGTCACTGGG | 2320 | GAGACCGCGGUCGCGUCACU | 4021 |
| | GGCGCTTTGAGACTCCGGTAGGG | 2321 | GGCGCUUUGAGACUCCGGUA | 4022 |
| | CCACACGCGCCGCCCTTCGCCGG | 2322 | CCACACGCGCCGCCCUUCGC | 4023 |
| | GTGCCCAGCCGTCCAAGCGAGGG | 2323 | GUGCCCAGCCGUCCAAGCGA | 4024 |
| | GTTCCATTGGCCCGACGGCGGGG | 2324 | GUUCCAUUGGCCCGACGGCG | 4025 |
| | GGGGTTTCTTCGCACATACTGGG | 2325 | GGGGUUUCUUCGCACAUACU | 4026 |
| | TGCCTGGTCGCGCGTTCTCGGGG | 2326 | UGCCUGGUCGCGCGUUCUCG | 4027 |
| | CACGACGACGCCTACACCCCCGG | 2327 | CACGACGACGCCUACACCCC | 4028 |
| | CGTTGCTGTTGGGGACAATCAGG | 2328 | CGUUGCUGUUGGGGACAAUC | 4029 |
| | CGTGTCCCTGGGCGCTACCGGGG | 2329 | CGUGUCCCUGGGCGCUACCG | 4030 |
| | TTCCATTGGCCCGACGGCGGGGG | 2330 | UUCCAUUGGCCCGACGGCGG | 4031 |
| | TACTTTTACCCCCGCGGGGGTGG | 2331 | UACUUUUACCCCCGCGGGGG | 4032 |
| | ACTCGACAGCCCCGCGTTCTCGG | 2332 | ACUCGACAGCCCCGCGUUCU | 4033 |
| | TGGCCCGCCTAGAGGGAGTCTGG | 2333 | UGGCCCGCCUAGAGGGAGUC | 4034 |
| | GTCCCCCGCCGTCGGGCCAATGG | 2334 | GUCCCCCGCCGUCGGGCCAA | 4035 |
| | TGTGCCCTAATATGGGCGGCGGG | 2335 | UGUGCCCUAAUAUGGGCGGC | 4036 |
| | TTTACGGACACCCCCTCGCTTGG | 2336 | UUUACGGACACCCCCUCGCU | 4037 |
| | TCTCGTATTCTGGGTACCTCAGG | 2337 | UCUCGUAUUCUGGGUACCUC | 4038 |
| | TGGTCGGGACTAGCAGTCTGGGG | 2338 | UGGUCGGGACUAGCAGUCUG | 4039 |
| | CGTGATCACGCCGTTGCTGTTGG | 2339 | CGUGAUCACGCCGUUGCUGU | 4040 |
| | TCTCCCCGCCGCCCATATTAGGG | 2340 | UCUCCCCGCCGCCCAUAUUA | 4041 |
| | AGAGACCGCGGTCGCGTCACTGG | 2341 | AGAGACCGCGGUCGCGUCAC | 4042 |
| | ACCCCCTCGCTTGGACGGCTGGG | 2342 | ACCCCCUCGCUUGGACGGCU | 4043 |
| | ACACCGGCGGCGTGGCGTCCCGG | 2343 | ACACCGGCGGCGUGGCGUCC | 4044 |
| | AGCGAGGGGGTGTCCGTAAAGGG | 2344 | AGCGAGGGGGUGUCCGUAAA | 4045 |
| | GGCAGAATCGGTCCTTGTATGGG | 2345 | GGCAGAAUCGGUCCUUGUAU | 4046 |
| | CTAAGAGGTTACCATCGAGGGGG | 2346 | CUAAGAGGUUACCAUCGAGG | 4047 |
| | TATACCGCGCTAGTCAGCCACGG | 2347 | UAUACCGCGCUAGUCAGCCA | 4048 |
| | GAAAGCTAGTAAGCGGCCTGGGG | 2348 | GAAAGCUAGUAAGCGGCCUG | 4049 |
| | TCCGTCTGACCTGACCGGGGCGG | 2349 | UCCGUCUGACCUGACCGGGG | 4050 |
| | ACGCCCAGGTTCCTCTTCCGAGG | 2350 | ACGCCCAGGUUCCUCUUCCG | 4051 |
| | GCCTGGTACTCAAGCCCGCGGGG | 2351 | GCCUGGUACUCAAGCCCGCG | 4052 |
| | GCCTGCGCCGAACCGACGAGGGG | 2352 | GCCUGCGCCGAACCGACGAG | 4053 |
| | CAGTACTTTTACCCCCGCGGGGG | 2353 | CAGUACUUUUACCCCCGCGG | 4054 |
| | GCCCCGGTCAGGTCAGACGGAGG | 2354 | GCCCCGGUCAGGUCAGACGG | 4055 |
| | ACTAAGAGGTTACCATCGAGGGG | 2355 | ACUAAGAGGUUACCAUCGAG | 4056 |
| | ACCCAGTCCGGTCACCGCAGCGG | 2356 | ACCCAGUCCGGUCACCGCAG | 4057 |
| | GACGACTCATACACAGCTACGGG | 2357 | GACGACUCAUACACAGCUAC | 4058 |
| | AGGCTCTCCCGTAAGCGGGAAGG | 2358 | AGGCUCUCCCGUAAGCGGGA | 4059 |
| | TCCTTGTAAACAGCGGCCACGGG | 2359 | UCCUUGUAAACAGCGGCCAC | 4060 |
| | GGTTCGGCGCAGGCATCTTGTGG | 2360 | GGUUCGGCGCAGGCAUCUUG | 4061 |
| | GGAAAGCTATCGCGCCAGAGAGG | 2361 | GGAAAGCUAUCGCGCCAGAG | 4062 |
| | CGCTCAAGGCCGAGAACGCGGGG | 2362 | CGCUCAAGGCCGAGAACGCG | 4063 |
| | CACAGCTACGGGATACGGCCGGG | 2363 | CACAGCUACGGGAUACGGCC | 4064 |
| | TGCCCAGCCGTCCAAGCGAGGGG | 2364 | UGCCCAGCCGUCCAAGCGAG | 4065 |
| | TGTGCCCAGCCGTCCAAGCGAGG | 2365 | UGUGCCCAGCCGUCCAAGCG | 4066 |
| | CGGCCAGACTCCCTCTAGGCGGG | 2366 | CGGCCAGACUCCCUCUAGGC | 4067 |
| | CGCCTGGTACTCAAGCCCGCGGG | 2367 | CGCCUGGUACUCAAGCCCGC | 4068 |
| | GGGGAACCCGACTATCTGCCAGG | 2368 | GGGGAACCCGACUAUCUGCC | 4069 |
| | CTCGTATTCTGGGTACCTCAGGG | 2369 | CUCGUAUUCUGGGUACCUCA | 4070 |
| | GACAGTACTTTTACCCCCGCGGG | 2370 | GACAGUACUUUUACCCCCGC | 4071 |
| | CCGGGGGCGAAGTCCGACCCAGG | 2371 | CCGGGGGCGAAGUCCGACCC | 4072 |
| | GGACTTCGCCCCCGGCCCGACGG | 2372 | GGACUUCGCCCCCGGCCCGA | 4073 |
| | ACTGTAAATCGGGAGGGTTAAGG | 2373 | ACUGUAAAUCGGGAGGGUUA | 4074 |
| | TAGCGCCCAGGGACACGTTGGGG | 2374 | UAGCGCCCAGGGACACGUUG | 4075 |
| | AGGGTGCTCCGGCCGAGACTTGG | 2375 | AGGGUGCUCCGGCCGAGACU | 4076 |
| | TGATCACGCCGTTGCTGTTGGGG | 2376 | UGAUCACGCCGUUGCUGUUG | 4077 |
| | GCCCGTGGCCGCTGTTTACAAGG | 2377 | GCCCGUGGCCGCUGUUUACA | 4078 |
| REL | GTGAGCCGCAAACCCAGCGGAGG | 2378 | GUGAGCCGCAAACCCAGCGG | 4079 |
| | CGACGGCCGGGGTTTTCGAGAGG | 2379 | CGACGGCCGGGGUUUUCGAG | 4080 |
| | CGTCGGGCCTACGTCAGCCGCGG | 2380 | CGUCGGGCCUACGUCAGCCG | 4081 |
| | CGGCCGGGGTTTTCGAGAGGTGG | 2381 | CGGCCGGGGUUUUCGAGAGG | 4082 |
| | CAGCGTCGCCGTCCACCGTACGG | 2382 | CAGCGUCGCCGUCCACCGUA | 4083 |
| | GGCGGGACGTTGCGCCCTGTAGG | 2383 | GGCGGGACGUUGCGCCCUGU | 4084 |
| | CGGGACGTTGCGCCCTGTAGGGG | 2384 | CGGGACGUUGCGCCCUGUAG | 4085 |
| | GCGGGACGTTGCGCCCTGTAGGG | 2385 | GCGGGACGUUGCGCCCUGUA | 4086 |
| | CGTACGGTGGACGGCGACGCTGG | 2386 | CGUACGGUGGACGGCGACGC | 4087 |
| | CGTCCACCGTACGGGAGCCAGGG | 2387 | CGUCCACCGUACGGGAGCCA | 4088 |
| | AGGGCGCAACGTCCCGCCGCTGG | 2388 | AGGGCGCAACGUCCCGCCGC | 4089 |
| | AGCGTCGCCGTCCACCGTACGGG | 2389 | AGCGUCGCCGUCCACCGUAC | 4090 |
| | CGCCGGGGGCGTATGCGTGGGGG | 2390 | CGCCGGGGGCGUAUGCGUGG | 4091 |
| | GGGTCCCCGTATGCAAATACAGG | 2391 | GGGUCCCCGUAUGCAAAUAC | 4092 |
| | GCGGCCGCAGTCAGTCAGTCAGG | 2392 | GCGGCCGCAGUCAGUCAGUC | 4093 |
| | GCAACGTCCCGCCGCTGGCGCGG | 2393 | GCAACGUCCCGCCGCUGGCG | 4094 |
| | ACGCAGCAACCCTCACCCGGAGG | 2394 | ACGCAGCAACCCUCACCCGG | 4095 |
| | CGCGCCCCATGAACACTCACCGG | 2395 | CGCGCCCCAUGAACACUCAC | 4096 |
| | GGGACGTTGCGCCCTGTAGGGGG | 2396 | GGGACGUUGCGCCCUGUAGG | 4097 |
| | GAATTTCCCGCGGCTGACGTAGG | 2397 | GAAUUUCCCGCGGCUGACGU | 4098 |
| | GTACGGTGGACGGCGACGCTGGG | 2398 | GUACGGUGGACGGCGACGCU | 4099 |
| | GCCGCAAACCCAGCGGAGGGCGG | 2399 | GCCGCAAACCCAGCGGAGGG | 4100 |
| | TGACTGACTGCGGCCGCCTCCGG | 2400 | UGACUGACUGCGGCCGCCUC | 4101 |
| | CAGTACCCTCGCAATTTAGATGG | 2401 | CAGUACCCUCGCAAUUUAGA | 4102 |
| | CGTATGCGTGGGGGCCGGCGGGG | 2402 | CGUAUGCGUGGGGGCCGGCG | 4103 |
| | AACGTCCCGCCGCTGGCGCGGGG | 2403 | AACGUCCCGCCGCUGGCGCG | 4104 |
| | AGCGCCGGGGGCGTATGCGTGGG | 2404 | AGCGCCGGGGGCGUAUGCGU | 4105 |
| | GCGTATGCGTGGGGGCCGGCGGG | 2405 | GCGUAUGCGUGGGGGCCGGC | 4106 |
| | CGGCGACGCTGGGTGACCCGGGG | 2406 | CGGCGACGCUGGGUGACCCG | 4107 |
| | TGACGGCTAGCAGCGTGAGAAGG | 2407 | UGACGGCUAGCAGCGUGAGA | 4108 |
| | GTCGGGCCTACGTCAGCCGCGGG | 2408 | GUCGGGCCUACGUCAGCCGC | 4109 |
| | GGCCCCCACGCATACGCCCCCGG | 2409 | GGCCCCCACGCAUACGCCCC | 4110 |
| | CCCCGCCGGCAGAGGTCCCTCGG | 2410 | CCCCGCCGGCAGAGGUCCCU | 4111 |
| | GAACCACCTCTCGAAAACCCCGG | 2411 | GAACCACCUCUCGAAAACCC | 4112 |
| | CCACACTCGGAAGAACAACCTGG | 2412 | CCACACUCGGAAGAACAACC | 4113 |
| | TTGCGCCCTGTAGGGGGAAGTGG | 2413 | UUGCGCCCUGUAGGGGGAAG | 4114 |
| | TACCCTCGCAATTTAGATGGAGG | 2414 | UACCCUCGCAAUUUAGAUGG | 4115 |
| | GTCCACCGTACGGGAGCCAGGGG | 2415 | GUCCACCGUACGGGAGCCAG | 4116 |
| | CCTGGCTCCCGTACGGTGGACGG | 2416 | CCUGGCUCCCGUACGGUGGA | 4117 |
| | CAACCCTCACCCGGAGGCGTGGG | 2417 | CAACCCUCACCCGGAGGCGU | 4118 |
| | CGCTGCTAGCCGTCACCTCCCGG | 2418 | CGCUGCUAGCCGUCACCUCC | 4119 |
| | GCGCCGGGGGCGTATGCGTGGGG | 2419 | GCGCCGGGGGCGUAUGCGUG | 4120 |
| | CAACGTCCCGCCGCTGGCGCGGG | 2420 | CAACGUCCCGCCGCUGGCGC | 4121 |
| | CGTAGAGAGGGCCGGCCGCTGGG | 2421 | CGUAGAGAGGGCCGGCCGCU | 4122 |
| | CCCGGGGTGCAAGAATTCAGGGG | 2422 | CCCGGGGUGCAAGAAUUCAG | 4123 |
| | TGAGCCGCAAACCCAGCGGAGGG | 2423 | UGAGCCGCAAACCCAGCGGA | 4124 |
| | GTTTAAAGTTCAGGAGCGGGCGG | 2424 | GUUUAAAGUUCAGGAGCGGG | 4125 |
| | GGCGGGAGGGGAATTTCCCGCGG | 2425 | GGCGGGAGGGGAAUUUCCCG | 4126 |
| | AGGGTGCGGATGACGTAGAGAGG | 2426 | AGGGUGCGGAUGACGUAGAG | 4127 |
| | CCGCCCACGCCTCCGGGTGAGGG | 2427 | CCGCCCACGCCUCCGGGUGA | 4128 |
| TOX | TTTCCCGTGGAATGCACCGAGGG | 2428 | UUUCCCGUGGAAUGCACCGA | 4129 |
| | GGTGGCGAGTCATCACCAAACGG | 2429 | GGUGGCGAGUCAUCACCAAA | 4130 |
| | AGGGACTCGAGCCGATCGAAGGG | 2430 | AGGGACUCGAGCCGAUCGAA | 4131 |
| | ACGTGTCTAAGCAGTCCCGTTGG | 2431 | ACGUGUCUAAGCAGUCCCGU | 4132 |
| | AAACGCCCCCCGGCAAACCTAGG | 2432 | AAACGCCCCCCGGCAAACCU | 4133 |
| | TTGAACGCGACGTGCTCGCCCGG | 2433 | UUGAACGCGACGUGCUCGCC | 4134 |
| | GTCGCGTGGTGCGGAGTCCAGGG | 2434 | GUCGCGUGGUGCGGAGUCCA | 4135 |
| | CGCGGTGTTTGGCAAGCCCCCGG | 2435 | CGCGGUGUUUGGCAAGCCCC | 4136 |
| | GATCCTTAGCCGCGAACAGCAGG | 2436 | GAUCCUUAGCCGCGAACAGC | 4137 |
| | TTCCGCACAATCGCGGTGTTTGG | 2437 | UUCCGCACAAUCGCGGUGUU | 4138 |
| | TCCGCGCGCACCCCTTAAACAGG | 2438 | UCCGCGCGCACCCCUUAAAC | 4139 |
| | GCGAGAGTTGGGCGTCTAAAAGG | 2439 | GCGAGAGUUGGGCGUCUAAA | 4140 |
| | AAGCCGCGGCGCGCACCCGTCGG | 2440 | AAGCCGCGGCGCGCACCCGU | 4141 |
| | ATATTGTGGAGTAGCTCCGGGGG | 2441 | AUAUUGUGGAGUAGCUCCGG | 4142 |
| | TACACTTCGAATCACCCCTGTGG | 2442 | UACACUUCGAAUCACCCCUG | 4143 |
| | AGTCCCAACGATTTTTCCCGTGG | 2443 | AGUCCCAACGAUUUUUCCCG | 4144 |
| | TTTACTACCCAAGCGCACGCAGG | 2444 | UUUACUACCCAAGCGCACGC | 4145 |
| | CGTCCAACTAGCCCTAGGCGTGG | 2445 | CGUCCAACUAGCCCUAGGCG | 4146 |
| | GTCCAACTAGCCCTAGGCGTGGG | 2446 | GUCCAACUAGCCCUAGGCGU | 4147 |
| | AGTGGGGCACGAATCTCGGAGGG | 2447 | AGUGGGGCACGAAUCUCGGA | 4148 |
| | GCCTTCGCAAACCGTCCAGTGGG | 2448 | GCCUUCGCAAACCGUCCAGU | 4149 |
| | TCAGCACACAATCCGGCTAAAGG | 2449 | UCAGCACACAAUCCGGCUAA | 4150 |
| | TGAAGTTACCTGCCCGGCGGCGG | 2450 | UGAAGUUACCUGCCCGGCGG | 4151 |
| | TGCGACTCGGTCGCGTGGTGCGG | 2451 | UGCGACUCGGUCGCGUGGUG | 4152 |
| | TTAGACGCCCAACTCTCGCTTGG | 2452 | UUAGACGCCCAACUCUCGCU | 4153 |
| | TAGAGCCCGAGCGCGTGTGCCGG | 2453 | UAGAGCCCGAGCGCGUGUGC | 4154 |
| | TTCGATCGGCTCGAGTCCCTCGG | 2454 | UUCGAUCGGCUCGAGUCCCU | 4155 |
| | GCGCTCGGGCTCTAGGTACTGGG | 2455 | GCGCUCGGGCUCUAGGUACU | 4156 |
| | TCCACTACGGGCCGGGAGTAGGG | 2456 | UCCACUACGGGCCGGGAGUA | 4157 |
| | CGAGTCGCAGCTCCGAGTCTTGG | 2457 | CGAGUCGCAGCUCCGAGUCU | 4158 |
| | GGCCGGACGCGGGCTCGTCAAGG | 2458 | GGCCGGACGCGGGCUCGUCA | 4159 |
| | CGCGGAAATTGCAAGTTTGTTGG | 2459 | CGCGGAAAUUGCAAGUUUGU | 4160 |
| | GCATGAAGTTACCTGCCCGGCGG | 2460 | GCAUGAAGUUACCUGCCCGG | 4161 |
| | TGTCACTTTCCGCACAATCGCGG | 2461 | UGUCACUUUCCGCACAAUCG | 4162 |
| | CAGTCCCGTTGGATGAACGTTGG | 2462 | CAGUCCCGUUGGAUGAACGU | 4163 |
| | CCAGCGCGTCGCACACAAAGGGG | 2463 | CCAGCGCGUCGCACACAAAG | 4164 |
| | GGAGCTGCGACTCGGTCGCGTGG | 2464 | GGAGCUGCGACUCGGUCGCG | 4165 |
| | GACCAGCGCGTCGCACACAAAGG | 2465 | GACCAGCGCGUCGCACACAA | 4166 |
| | CAAGACTCGGAGCTGCGACTCGG | 2466 | CAAGACUCGGAGCUGCGACU | 4167 |
| | CTAACTTGCCTAAACACCATCGG | 2467 | CUAACUUGCCUAAACACCAU | 4168 |
| | CCTTTGAGTGGGTCTCACACTGG | 2468 | CCUUUGAGUGGGUCUCACAC | 4169 |
| | ATTCCACGGGAAAAATCGTTGGG | 2469 | AUUCCACGGGAAAAAUCGUU | 4170 |
| | GAATGCACCGAGGGTCGCCATGG | 2470 | GAAUGCACCGAGGGUCGCCA | 4171 |
| | GGGTTCGGACACAGGTCCGCGGG | 2471 | GGGUUCGGACACAGGUCCGC | 4172 |
| | GAGGGACTCGAGCCGATCGAAGG | 2472 | GAGGGACUCGAGCCGAUCGA | 4173 |
| | TGGACGGTTTGCGAAGGCTGAGG | 2473 | UGGACGGUUUGCGAAGGCUG | 4174 |
| | CTCGAGCCGATCGAAGGGTGAGG | 2474 | CUCGAGCCGAUCGAAGGGUG | 4175 |
| | ATTTATCACCAAGCGAGAGTTGG | 2475 | AUUUAUCACCAAGCGAGAGU | 4176 |
| | GTTCGGCTGGGTCCACCTATAGG | 2476 | GUUCGGCUGGGUCCACCUAU | 4177 |
| | CAGCACACAATCCGGCTAAAGGG | 2477 | CAGCACACAAUCCGGCUAAA | 4178 |
| | GGCACGACTGCCCACGCCTAGGG | 2478 | GGCACGACUGCCCACGCCUA | 4179 |
| | AGAGCCCGAGCGCGTGTGCCGGG | 2479 | AGAGCCCGAGCGCGUGUGCC | 4180 |
| | CACGAATCTCGGAGGGGTGCGGG | 2480 | CACGAAUCUCGGAGGGGUGC | 4181 |
| | GCCCCCCGGCAAACCTAGGCAGG | 2481 | GCCCCCCGGCAAACCUAGGC | 4182 |
| | GAGAAAACCGTGGAATACTTTGG | 2482 | GAGAAAACCGUGGAAUACUU | 4183 |
| | CTCCGCGAGTGCGGGAGCTTTGG | 2483 | CUCCGCGAGUGCGGGAGCUU | 4184 |
| | CGCGGACCTGTGTCCGAACCCGG | 2484 | CGCGGACCUGUGUCCGAACC | 4185 |
| | AGCTCCCGCACTCGCGGAGCAGG | 2485 | AGCUCCCGCACUCGCGGAGC | 4186 |
| | CGCGCTCGGGCTCTAGGTACTGG | 2486 | CGCGCUCGGGCUCUAGGUAC | 4187 |
| | ACGGGTGCGCGCCGCGGCTTGGG | 2487 | ACGGGUGCGCGCCGCGGCUU | 4188 |
| | AATATTGTGGAGTAGCTCCGGGG | 2488 | AAUAUUGUGGAGUAGCUCCG | 4189 |
| | TTCTCCGGATTAGTTGCCAGGGG | 2489 | UUCUCCGGAUUAGUUGCCAG | 4190 |
| | AGACACGTCCAACTAGCCCTAGG | 2490 | AGACACGUCCAACUAGCCCU | 4191 |
| | TCCCTGCCTAGGTTTGCCGGGGG | 2491 | UCCCUGCCUAGGUUUGCCGG | 4192 |
| | TGTGCGACGCGCTGGTCCCGAGG | 2492 | UGUGCGACGCGCUGGUCCCG | 4193 |
| | AAAGGGGTCGCACTCCCTGTGGG | 2493 | AAAGGGGUCGCACUCCCUGU | 4194 |
| | GCGACCCTCGGTGCATTCCACGG | 2494 | GCGACCCUCGGUGCAUUCCA | 4195 |
| | CGCTTGGGTAGTAAATATTGTGG | 2495 | CGCUUGGGUAGUAAAUAUUG | 4196 |
| | CGGGTTCGGACACAGGTCCGCGG | 2496 | CGGGUUCGGACACAGGUCCG | 4197 |
| | CATTCCACGGGAAAAATCGTTGG | 2497 | CAUUCCACGGGAAAAAUCGU | 4198 |
| | GTCCAAAGCTCCCGCACTCGCGG | 2498 | GUCCAAAGCUCCCGCACUCG | 4199 |
| | AGCGCATGAAGTTACCTGCCCGG | 2499 | AGCGCAUGAAGUUACCUGCC | 4200 |
| | CGCGCTGGTCCCGAGGAGCGCGG | 2500 | CGCGCUGGUCCCGAGGAGCG | 4201 |
| | ACAGTCAGGGGGTACGAGGGAGG | 2501 | ACAGUCAGGGGGUACGAGGG | 4202 |
| | CCCCACTGGACGGTTTGCGAAGG | 2502 | CCCCACUGGACGGUUUGCGA | 4203 |
| | CCTGTGTCCGAACCCGGGCTCGG | 2503 | CCUGUGUCCGAACCCGGGCU | 4204 |
| | GTACCCCCTGACTGTCCTATAGG | 2504 | GUACCCCCUGACUGUCCUAU | 4205 |
| | TCTGCCAACGTTCATCCAACGGG | 2505 | UCUGCCAACGUUCAUCCAAC | 4206 |
| | ACCCGAGGTCAGCGGGCCGTGGG | 2506 | ACCCGAGGUCAGCGGGCCGU | 4207 |
| | ACTACGGGCCGGGAGTAGGGAGG | 2507 | ACUACGGGCCGGGAGUAGGG | 4208 |
| | GTCGCCATGGATGTGCCTGCAGG | 2508 | GUCGCCAUGGAUGUGCCUGC | 4209 |
| | GGCCTTGACGAGCCCGCGTCCGG | 2509 | GGCCUUGACGAGCCCGCGUC | 4210 |
| | TGCCCACGCCTAGGGCTAGTTGG | 2510 | UGCCCACGCCUAGGGCUAGU | 4211 |
| | ACCAGCGCGTCGCACACAAAGGG | 2511 | ACCAGCGCGUCGCACACAAA | 4212 |
| | TGGTCCCGAGGAGCGCGGCACGG | 2512 | UGGUCCCGAGGAGCGCGGCA | 4213 |
| | TGTGCCCGTGCCGCGCTCCTCGG | 2513 | UGUGCCCGUGCCGCGCUCCU | 4214 |
| | CCGGGCTCGGCTGCCGGAACCGG | 2514 | CCGGGCUCGGCUGCCGGAAC | 4215 |
| | CGCAGCCCGGCACACGCGCTCGG | 2515 | CGCAGCCCGGCACACGCGCU | 4216 |
| | TTTTCCCGTGGAATGCACCGAGG | 2516 | UUUUCCCGUGGAAUGCACCG | 4217 |
| | CGACCCTCGGTGCATTCCACGGG | 2517 | CGACCCUCGGUGCAUUCCAC | 4218 |
| | CTCCACTACGGGCCGGGAGTAGG | 2518 | CUCCACUACGGGCCGGGAGU | 4219 |
| | GGTGATTCGAAGTGTAAATAGGG | 2519 | GGUGAUUCGAAGUGUAAAUA | 4220 |
| | TGCGCCCGACGCTCCCTGTCTGG | 2520 | UGCGCCCGACGCUCCCUGUC | 4221 |
| | CTTAGCCGCGAACAGCAGGAAGG | 2521 | CUUAGCCGCGAACAGCAGGA | 4222 |
| | TCCTGTTTAAGGGGTGCGCGCGG | 2522 | UCCUGUUUAAGGGGUGCGCG | 4223 |
| | CCCCCCGGCAAACCTAGGCAGGG | 2523 | CCCCCCGGCAAACCUAGGCA | 4224 |
| | TAGGACAGTCAGGGGGTACGAGG | 2524 | UAGGACAGUCAGGGGGUACG | 4225 |
| | AAGTCACCTCCACTACGGGCCGG | 2525 | AAGUCACCUCCACUACGGGC | 4226 |
| | CCCCCTGACTGTCCTATAGGTGG | 2526 | CCCCCUGACUGUCCUAUAGG | 4227 |
| | GCGGGCTCGTCAAGGCCCAATGG | 2527 | GCGGGCUCGUCAAGGCCCAA | 4228 |
| TOX2 | CCACCCGTGCGACGACACAGTGG | 2528 | CCACCCGUGCGACGACACAG | 4229 |
| | TCATCCACACTCGCGCGTCGAGG | 2529 | UCAUCCACACUCGCGCGUCG | 4230 |
| | GTGACTCGTCTGTGGCGGTGAGG | 2530 | GUGACUCGUCUGUGGCGGUG | 4231 |
| | CGCAGCCTACTCGGAATCCGAGG | 2531 | CGCAGCCUACUCGGAAUCCG | 4232 |
| | TAAACCTCGACGCGCGAGTGTGG | 2532 | UAAACCUCGACGCGCGAGUG | 4233 |
| | AAGCGCGGGTTTTCGTCACTCGG | 2533 | AAGCGCGGGUUUUCGUCACU | 4234 |
| | CTGTCCGCGCGTCCGCCAGTCGG | 2534 | CUGUCCGCGCGUCCGCCAGU | 4235 |
| | GGTCTCGCGAAGAGTGGCGGTGG | 2535 | GGUCUCGCGAAGAGUGGCGG | 4236 |
| | TGTGAGCCGCCCGTGCCCGTCGG | 2536 | UGUGAGCCGCCCGUGCCCGU | 4237 |
| | TGCCGCCGTGGTAATAGTCCAGG | 2537 | UGCCGCCGUGGUAAUAGUCC | 4238 |
| | AGGGGACGCGGACTGCTTAGAGG | 2538 | AGGGGACGCGGACUGCUUAG | 4239 |
| | TACAGGCGGACGTCCATGGCGGG | 2539 | UACAGGCGGACGUCCAUGGC | 4240 |
| | TCGTCGCACGGGTGGCTGTCGGG | 2540 | UCGUCGCACGGGUGGCUGUC | 4241 |
| | TCGCAGTCCCGCTCGCACACTGG | 2541 | UCGCAGUCCCGCUCGCACAC | 4242 |
| | TAAGCAGTCCGCGTCCCCTTCGG | 2542 | UAAGCAGUCCGCGUCCCCUU | 4243 |
| | GGCCGGAACAATAGCGCGCGCGG | 2543 | GGCCGGAACAAUAGCGCGCG | 4244 |
| | GCCGGAACAATAGCGCGCGCGGG | 2544 | GCCGGAACAAUAGCGCGCGC | 4245 |
| | CTGTCAGGGGGACGCGAGTGAGG | 2545 | CUGUCAGGGGGACGCGAGUG | 4246 |
| | TCGATTGGCCGCAGCCTACTCGG | 2546 | UCGAUUGGCCGCAGCCUACU | 4247 |
| | GTCGTCGCACGGGTGGCTGTCGG | 2547 | GUCGUCGCACGGGUGGCUGU | 4248 |
| | CGGGACGGAAAAGCGCCGTCTGG | 2548 | CGGGACGGAAAAGCGCCGUC | 4249 |
| | AGCGCCGCAGCACACTAATTGGG | 2549 | AGCGCCGCAGCACACUAAUU | 4250 |
| | GGAACAATAGCGCGCGCGGGCGG | 2550 | GGAACAAUAGCGCGCGCGGG | 4251 |
| | CGCGCGAGTGTGGATGACCGAGG | 2551 | CGCGCGAGUGUGGAUGACCG | 4252 |
| | GTACAGGCGGACGTCCATGGCGG | 2552 | GUACAGGCGGACGUCCAUGG | 4253 |
| | GGACTATTACCACGGCGGCAAGG | 2553 | GGACUAUUACCACGGCGGCA | 4254 |
| | TCCCTCTCCGGCGACCGAAGGGG | 2554 | UCCCUCUCCGGCGACCGAAG | 4255 |
| | GCGTCCGCCAGTCGGTGCGTCGG | 2555 | GCGUCCGCCAGUCGGUGCGU | 4256 |
| | AACAACTCAGGGCGTGAGCGTGG | 2556 | AACAACUCAGGGCGUGAGCG | 4257 |
| | GCCTTTCGCCACCCACGGTGAGG | 2557 | GCCUUUCGCCACCCACGGUG | 4258 |
| | CTCGGATTCCGAGTAGGCTGCGG | 2558 | CUCGGAUUCCGAGUAGGCUG | 4259 |
| | GGACGTCCGCCTGTACCCCTCGG | 2559 | GGACGUCCGCCUGUACCCCU | 4260 |
| | TCCGGCGACCGAAGGGGACGCGG | 2560 | UCCGGCGACCGAAGGGGACG | 4261 |
| | TTCCCTCTCCGGCGACCGAAGGG | 2561 | UUCCCUCUCCGGCGACCGAA | 4262 |
| | GGTTTTCGTCACTCGGAGCCCGG | 2562 | GGUUUUCGUCACUCGGAGCC | 4263 |
| | AACCTCGCAGGCTTTTCGTCAGG | 2563 | AACCUCGCAGGCUUUUCGUC | 4264 |
| | CCCGGATTGAACAGCGCGCGTGG | 2564 | CCCGGAUUGAACAGCGCGCG | 4265 |
| | ACGCTCACGCCCTGAGTTGTTGG | 2565 | ACGCUCACGCCCUGAGUUGU | 4266 |
| | TTCGTCAGGCCCCTGGTAGTGGG | 2566 | UUCGUCAGGCCCCUGGUAGU | 4267 |
| | GCGCGCTATTGTTCCGGCCTCGG | 2567 | GCGCGCUAUUGUUCCGGCCU | 4268 |
| | GCCCGCGCGCGCTATTGTTCCGG | 2568 | GCCCGCGCGCGCUAUUGUUC | 4269 |
| | AGCGCCGCCTCAAATATTTAGGG | 2569 | AGCGCCGCCUCAAAUAUUUA | 4270 |
| | AACAATAGCGCGCGCGGGCGGGG | 2570 | AACAAUAGCGCGCGCGGGCG | 4271 |
| | CAGCGCCGCAGCACACTAATTGG | 2571 | CAGCGCCGCAGCACACUAAU | 4272 |
| | TAGCGCCGCCTCAAATATTTAGG | 2572 | UAGCGCCGCCUCAAAUAUUU | 4273 |
| | GTGGGCTCCGTGGCGATGCGGGG | 2573 | GUGGGCUCCGUGGCGAUGCG | 4274 |
| | CCGGATTGAACAGCGCGCGTGGG | 2574 | CCGGAUUGAACAGCGCGCGU | 4275 |
| | GAGTAGGCTGCGGCCAATCGAGG | 2575 | GAGUAGGCUGCGGCCAAUCG | 4276 |
| | GCGTGGGCTCCGTGGCGATGCGG | 2576 | GCGUGGGCUCCGUGGCGAUG | 4277 |
| | TATTACCACGGCGGCAAGGTAGG | 2577 | UAUUACCACGGCGGCAAGGU | 4278 |
| | GACCTGACGCCTCGGGTTCGGGG | 2578 | GACCUGACGCCUCGGGUUCG | 4279 |
| | ACCACCGCCCCACATCGCGCAGG | 2579 | ACCACCGCCCCACAUCGCGC | 4280 |
| | GAGTGACGAAAACCCGCGCTTGG | 2580 | GAGUGACGAAAACCCGCGCU | 4281 |
| | CGGGCGCCGAGGGGTACAGGCGG | 2581 | CGGGCGCCGAGGGGUACAGG | 4282 |
| | GTTCCCTCTCCGGCGACCGAAGG | 2582 | GUUCCCUCUCCGGCGACCGA | 4283 |
| | CGGAAAAGCGCCGTCTGGACAGG | 2583 | CGGAAAAGCGCCGUCUGGAC | 4284 |
| | TGACGAAAAGCCTGCGAGGTTGG | 2584 | UGACGAAAAGCCUGCGAGGU | 4285 |
| | GGGGTACAGGCGGACGTCCATGG | 2585 | GGGGUACAGGCGGACGUCCA | 4286 |
| | TCCCCTTCGGTCGCCGGAGAGGG | 2586 | UCCCCUUCGGUCGCCGGAGA | 4287 |
| | CGCACATCAGCCCCGCCGACGGG | 2587 | CGCACAUCAGCCCCGCCGAC | 4288 |
| | GTGAGTGCGCGTCCAGTGGCTGG | 2588 | GUGAGUGCGCGUCCAGUGGC | 4289 |
| | TTTCGTCAGGCCCCTGGTAGTGG | 2589 | UUUCGUCAGGCCCCUGGUAG | 4290 |
| | AACCCCGAACCCGAGGCGTCAGG | 2590 | AACCCCGAACCCGAGGCGUC | 4291 |
| | CACCTGGACTATTACCACGGCGG | 2591 | CACCUGGACUAUUACCACGG | 4292 |
| | ACGGCTTGTGAATGACTGCGAGG | 2592 | ACGGCUUGUGAAUGACUGCG | 4293 |
| | TGAGGTCTCGCGAAGAGTGGCGG | 2593 | UGAGGUCUCGCGAAGAGUGG | 4294 |
| | AAGTGAGGTCTCGCGAAGAGTGG | 2594 | AAGUGAGGUCUCGCGAAGAG | 4295 |
| | TGCCTGGAACCCCGAACCCGAGG | 2595 | UGCCUGGAACCCCGAACCCG | 4296 |
| | GCACTCACCCCGCATCGCCACGG | 2596 | GCACUCACCCCGCAUCGCCA | 4297 |
| | GCGCACCTGGACTATTACCACGG | 2597 | GCGCACCUGGACUAUUACCA | 4298 |
| | GCTGCGAGAGTGTGACTGTCGGG | 2598 | GCUGCGAGAGUGUGACUGUC | 4299 |
| | GTCCCCTTCGGTCGCCGGAGAGG | 2599 | GUCCCCUUCGGUCGCCGGAG | 4300 |
| | CGGGGCCTCGGATTCCGAGTAGG | 2600 | CGGGGCCUCGGAUUCCGAGU | 4301 |
| | TACCACGGCGGCAAGGTAGGCGG | 2601 | UACCACGGCGGCAAGGUAGG | 4302 |
| | TCGGTGCGTCGGTCCCGGGCCGG | 2602 | UCGGUGCGUCGGUCCCGGGC | 4303 |
| | GGTAATAGTCCAGGTGCGCCAGG | 2603 | GGUAAUAGUCCAGGUGCGCC | 4304 |
| | GGGTGACTTGCGTGGGACGGCGG | 2604 | GGGUGACUUGCGUGGGACGG | 4305 |
| | CAGCCCTGGCGCAGACGCGTGGG | 2605 | CAGCCCUGGCGCAGACGCGU | 4306 |
| | TGGCGGACGCGCGGACAGTCTGG | 2606 | UGGCGGACGCGCGGACAGUC | 4307 |
| | GCGGACGTCCATGGCGGGCGCGG | 2607 | GCGGACGUCCAUGGCGGGCG | 4308 |
| | GCGCACATCAGCCCCGCCGACGG | 2608 | GCGCACAUCAGCCCCGCCGA | 4309 |
| | TGGACAGGCGCGCCCCCCTCAGG | 2609 | UGGACAGGCGCGCCCCCCUC | 4310 |
| | GCCAGTCGGTGCGTCGGTCCCGG | 2610 | GCCAGUCGGUGCGUCGGUCC | 4311 |
| | GACGAAAAGCCTGCGAGGTTGGG | 2611 | GACGAAAAGCCUGCGAGGUU | 4312 |
| | CGCTCACGCCCTGAGTTGTTGGG | 2612 | CGCUCACGCCCUGAGUUGUU | 4313 |
| | TCCTCACCGTGGGTGGCGAAAGG | 2613 | UCCUCACCGUGGGUGGCGAA | 4314 |
| | TCAGACACAGATCGTCCTGACGG | 2614 | UCAGACACAGAUCGUCCUGA | 4315 |
| | CAAAGTTAGAAGCCGATGAGGGG | 2615 | CAAAGUUAGAAGCCGAUGAG | 4316 |
| | CCACGGCGGCAAGGTAGGCGGGG | 2616 | CCACGGCGGCAAGGUAGGCG | 4317 |
| | TGTCAGGGGGACGCGAGTGAGGG | 2617 | UGUCAGGGGGACGCGAGUGA | 4318 |
| | TCGTCAGGCCCCTGGTAGTGGGG | 2618 | UCGUCAGGCCCCUGGUAGUG | 4319 |
| | AGGCTACCATCCCCCCTCATCGG | 2619 | AGGCUACCAUCCCCCCUCAU | 4320 |
| | GGGCCACTGTGTCGTCGCACGGG | 2620 | GGGCCACUGUGUCGUCGCAC | 4321 |
| | ACGAAAAGCCTGCGAGGTTGGGG | 2621 | ACGAAAAGCCUGCGAGGUUG | 4322 |
| | GGTGACTTGCGTGGGACGGCGGG | 2622 | GGUGACUUGCGUGGGACGGC | 4323 |
| | CCAGTCGGTGCGTCGGTCCCGGG | 2623 | CCAGUCGGUGCGUCGGUCCC | 4324 |
| | TCCGCGTCCCCTTCGGTCGCCGG | 2624 | UCCGCGUCCCCUUCGGUCGC | 4325 |
| | TGCGCGGCCCCGTGTGACCCCGG | 2625 | UGCGCGGCCCCGUGUGACCC | 4326 |
| | CACTAGGGCCTGCGCGATGTGGG | 2626 | CACUAGGGCCUGCGCGAUGU | 4327 |
| | TACCCCTCGGCGCCCGCGGTGGG | 2627 | UACCCCUCGGCGCCCGCGGU | 4328 |
| IRF4 | ATGAGCTAACCGGACTGTCGGGG | 2628 | AUGAGCUAACCGGACUGUCG | 4329 |
| | ACGCGGGGCATGAACCTGGAGGG | 2629 | ACGCGGGGCAUGAACCUGGA | 4330 |
| | GCCGGAGACCTTGAAGAGCGCGG | 2630 | GCCGGAGACCUUGAAGAGCG | 4331 |
| | GGGGTCCTATTCGGGGCGAAGGG | 2631 | GGGGUCCUAUUCGGGGCGAA | 4332 |
| | GCGCGGAATCCCCCGTACTGGGG | 2632 | GCGCGGAAUCCCCCGUACUG | 4333 |
| | AACGACAAGTGGCGCAGACGCGG | 2633 | AACGACAAGUGGCGCAGACG | 4334 |
| | GTCGCTCCGAGCCTTGCGTGCGG | 2634 | GUCGCUCCGAGCCUUGCGUG | 4335 |
| | ACAGGCGCGGACGCACGGAGAGG | 2635 | ACAGGCGCGGACGCACGGAG | 4336 |
| | GCAGAGCGTGTAACGGAAGACGG | 2636 | GCAGAGCGUGUAACGGAAGA | 4337 |
| | TGCGGTGCCTCGTGGCTGAAGGG | 2637 | UGCGGUGCCUCGUGGCUGAA | 4338 |
| | CGTCTGCCGCCTCCGTCCGTGGG | 2638 | CGUCUGCCGCCUCCGUCCGU | 4339 |
| | GCGAATCTCGCCTTTGCGCCAGG | 2639 | GCGAAUCUCGCCUUUGCGCC | 4340 |
| | CCCGGTGATGGCCTTGCCGAGGG | 2640 | CCCGGUGAUGGCCUUGCCGA | 4341 |
| | CAAGACGAGCGGCGCGTGTCGGG | 2641 | CAAGACGAGCGGCGCGUGUC | 4342 |
| | GCGCGCGGAATCCCCCGTACTGG | 2642 | GCGCGCGGAAUCCCCCGUAC | 4343 |
| | CGCGCGGAATCCCCCGTACTGGG | 2643 | CGCGCGGAAUCCCCCGUACU | 4344 |
| | TTAGTGCGCGCTAGCTGGGCAGG | 2644 | UUAGUGCGCGCUAGCUGGGC | 4345 |
| | CCCACTTAGTGCGCGCTAGCTGG | 2645 | CCCACUUAGUGCGCGCUAGC | 4346 |
| | CCACTTAGTGCGCGCTAGCTGGG | 2646 | CCACUUAGUGCGCGCUAGCU | 4347 |
| | GCGATGTTCTCTAAACACCGCGG | 2647 | GCGAUGUUCUCUAAACACCG | 4348 |
| | GAGCGTGTAACGGAAGACGGAGG | 2648 | GAGCGUGUAACGGAAGACGG | 4349 |
| | CGGTGGGTCCCAAGATCGAGCGG | 2649 | CGGUGGGUCCCAAGAUCGAG | 4350 |
| | AGAGCGCGGCGTCCTCCTCGCGG | 2650 | AGAGCGCGGCGUCCUCCUCG | 4351 |
| | GCGAAGGTGCCTTCTTCCGGGGG | 2651 | GCGAAGGUGCCUUCUUCCGG | 4352 |
| | CTAAACACCGCGGAGAGGCAGGG | 2652 | CUAAACACCGCGGAGAGGCA | 4353 |
| | GCGAGGTCCTCCGCGCGTGGAGG | 2653 | GCGAGGUCCUCCGCGCGUGG | 4354 |
| | ATCGACAGCGGCAAGTACCCCGG | 2654 | AUCGACAGCGGCAAGUACCC | 4355 |
| | CGACAAGTGGCGCAGACGCGGGG | 2655 | CGACAAGUGGCGCAGACGCG | 4356 |
| | AGTACCCGCAGAGAGCTAGCAGG | 2656 | AGUACCCGCAGAGAGCUAGC | 4357 |
| | AATGGGGGGCGTGTAGTAGCGGG | 2657 | AAUGGGGGGCGUGUAGUAGC | 4358 |
| | ACGACAAGTGGCGCAGACGCGGG | 2658 | ACGACAAGUGGCGCAGACGC | 4359 |
| | GATGAGCTAACCGGACTGTCGGG | 2659 | GAUGAGCUAACCGGACUGUC | 4360 |
| | AGGGGTCCTATTCGGGGCGAAGG | 2660 | AGGGGUCCUAUUCGGGGCGA | 4361 |
| | CGAACCTCTGGTTCGCGCTCCGG | 2661 | CGAACCUCUGGUUCGCGCUC | 4362 |
| | CAGTTTCACCGCTCGATCTTGGG | 2662 | CAGUUUCACCGCUCGAUCUU | 4363 |
| | ACCTCGCCCTTCGCGGGAAACGG | 2663 | ACCUCGCCCUUCGCGGGAAA | 4364 |
| | AAGCGCGCGCGTGCCGTGTCAGG | 2664 | AAGCGCGCGCGUGCCGUGUC | 4365 |
| | TTGGGCTGCGGGTGCGTTACAGG | 2665 | UUGGGCUGCGGGUGCGUUAC | 4366 |
| | GCGACCCCGTCGCAGGAGCGCGG | 2666 | GCGACCCCGUCGCAGGAGCG | 4367 |
| | TAAGGGGCCCAAGCTCACGGCGG | 2667 | UAAGGGGCCCAAGCUCACGG | 4368 |
| | CTGATCGACCAGATCGACAGCGG | 2668 | CUGAUCGACCAGAUCGACAG | 4369 |
| | CAAGCAGGACTACAACCGCGAGG | 2669 | CAAGCAGGACUACAACCGCG | 4370 |
| | GTTCTCTAAACACCGCGGAGAGG | 2670 | GUUCUCUAAACACCGCGGAG | 4371 |
| | CGGAGAGTTCGGCATGAGCGCGG | 2671 | CGGAGAGUUCGGCAUGAGCG | 4372 |
| | TGCGTGGAAACGAGAACGCACGG | 2672 | UGCGUGGAAACGAGAACGCA | 4373 |
| | GTAACGCACCCGCAGCCCAAAGG | 2673 | GUAACGCACCCGCAGCCCAA | 4374 |
| | GGACCCGGAGCGCGAACCAGAGG | 2674 | GGACCCGGAGCGCGAACCAG | 4375 |
| | TAGCGGGAATCTGGTGCGAAGGG | 2675 | UAGCGGGAAUCUGGUGCGAA | 4376 |
| | GGGGTCGCCACAAGCTGGACGGG | 2676 | GGGGUCGCCACAAGCUGGAC | 4377 |
| | CTGGGGCCGTTTCCCGCGAAGGG | 2677 | CUGGGGCCGUUUCCCGCGAA | 4378 |
| | TCCGCGCGCAGAGCGTCCGCCGG | 2678 | UCCGCGCGCAGAGCGUCCGC | 4379 |
| | AGCTCATCCCGTCCAGCTTGTGG | 2679 | AGCUCAUCCCGUCCAGCUUG | 4380 |
| | TCCCGGTGATGGCCTTGCCGAGG | 2680 | UCCCGGUGAUGGCCUUGCCG | 4381 |
| | GCCGTTTCCCGCGAAGGGCGAGG | 2681 | GCCGUUUCCCGCGAAGGGCG | 4382 |
| | GCGACGGGGTCGCCACAAGCTGG | 2682 | GCGACGGGGUCGCCACAAGC | 4383 |
| | TTTCGCACCTCGCCCTTCGCGGG | 2683 | UUUCGCACCUCGCCCUUCGC | 4384 |
| | ACCCTCGGCAAGGCCATCACCGG | 2684 | ACCCUCGGCAAGGCCAUCAC | 4385 |
| | GGTACTTGCCGCTGTCGATCTGG | 2685 | GGUACUUGCCGCUGUCGAUC | 4386 |
| | TGCGTCCGCGCCTGTGCCGGCGG | 2686 | UGCGUCCGCGCCUGUGCCGG | 4387 |
| | CACGGACGGAGGCGGCAGACGGG | 2687 | CACGGACGGAGGCGGCAGAC | 4388 |
| | GGCGCGTGTCGGGAGCCTTTGGG | 2688 | GGCGCGUGUCGGGAGCCUUU | 4389 |
| | GCTTGTGGCGACCCCGTCGCAGG | 2689 | GCUUGUGGCGACCCCGUCGC | 4390 |
| | GCCTGCGGCCGGGCGTTCCAGGG | 2690 | GCCUGCGGCCGGGCGUUCCA | 4391 |
| | CGTGCCGTGTCAGGGTCGTCCGG | 2691 | CGUGCCGUGUCAGGGUCGUC | 4392 |
| | ACGAAAACAGCCGCCGGCACAGG | 2692 | ACGAAAACAGCCGCCGGCAC | 4393 |
| | TTCGCGCTCCGGGTCCTCTCTGG | 2693 | UUCGCGCUCCGGGUCCUCUC | 4394 |
| | AGCCGTCCGCCTTCCGAGCTCGG | 2694 | AGCCGUCCGCCUUCCGAGCU | 4395 |
| | CGGCGCGTGTCGGGAGCCTTTGG | 2695 | CGGCGCGUGUCGGGAGCCUU | 4396 |
| | AGGCACCTTCGCGGCCGGCCCGG | 2696 | AGGCACCUUCGCGGCCGGCC | 4397 |
| | GCGGTGAAACTGAGAGTGCGAGG | 2697 | GCGGUGAAACUGAGAGUGCG | 4398 |
| | CCTCGTGGTCACTGGCGCAGGGG | 2698 | CCUCGUGGUCACUGGCGCAG | 4399 |
| | AGGGTACCCCGGCTTCGGAGCGG | 2699 | AGGGUACCCCGGCUUCGGAG | 4400 |
| | TCGACAGCGGCAAGTACCCCGGG | 2700 | UCGACAGCGGCAAGUACCCC | 4401 |
| | CGCGAAGGTGCCTTCTTCCGGGG | 2701 | CGCGAAGGUGCCUUCUUCCG | 4402 |
| | GCTCCGAGCCTTGCGTGCGGTGG | 2702 | GCUCCGAGCCUUGCGUGCGG | 4403 |
| | GCCGCGCTCTTCAAGGTCTCCGG | 2703 | GCCGCGCUCUUCAAGGUCUC | 4404 |
| | TCGCTTTGCAGAGCGTGTAACGG | 2704 | UCGCUUUGCAGAGCGUGUAA | 4405 |
| | CTCGGCTCTCAGCGGGACCGCGG | 2705 | CUCGGCUCUCAGCGGGACCG | 4406 |
| | GTGCCGTGTCAGGGTCGTCCGGG | 2706 | GUGCCGUGUCAGGGUCGUCC | 4407 |
| | GCAAGACGAGCGGCGCGTGTCGG | 2707 | GCAAGACGAGCGGCGCGUGU | 4408 |
| | AACTGACAGAGTCGCGGGGAAGG | 2708 | AACUGACAGAGUCGCGGGGA | 4409 |
| | CAGGCGGGTAGGAGCCTTCGCGG | 2709 | CAGGCGGGUAGGAGCCUUCG | 4410 |
| | GGGTACCCCGGCTTCGGAGCGGG | 2710 | GGGUACCCCGGCUUCGGAGC | 4411 |
| | GAGGCATCAGGTGGCGTCGCCGG | 2711 | GAGGCAUCAGGUGGCGUCGC | 4412 |
| | CCGTCTGCCGCCTCCGTCCGTGG | 2712 | CCGUCUGCCGCCUCCGUCCG | 4413 |
| | GCCGTCTTGTGTGGGTGCCTTGG | 2713 | GCCGUCUUGUGUGGGUGCCU | 4414 |
| | GAACCTCTGGTTCGCGCTCCGGG | 2714 | GAACCUCUGGUUCGCGCUCC | 4415 |
| | GCGCGGTGAGCTGCGGCAACGGG | 2715 | GCGCGGUGAGCUGCGGCAAC | 4416 |
| | GCCTCCGGCTCAGCGCAGATGGG | 2716 | GCCUCCGGCUCAGCGCAGAU | 4417 |
| | GGCGTGTAGTAGCGGGAATCTGG | 2717 | GGCGUGUAGUAGCGGGAAUC | 4418 |
| | GGAGGACGCCGCGCTCTTCAAGG | 2718 | GGAGGACGCCGCGCUCUUCA | 4419 |
| | CGGCACGCGGGGCATGAACCTGG | 2719 | CGGCACGCGGGGCAUGAACC | 4420 |
| | CCGCGAAGGTGCCTTCTTCCGGG | 2720 | CCGCGAAGGUGCCUUCUUCC | 4421 |
| | CGGGGTCGCCACAAGCTGGACGG | 2721 | CGGGGUCGCCACAAGCUGGA | 4422 |
| | TCCGGCGGACGCTCTGCGCGCGG | 2722 | UCCGGCGGACGCUCUGCGCG | 4423 |
| | AGCGCAGGGTACCCCGGCTTCGG | 2723 | AGCGCAGGGUACCCCGGCUU | 4424 |
| | CCTATTCGGGGCGAAGGGTCTGG | 2724 | CCUAUUCGGGGCGAAGGGUC | 4425 |
| | CTCTTCAAGGTCTCCGGCCTCGG | 2725 | CUCUUCAAGGUCUCCGGCCU | 4426 |
| | TCGGCTCTCAGCGGGACCGCGGG | 2726 | UCGGCUCUCAGCGGGACCGC | 4427 |
| | TGGAAACTGACAGAGTCGCGGGG | 2727 | UGGAAACUGACAGAGUCGCG | 4428 |
| TET2 | TGCGCGGGACCTCGAAGTGGTGG | 2728 | UGCGCGGGACCUCGAAGUGG | 4429 |
| | GCACCGGGCGTCCAGCACAAAGG | 2729 | GCACCGGGCGUCCAGCACAA | 4430 |
| | AGGGAATTAGCCCCCCGCACCGG | 2730 | AGGGAAUUAGCCCCCCGCAC | 4431 |
| | ACTTGCATGCGAGCGGGACCCGG | 2731 | ACUUGCAUGCGAGCGGGACC | 4432 |
| | TCACGCCGTGCAGTGGCGCGGGG | 2732 | UCACGCCGUGCAGUGGCGCG | 4433 |
| | CGCGGGCAACGGGATCTAAAGGG | 2733 | CGCGGGCAACGGGAUCUAAA | 4434 |
| | GCGCGGGCAACGGGATCTAAAGG | 2734 | GCGCGGGCAACGGGAUCUAA | 4435 |
| | GACGTGACTTGCATGCGAGCGGG | 2735 | GACGUGACUUGCAUGCGAGC | 4436 |
| | ATAGAGACGCGGGCCTCTGAGGG | 2736 | AUAGAGACGCGGGCCUCUGA | 4437 |
| | GTGCGGGTACACTCCGGAGGAGG | 2737 | GUGCGGGUACACUCCGGAGG | 4438 |
| | CACGCCGTGCAGTGGCGCGGGGG | 2738 | CACGCCGUGCAGUGGCGCGG | 4439 |
| | GGCATGCCCTCGGTGAAACAGGG | 2739 | GGCAUGCCCUCGGUGAAACA | 4440 |
| | GGGAATTAGCCCCCCGCACCGGG | 2740 | GGGAAUUAGCCCCCCGCACC | 4441 |
| | GGTGCCGCCGGCCTTTGTGCTGG | 2741 | GGUGCCGCCGGCCUUUGUGC | 4442 |
| | AGCGCTCCCCTGTTTCACCGAGG | 2742 | AGCGCUCCCCUGUUUCACCG | 4443 |
| | GCGCTCCCCTGTTTCACCGAGGG | 2743 | GCGCUCCCCUGUUUCACCGA | 4444 |
| | GTGTGCGCGGGACCTCGAAGTGG | 2744 | GUGUGCGCGGGACCUCGAAG | 4445 |
| | GTGCGGGGGGCTAATTCCCTGGG | 2745 | GUGCGGGGGGCUAAUUCCCU | 4446 |
| | ACCCGCACGTGCCCTCGCTCTGG | 2746 | ACCCGCACGUGCCCUCGCUC | 4447 |
| | CTCACGCCGTGCAGTGGCGCGGG | 2747 | CUCACGCCGUGCAGUGGCGC | 4448 |
| | GTGGTGCGCCCGGACCAGCGCGG | 2748 | GUGGUGCGCCCGGACCAGCG | 4449 |
| | CACGTGCGGGTACACTCCGGAGG | 2749 | CACGUGCGGGUACACUCCGG | 4450 |
| | GCGTCCAGCACAAAGGCCGGCGG | 2750 | GCGUCCAGCACAAAGGCCGG | 4451 |
| | TTTGTGCTGGACGCCCGGTGCGG | 2751 | UUUGUGCUGGACGCCCGGUG | 4452 |
| | TGTACGGCCCCAGGTGCCGCCGG | 2752 | UGUACGGCCCCAGGUGCCGC | 4453 |
| | CCGCGCCACTGCACGGCGTGAGG | 2753 | CCGCGCCACUGCACGGCGUG | 4454 |
| | GGGCATGCCCTCGGTGAAACAGG | 2754 | GGGCAUGCCCUCGGUGAAAC | 4455 |
| | TTGTGCTGGACGCCCGGTGCGGG | 2755 | UUGUGCUGGACGCCCGGUGC | 4456 |
| | GGGCACGTGCGGGTACACTCCGG | 2756 | GGGCACGUGCGGGUACACUC | 4457 |
| | GGACGTGACTTGCATGCGAGCGG | 2757 | GGACGUGACUUGCAUGCGAG | 4458 |
| | TCACGTCCGCCCCCTCGGCGCGG | 2758 | UCACGUCCGCCCCCUCGGCG | 4459 |
| | ACGCCGTGCAGTGGCGCGGGGGG | 2759 | ACGCCGUGCAGUGGCGCGGG | 4460 |
| | GCACCTGGGGCCGTACAGCGGGG | 2760 | GCACCUGGGGCCGUACAGCG | 4461 |
| | CGCGCCACTGCACGGCGTGAGGG | 2761 | CGCGCCACUGCACGGCGUGA | 4462 |
| | GGTAAGGTGGGCGCAAGCGGAGG | 2762 | GGUAAGGUGGGCGCAAGCGG | 4463 |
| | CTTGCATGCGAGCGGGACCCGGG | 2763 | CUUGCAUGCGAGCGGGACCC | 4464 |
| | GGAGACCCGCCGAGGTCCCCGGG | 2764 | GGAGACCCGCCGAGGUCCCC | 4465 |
| | CGCAAGCGGAGGTGTGGTGCGGG | 2765 | CGCAAGCGGAGGUGUGGUGC | 4466 |
| | GGTGCGGGGGGCTAATTCCCTGG | 2766 | GGUGCGGGGGGCUAAUUCCC | 4467 |
| | TAGATGTCACGTCTTTGTCCAGG | 2767 | UAGAUGUCACGUCUUUGUCC | 4468 |
| | AGCAGAGCAAGCGCGAAGGTTGG | 2768 | AGCAGAGCAAGCGCGAAGGU | 4469 |
| | GCATGCCCTCGGTGAAACAGGGG | 2769 | GCAUGCCCUCGGUGAAACAG | 4470 |
| | CTAAAGGGAGATAGAGACGCGGG | 2770 | CUAAAGGGAGAUAGAGACGC | 4471 |
| | CCACTGCGCGCCCCGCTGTACGG | 2771 | CCACUGCGCGCCCCGCUGUA | 4472 |
| | GACGCGGGCCTCTGAGGGTAAGG | 2772 | GACGCGGGCCUCUGAGGGUA | 4473 |
| | AGTGGCAGCGGCGAGAGCTTGGG | 2773 | AGUGGCAGCGGCGAGAGCUU | 4474 |
| | GCAGAGCAAGCGCGAAGGTTGGG | 2774 | GCAGAGCAAGCGCGAAGGUU | 4475 |
| | AAGCACTAAGGGCATGCCCTCGG | 2775 | AAGCACUAAGGGCAUGCCCU | 4476 |
| | TACAGGCCCCTAAAGCACTAAGG | 2776 | UACAGGCCCCUAAAGCACUA | 4477 |
| | CCTTATGAATATTGATGCGGAGG | 2777 | CCUUAUGAAUAUUGAUGCGG | 4478 |
| | GGAATTAGCTCTGTATCGGTCGG | 4547 | GGAAUUAGCUCUGUAUCGGU | 4560 |
| | AAAGTAAGGGCTCTTACGAGAGG | 4548 | AAAGUAAGGGCUCUUACGAG | 4561 |
| | GGCGTCTCACAGATTGAAATAGG | 4549 | GGCGUCUCACAGAUUGAAAU | 4562 |
| | CGGTCAATTTCCCAGTTTGTCGG | 4550 | CGGUCAAUUUCCCAGUUUGU | 4563 |
| | TGCAGCCCTCGGGAACCCCGGGG | 4551 | UGCAGCCCUCGGGAACCCCG | 4564 |
| | ACTCAGCGGGGCCGGCGTCTCGG | 4552 | ACUCAGCGGGGCCGGCGUCU | 4565 |

It will be appreciated that it may be beneficial to increase the expression of certain targets. For example, c-jun is a gene that when activated, may be beneficial; for example, increased expression in T cells may increase cell viability.

### Cell

In one aspect, the present invention provides a cell comprising an ETM (e.g., ETR) according to the present invention, at least one gRNA according the present invention, a combination according to the present invention, a polynucleotide according to the present invention, a nucleic acid construct according to the present invention, a vector according to the present invention or a kit of polynucleotides according to the present invention.

The cell may be any cell which can be used to express the product of the invention.

The cell may be an immune effector cell. An “immune effector cell” is a cell which has differentiated into a form capable of modulating or effecting a specific immune response. Immune effector cells may include alpha/beta T cells, gamma/delta T cells, B cells, natural killer (NK) cells, neutrophils, basophils, eosinophils, and macrophages. Suitably, the cell may be an alpha/beta T cell. Suitably, the cell may be a B cell. Suitably, the cell may be a gamma/delta T cell. Suitably, the cell may be a T cell, such as a cytolytic T cell, e.g., a CD8+ T cell. Suitably, the cell may be an NK cell, such as a cytolytic NK cell. Suitably, the cell may be a macrophage.

In one aspect, the cell may be a stem cell. A “stem cell” refers to an undifferentiated cell which is capable of indefinitely giving rise to more stem cells of the same type, and from which other, specialised cells may arise by differentiation. Adult stem cells are usually multipotent, while induced or embryonic-derived stem cells are pluripotent.

In another aspect, the cell may be a progenitor cell. A “progenitor cell” refers to a cell which is able to differentiate to form one or more types of cells but has limited self-renewal in vitro and in vivo.

Suitably, the cell may be capable of being differentiated into a T cell. Suitably, the cell may be capable of being differentiated into an NK cell. Suitably, the cell may be capable of being differentiated into a macrophage. Suitably, the cell may be an embryonic stem cell (ESC). Suitably, the cell may be a haematopoietic stem cell or haematopoietic progenitor cell. Suitably, the cell may be an induced pluripotent stem cell (iPSC). Suitably, the cell may be obtained from umbilical cord blood. Suitably, the cell may be obtained from adult peripheral blood or mobilized form the bone marrow.

A “hematopoietic stem and progenitor cell” or “HSPC” refers to a cell which expresses the antigenic marker CD34 (CD34+) and populations of such cells. In particular embodiments, the term “HSPC” refers to a cell identified by the presence of the antigenic marker CD34 (CD34+) and the absence of lineage (lin) markers. The population of cells comprising CD34+ and/or Lin(−) cells includes haematopoietic stem cells and hematopoietic progenitor cells.

HSPCs can be obtained or isolated from bone marrow of adults, which includes femurs, hip, ribs, sternum, and other bones. Bone marrow aspirates containing HSPCs can be obtained or isolated directly from the hip using a needle and syringe. Other sources of HSPCs include umbilical cord blood, placental blood, mobilized peripheral blood, Wharton's jelly, placenta, fetal blood, fetal liver, or fetal spleen. In particular embodiments, harvesting a sufficient quantity of HSPCs for use in therapeutic applications may require mobilizing the stem and progenitor cells in the subject.

As used herein, the term “induced pluripotent stem cell” or “iPSC” refers to a non-pluripotent cell that has been reprogrammed to a pluripotent state. Once the cells of a subject have been reprogrammed to a pluripotent state, the cells can then be programmed to a desired cell type, such as a hematopoietic stem or progenitor cell (HSC and HPC respectively).

As used herein, the term “reprogramming” refers to a method of increasing the potency of a cell to a less differentiated state and “programming” refers to a method of decreasing the potency of a cell or differentiating the cell to a more differentiated state.

Suitably, the cell may be matched or is autologous to the subject. The cell may be generated ex vivo either from a patient's own peripheral blood, or from donor peripheral blood.

Suitably, the cell may be autologous to the subject. In some aspects, the cell may be derived from ex vivo differentiation of inducible progenitor cells or embryonic progenitor cells to the immune cell.

In these instances, cells are generated by introducing DNA or RNA coding for the ETM (e.g., ETR) of the present invention by one of any means including transduction with a viral vector or transfection with DNA or RNA.

In some aspects, the cell further comprises a polynucleotide, such as an integrating vector, which encodes an agent:
i) which promotes the survival, proliferation and/or activity of a cell, such as a cell which comprises the polynucleotide or a cell which does not comprise the polynucleotide; and/or
ii) which is detrimental to the survival, proliferation, activity, chemoresistance and/or chemotaxis of a cell, such as a cell which comprises the polynucleotide or a cell which does not comprise the polynucleotide and/or
iii) which enables selection of a cell, such as a cell which comprises the polynucleotide or a cell which does not comprise the polynucleotide.

### Combinations

In one aspect, the present invention provides a combination (e.g., a system) comprising an ETM (e.g., ETR) according to the present invention, and at least one gRNA which targets the endonuclease of the ETM (e.g., ETR) to a target gene.

The combination may comprise at least two gRNAs (such as at least three, at least four, at least five, at least six, at least seven. or at least eight gRNAs).

The combination may comprise gRNAs which target the endonuclease to at least two different target genes.

In some embodiments, one target gene may be targeted with two or more gRNAs. For example, it may be beneficial to target the same gene with several gRNAs for optimal epigenetic modification, e.g., epigenetic silencing.

The combination may comprise at least two gRNAs which comprise spacer sequences of different lengths. Suitably, at least one gRNA comprises a spacer sequence which is 15, 16, 17, 18, 19 or 20 nucleotides in length. Suitably, at least one of the at least two gRNAs comprises a spacer sequence which is less than or equal to 17 (e.g., less than or equal to 16) nucleotides in length. Suitably, at least one of the at least two gRNAs comprises a spacer sequence which is less than or equal to 17 (e.g., less than or equal to 16) nucleotides in length and at least one of the at least two gRNAs comprises a spacer sequence which is more than 17 nucleotides in length.

Without wishing to be bound by theory, the gRNAs comprising spacer sequences of different lengths may target the ETM (e.g., ETR) to different target genes, wherein a first target gene is modified by gene editing and at least a second target gene is modified by epigenetic editing.

In one aspect, the combination comprises at least one gRNA according to the present invention. Suitably, the combination may comprise at least two gRNAs according to the present invention.

Suitably, the combination may comprise a first gRNA and a second gRNA having the sequences of C8 and F4, respectively, optionally wherein the combination further comprises a third gRNA having the sequence of H8, H10, H11, or H12.

Suitably, the combination may comprise a first gRNA and a second gRNA having the sequences of C8 and H8, respectively, optionally wherein the combination further comprises a third gRNA having the sequence of F4, H10, H11, or H12.

Suitably, the combination may comprise a first gRNA and a second gRNA having the sequences of C8 and H10, respectively, optionally wherein the combination further comprises a third gRNA having the sequence of F4, H8, H11, or H12.

Suitably, the combination may comprise a first gRNA and a second gRNA having the sequences of C8 and H11, respectively, optionally wherein the combination further comprises a third gRNA having the sequence of F4, H8, H10, or H12.

Suitably, the combination may comprise a first gRNA and a second gRNA having the sequences of C8 and H12, respectively, optionally wherein the combination further comprises a third gRNA having the sequence of F4, H8, H10, or H11.

Suitably, the combination may comprise a first gRNA and a second gRNA having the sequences of F4 and H8, respectively, optionally wherein the combination further comprises a third gRNA having the sequence of C8, H10, H11, or H12.

Suitably, the combination may comprise a first gRNA and a second gRNA having the sequences of F4 and H10, respectively, optionally wherein the combination further comprises a third gRNA having the sequence of C8, H8, H11, or H12.

Suitably, the combination may comprise a first gRNA and a second gRNA having the sequences of F4 and H11, respectively, optionally wherein the combination further comprises a third gRNA having the sequence of C8, H8, H10, or H12.

Suitably, the combination may comprise a first gRNA and a second gRNA having the sequences of F4 and H12, respectively, optionally wherein the combination further comprises a third gRNA having the sequence of C8, H8, H10, or H11.

Suitably, the combination may comprise a first gRNA and a second gRNA having the sequences of H8 and H10, respectively, optionally wherein the combination further comprises a third gRNA having the sequence of C8, F4, H11, or H12.

Suitably, the combination may comprise a first gRNA and a second gRNA having the sequences of H10 and H11, respectively, optionally wherein the combination further comprises a third gRNA having the sequence of C8, F4, H8, or H12.

Suitably, the combination may comprise a first gRNA and a second gRNA having the sequences of H10 and H12, respectively, optionally wherein the combination further comprises a third gRNA having the sequence of C8, F4, H8, or H11.

Suitably, the combination may comprise a first gRNA and a second gRNA having the sequences of H11 and H12, respectively, optionally wherein the combination further comprises a third gRNA having the sequence of C8, F4, H8, or H10.

The combination may, for example, have gRNAs comprising or consisting of H8+F4, H8+H10, C8+H10, F4+H10, F4+H8+H10, or C8+F4+H10. In a particular case, the gRNAs may comprise or consist of F4+H8+H10.

In one aspect, the combination further comprises an agent:
i) which promotes the survival, proliferation and/or activity of a cell, such as a cell which comprises the combination or a cell which does not comprise the combination; and/or
ii) which is detrimental to the survival, proliferation, activity, chemoresistance and/or chemotaxis of a cell, such as a cell which comprises the combination or a cell which does not comprise the combination; and/or
iii) which enables selection of a cell, such as a cell which comprises the combination or a cell which does not comprise the combination.

The combination may further comprise an agent which modifies the tissue microenvironment.

The agent may be a protein, such as a cytokine or chemokine, which promotes the survival, proliferation and/or activity of a cell according to the present invention.

As used herein, “agent which promotes the survival, proliferation and/or activity of a cell” means that in the presence of the agent, the survival, proliferation, or activity of a cell which comprises a product according to the present invention is increased.

The agent may be, for example, beneficial for certain cells and detrimental to other cells.

The agent may play a role in homeostasis, for example, blood coagulation; an example of a suitable agent may be coagulation factor IX or FVIII.

The agent may, for example, allow selection of cells. An example of a suitable agent is Delta low-affinity nerve growth factor (LNGFR).

The agent may, for example, be detrimental for the cell. The agent may be a thymidine kinase (TK) or a caspase, such as CASP9. Activation of these agents can be used for in vivo removal of cells which comprise the agent, e.g., if it is desirable to remove engineered T cells from a subject.

Suitably, in the presence of the agent, the survival, proliferation and/or activity of the cell which comprises a product according to the present invention (e.g., a cell according to the present invention) may be increased by at least 5%, at least 10%, at least 20%, at least 30%, at least 40%, at least 50%, at least 60%, at least 70%, at least 80%, at least 90%, at least 95% or at least 99%.

The combination may comprise an agent which is detrimental to the survival, proliferation, activity, chemoresistance and/or chemotaxis of a cell such as a tumour cell.

As used herein “agent which is detrimental to” means that in the presence of the agent, the survival, proliferation, or activity of a cell which does not comprise a product according to the present invention (e.g., a tumour cell) is compromised, reduced, or completely abolished.

Suitably, in the presence of the detrimental agent, the survival, proliferation and/or activity of the cell which does not comprise a product according to the present invention (e.g., a tumour cell) may be reduced by at least 5%, at least 10%, at least 20%, at least 30%, at least 40%, at least 50%, at least 60%, at least 70%, at least 80%, at least 90%, at least 95% or at least 99%.

Cell survival and proliferation may be measured by methods known in the art. Suitable methods include measuring the size of the cell population (e.g., by counting cells using a marker specific for the cell population, i.e., a tumour specific marker or an engineered cell specific marker, such as a CAR or transgenic TCR); by performing cell cycle analysis using 5-bromo-2′-deoxyuridine (BrdU) which becomes incorporated into newly made DNA and/or propidium iodide (PI) and analysing by flow cytometry in combination with a cell population specific marker; and/or by measuring the number of viable cells, e.g., by measuring apoptosis by 7AAD and/or Annexin V staining using flow cytometry.

In one aspect, the combination further comprises a CAR. In one aspect, the combination further comprises a transgenic TCR.

The agent, e.g., which promotes the survival, proliferation and/or activity of a cell (or population of cells) or allows selection of the cell, such as the cell (or population of cells) which expresses an ETM (e.g., ETR); and/or which is detrimental to the survival, proliferation, activity, chemoresistance and/or chemotaxis of a cell which does not express an ETM (e.g., ETR), may be introduced into the genome of the cell by any method. The method may include, for example, using an integrating vector (a procedure independent from the multiplexing strategy performed by the ETM (e.g., ETR) according to the invention); or by targeting the agent (e.g., CAR or transgenic TCR) within the site recognized by the nuclease (a procedure depending on the nuclease activity of the ETM (e.g., ETR) according to the present invention).

Thus in some aspects, the combination further comprises a polynucleotide, such as an integrating vector which encodes an agent which allows selection or promotes the survival, proliferation and/or activity of a cell (or population of cells), such as the cell (or population of cells) which comprises the polynucleotide; and/or which is detrimental to the survival, proliferation, activity, chemoresistance and/or chemotaxis of a cell which does not comprise the polynucleotide; and/or which is beneficial for the survival, proliferation and/or activity of a cell, tissue or organ, such as a cell, tissue or organ which does not comprise the combination.

### Polynucleotides

In one aspect, the present invention provides a polynucleotide encoding at least one ETM (e.g., ETR) according to the present invention.

Polynucleotides of the invention may comprise DNA or RNA. They may be single-stranded or double-stranded. It will be understood by a skilled person that numerous different polynucleotides can encode the same polypeptide as a result of the degeneracy of the genetic code. In addition, it is to be understood that the skilled person may, using routine techniques, make nucleotide substitutions that do not affect the polypeptide sequence encoded by the polynucleotides of the invention to reflect the codon usage of any particular host organism in which the polypeptides of the invention are to be expressed.

The polynucleotides may be modified by any method available in the art. Such modifications may be carried out in order to enhance the in vivo activity or lifespan of the polynucleotides of the invention.

Polynucleotides such as DNA polynucleotides may be produced recombinantly, synthetically or by any means available to those of skill in the art. They may also be cloned by standard techniques.

Longer polynucleotides will generally be produced using recombinant means, for example using PCR cloning techniques. This will involve making a pair of primers (e.g., of about 15 to 30 nucleotides) flanking the target sequence which it is desired to clone, bringing the primers into contact with mRNA or cDNA obtained from an animal or human cell, performing a polymerase chain reaction under conditions which bring about amplification of the desired region, isolating the amplified fragment (e.g., by purifying the reaction mixture with an agarose gel) and recovering the amplified DNA. The primers may be designed to contain suitable restriction enzyme recognition sites so that the amplified DNA can be cloned into a suitable vector.

### Constructs

In one aspect, the present invention provides a nucleic acid construct comprising a nucleic acid sequence encoding at least one ETM (e.g., ETR) according to the present invention.

The nucleic acid construct may further comprise a nucleic acid sequence which encodes an agent:
i) which promotes the survival, proliferation and/or activity of a cell, such as a cell which expresses said nucleic acid construct or a cell which does not express said nucleic acid construct; and/or
ii) which is detrimental to the survival, proliferation, activity, chemoresistance and/or chemotaxis of a cell, such as a cell which expresses said nucleic acid construct or a cell which does not express said nucleic acid construct; and/or
iii) which enables selection of a cell, such as a cell which comprises the nucleic acid construct or a cell which does not comprise the construct.

### Proteins

As used herein, the term “protein” includes single-chain polypeptide molecules as well as multiple-polypeptide complexes where individual constituent polypeptides are linked by covalent or non-covalent means. As used herein, the terms “polypeptide” and “peptide” refer to a polymer in which the monomers are amino acids and are joined together through peptide or disulfide bonds.

### Variants, Derivatives, Analogues, Homologues and Fragments

In addition to the specific proteins and nucleotides mentioned herein, the present invention also encompasses the use of variants, derivatives, analogues, homologues, and fragments thereof.

In the context of the present invention, a variant of any given sequence is a sequence in which the specific sequence of residues (whether amino acid or nucleic acid residues) has been modified in such a manner that the polypeptide or polynucleotide in question substantially retains at least one of its endogenous functions. A variant sequence can be obtained by addition, deletion, substitution, modification, replacement and/or variation of at least one residue present in the naturally-occurring protein.

The term “derivative” as used herein, in relation to proteins or polypeptides of the present invention, includes any substitution of, variation of, modification of, replacement of, deletion of and/or addition of one (or more) amino acid residues from or to the sequence providing that the resultant protein or polypeptide substantially retains at least one of its endogenous functions.

The term “analogue” as used herein, in relation to polypeptides or polynucleotides, includes any mimetic, that is, a chemical compound that possesses at least one of the endogenous functions of the polypeptides or polynucleotides which it mimics.

Typically, amino acid substitutions may be made, for example from 1, 2 or 3 to 10 or 20 substitutions provided that the modified sequence substantially retains the required activity or ability. Amino acid substitutions may include the use of non-naturally occurring analogues.

Proteins used in the present invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity, and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine, and tyrosine.

Conservative substitutions may be made, for example according to the table below. Amino acids in the same block in the second column and, in particular examples, in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R H |
| AROMATIC | | F W Y |

The term “homologue” as used herein means an entity having a certain homology with the wild type amino acid sequence or the wild type nucleotide sequence. The term “homology” can be equated with “identity”.

A homologous sequence may include an amino acid sequence which may be at least 50%, 55%, 65%, 75%, 85% or 90% identical, for example at least 95% or 97% or 99% identical, to the subject sequence. Typically, the homologues will comprise the same active sites, etc., as the subject amino acid sequence. Although homology can also be considered in terms of similarity (i.e., amino acid residues having similar chemical properties/functions), in the context of the present invention it is preferred to express homology in terms of sequence identity.

A homologous sequence may include a nucleotide sequence which may be at least 50%, 55%, 65%, 75%, 85% or 90% identical, for example at least 95% or 97% or 99% identical, to the subject sequence. Although homology can also be considered in terms of similarity, in the context of the present invention it is preferred to express homology in terms of sequence identity.

Reference to a sequence which has a percent identity to any one of the SEQ ID NOs detailed herein may refer, for example to a sequence which has the stated percent identity over the entire length of the SEQ ID NO referred to.

Homology comparisons can be conducted by eye or, more usually, with the aid of readily available sequence comparison programs. These commercially available computer programs can calculate percentage homology or identity between two or more sequences.

Percentage homology may be calculated over contiguous sequences, i.e., one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the nucleotide sequence may cause the following codons to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example, when using the GCG Wisconsin Bestfit package, the default gap penalty for amino acid sequences is −12 for a gap and −4 for each extension.

Calculation of maximum percentage homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al. (1984) Nucleic Acids Res. 12: 387). Examples of other software that can perform sequence comparisons include, but are not limited to, the BLAST package (see Ausubel et al. (1999) ibid—Ch. 18), FASTA (Atschul et al. (1990) J. Mol. Biol. 403-410) and the GENEWORKS suite of comparison tools. Both BLAST and FASTA are available for offline and online searching (see Ausubel et al. (1999) ibid, pages 7-58 to 7-60). However, for some applications, it is preferred to use the GCG Bestfit program. Another tool, called BLAST 2 Sequences is also available for comparing protein and nucleotide sequences (see FEMS Microbiol. Lett. (1999) 174: 247-50; FEMS Microbiol. Lett. (1999) 177: 187-8).

Although the final percentage homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see the user manual for further details). For some applications, it is preferred to use the public default values for the GCG package, or in the case of other software, the default matrix, such as BLOSUM62.

Once the software has produced an optimal alignment, it is possible to calculate percentage homology, e.g., percentage sequence identity. The software typically does this as part of the sequence comparison and generates a numerical result.

“Fragments” are also variants and the term typically refers to a selected region of the polypeptide or polynucleotide that is of interest either functionally or, for example, in an assay. “Fragment” thus refers to an amino acid or nucleic acid sequence that is a portion of a full-length polypeptide or polynucleotide.

Such variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

### Codon Optimisation

The polynucleotides used in the present invention may be codon-optimised. Codon optimisation has previously been described in WO 1999/41397 and WO 2001/79518. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available.

### Vectors

In one aspect, the present invention provides a vector comprising a polynucleotide according the present invention, or a nucleic acid construct according to the present invention.

A vector is a tool that allows or facilitates the transfer of an entity from one environment to another. In accordance with the present invention, and by way of example, some vectors used in recombinant nucleic acid techniques allow entities, such as a segment of nucleic acid (e.g., a heterologous DNA segment, such as a heterologous cDNA segment), to be transferred into a target cell. The vector may serve the purpose of maintaining the heterologous nucleic acid (DNA or RNA) within the cell, facilitating the replication of the vector comprising a segment of nucleic acid, or facilitating the expression of the protein encoded by a segment of nucleic acid. Vectors may be non-viral or viral. Examples of vectors used in recombinant nucleic acid techniques include, but are not limited to, plasmids, mRNA molecules (e.g., in vitro transcribed mRNAs), chromosomes, artificial chromosomes, and viruses. The vector may also be, for example, a naked nucleic acid (e.g., DNA). In its simplest form, the vector may itself be a nucleotide of interest.

The vectors used in the invention may be, for example, plasmid, mRNA, or virus vectors and may include a promoter for the expression of a polynucleotide and optionally a regulator of the promoter.

Vectors comprising polynucleotides used in the invention may be introduced into cells using a variety of techniques known in the art, such as transfection, transformation, and transduction. Several such techniques are known in the art, for example infection with recombinant viral vectors, such as retroviral, lentiviral (e.g., integration-defective lentiviral), adenoviral, adeno-associated viral, baculoviral and herpes simplex viral vectors; direct injection of nucleic acids and biolistic transformation.

Non-viral delivery systems include but are not limited to DNA or RNA transfection methods. Here, transfection includes a process using a non-viral vector to deliver a gene to a target cell. Typical transfection methods include electroporation, DNA biolistics, lipid-mediated transfection, compacted DNA-mediated transfection, liposomes, immunoliposomes, lipofectin, cationic agent-mediated transfection, cationic facial amphiphiles (CFAs) (Nat. Biotechnol. (1996) 14: 556) and combinations thereof.

The term “transfection” is to be understood as encompassing the delivery of polynucleotides to cells by both viral and non-viral delivery.

### Protein Transduction

As an alternative to the delivery of polynucleotides to cells, the products and ETMs (e.g., ETRs) of the present invention may be delivered to cells by protein transduction.

Protein transduction may be via vector delivery (Cai, Y. et al. (2014) Elife 3: e01911; Maetzig, T. et al. (2012) Curr. Gene Ther. 12: 389-409). Vector delivery involves the engineering of viral particles (e.g., lentiviral particles) to comprise the proteins to be delivered to a cell. Accordingly, when the engineered viral particles enter a cell as part of their natural life cycle, the proteins comprised in the particles are carried into the cell.

Protein transduction may be via protein delivery (Gaj, T. et al. (2012) Nat. Methods 9: 805-7). Protein delivery may be achieved, for example, by utilising a vehicle (e.g., liposomes) or even by administering the protein itself directly to a cell.

### Composition

The products of the invention such as ETMs (e.g., ETRs), gRNAs, combinations, polynucleotides, nucleic acid constructs, vectors, cells, and kits of polynucleotides of the present invention may be provided in a composition.

The products of the invention such as combinations, ETMs (e.g., ETRs), gRNAs, polynucleotides, nucleic acid constructs, vectors, compositions, and cells of the present invention may be formulated for administration to subjects with a pharmaceutically acceptable carrier, diluent, or excipient. Suitable carriers and diluents include isotonic saline solutions, for example, phosphate-buffered saline, and potentially contain human serum albumin.

Handling of the cell therapy products may be performed in compliance with the Foundation for the Accreditation of Cellular Therapy and the Joint Accreditation Committee—International Society Cell & Gene Therapy (ISCT) and European Society for Blood and Marrow Transplantation (EBMT) (FACT-JACIE) International Standards for cellular therapy.

In one aspect, there is provided a combination of chemically modified mRNA encoding for an ETM or ETR plus a chemically modified gRNA.

In another aspect, there is provided a ribonucleic complex of protein-RNA that includes the ETR protein attached to a chemically modified gRNA.

### Kit

In one aspect, the present invention provides a kit of polynucleotides comprising:
a) at least one polynucleotide encoding at least one ETM (e.g., ETR) according to the present invention; and
b) a polynucleotide providing at least one gRNA as described herein; and optionally,
c) further comprising a nucleic acid sequence which encodes an agent:
   i) which promotes the survival, proliferation and/or activity of a cell, such as a cell which comprises the polynucleotides or a cell which does not comprise the polynucleotides; and/or
   ii) which is detrimental to the survival, proliferation, activity, chemoresistance and/or chemotaxis of a cell, such as a cell which comprises said polynucleotides or a cell which does not comprise said polynucleotides; and/or
   iii) which enables selection of a cell, such as a cell which comprises the polynucleotides or a cell which does not comprise the polynucleotides.

The kit may also include instructions for use, for example instructions for the simultaneous, sequential, or separate administration of at least one ETM (e.g., ETR) and at least two gRNAs, to a subject in need thereof.

### Use

In one aspect, the present invention provides the use of an ETM (e.g., ETR) according to the present invention, at least one gRNA according to the present invention, a combination according to the present invention, a polynucleotide according to the present invention, a nucleic acid construct according to the present invention, a vector according to the present invention or a kit of polynucleotides according to the present invention for modifying the activity and/or expression of at least one target gene, e.g., wherein the use is in vitro or ex vivo use.

Suitably, the use may repress transcription and/or expression of (e.g., silence) at least one target gene. Suitably, the use may repress transcription and/or expression of (e.g., silence) at least two target genes. For example, transcription and/or expression of a first gene may be repressed (e.g., silenced) by gene editing and transcription and/or expression of a second target gene may be repressed (e.g., silenced) by epigenetic editing.

Suitably, the use may enhance at least one target gene.

In another aspect, the present invention provides a method of repressing transcription and/or expression of (e.g., silencing) at least one target gene in a cell comprising the step of administering an ETM (e.g., ETR) according to the present invention, at least one gRNA according to the present invention, a combination according to the present invention, a polynucleotide according to the present invention, a nucleic acid construct according to the present invention, a vector according to the present invention or a kit of polynucleotides according to the present invention to a cell.

Suitably, transcription and/or expression of at least two target genes may be repressed (e.g., silenced), wherein at least one of the at least two target genes is epigenetically repressed (e.g., silenced) and at least one of the at least two target genes is repressed (e.g., silenced) by gene editing, wherein at least one ETM (e.g., ETR) and at least two gRNAs are administered to said cell simultaneously, sequentially, or separately.

In another aspect, the present invention provides the products, ETMs (e.g., ETRs), gRNAs, combinations, polynucleotides, nucleic acid constructs, vectors, kits of polynucleotides, cells, and pharmaceutical compositions of the present invention for use in therapy.

The use in therapy may, for example, be a use for the preparation of “universally” allogeneic transplantable cells (e.g., by the silencing of β2-microglobulin, B2M). This use may, for example, be applied to the preparation of haematopoietic stem and/or progenitor cells (HSPCs), whole organ transplantation and cancer immunotherapy.

The ETM (e.g., ETR) (or polynucleotide, nucleic acid construct, or vector encoding therefor) and gRNAs may be administered simultaneously, in combination, sequentially or separately (as part of a dosing regimen).

By “simultaneously”, it is to be understood that the two or more agents are administered concurrently, whereas the term “in combination” is used to mean they are administered, if not simultaneously, then “sequentially” within a time frame that they both are available to act therapeutically within the same time frame. Thus, administration “sequentially” may permit one agent to be administered within 5 minutes, 10 minutes, or a matter of hours after the other provided the circulatory half-life of the first administered agent is such that they are both concurrently present in therapeutically effective amounts. The time delay between administration of the components will vary depending on the exact nature of the components, the interaction there-between, and their respective half-lives.

In contrast to “in combination” or “sequentially”, “separately” is to be understood as meaning that the gap between administering one agent and the other agent is significant, i.e., the first administered agent may no longer be present in the bloodstream in a therapeutically effective amount when the second agent is administered.

In another aspect, the present invention provides a method for treating and/or preventing a disease or condition, which comprises the step of administering any of the products of the invention (e.g., ETMs (e.g., ETRs), gRNAs, combinations, polynucleotides, nucleic acid constructs, vectors, kits of polynucleotides, cells, or pharmaceutical compositions according to the present invention) to a subject in need thereof.

Suitably, the ETM (e.g., ETR) and gRNAs may be administered to a subject simultaneously, sequentially, or separately.

In one aspect, the present invention provides a method of gene therapy which comprises the steps of:
(i) isolation of a cell containing sample;
(ii) introduction of a polynucleotide according to the present invention, a nucleic acid construct according to the present invention, at least one gRNA according to the present invention, an ETM (e.g., ETR) according to the present invention, a vector according to the present invention or a kit of polynucleotides according to the present invention to the cell(s); and
(iii) administering the cell(s) from step (ii) to a subject.

The nucleic acid construct or vector may be introduced by transduction or transfection.

The cell may, for example, be autologous. The cell may, for example, be allogeneic.

It is to be appreciated that all references herein to treatment include curative, palliative and prophylactic treatment; although in the context of the present invention references to preventing are more commonly associated with prophylactic treatment. The treatment of mammals, particularly humans, is preferred. Both human and veterinary treatments are within the scope of the present invention.

### Diseases and Conditions

By way of example, the products, ETMs (e.g., ETRs), polynucleotides and cells of the present invention may be used in the treatment of, for example, Huntington's disease, spinocerebellar ataxias, collagenopathies, haemaglobinopathies, and diseases caused by trinucleotide expansions. Furthermore, the product of the present invention may be used in the treatment or prevention of certain infectious diseases (e.g., CCR5-tropic HIV infections) by inactivating either pathogen-associated gene products or host genes that are necessary for the pathogen life cycle.

In addition, or in the alternative, the products, ETMs (e.g., ETRs), polynucleotides and cells of the present invention may be useful in the treatment of the disorders listed in WO 1998/005635. For ease of reference, part of that list is now provided: cancer, inflammation or inflammatory disease, dermatological disorders, fever, cardiovascular effects, haemorrhage, coagulation and acute phase response, cachexia, anorexia, acute infection, HIV infection, shock states, graft-versus-host reactions, autoimmune disease, reperfusion injury, meningitis, migraine and aspirin-dependent anti-thrombosis; tumour growth, invasion and spread, angiogenesis, metastases, malignant, ascites and malignant pleural effusion; cerebral ischaemia, ischaemic heart disease, osteoarthritis, rheumatoid arthritis, osteoporosis, asthma, multiple sclerosis, neurodegeneration, Alzheimer's disease, atherosclerosis, stroke, vasculitis, Crohn's disease and ulcerative colitis; periodontitis, gingivitis; psoriasis, atopic dermatitis, chronic ulcers, epidermolysis bullosa; corneal ulceration, retinopathy and surgical wound healing; rhinitis, allergic conjunctivitis, eczema, anaphylaxis; restenosis, congestive heart failure, endometriosis, atherosclerosis or endosclerosis.

In addition, or in the alternative, the products, ETMs (e.g., ETRs), polynucleotides and cells of the present invention may be useful in the treatment of the disorders listed in WO 1998/007859. For ease of reference, part of that list is now provided: cytokine and cell proliferation/differentiation activity; immunosuppressant or immunostimulant activity (e.g., for treating immune deficiency, including infection with human immune deficiency virus; regulation of lymphocyte growth; treating cancer and many autoimmune diseases, and to prevent transplant rejection or induce tumour immunity); regulation of haematopoiesis, e.g., treatment of myeloid or lymphoid diseases; promoting growth of bone, cartilage, tendon, ligament and nerve tissue, e.g., for healing wounds, treatment of burns, ulcers and periodontal disease and neurodegeneration; inhibition or activation of follicle-stimulating hormone (modulation of fertility); chemotactic/chemokinetic activity (e.g., for mobilising specific cell types to sites of injury or infection); haemostatic and thrombolytic activity (e.g., for treating haemophilia and stroke); anti-inflammatory activity (for treating e.g., septic shock or Crohn's disease); as antimicrobials; modulators of e.g., metabolism or behaviour; as analgesics; treating specific deficiency disorders; in treatment of e.g., psoriasis, in human or veterinary medicine.

In addition, or in the alternative, the products, ETMs (e.g., ETRs), polynucleotides and cells of the present invention may be useful in the treatment of the disorders listed in WO 1998/009985. For ease of reference, part of that list is now provided: macrophage inhibitory and/or T cell inhibitory activity and thus, anti-inflammatory activity; anti-immune activity, i.e., inhibitory effects against a cellular and/or humoral immune response, including a response not associated with inflammation; inhibit the ability of macrophages and T cells to adhere to extracellular matrix components and fibronectin, as well as up-regulated fas receptor expression in T cells; inhibit unwanted immune reaction and inflammation including arthritis, including rheumatoid arthritis, inflammation associated with hypersensitivity, allergic reactions, asthma, systemic lupus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynaecological diseases, posterior uveitis, intermediate uveitis, anterior uveitis, conjunctivitis, chorioretinitis, uveoretinitis, optic neuritis, intraocular inflammation, e.g., retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g., following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of stokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, myasthenia gravis, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery, bone marrow transplantation or other transplantation complications and/or side effects, inflammatory and/or immune complications and side effects of gene therapy, e.g., due to infection with a viral carrier, or inflammation associated with AIDS, to suppress or inhibit a humoral and/or cellular immune response, to treat or ameliorate monocyte or leukocyte proliferative diseases, e.g., leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue.

For example, the present invention may be used to treat inherited disease such as β-haemoglobinopathies by targeting hemoglobin F (HBF) or haemoglobin subunit beta (HBB); or to treat severe combined immunodeficiency disease (SCID), Wiskott-Aldrich syndrome protein (WASP), sickle cell disease (SCD) or adenosine deaminase deficiency (ADA).

The skilled person will understand that they can combine any or all features of the invention disclosed herein without departing from the scope of the invention as disclosed.

### Further Aspects

The present invention also provides further aspects as defined in the following numbered paragraphs.
1. An engineered transcriptional modulator (ETM) comprising: (a) at least one epigenetic effector domain; operably linked to (b) an endonuclease.
2. An ETM according to paragraph 1, wherein the at least one epigenetic effector domain comprises a Kruppel-associated box (KRAB) domain, a DNA methyltransferase (DNMT) domain, a DNMT-like domain, and/or a histone methyltransferase (HMT) domain.
3. An ETM according to paragraph 1 or paragraph 2, wherein the at least one epigenetic effector domain is selected from the group consisting of: DNMT1, DNMT3A, DNMT3B, DNMT3L and SETDB1.
4. An ETM according to any preceding paragraph, wherein the endonuclease comprises an RNA binding domain.
5. An ETM according to any preceding paragraph, wherein the endonuclease is a Clustered Regularly Interspaced Short Palindromic Repeats (CRISPR)/Cas system.
6. An ETM according to any preceding paragraph, wherein the endonuclease is a Cas9 endonuclease.
7. An ETM according to any preceding paragraph, wherein the ETM comprises or consists of: a Cas9-KRAB, Cas9-DNMT3A or Cas9-DNMT3L fusion protein.
8. An ETM according to any preceding paragraph, wherein the ETM is bi- or tri-partite fusion protein.
9. A gRNA comprising a spacer sequence which comprises or consists of the sequence of any one of SEQ ID NOs: 23-46, 562-1076, 2778-4478, and 4553-4565, or a fragment thereof.
10. A combination comprising an ETM according to any one of paragraphs 1-8, and at least one guide RNA (gRNA).
11. A combination according to paragraph 10, which comprises one or more ETMs, wherein each ETM is a fusion protein comprising a catalytically active CRISPR/Cas endonuclease domain.
12. A combination according to paragraph 10 or paragraph 11, which comprises one to three ETMs.
13. A combination according to any one of paragraphs 10-12, wherein at least one epigenetic effector domain is a transcriptional repressor domain, and/or wherein at least one epigenetic effector domain is a DNMT3L domain.
14. A combination according to any one of paragraphs 10-13, wherein the one or more ETMs collectively comprise a transcriptional repressor domain and a DNMT3L domain.
15. A combination according to any one of paragraphs 10-14, which comprises at least two gRNAs.
16. A combination according to paragraph 15, wherein the gRNAs target the ETM to at least two different target genes.
17. A combination according to paragraph 15 or paragraph 16, wherein the at least two gRNAs comprise spacer sequences which are of different lengths.
18. A combination according to any one of paragraphs 10-13, wherein at least one gRNA comprises a spacer sequence which is 15, 16, 17, 18, 19 or 20 nucleotides in length.
19. A combination according to any one of paragraphs 15-18, wherein one of the at least two gRNAs comprises a spacer sequence which is less than or equal to 17 (e.g., less than or equal to 16) nucleotides in length.
20. A combination according to any one of paragraphs 10-19, wherein the at least one target gene is selected from: genes without CpG Islands (CGI), such as: TRAC; TRBC; PDCD1; TIM-3; TIGIT; LAG3; CTLA4; AAVS1 and CCR5; and/or genes having CGI, such as: B2M; TET2; TGFBR2; A2AR; CISH; PTPN11; PTPN6; PTPA; PTPN2; JUNB; TOX; TOX2; NR4A1; NR4A2; NR4A3; MAP4K1; REL; IRF4; DGKA; PIK3CD; HLA-A; USP16; DCK and FAS.
21. A combination according to any one of paragraphs 10-20, which comprises: one or more guide RNAs (gRNAs) having a spacer sequence with a length that allows epigenetic editing and not gene editing of a first gene in the cell, optionally wherein the first gene comprises a CpG island (CGI); and one or more gRNAs having a spacer sequence with a length that allows gene editing of a second gene in the cell.
22. A combination according to paragraph 21, wherein the one or more guide RNAs (gRNAs) having a spacer sequence with a length that allows epigenetic editing and not gene editing of a first gene in the cell has a spacer sequence of:
   (a) less than or equal to 17 nucleotides (e.g., less than or equal to 16 nucleotides); or
   (b) 11 to 17 nucleotides (e.g., 11 to 16 nucleotides).
23. A combination according to paragraph 21 or paragraph 22, wherein the one or more gRNAs having a spacer sequence with a length that allows gene editing of a second gene in the cell has a spacer sequence of:
   (a) 17 or more nucleotides (e.g., 18 or more nucleotides); or
   (b) 17 to 30 nucleotides, optionally 18 to 25 nucleotides (e.g., 18 to 21 nucleotides).
24. A combination comprising one or more polynucleotides coding for the ETM(s) (e.g., fusion proteins) and/or gRNAs as defined in any one of paragraphs 10-23.
25. A combination according to any one of paragraphs 21-24, further comprising a donor DNA comprising 5′ and 3′ arms that are homologous to sequences in the second gene.
26. A combination according to any one of paragraphs 10-25, wherein the endonuclease domain is derived from a Cas9 protein, optionally SpCas9.
27. A combination according to any one of paragraphs 21-26, wherein
   the first gene is selected from B2M, TET2, TGFBR2, A2AR, CISH, PTPN11, PTPN6, PTPA, PTPN2, JUNB, TOX, TOX2, NR4A1, NR4A2, NR4A3, MAP4K1, REL, IRF4, DGKA, PIK3CD, HLA-A, USP16, DCK, and FAS; and/or
   the second gene is selected from TRAC, TRBC, PDCD1, TIM-3, TIGIT, LAG3, CTLA4, AAVS1, and CCR5.
28. A combination according to any one of paragraphs 21-27, wherein the second gene is a TRAC gene, optionally wherein the one or more gRNAs targeting the TRAC gene comprise a spacer having the sequence of one of SEQ ID NOs: 562-611, optionally SEQ ID NO: 604.
29. A combination according to any one of paragraphs 21-28, wherein the first gene is a B2M gene, optionally wherein the one or more gRNAs targeting the B2M gene each comprise a spacer having the sequence of one of SEQ ID NOs: 28-33 and 39-44; or the sequence of one of SEQ ID NOs: 2778-2878 with a 3 to 9 nucleotide truncation at the 5′ end, optionally one of SEQ ID NOs: 2778, 2780, 2801, and 2863 with a 3 to 9 nucleotide truncation at the 5′ end, selected from SEQ ID NOs: 4486-4492, 4497-4503, 4508-4514, and 4519-4525.
30. A combination according to any one of paragraphs 21-28, wherein the first gene is a TGFBR2 gene, optionally wherein the one or more gRNAs targeting the TGFBR2 gene each comprise a spacer having the sequence of one of SEQ ID NOs: 2929-2978 and 4553-4559 with a 3 to 9 nucleotide truncation at the 5′ end.
31. A combination according to any one of paragraphs 21-28, wherein the first gene is a TET2 gene, optionally wherein the one or more gRNAs targeting the TET2 gene each comprise a spacer having the sequence of one of SEQ ID NOs: 4429-4478 and 4560-4565 with a 3 to 9 nucleotide truncation at the 5′ end.
32. A combination according to any one of paragraphs 10-31 for modifying transcription, expression and/or activity of one or more (e.g. two or more) gene in a cell, wherein the cell is a mammalian cell, optionally a human cell, optionally wherein the cell is a human immune cell, or a human T cell.
33. A combination according to any one of paragraphs 10 to 32, further comprising an agent:
   i) which promotes the survival, proliferation and/or activity of a cell, such as a cell which comprises the combination or a cell which does not comprise the combination; and/or
   ii) which is detrimental to the survival, proliferation, activity, chemoresistance and/or chemotaxis of a cell, such as a cell which comprises the combination or a cell which does not comprise the combination and/or
   iii) which enables selection of a cell, such as a cell which comprises the combination or a cell which does not comprise the combination.
34. A combination according to any one of paragraphs 10 to 33, comprising at least one gRNA according to paragraph 9.
35. The combination of any one of paragraphs 20-34, wherein the gene comprising a CGI is a B2M gene and the gRNAs targeting it are two or three gRNAs each independently comprising a spacer having the sequence of
   C8 (SEQ ID NO: 35),
   F4 (SEQ ID NO: 24),
   H8 (SEQ ID NO: 2780),
   H10 (SEQ ID NO: 2863),
   H11 (SEQ ID NO: 2778), or
   H12 (SEQ ID NO: 2801),
optionally with a 1 to 9, optionally 3 to 9, nucleotide truncation at the 5′ end.
36. The combination of paragraph 35, wherein the B2M-targeting gRNAs comprise
(i) a gRNA comprising a spacer having the sequence of F4 optionally with a 1 to 9, optionally 3 to 9, nucleotide truncation at the 5′ end,
   a gRNA comprising a spacer having the sequence of H8 optionally with a 1 to 9, optionally 3 to 9, nucleotide truncation at the 5′ end, and
   a gRNA comprising a spacer having the sequence of H10 optionally with a 1 to 9, optionally 3 to 9, nucleotide truncation at the 5′ end;
(ii) a gRNA comprising a spacer having the sequence of C8 optionally with a 1 to 9, optionally 3 to 9, nucleotide truncation at the 5′ end,
   a gRNA comprising a spacer having the sequence of H8 optionally with a 1 to 9, optionally 3 to 9, nucleotide truncation at the 5′ end, and
   a gRNA comprising a spacer having the sequence of H10 optionally with a 1 to 9, optionally 3 to 9, nucleotide truncation at the 5′ end;
(iii) a gRNA comprising a spacer having the sequence of F4 optionally with a 1 to 9, optionally 3 to 9, nucleotide truncation at the 5′ end, and
   a gRNA comprising a spacer having the sequence of H8 optionally with a 1 to 9, optionally 3 to 9, nucleotide truncation at the 5′ end;
(iv) a gRNA comprising a spacer having the sequence of F4 optionally with a 1 to 9, optionally 3 to 9, nucleotide truncation at the 5′ end, and
   a gRNA comprising a spacer having the sequence of H10 optionally with a 1 to 9, optionally 3 to 9, nucleotide truncation at the 5′ end; or
(v) a gRNA comprising a spacer having the sequence of H8 optionally with a 1 to 9, optionally 3 to 9, nucleotide truncation at the 5′ end, and
   a gRNA comprising a spacer having the sequence of H10 optionally with a 1 to 9, optionally 3 to 9, nucleotide truncation at the 5′ end.
37. The combination of any one of paragraphs 21-36, wherein the ETM(s) (e.g., one or more fusion proteins) collectively further comprise a DNMT1, DNMT3A, DNMT3B, or SETDB1 domain, optionally DNMT3A.
38. The combination of any one of paragraphs 10-37, wherein the combination comprises
   (i) a first fusion protein comprising a transcriptional repressor domain and a Cas endonuclease domain, and a second fusion protein comprising a DNMT3L domain and a Cas endonuclease domain, or
   (ii) a fusion protein comprising, optionally from N-terminus to C-terminus, a transcriptional repressor domain, a Cas endonuclease domain, and a DNMT3L domain.
39. The combination of any one of paragraphs 10-37, wherein the combination comprises
   (i) a first fusion protein comprising a transcriptional repressor domain and a Cas endonuclease domain, a second fusion protein comprising a DNMT3L domain and a Cas endonuclease domain, and a third fusion protein comprising a DNMT3A domain and a Cas endonuclease domain, or
   (ii) a fusion protein comprising a transcriptional repressor domain, a Cas endonuclease domain, a DNMT3L domain, and a DNMT3A domain.
40. The combination of any one of paragraphs 10-39, wherein the epigenetic effector domain (e.g. transcriptional repressor domain) is a Krüppel-associated box (KRAB) domain, optionally derived from human Kox1 or ZIM3.
41. The combination of any one of paragraphs 10-40, wherein the combination comprises a fusion protein comprising, optionally from N terminus to C terminus, a KRAB domain derived from ZIM3, a catalytically active Cas9 domain, and a DNMT3L domain, optionally comprising an amino acid sequence of SEQ ID NO: 4482.
42. The combination of any one of paragraphs 10-41, further comprising gRNAs for targeting one or more additional genes in the cell.
43. The combination of any one of paragraphs 10-42, wherein the gRNA(s) are chemically modified, optionally wherein the chemically modified gRNA(s) comprise phosphorothioate internucleoside linkages at the 5′ and/or 3′ ends, and/or 2′-O-methyl nucleotides.
44. A polynucleotide encoding at least one ETM according to any one of paragraphs 1 to 8 or as defined in any one of paragraphs 10-43.
45. A nucleic acid construct comprising a nucleic acid sequence encoding at least one ETM according to any one of paragraphs 1 to 8 or as defined in any one of paragraphs 10-43.
46. A nucleic acid construct according to paragraph 45, further comprising a nucleic acid sequence:
   i) which promotes the survival, proliferation and/or activity of a cell, such as a cell which expresses said nucleic acid construct or a cell which does not express said nucleic acid construct; and/or
   ii) which is detrimental to the survival, proliferation, activity, chemoresistance and/or chemotaxis of a cell, such as a cell which expresses said nucleic acid construct or a cell which does not express said nucleic acid construct; and/or
   iii) which enables selection of a cell, such as a cell which comprises the nucleic acid construct or a cell which does not comprise the construct.
47. A vector comprising a polynucleotide according to paragraph 44 or a nucleic acid construct according to paragraph 45 or 46.
48. A kit of polynucleotides comprising:
   a) at least one polynucleotide encoding at least one ETM according to any one of paragraphs 1 to 8 or as defined in any one of paragraphs 10-43; and
   b) a polynucleotide providing at least one gRNA as described in any one of paragraphs 9 or 10 to 32 or 35 to 43; and optionally,
   c) a further polynucleotide comprising a nucleic acid sequence which encodes an agent:
      i) which promotes the survival, proliferation and/or activity of a cell, such as a cell which comprises the polynucleotides or a cell which does not comprise the polynucleotides; and/or
      ii) which is detrimental to the survival, proliferation, activity, chemoresistance and/or chemotaxis of a cell, such as a cell which comprises said polynucleotides or a cell which does not comprise said polynucleotides; and/or
      iii) which enables selection of a cell, such as a cell which comprises the polynucleotides or a cell which does not comprise the polynucleotides.
49. A cell comprising an ETM according to any one of paragraphs 1 to 8, at least one gRNA according to paragraph 9, a combination according to any one of paragraphs 10 to 43, a polynucleotide according to paragraph 44, a nucleic acid construct according to paragraph 45 or paragraph 46, a vector according to paragraph 47 or a kit of polynucleotides according to paragraph 48.
50. A cell wherein the cell is a progeny of the cell of paragraph 49.
51. A composition comprising an ETM according to any one of paragraphs 1 to 8, at least one gRNA according to paragraph 9, a combination according to any one of paragraphs 10 to 43, a polynucleotide according to paragraph 44, a nucleic acid construct according to paragraph 45 or paragraph 46, a vector according to paragraph 47, a kit of polynucleotides according to paragraph 48 or a cell according to paragraph 49 or paragraph 50.
52. A pharmaceutical composition comprising an ETM according to any one of paragraphs 1 to 8, at least one gRNA according to paragraph 9, a combination according to any one of paragraphs 10 to 43, a polynucleotide according to paragraph 44, a nucleic acid construct according to paragraph 45 or paragraph 46, a vector according to paragraph 47, a kit of polynucleotides according to paragraph 48 or a cell according to paragraph 49 or paragraph 50.
53. Use of an ETM according to any one of paragraphs 1 to 8, at least one gRNA according to paragraph 9, a combination according to any one of paragraphs 10 to 43, a polynucleotide according to paragraph 44, a nucleic acid construct according to paragraph 45 or paragraph 46, a vector according to paragraph 47, a kit of polynucleotides according to paragraph 48 or a cell according to paragraph 49 or paragraph 50 for modifying the transcription, expression and/or activity of (e.g. repressing or silencing) at least one target gene in a cell.
54. A method of modifying the transcription, expression and/or activity of (e.g. repressing or silencing) at least one target gene in a cell comprising the step of administering an ETM according to any one of paragraphs 1 to 8, at least one gRNA according to paragraph 9, a combination according to any one of paragraphs 10 to 43, a polynucleotide according to paragraph 44, a nucleic acid construct according to paragraph 45 or paragraph 46, a vector according to paragraph 47 or a kit of polynucleotides according to paragraph 48 to a cell.
55. The use or method of paragraph 53 or 54, wherein the cell is a T cell.
56. The use or method of any one or paragraphs 53-55, wherein the ETM, at least one gRNA, combination, polynucleotide, nucleic acid construct, vector or a kit of polynucleotides is introduced into the cell in vitro or ex vivo.
57. A method according to any one of paragraphs 54-56, wherein at least two target genes are silenced, wherein at least one of the at least two target genes is epigenetically silenced and at least one of the at least two target genes is silenced by gene editing, wherein at least one ETM and at least two gRNAs are administered to said cell simultaneously, sequentially or separately.
58. A cell obtained by the use or method of any one of paragraphs 53-57, or a progeny of the cell.
59. The cell of any one of paragraphs 49, 50 or 58, wherein the cell is a human T cell, optionally engineered to express a recombinant antigen receptor, optionally selected from a recombinant T cell receptor (TCR) or a chimeric antigen receptor (CAR).
60. An ETM according to any one of paragraphs 1 to 8, at least one gRNA according to paragraph 9, a combination according to any one of paragraphs 10 to 43, a polynucleotide according to paragraph 44, a nucleic acid construct according to paragraph 45 or paragraph 46, a vector according to paragraph 47, a kit of polynucleotides according to paragraph 48, a cell according to paragraph 49, 50, 58 or 59 or a pharmaceutical composition according to paragraph 52 for use in therapy (e.g. for use in treating a human in need thereof).
61. Use of an ETM according to any one of paragraphs 1 to 8, at least one gRNA according to paragraph 9, a combination according to any one of paragraphs 10 to 43, a polynucleotide according to paragraph 44, a nucleic acid construct according to paragraph 45 or paragraph 46, a vector according to paragraph 47, a kit of polynucleotides according to paragraph 48, a cell according to paragraph 49, 50, 58 or 59 or a pharmaceutical composition according to paragraph 52 in the manufacture of medicament for treating a human in need thereof.
62. An ETM, combination, polynucleotide, nucleic acid construct, vector, kit of polynucleotides, cell or pharmaceutical composition for use according to paragraph 60, or the use of paragraph 61, wherein at least one ETM (e.g. fusion protein) and at least two gRNAs are administered to a cell or subject simultaneously, sequentially or separately.
63. A method for treating and/or preventing a disease (e.g. in a human in need thereof), which comprises the step of administering an ETM according to any one of paragraphs 1 to 8, at least one gRNA according to paragraph 9, a combination according to any one of paragraphs 10 to 43, a polynucleotide according to paragraph 44, a nucleic acid construct according to paragraph 45 or paragraph 46, a vector according to paragraph 47, a kit of polynucleotides according to paragraph 48, a cell according to paragraph 49, 50, 58 or 59 or a pharmaceutical composition according to paragraph 52 to a subject in need thereof.
64. A method for treating and/or preventing a disease according to paragraph 63, wherein at least one ETM (e.g. fusion protein) and at least two gRNAs are administered to a cell or subject simultaneously, sequentially or separately.
65. A method of gene therapy which comprises the steps:
   (i) isolation of a cell containing sample,
   (ii) introduction of an ETM according to any one of paragraphs 1 to 8, at least one gRNA according to paragraph 9, the combination according to any one of paragraphs 10 to 43, the polynucleotide as defined in paragraph 44, the nucleic acid construct according to paragraph 45 or paragraph 46, a vector according to paragraph 47 and/or a kit of polynucleotides according to paragraph 48 to the cell(s); and
   (iii) administering the cell(s) from step (ii) to a subject.
66. The method according to paragraph 65, wherein the polynucleotide, nucleic acid construct and/or vector is introduced by transduction or transfection.
67. An ETM, combination, polynucleotide, nucleic acid construct, vector, kit of polynucleotides, cell or pharmaceutical composition for use according to paragraph 60 or 62, the use of paragraph 61 or 62, or the method according to any one of paragraphs 63-66, wherein the cell is autologous.
68. An ETM, combination, polynucleotide, nucleic acid construct, vector, kit of polynucleotides, cell or pharmaceutical composition for use according to paragraph 60 or 62, the use of paragraph 61 or 62, or the method according to any one of paragraphs 63-66, wherein the cell is allogeneic.

Unless otherwise defined herein, scientific, and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of, medicine, medicinal and pharmaceutical chemistry, and cell biology described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. It should also be noted that the term “or” is generally employed in its sense including “and/or” unless the content clearly dictates otherwise. As used herein the term “about” refers to a numerical range that is 10%, 5%, or 1% plus or minus from a stated numerical value within the context of the particular usage. Further, headings provided herein are for convenience only and do not interpret the scope or meaning of the claimed embodiments.

The practice of the present invention will employ, unless otherwise indicated, conventional techniques of chemistry, biochemistry, molecular biology, microbiology, and immunology, which are within the capabilities of a person of ordinary skill in the art. Such techniques are explained in the literature. See, for example, Sambrook, J., Fritsch, E. F. and Maniatis, T. (1989) Molecular Cloning: A Laboratory Manual, 2nd Edition, Cold Spring Harbor Laboratory Press; Ausubel, F. M. et al. (1995 and periodic supplements) Current Protocols in Molecular Biology, Ch. 9, 13 and 16, John Wiley & Sons; Roe, B., Crabtree, J. and Kahn, A. (1996) DNA Isolation and Sequencing: Essential Techniques, John Wiley & Sons; Polak, J. M. and McGee, J.O'D. (1990) In Situ Hybridization: Principles and Practice, Oxford University Press; Gait, M. J. (1984) Oligonucleotide Synthesis: A Practical Approach, IRL Press; and Lilley, D. M. and Dahlberg, J. E. (1992) Methods in Enzymology: DNA Structures Part A: Synthesis and Physical Analysis of DNA, Academic Press.

All publications and other references mentioned herein are incorporated by reference in their entirety. Although a number of documents are cited herein, this citation does not constitute an admission that any of these documents forms part of the common general knowledge in the art.

In order that this invention may be better understood, the following examples are set forth. These examples are for purposes of illustration only and are not to be construed as limiting the scope of the invention in any manner.

### EXAMPLES

### Example 1—gRNAs Comprising Truncated Spacer Sequences Promote Epigenetic Silencing without Causing Mutagenesis

To assess the feasibility of using gRNAs comprising truncated spacer sequences to promote ETR-mediated epi-silencing of B2M while sparing the gene from mutagenesis, we first designed a 20 nt-long gRNA against B2M (named F4; SEQ ID NO: 24) and a corresponding panel of 5′-truncated B2M gRNAs with spacer sequences of different lengths, and then we tested them in the B2M^{tdTomato} K-562 cell line (Amabile et al., supra).

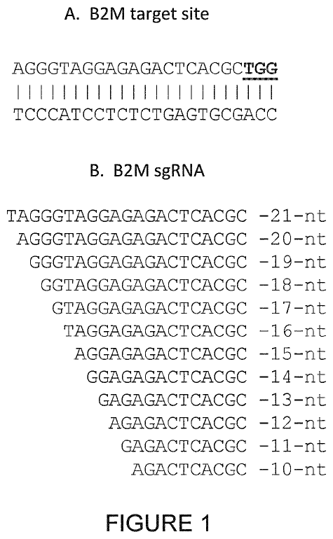

In particular, the gRNAs comprising spacer sequences spanning from 21 to 10 nt in length and comprising the same seed and PAM sequence (FIG. 1) were individually delivered into the cells together with either Cas9 or the dCas9-based ETR combination, the latter containing KRAB, DNMT3A or DNMT3L. The cells were then analysed for genetic traces of Cas9 activity at the B2M gene or expression of tdTomato, the latter used as a proxy for B2M epigenetic silencing.

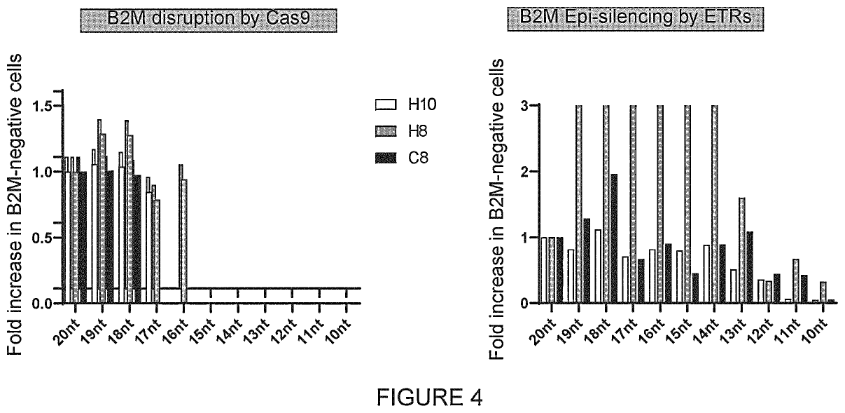

gRNAs comprising the standard 20 nt-long B2M spacer sequence plus Cas9 or dCas9-ETRs were included as positive controls for gene disruption or epigenetic silencing, respectively. Molecular analyses of the B2M target site in Cas9-treated cells showed a threshold effect: gRNAs comprising a spacer sequence of ≥17 nt in length mediated high and comparable levels of B2M editing (˜30%) while gRNAs comprising a spacer sequence ≤16 nt resulted in undetectable gene editing (FIG. 2). Flow cytometry analyses of ETR-treated cells showed a different trend: all gRNAs except the gRNA comprising the 10 nt-long spacer sequence were able to induce efficient epigenetic silencing of B2M, although at different levels (from 30 to 48% of tdTomato-negative cells; FIG. 3). Importantly, the gRNAs comprising truncated spacer sequences that were ineffective in promoting gene editing with Cas9 (i.e., ≤16 nt) were highly effective in mediating epigenetic silencing with the dCas9-ETRs. To assess if these findings were portable to other gRNAs, we performed a similar truncation experiment using three other gRNAs (named H8_20 (spacer SEQ ID NO: 2780; gRNA SEQ ID NO: 4570), C8_20 (spacer SEQ ID NO: 2813; gRNA SEQ ID NO: 4569) and H10_20 (spacer SEQ ID NO: 2863; gRNA SEQ ID NO: 4571) and found that the spacer length at which Cas9 lost its activity depended on the specific gRNA used, ranging between 15 and 17 nt in length (FIG. 4; left panel). In accordance to what we showed above, ETRs were able to induce epi-silencing of B2M even with truncated gRNAs (FIG. 4; right panel).

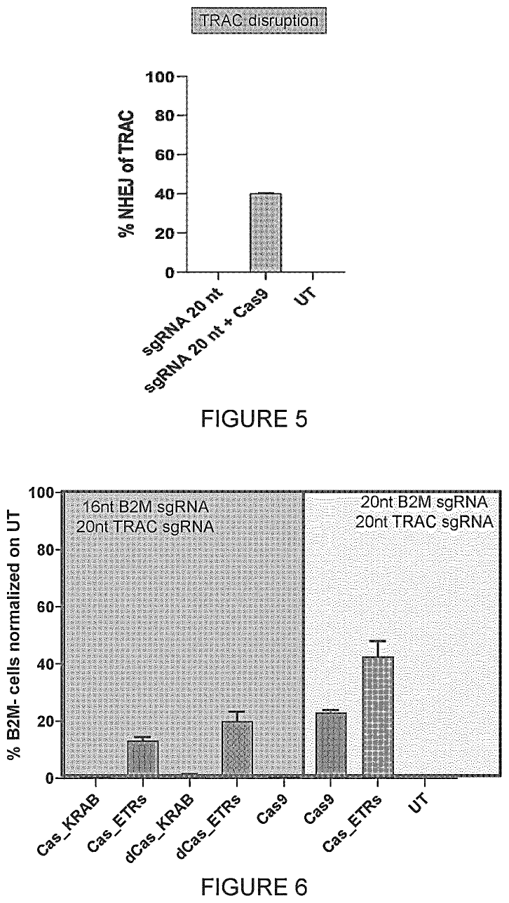

Overall, these data indicate that gRNAs comprising a truncated spacer sequence of ≤17 nt promote epigenetic silencing of B2M while sparing this gene from mutagenesis induced by Cas9-based ETRs. Furthermore, they provide the first demonstration that epi-silencing can be imposed also when using gRNAs comprising truncated spacer sequences. In parallel to these experiments, we also produced a gRNA comprising a 20 nt spacer sequence capable of inducing gene editing at the TRAC locus (FIG. 5).

### Example 2—a Combination of ETR and gRNAs Enables Simultaneous Inactivation of Two Genes without Inducing Chromosomal Translocations

Based on these data, we then constructed ETRs equipped with a catalytically active Cas9 (hereafter referred as to Cas9-ETRs, containing KRAB, DNMT3A or DNMT3L) and assessed their multiplexing efficiency with gRNAs comprising truncated or full-length spacer sequences in the B2M^{dTomato} K-562 cells. In particular, we co-transfected the cells with the triple Cas9-ETR combination plus the F4-derived B2M gRNA comprising 16 nt-long spacer sequences (see FIGS. 2 and 3) and the TRAC gRNA with the 20 nt-long spacer. The following controls were also included in the experiment: (i) cells co-transfected with the just mentioned gRNA combination plus either Cas9 or the standard triple dCas9-ETRs, used as positive control for either genetic disruption of TRAC or epi-silencing of B2M and disruption of TRAC, respectively; (ii) cells co-transfected with two gRNAs comprising 20 nt-long spacer sequences, one against B2M and the other against TRAC, plus either Cas9 or Cas9-ETRs, were used here as positive controls for co-disruption of B2M and TRAC. The latter conditions were also included to assess if simultaneous gene editing of the two loci may lead to reciprocal chromosomal translocations. Upon transfection, the cells were longitudinally monitored by flow cytometry for tdTomato expression for up to 25 days.

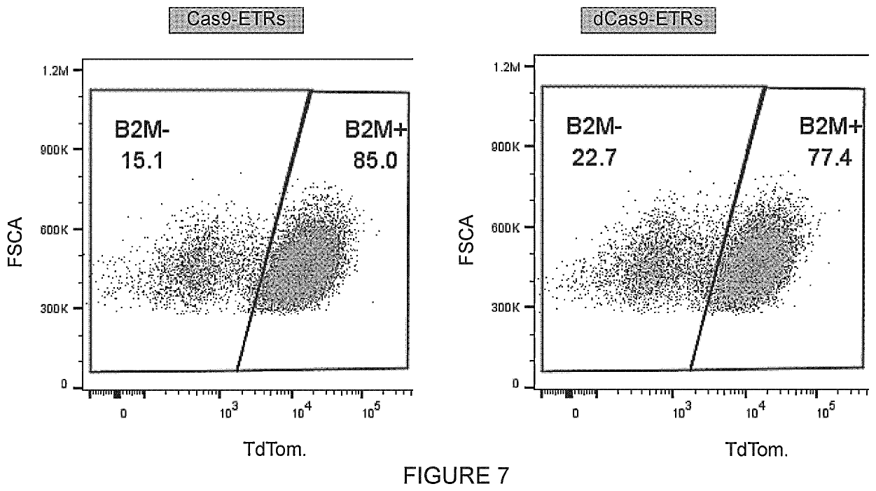

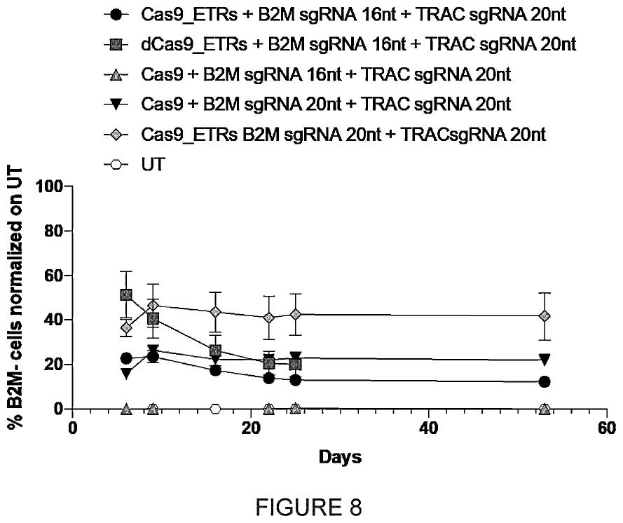

As shown in FIG. 6 and FIG. 7, when delivered with the gRNA comprising a 16 nt spacer sequence, Cas9- and dCas9-based ETRs performed equally in terms of B2M epi-silencing (14 vs. 21%, respectively). Cas9 promoted B2M inactivation only when coupled with the gRNA comprising a 20 nt-long spacer sequence and not with its truncated counterpart (24% vs. 1.7% of tdTomato negative cells), further confirming the results of FIG. 2 and FIG. 3. The use of the gRNA comprising a 20 nt-long B2M spacer sequence with Cas9-ETRs resulted in a percentage of tdTomato negative cells that was higher than that found in all other conditions (up to 44%), a finding expected considering the additive effect of gene and epigenetic editing on this locus. Of note, silencing was stable long-term in all analyzed conditions (FIG. 8), indicating that also the Cas9-ETRs with the gRNA comprising a 16 nt spacer sequence are able to instruct mitotically inherited epigenetic modifications.

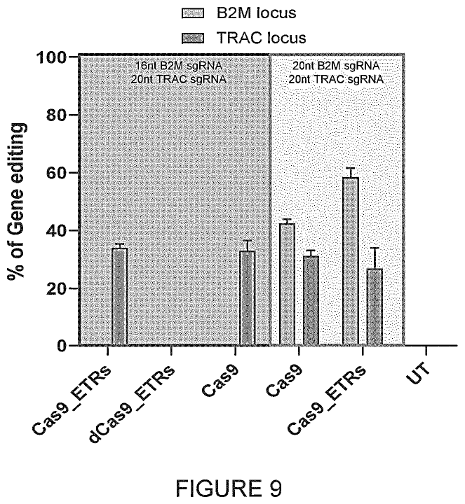

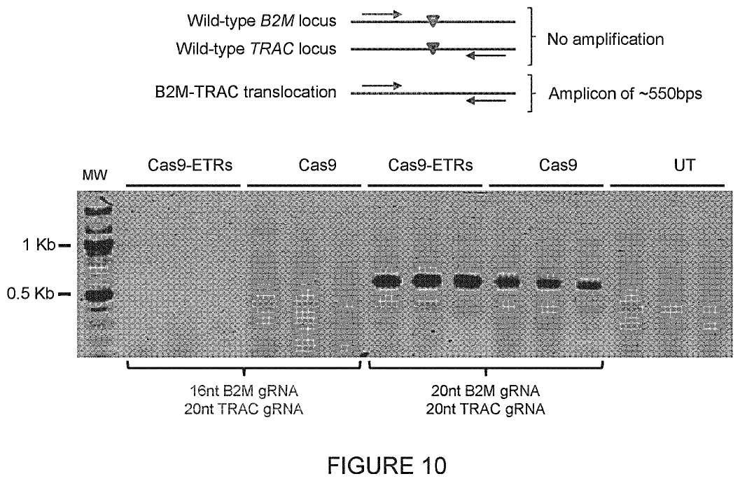

We then analyzed the cells for gene editing (FIG. 9) and found that both Cas9 and Cas9-ETRs induced efficient editing of TRAC (up to 37%). On the other hand, gene editing of B2M was limited to the conditions in which Cas9 or Cas9-ETRs were co-delivered with the gRNA comprising the 20 nt-long B2M spacer sequence. Finally, we performed a PCR analysis with primers specific for reciprocal chromosomal translocations between B2M and TRAC and found occurrence of these events exclusively in the conditions co-treated with the two gRNAs comprising 20 nt-long spacer sequences, but not when the gRNA comprising the 16 nt spacer sequence was used (FIG. 10).

Overall, these data show that Cas9-ETRs perform as their dCas9-based counterparts in terms of silencing efficiency and stability. Yet, adoption of Cas9-ETRs in combination with gRNAs comprising a truncated and a full-length spacer sequence can be safely used to inactivate simultaneously two genes without inducing chromosomal translocations.

### Example 3—Optimization of the B2M Epi-Silencing Procedure in Human Primary T Lymphocytes

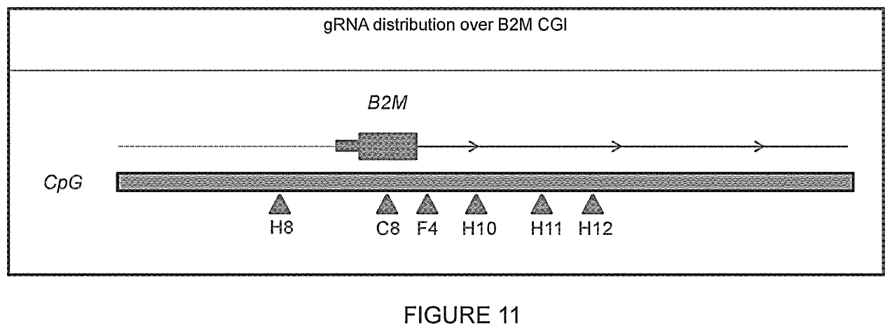

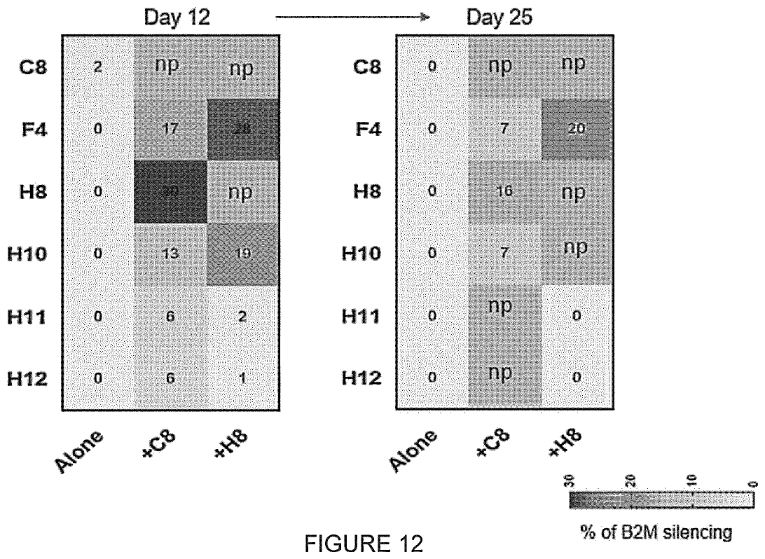

Inactivation of B2M is emerging as a promising approach to generate allogenic T cell products. To assess feasibility of B2M epi-silencing in human primary T cells, we first expanded our repertoire of gRNAs against this gene to include 2 other guides: H11_20 (spacer SEQ ID NO: 2778; gRNA SEQ ID NO: 4572) and H12_20 (spacer SEQ ID NO: 2801; gRNA SEQ ID NO: 4573) (FIG. 11). We then delivered each of these 6 gRNAs with mRNAs encoding for the triple ETR combination in T cells. Time course flow cytometry analyses of treated cells were then used to assess efficiency and stability of B2M epi-silencing. Unexpectedly, at day 12 post-treatment, all but one of the tested gRNAs failed to induce epi-silencing of B2M (FIG. 12). The only working gRNAs (namely gRNA C8) resulted in up to 2% of B2M-negative cells, which, however, were lost upon T cell restimulation (analysis at day 25).

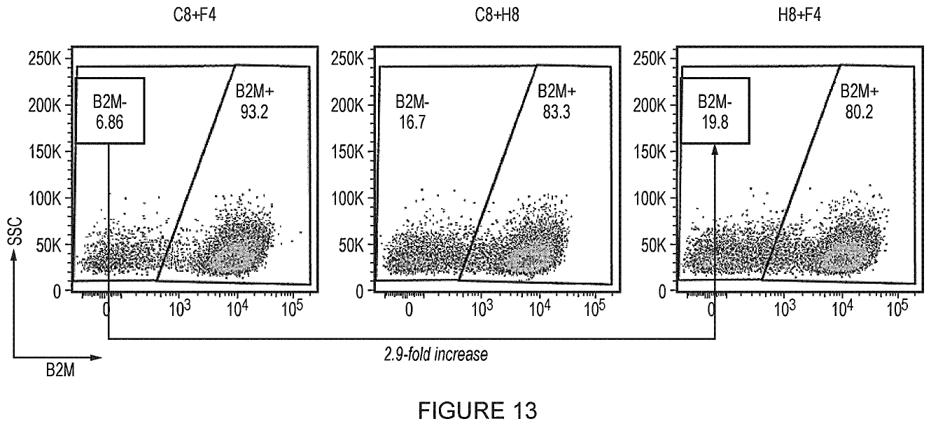

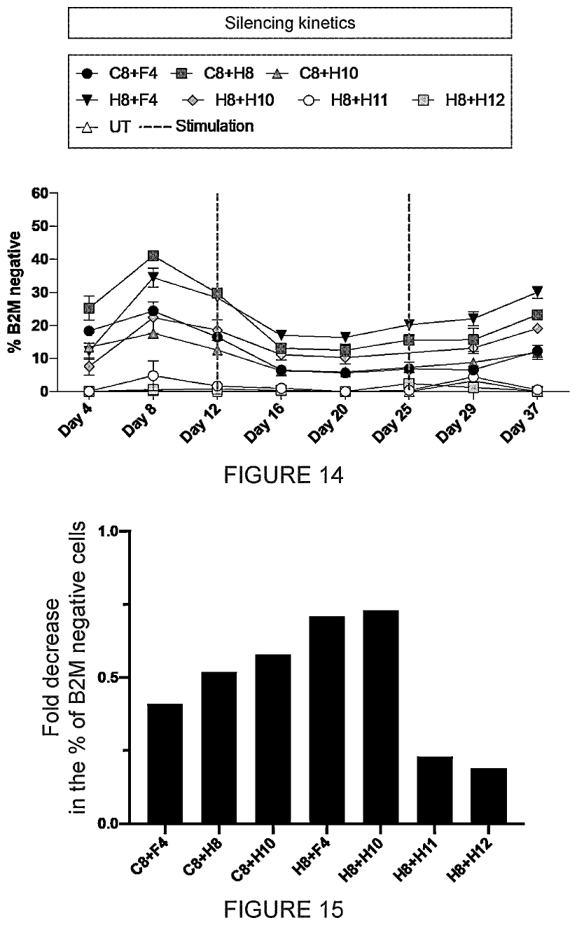

We then tested whether combined delivery of gRNAs would improve epi-silencing efficiency. To this end, we combined either gRNA C8 or H8 with all other gRNAs and delivered these dual gRNA combinations together with the triple ETR combination in T cells. Flow cytometry analyses at day 12 post-treatment revealed that all gRNA combinations were able to induce epi-silencing of B2M, although at different levels (FIG. 12). For instance, gRNA combination H12+H8 induced limited silencing, while the F4+H8 combination resulted in up to 28% of B2M-negative cells. Importantly, for some of these gRNA combinations, epi-silencing resisted the T cell restimulation process, ranging from 11 to 20% of long-term stable B2M-negative cells (FIGS. 12 and 13). Extended time course flow cytometry analyses over a timeframe of 37 days and spanning two rounds of T cell restimulations showed that most of the tested gRNA combinations induced an initial wave of B2M epi-silencing, which then declined after the first T cell restimulation (day 12) to reach near stability until the second round of T cell restimulation (day 25) (FIG. 14). Then, the percentage of B2M increased until termination of the experiment (day 37). Of note, the efficiency of epi-silencing was dependent on the combination of gRNAs used, with H8+F4 being the most effective at long-term (up to 30% of B2M-negative cells) while H8+H11 and H8+H12 resulting in barely detectable, if any, epi-silencing.

Epi-silencing stability was also dependent on gRNA combination (FIG. 15). Indeed, by comparing the percentage of B2M-negative cells between day 25 (just before to the second round of T cell restimulation) and day 12 (just before the first round of T cell restimulation), we found that some gRNA combinations were poorly resistant (fold reduction in B2M-negative cells <0.5) while others were more resistant (fold reduction in B2M-negative cells 0.5), although none of them were able to result in fully stable gene silencing. Among the most stable, combinations H8+F4 and H8+H10 were the best performing ones.

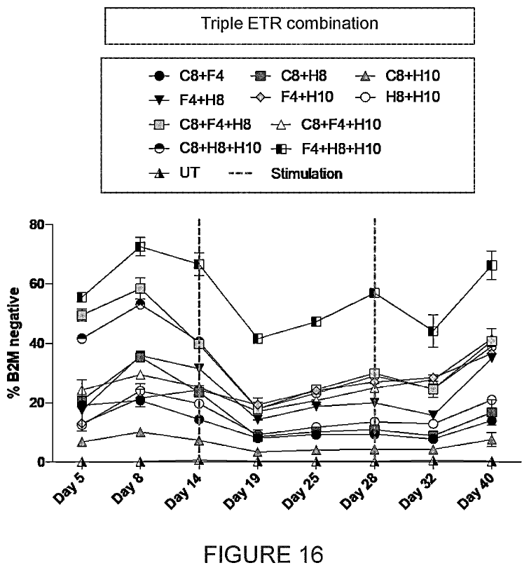

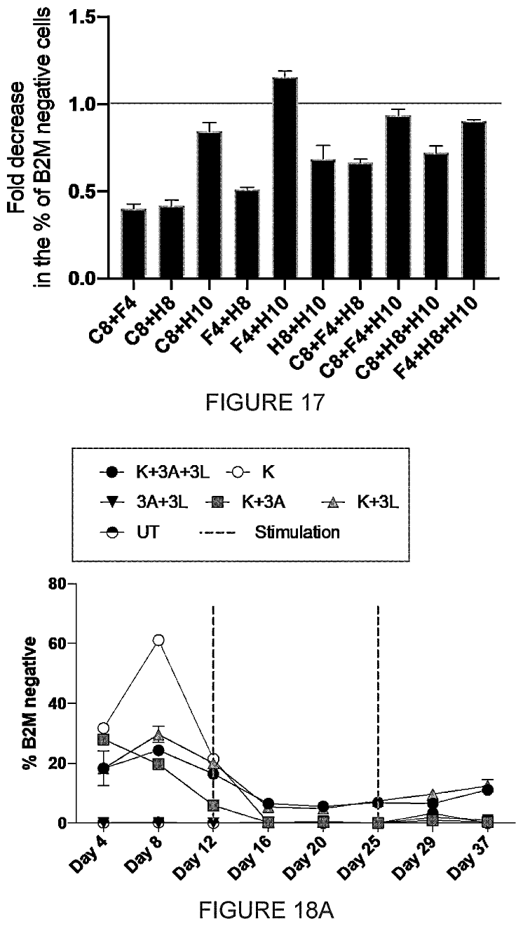

We then performed a similar experiment, in which we excluded the ineffective gRNA combinations H8+H11 and H8+H12 and included the new dual-gRNA combination F4+H10. Furthermore, we also included triple gRNA combinations (namely, C8+F4+H8, C8+F4+H10, C8+H8+H10 and F4+H8+H10) to assess if these were able to further improve epi-silencing efficiency and stability. Among the dual-gRNA combinations tested, the most effective at long-term (day 32) was F4+H10, reaching up to 36.5% of B2M-negative cells. Among the triple gRNA combinations tested, the F4+H8+H10 outperformed the others by 1.6-fold, reaching up to 66% of B2M-negative cells at termination of the experiment (FIG. 16). As observed in the previous experiment, the first round of T cell restimulation caused a marked reduction in the percentage of B2M negative cells for most of the gRNA combinations (FIG. 16). Noticeable exceptions to this were the gRNA combinations containing F4+H10 (including the triple C8+F4+H10 and F4+H8+H10), for which the percentage of B2M-negative cells at day 28 and 14 were nearly superimposable (fold reduction in B2M-negative cells ˜1; FIG. 17). Similar findings were obtained for the dual C8+H10 gRNA combination (FIG. 17). Overall, these data show that epi-silencing efficiency and durability of B2M depends on which gRNA combination is used, with the triple based on the F4+H8+H10 being the best-preforming one.

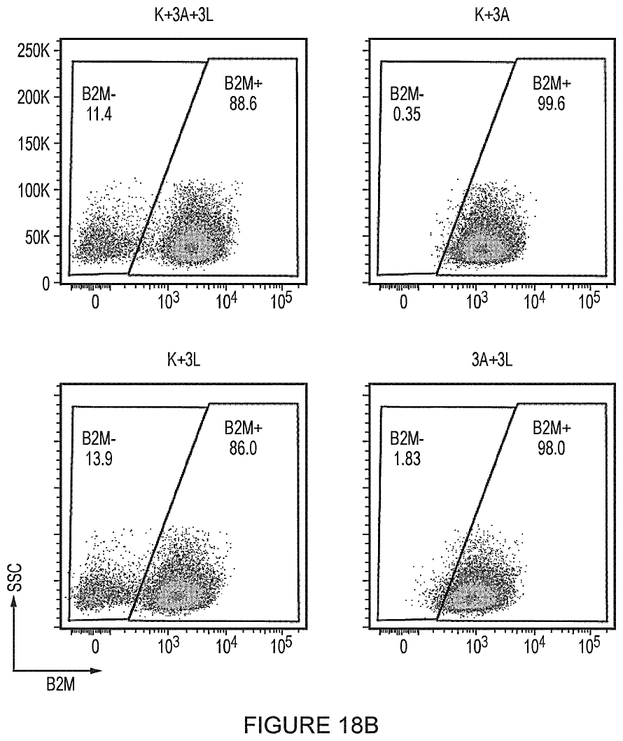

With the aim of reducing the molecular complexity of the technology, we then asked whether all the components of the triple ETR combination were required for epi-silencing of B2M. To this end, we transiently delivered to T cells the dual-gRNA combination containing C8 and F4 together with mRNAs encoding either: (i) the triple ETR combination, taken here as reference for epi-silencing efficiency of B2M; (ii) the double ETR combination containing the KRAB and DNMT3L effector domains; (iii) the double ETR combination containing the DNMT3A and DNMT3L effector domains; or (iv) the double ETR combination containing the KRAB and DNMT3L effector domains. The T cells were then analysed for B2M expression by flow cytometry until day 37 post-treatment (FIGS. 18A and 18B). This experiment showed that, among all the double ETR combinations tested, only the one based on the KRAB and DNMT3L effector domains induced long-term silencing, at efficiencies superimposable to those observed with the triple ETR combination (up to 14% of B2M-negative cells). The double ETR combination based on KRAB and DNMT3A induced only transient B2M repression, which, after the first round of T cell restimulation, returned to the levels observed in untreated T cells. Unexpectedly, the double ETR combination based on DNMT3A and DNMT3L failed to induce any B2M silencing, even at early time points post-treatment.

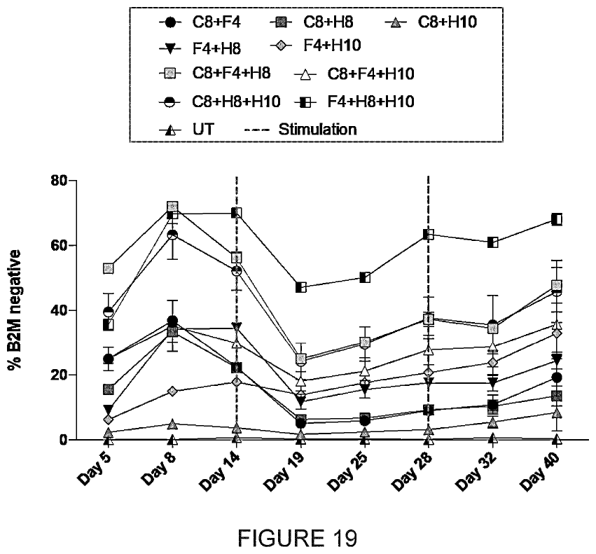

Based on these results, we then performed a similar experiment to that shown in FIG. 16 but using the double ETR combination containing KRAB and DNMT3L, confirming that this combination performed as efficiently as the triple one for all gRNA combinations tested (FIG. 19). As for the triple ETR combination, the conditions in which the gRNAs F4 and H10 were co-present were the most resistant to T cell restimulation (FIG. 19). Overall, these data show that the double ETR combination containing KRAB and DNMT3L performs as efficiently as the canonical triple ETR combination in silencing B2M in T cells.

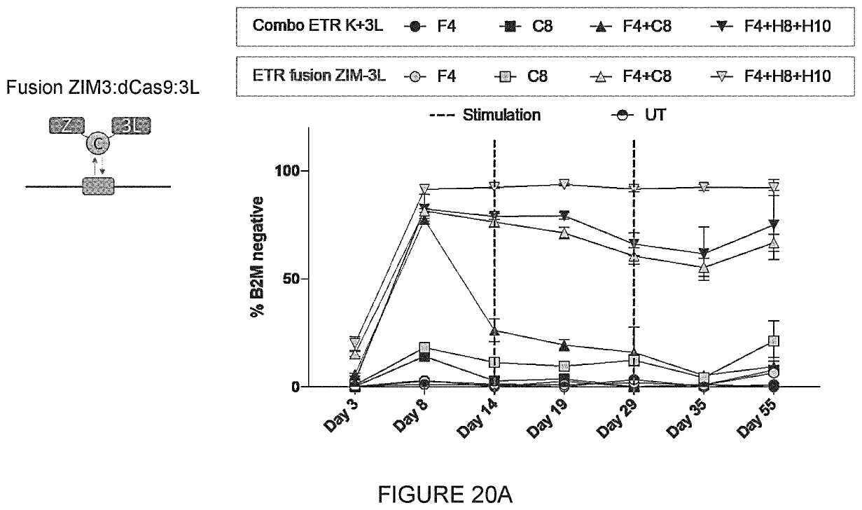

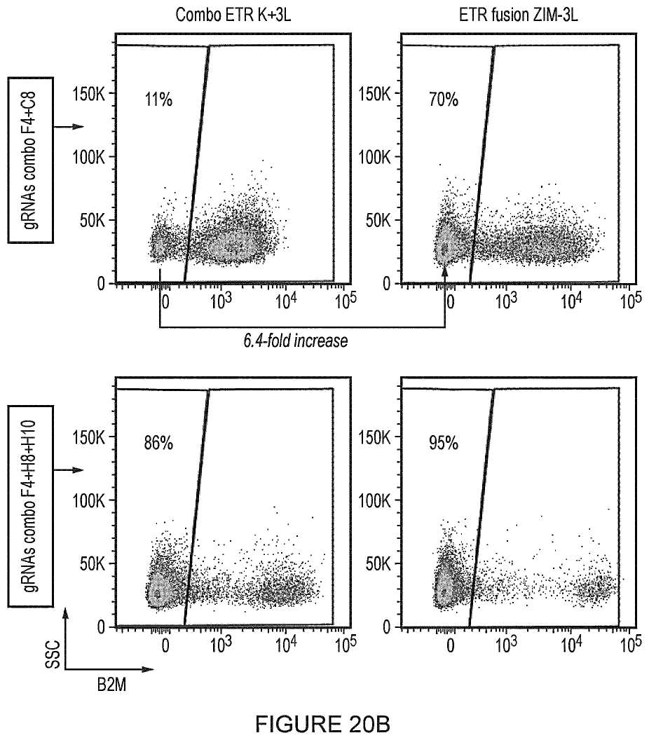

Based on these results, we then compared the efficiencies of B2M epi-silencing between the double ETR combination containing KRAB and DNMT3L and an all-in-one bi-partite ETR equipped with the KRAB domain homolog of the Zinc finger imprinted 3 (ZIM3) protein (Alerasool et al., Nat Methods (2020) 17(11):1093-6) and DNMT3L (FIG. 20A, left schematic), hereafter referred as to the ZIM:dCas9:DNMT3L fusion. The amino acid sequence of this fusion protein is shown below, wherein the SV40 nuclear localization signals (NLSs) are in box, the ZIM3 KRAB repressor domain is in boldface, the flexible linkers are in underlined boldface, dCas9 is underlined and the DNMT3L domain is in italics (only):

(SEQ ID NO: 4481)

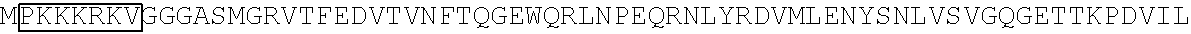

RLEQGKEPWLEEEEVLGSGRAEKNGDIGGQIWKPKDVKESLGGGGSGGGGSGGGGSGGGGSLEDKKYS
IGLAIGTNSVGWAVITDEYKVPSKKFKVLGNTDRHSIKKNLIGALLFDSGETAEATRLKRTARRRYTR
RKNRICYLQEIFSNEMAKVDDSFFHRLEESELVEEDKKHERHPIFGNIVDEVAYHEKYPTIYHLRKKL
VDSTDKADLRLIYLALAHMIKERGHFLIEGDLNPDNSDVDKLFIQLVQTYNQLFEENPINASGVDAKA
ILSARLSKSRRLENLIAQLPGEKKNGLFGNLIALSLGLTPNEKSNEDLAEDAKLQLSKDTYDDDLDNL
LAQIGDQYADLFLAAKNLSDAILLSDILRVNTEITKAPLSASMIKRYDEHHQDLTLLKALVRQQLPEK
YKEIFFDQSKNGYAGYIDGGASQEEFYKFIKPILEKMDGTEELLVKLNREDLLRKQRTEDNGSIPHQI
HLGELHAILRRQEDFYPELKDNREKIEKILTFRIPYYVGPLARGNSRFAWMTRKSEETITPWNFEEVV
DKGASAQSFIERMTNFDKNLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKIIKDKDELDNEENEDILED
IVLTLTLFEDREMIEERLKTYAHLEDDKVMKQLKRRRYTGWGRLSRKLINGIRDKQSGKTILDELKSD
GFANRNFMQLIHDDSLTFKEDIQKAQVSGQGDSLHEHIANLAGSPAIKKGILQTVKVVDELVKVMGRH
KPENIVIEMARENQTTQKGQKNSRERMKRIEEGIKELGSQILKEHPVENTQLQNEKLYLYYLQNGRDM
YVDQELDINRLSDYDVDAIVPQSFLKDDSIDNKVLTRSDKNRGKSDNVPSEEVVKKMKNYWRQLLNAK
LITQRKFDNLTKAERGGLSELDKAGFIKRQLVETRQITKHVAQILDSRMNTKYDENDKLIREVKVITL
KSKLVSDFRKDFQFYKVREINNYHHAHDAYLNAVVGTALIKKYPKLESEFVYGDYKVYDVRKMIAKSE
QEIGKATAKYFFYSNIMNFFKTEITLANGEIRKRPLIETNGETGEIVWDKGRDFATVRKVLSMPQVNI
VKKTEVQTGGFSKESILPKRNSDKLIARKKDWDPKKYGGEDSPTVAYSVLVVAKVEKGKSKKLKSVKE
LLGITIMERSSFEKNPIDFLEAKGYKEVKKDLIIKLPKYSLFELENGRKRMLASAGELQKGNELALPS
KYVNFLYLASHYEKLKGSPEDNEQKQLFVEQHKHYLDEIIEQISEFSKRVILADANLDKVLSAYNKHR
DKPIREQAENIIHLFTLTNLGAPAAFKYEDTTIDRKRYTSTKEVLDATLIHQSITGLYETRIDLSQLG

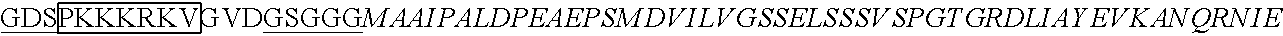

DICICCGSLQVHTQHPLFEGGICAPCKDKFLDALFLYDDDGYQSYCSICCSGETLLICGNPDCTRCYC
FECVDSLVGPGTSGKVHAMSNWVCYLCLPSSRSGLLORRRKWRSQLKAFYDRESENPLEMFETVPVWR
RQPVRVLSLFEDIKKELTSLGFLESGSDPGQLKHVVDVTDTVRKDVEEWGPEDLVYGATPPLGHTCDR
PPSWYLFQFHRLLQYARPKPGSPRPFFWMFVDNLVLNKEDLDVASRFLEMEPVTIPDVHGGSLQNAVR
VWSNIPAIRSRHWALVSEEELSLLAQNKQSSKLAAKWPTKLVKNCELPLREYFKYESTELTSSL

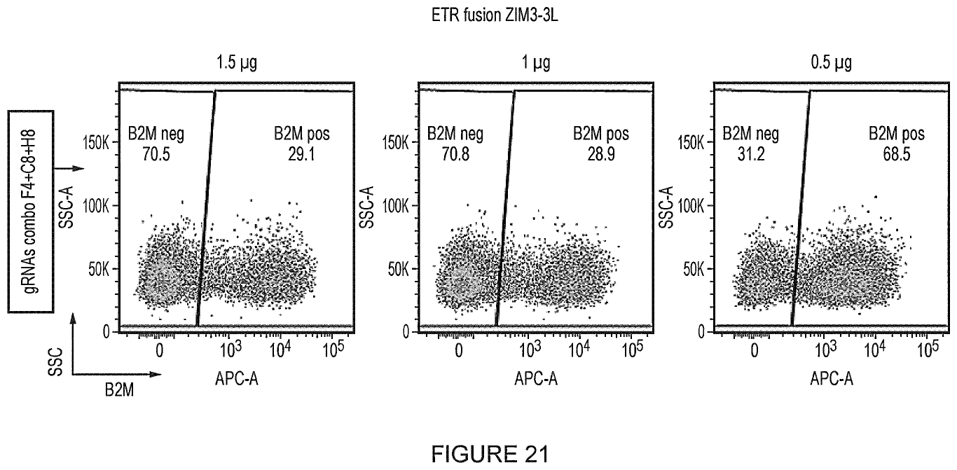

In these experiments, the T cells were co-transfected with the mRNAs encoding the ETRs and (i) the gRNAs F4 or C8, to assess if the bi-partite ETR was able to rescue epi-silencing efficiency of individual gRNAs; (ii) the dual-gRNA combination C8+F4; or (iii) the best-performing triple gRNA combination F4+H8+H10. Cells were then analysed by flow cytometry until day 55. To avoid any confounding effects due to the delivery of different amounts of mRNAs encoding the ETRs, these experiments were performed by using 1.5 μg of each ETR for the double combination and 1.5 μg of the ZIM:dCas9:DNMT3L fusion. As such, matched amounts of epigenetic effectors were used. In accordance with our previous data, individual gRNAs were ineffective with the double ETR combination, and adoption of the ZIM:dCas9:DNMT3L fusion only slightly increased B2M epi-silencing efficiency and exclusively for gRNA C8 (FIG. 20A, right graph). On the other hand, a marked increase in B2M epi-silencing was found when comparing the double ETR combination and the ZIM:dCas9:DNMT3L fusion with the dual-gRNA combination C8+F4 (from 11% to 70%, respectively; FIGS. 20A-B). Similar results were obtained for the triple gRNA combination F4+H8+H10, although the differences between the double ETR combination and the ZIM:dCas9:DNMT3L fusion were less pronounced (86 and 95% of B2M-negative cells for the double ETR combination and the ZIM:dCas9:DNMT3L fusion, respectively; FIGS. 20A-B). This effect was likely due to the already high epi-silencing efficiency of the double ETR combination. For all conditions with the triple gRNA combination, B2M epi-silencing proved to be durable, resisting 2 rounds of T cell restimulations. Notable was the stability observed with the ZIM:dCas9:DNMT3L fusion and the triple gRNA combination, which reached 95% of B2M-negative cells at day 8 post-treatment to then remain stable until day 55. Finally, to assess if the mRNA dose of the ZIM:dCas9:DNMT3L fusion was at saturation, we performed a dose titration experiment in T cells and found that one third of the standard doses (1 vs. 1.5 μg) was already sufficient to obtain efficient epi-silencing of B2M (FIG. 21).

Overall, these data show that adoption of the fusion protein ZIM:dCas9:DNMT3L improves epi-silencing in T cells, achieving up to 95% of B2M-negative cells. Interesting features of this fusion protein include the reduced costs of production as compared to the triple or double ETR combinations and the fact that it can depose efficient silencing at one third of the dose of the double ETR combination.

### Example 4—Orthogonal Editing of B2M and TRAC in Human Primary T Cells without Inducing Reciprocal Chromosomal Translocations

Based on the above data, we then tested if co-delivery of Cas9-based ETRs together with truncated gRNAs against B2M and the full-length gRNA against TRAC (SEQ ID NO: 4575) can induce orthogonal edits (namely epi-silencing of B2M and targeted integration into the TRAC gene) in human primary T cells without causing reciprocal chromosomal translocations. To mediate epi-silencing of B2M, we used some of the truncated gRNAs described above (see FIGS. 2-4), namely truncated C8 (C8_16; 16 nt-long spacer; gRNA SEQ ID NO: 4578), truncated F4 (F4_16; 16 nt-long spacer; gRNA SEQ ID NO: 4579) and truncated H8 (H8_15; 15 nt-long spacer; gRNA SEQ ID NO: 4577), which we co-delivered in T cells as a triple combination. All truncations herein start from the 5′ end of the full-length sequence. In these experiments, we also used the reduced ETR combination/architecture identified above, namely the double ETRs containing KRAB and DNMT3L or the cognate all-in-one fusion protein with a ZIM3 KRAB domain and DNMT3L, both of which were modified to contain the catalytically active Cas9. The all-in-one fusion with ZIM3 KRAB, active Cas9 and DNMT3L domains has the following amino acid sequence, wherein the SV40 NLSs are in box, the ZIM3 KRAB repressor domain is in boldface, the flexible linkers are in underlined boldface, Cas9 is underlined and the DNMT3L domain is in italics (only):

(SEQ ID NO: 4482)

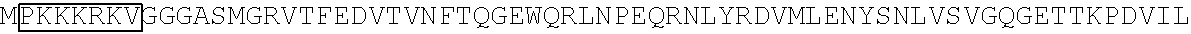

RLEQGKEPWLEEEEVLGSGRAEKNGDIGGQIWKPKDVKESLGGGGSGGGGSGGGGSGGGGSLEDKKYS
IGLDIGTNSVGWAVITDEYKVPSKKFKVLGNTDRHSIKKNLIGALLEDSGETAEATRLKRTARRRYTR
RKNRICYLQEIFSNEMAKVDDSFFHRLEESFLVEEDKKHERHPIFGNIVDEVAYHEKYPTIYHLRKKL
VDSTDKADLRLIYLALAHMIKFRGHFLIEGDLNPDNSDVDKLFIQLVQTYNQLFEENPINASGVDAKA
ILSARLSKSRRLENLIAQLPGEKKNGLFGNLIALSLGLTPNEKSNFDLAEDAKLQLSKDTYDDDLDNL
LAQIGDQYADLFLAAKNLSDAILLSDILRVNTEITKAPLSASMIKRYDEHHQDLTLLKALVRQQLPEK
YKEIFFDQSKNGYAGYIDGGASQEEFYKFIKPILEKMDGTEELLVKLNREDLLRKORTEDNGSIPHQI
HLGELHAILRRQEDFYPFLKDNREKIEKILTFRIPYYVGPLARGNSRFAWMTRKSEETITPWNFEEVV
DKGASAQSFIERMTNFDKNLPNEKVLPKHSLLYEYFTVYNELTKVKYVTEGMRKPAFLSGEQKKAIVD
LLFKTNRKVTVKQLKEDYFKKIECFDSVEISGVEDRENASLGTYHDLLKIIKDKDELDNEENEDILED
IVLTLTLFEDREMIEERLKTYAHLEDDKVMKQLKRRRYTGWGRLSRKLINGIRDKQSGKTILDELKSD
GFANRNEMQLIHDDSLTFKEDIQKAQVSGQGDSLHEHIANLAGSPAIKKGILQTVKVVDELVKVMGRH
KPENIVIEMARENQTTQKGQKNSRERMKRIEEGIKELGSQILKEHPVENTQLQNEKLYLYYLQNGRDM
YVDQELDINRLSDYDVDHIVPQSFLKDDSIDNKVLTRSDKNRGKSDNVPSEEVVKKMKNYWRQLLNAK
LITQRKEDNLTKAERGGLSELDKAGFIKRQLVETROITKHVAQILDSRMNTKYDENDKLIREVKVITL
KSKLVSDERKDFQFYKVREINNYHHAHDAYLNAVVGTALIKKYPKLESEFVYGDYKVYDVRKMIAKSE
QEIGKATAKYFFYSNIMNFFKTEITLANGEIRKRPLIETNGETGEIVWDKGRDFATVRKVLSMPQVNI
VKKTEVQTGGFSKESILPKRNSDKLIARKKDWDPKKYGGFDSPTVAYSVLVVAKVEKGKSKKLKSVKE
LLGITIMERSSFEKNPIDFLEAKGYKEVKKDLIIKLPKYSLFELENGRKRMLASAGELQKGNELALPS
KYVNFLYLASHYEKLKGSPEDNEQKQLFVEQHKHYLDEIIEQISEFSKRVILADANLDKVLSAYNKHR
DKPIREQAENIIHLFTLTNLGAPAAFKYEDTTIDRKRYTSTKEVLDATLIHQSITGLYETRIDLSQLG

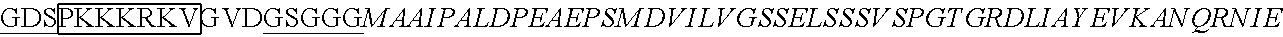

DICICCGSLOVHTQHPLFEGGICAPCKDKELDALFLYDDDGYQSYCSICCSGETLLICGNPDCTRCYC
FECVDSLVGPGTSGKVHAMSNWVCYLCLPSSRSGLLORRRKWRSQLKAFYDRESENPLEMFETVPVWR
RQPVRVLSLFEDIKKELTSLGFLESGSDPGQLKHVVDVTDTVRKDVEEWGPEDLVYGATPPLGHTCDR
PPSWYLFQFHRLLQYARPKPGSPRPFFWMFVDNLVLNKEDLDVASRFLEMEPVTIPDVHGGSLQNAVR
VWSNIPAIRSRHWALVSEEELSLLAQNKQSSKLAAKWPTKLVKNCELPLREYFKYFSTELTSSL

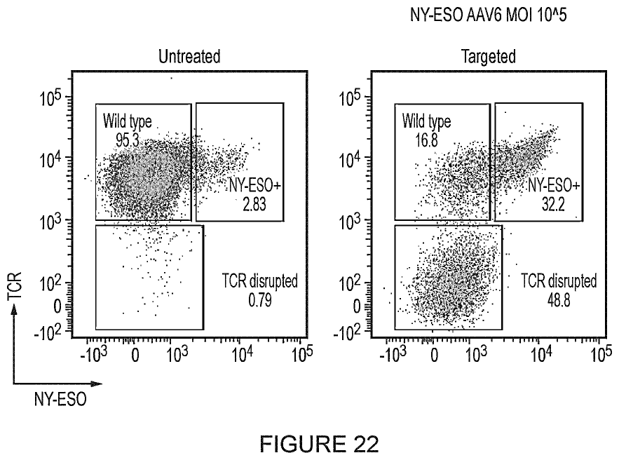

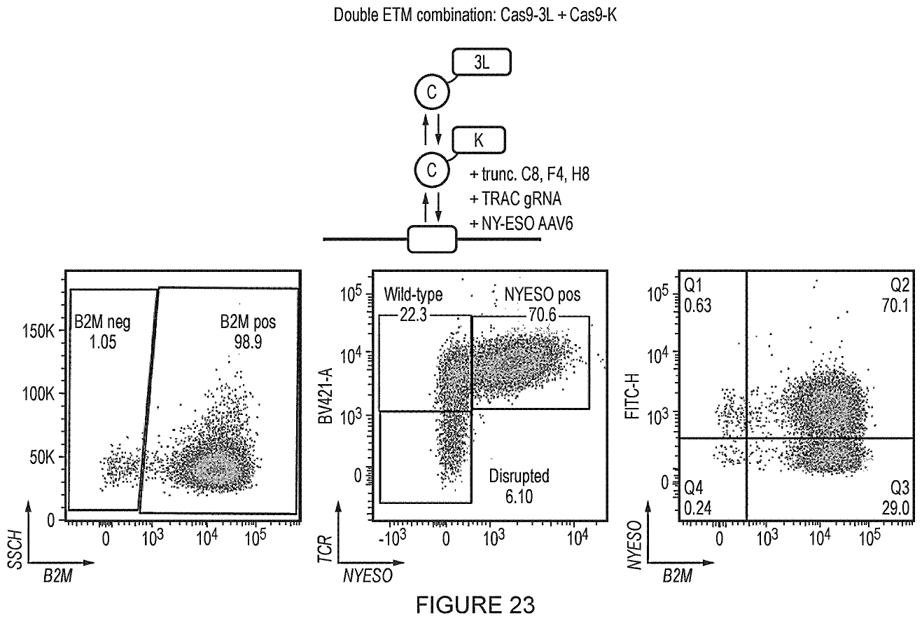

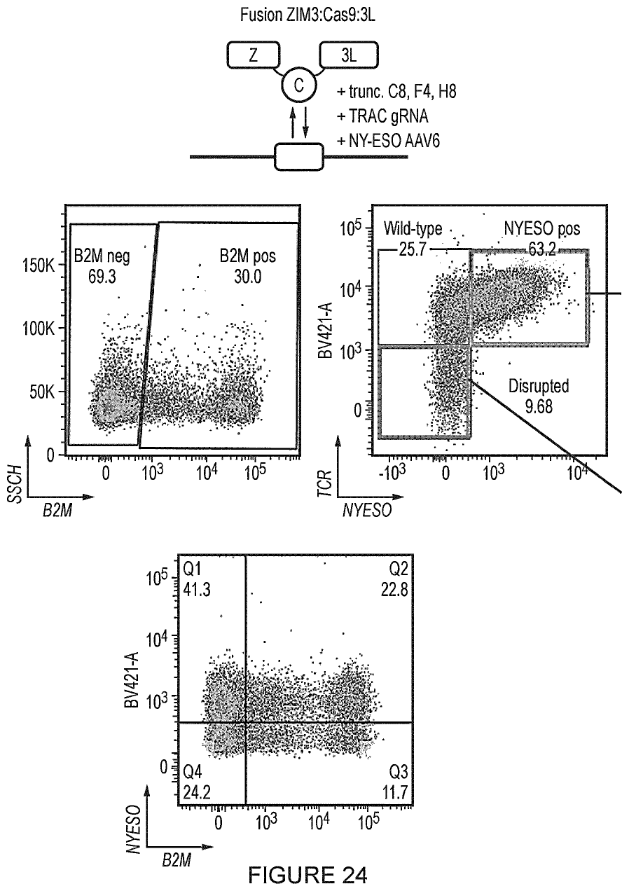

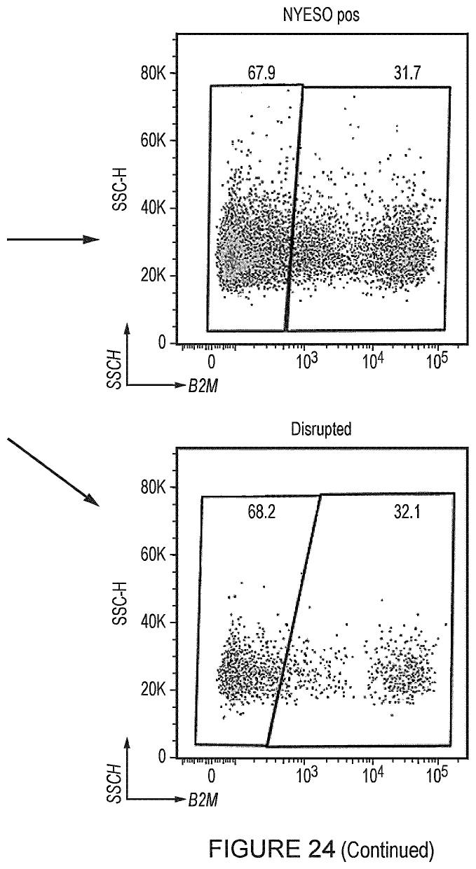

For Cas9-mediated targeted integration into the TRAC locus, we exploited a previously developed AAV6-based donor template, which contains the sequences encoding for a transgenic TCR against the tumour antigen NY-ESO embedded within TRAC homology arms (Roth et al., Nature (2018) 559(7714):405-9). Upon targeted integration, the transgenic TCR was expressed from the endogenous TRAC locus, and it can be measured by flow cytometry using a specific pentamer (FIG. 22). Concerning T cells transfected with the double Cas9-based ETR combination, this treatment unexpectedly resulted in little, if any, epi-silencing of B2M, while editing of TRAC was highly efficient, resulting in up to 70% of NY-ESO-positive cells and up to 6% of endogenous TCR disrupted cells (FIG. 23). Remarkably, the use of the ZIM3:Cas9:DNMT3L fusion protein rescued B2M epi-silencing efficiency, resulting in up to 70% of B2M-negative T cells (FIG. 24). Also in these conditions, high levels of editing of the TRAC locus were measured. Further analyses of the NY-ESO-positive T cells showed that 65% of them were also B2M-negative. A similar analysis focused on the TCR disrupted cells showed comparable efficiencies of co-editing. Analyses at day 34 post-treatment showed that orthogonal edits were resistant to T cell restimulation.

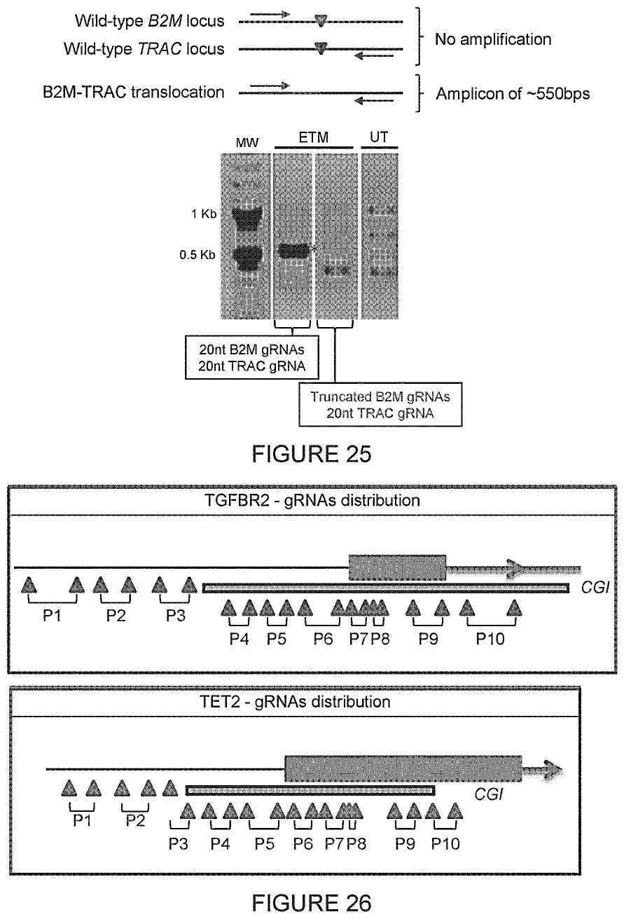

We then evaluated by PCR analyses the presence of reciprocal chromosomal translocations between B2M and TRAC. Of note, no signs of reciprocal translocations were found (FIG. 25), indicating that (i) the B2M gene was silenced through epigenetic mechanisms rather than by genetic inactivation and (ii) truncated gRNAs abolished Cas9 cleavage, confirming our previous findings in the B2M^{dTomato} K-562 cell line (see Example 2). At variance with these data, T cells co-transfected with Cas9-based ETRs and full-length gRNAs against both B2M and TRAC displayed high levels of co-editing together with clear signs of reciprocal chromosomal translocations (FIG. 25).

Overall, these data show that the co-adoption of gRNAs of different lengths and Cas9-based ETRs can promote orthogonal edits (i.e., epi-silencing and targeted integration or epi-silencing and gene disruption) at high efficiency in human primary T cells without inducing reciprocal chromosomal translocations.

### Example 5—Identification of gRNAs to Mediate High Levels of Epi-Silencing of TET2 and TGFBR2 in Human Primary T Lymphocytes

To expand the orthogonal editing approach to more than two genes, we designed a panel of gRNAs targeting TET2 and TGFBR2. Inactivation of these genes represents a potential therapeutic approach to either increasing persistency or protecting T cell products from immune-dampening signals originating from the tumour microenvironment (see, e.g., Fraietta et al., Nature (2018) 558(7709):307-12; Nobles et al., J Clin Invest (2020) 130(2):673-85; Li et al., Nature (2020) 587(7832):121-5; Alishah et al., J Transl Med (2021) 19(1):482). For each of these genes, we designed 20 gRNAs in a genomic window of 1 Kb around their transcription start site (FIG. 26). We then set out to test epi-silencing efficacy of these gRNAs directly in T cells, using the standard triple ETR combination containing KRAB, DNMT3A and DNMT3L effector domains. We pool contiguous gRNAs, and then coupled each of these pairs with the others to obtain any possible pair combinations. The tested pairs are shown in Table 3 below (SEQ: SEQ ID NO). The gRNAs used in this experiment contained 20-nucleotide (full-length) spacer sequences.

TABLE 3
TGFBR2 and TET2 gRNA Pairs

| | | | | | |
|---|---|---|---|---|---|
| | | | Target | Spacer | |
| gRNA | Pair | gRNA Target | Sequence | Sequence | gRNA |
| Spacer ID | No. | Sequence | SEQ | SEQ | SEQ |

TGFBR2 gRNAs

| | | | | | |
|---|---|---|---|---|---|
| TG1 | P1 | TTCTTTAGGTCG | 4539 | 4553 | 4608 |
| | | AAGTCTAGAGG | | | |
| TG2 | | GTGCTCGCGACT | 4540 | 4554 | 4609 |
| | | CAATAGATTGG | | | |
| TG3 | P2 | AACGCATCTCTA | 4541 | 4555 | 4610 |
| | | AAGCACCTAGG | | | |
| TG4 | | CTGATCTACTAG | 4542 | 4556 | 4611 |
| | | GGAAAACGTGG | | | |
| TG5 | P3 | TTGAGTAAATAC | 4543 | 4557 | 4612 |
| | | TTGGAGCGAGG | | | |
| TG6 | | AGTCGGCCAAAG | 1239 | 2940 | 4613 |
| | | CTCTCGGAGGG | | | |
| TG7 | P4 | GAAACTCCTCGC | 1236 | 2937 | 4614 |
| | | CAACAGCTGGG | | | |
| TG8 | | GAGTGAGTCACT | 1229 | 2930 | 4615 |
| | | CGCGCGCACGG | | | |
| TG9 | P5 | CGCGTGCACCCG | 1254 | 2955 | 4616 |
| | | CTCGGGACAGG | | | |
| TG10 | | GGGGCCTCCCCG | 4544 | 4558 | 4617 |
| | | CGCCTCGCCGG | | | |
| TG11 | P6 | TGGCGAGCGGGC | 1256 | 2957 | 4618 |
| | | GCCACATCTGG | | | |
| TG12 | | TCGGTCTATGAC | 1228 | 2929 | 4619 |
| | | GAGCAGCGGGG | | | |
| TG13 | P7 | CCTGAGCAGCCC | 4545 | 4559 | 4620 |
| | | CCGACCCATGG | | | |
| TG14 | | GGACGATGTGCA | 1244 | 2945 | 4621 |
| | | GCGGCCACAGG | | | |
| TG15 | P8 | TGCTGGCGATAC | 1230 | 2931 | 4622 |
| | | GCGTCCACAGG | | | |
| TG16 | | AACGTGCGGTGG | 1241 | 2942 | 4623 |
| | | GATCGTGCTGG | | | |
| TG17 | P9 | GACTGTCAAGCG | 1238 | 2939 | 4624 |
| | | CAGCGGAGAGG | | | |
| TG18 | | CTTTCCTCGTTT | 1234 | 2935 | 4625 |
| | | CCGCCCGGGGG | | | |
| TG19 | P10 | GCCCGACTCCCG | 1237 | 2938 | 4626 |
| | | TAGCTGCAGGG | | | |
| TG20 | | CGTTGTGTTGGC | 1231 | 2932 | 4627 |
| | | CGCGTTCGAGG | | | |

TET2 gRNAs

| | | | | | |
|---|---|---|---|---|---|
| TE1 | P1 | GGAATTAGCTCT | 4547 | 4560 | 4588 |
| | | GTATCGGTCGG | | | |
| TE2 | | AAAGTAAGGGCT | 4548 | 4561 | 4589 |
| | | CTTACGAGAGG | | | |
| TE3 | P2 | GGCGTCTCACAG | 4549 | 4562 | 4590 |
| | | ATTGAAATAGG | | | |
| TE4 | | CGGTCAATTTCC | 4550 | 4563 | 4591 |
| | | CAGTTTGTCGG | | | |
| TE5 | P3 | AGCGCTCCCCTG | 2742 | 4443 | 4592 |
| | | TTTCACCGAGG | | | |
| TE6 | | CGCGGGCAACGG | 2733 | 4434 | 4593 |
| | | GATCTAAAGGG | | | |
| TE7 | P4 | CGCAAGCGGAGG | 2765 | 4466 | 4594 |
| | | TGTGGTGCGGG | | | |
| TE8 | | GTGCGGGTACAC | 2737 | 4438 | 4595 |
| | | TCCGGAGGAGG | | | |
| TE9 | P5 | TGCGCGGGACCT | 2728 | 4429 | 4596 |
| | | CGAAGTGGTGG | | | |
| TE10 | | AGCAGAGCAAGC | 2768 | 4469 | 4597 |
| | | GCGAAGGTTGG | | | |
| TE11 | P6 | TGCAGCCCTCGG | 4551 | 4564 | 4598 |
| | | GAACCCCGGGG | | | |
| TE12 | | GTGGTGCGCCCG | 2748 | 4449 | 4599 |
| | | GACCAGCGCGG | | | |
| TE13 | P7 | TCACGCCGTGCA | 2732 | 4433 | 4600 |
| | | GTGGCGCGGGG | | | |
| TE14 | | GGTGCCGCCGGC | 2741 | 4442 | 4601 |
| | | CTTTGTGCTGG | | | |
| TE15 | P8 | GCACCGGGCGTC | 2729 | 4430 | 4602 |
| | | CAGCACAAAGG | | | |
| TE16 | | AGGGAATTAGCC | 2730 | 4431 | 4603 |
| | | CCCCGCACCGG | | | |
| TE17 | P9 | AGTGGCAGCGGC | 2773 | 4474 | 4604 |
| | | GAGAGCTTGGG | | | |
| TE18 | | ACTTGCATGCGA | 2731 | 4432 | 4605 |
| | | GCGGGACCCGG | | | |
| TE19 | P10 | ACTCAGCGGGGC | 4552 | 4565 | 4606 |
| | | CGGCGTCTCGG | | | |
| TE20 | | CCTTATGAATAT | 2777 | 4478 | 4607 |
| | | TGATGCGGAGG | | | |

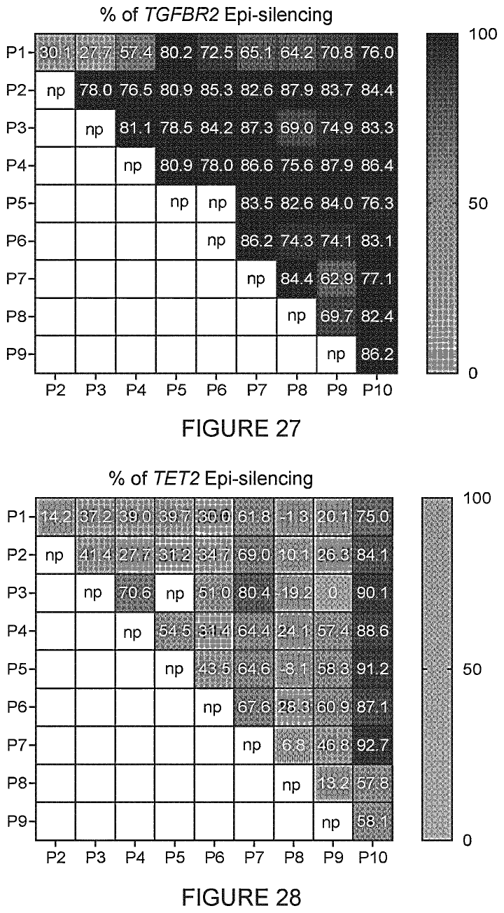

We then delivered these new pools individually to T cells together with the mRNAs encoding the triple ETR combination. The two genes were expressed at low levels and the detection of their protein products by flow cytometry was complicated by the nuclear localization of TET2 and the inducibility of TGFBR2. Thus, to quantify epi-silencing efficiencies, we used digital droplet PCR (ddPCR), a technique for measuring the expression profile of selected genes at high sensitivity. Finally, we analysed the cells at day 28 post-treatment. FIG. 27 shows the percentage of TGFBR2 epi-silencing for each pair combination upon normalization to the levels of TGFBR2 expression in mock-treated cells. This analysis shows that nearly all pair combinations were able to induce epi-silencing of TGFBR2 at high efficiencies (≥60%). On the other hand, a similar analysis performed for TET2 showed that epi-silencing of this gene was more variable, with some pair combinations displaying no activity while others being highly effective (FIG. 28). In this regard, combinations containing either pair number 7 (P7) or number 10 (P10) were the most effective ones, leading to up to 92% of TET2 reduction when coupled together.

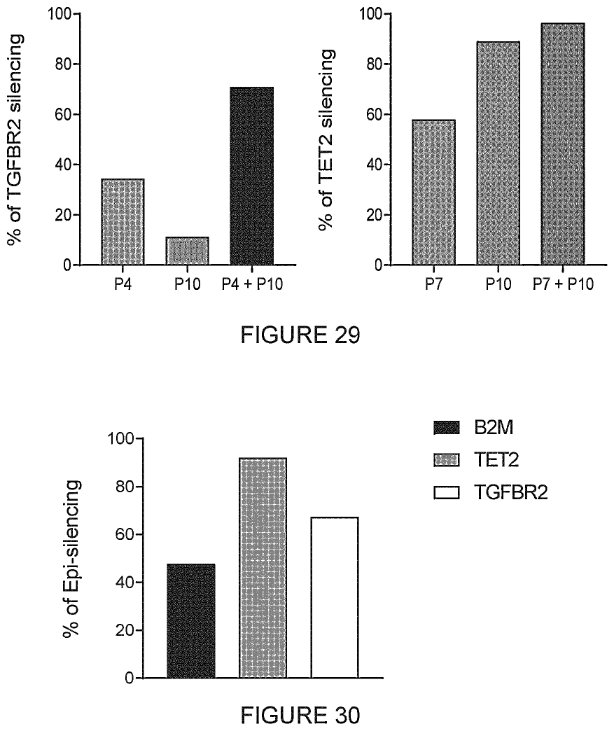

With the aim of reducing to 2 the number of gRNAs required to silence each of these genes, we evaluated epi-silencing efficiency of selected gRNA pairs. In this regard, we chose pairs number 4 (gRNA IDs TG7_20 and TG8_20) and 10 (gRNA IDs TG19_20 and TG20_20) for TGFBR2 and pairs number 7 (gRNAs TE13_20 and TE14_20) and 10 (gRNAs TE19_20 and TE20_20) for TET2. Co-delivery of these pairs individually together with the triple ETR combination followed by ddPCR analysis at day 22 post-treatment showed that pairs number 4 and number 10 were the most effective in promoting epi-silencing of TGFBR2 and TET2, respectively, leading to up to 35% and 90% of reduction of the two transcripts (FIG. 29). Of note, at variance with TGFBR2 for which the pairs combination led to 89% reduction, for TET2, the epi-silencing efficiency of pair number 10 was comparable to those observed when delivering the parental pairs combination. Overall, these data show that TET2 and TGFBR2 can be efficiently silenced by the ETRs technology.

We then tested multiplexed epigenetic silencing of B2M, TET2 and TGFBR2. To this end, we co-treated human primary T cells with: (i) the mRNA encoding for the ETR ZIM3:dCas9:DNMT3L fusion; (ii) the F4+H8+H10 combination of full-length gRNAs against B2M (i.e., gRNA IDs F4_20, H8_20, and H10_20); (iii) pair number 10 of full-length gRNAs against TET2; (iv) combination of pairs number 4 and 10 of full-length gRNAs against TGFBR2. We then measured the expression levels of these genes by ddPCR and found that they were all markedly downregulated, resulting in up to 47%, 92% and 67% of epi-silencing of B2M, TET2 and TGFBR2, respectively (FIG. 30). Overall, these data show that B2M, TET2 and TGFBR2 can be co-silenced by the ETRs technology.

### Example 6—Poly-Functional Orthogonal Editing of Multiple Genes with ETM without Causing Reciprocal Chromosomal Translocations in Human Primary T Lymphocytes

We decided to combine orthogonal editing of B2M and TRAC with epi-silencing of either TGFBR2 or TET2. To this end, we first truncated the gRNAs against TET2 and TGFBR2 from FIG. 30 to 15 nt in length. gRNAs with the truncated spacer are shown in the table below. We then co-transfected human primary T cells with the mRNA encoding for the ETM ZIM3:Cas9:DNMT3L fusion together with: (i) the truncated gRNAs against B2M, namely F4 (gRNA ID F4_16; 16 nt-long spacer; gRNA SEQ ID NO: 4579), H8 (H8_15; 15 nt-long spacer; gRNA SEQ ID NO: 4577) and H10 (H10_14; 14 nt-long spacer; gRNA: SEQ ID NO: 4576); (ii) the full-length gRNA against TRAC (SEQ ID NO: 4575); (iii) the truncated gRNAs corresponding either to pair number 10 (TE19_15 and TE 20_15) for TET2 or to pairs number 4 (TG7_15 and TG8_15) and 10 (TG19_15 and TG20_15) for TGFBR2 (see Table 4; SEQ: SEQ ID NO). Cells were either transduced or not with the AAV6 donor template for targeted integration of the NY-ESO TCR into the TRAC locus. Treated T cells were then analysed by (i) flow cytometry to measure epigenetic silencing of B2M and genetic editing of TRAC (i.e., disruption or targeted integration of the NY-ESO TCR, according to the absence or not of the AAV6 donor) and (ii) ddPCR to quantify the expression levels of TET2 and TGFBR2.

TABLE 4
Truncated TGFBR2 and TET2 gRNA Pairs

| | | | |
|---|---|---|---|
| gRNA Spacer ID | Pair No. | gRNA ID | gRNA SEQ |

TGFBR2 gRNAs

| | | | |
|---|---|---|---|
| TG7 | P4 | TG7_15 | 4584 |
| TG8 | | TG8_15 | 4585 |
| TG19 | P10 | TG19_15 | 4586 |
| TG20 | | TG20_15 | 4587 |

TET2 gRNAs

| | | | |
|---|---|---|---|
| TE19 | P10 | TE19_15 | 4582 |
| TE20 | | TE20_15 | 4583 |

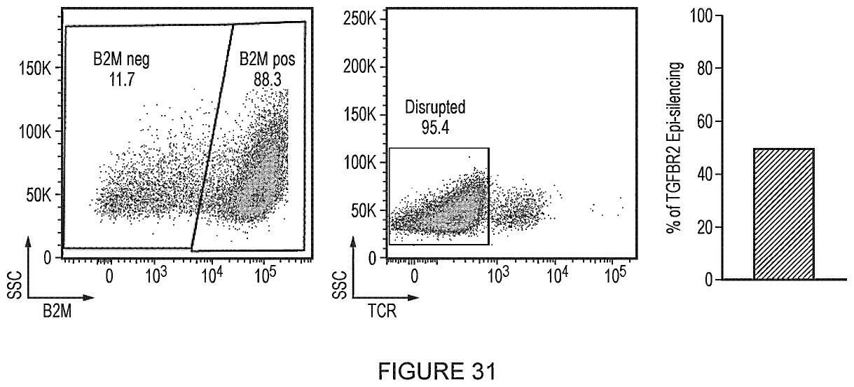

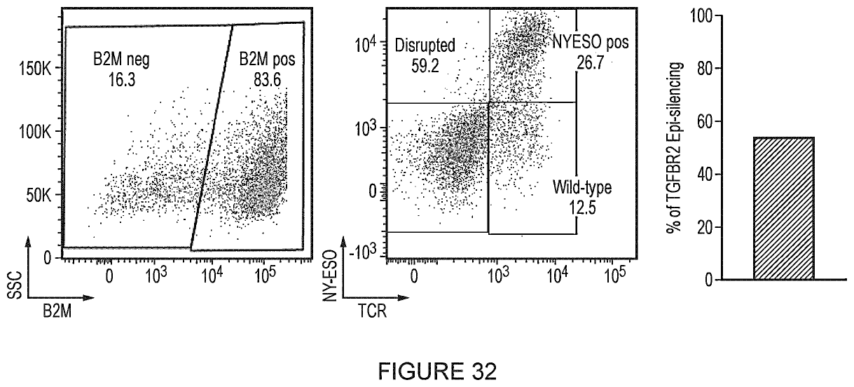

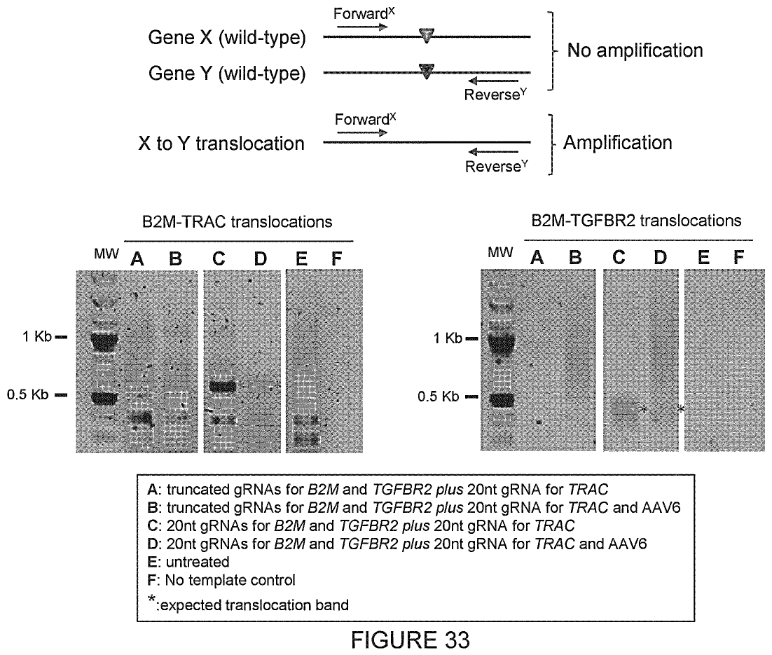

Concerning the experimental conditions of poly-functional editing of B2M, TRAC and TGFBR2 without the AAV6 donor, the analyses showed that ZIM3:Cas9:DNMT3L was able to induce up to 11% and 95% of cells negative for B2M and endogenous TCR, respectively (FIG. 31). ddPCR analyses of bulk-treated cells showed that the expression levels of TGFBR2 were markedly reduced in these samples, resulting up to 50% of epi-silencing (FIG. 31). Concerning the samples treated with the AAV6 donor, we found that up to 16% of treated T cells were negative for B2M, while 59% and 26.7% turned negative for the endogenous TCR and positive for NY-ESO, respectively (FIG. 32). In these cells, the epi-silencing efficiency of TGFBR2 was 54% (FIG. 32). Importantly, molecular analyses of treated T cells, either transduced or not with the AAV6 donor, did not show any sign of reciprocal chromosomal translocations among the three targeted genes (FIG. 33). At variance with this latter data, experiments performed with ZIM3:Cas9:DNMT3L and full-length gRNAs against the investigated genes showed clear evidence of reciprocal chromosomal translocations among B2M, TRAC and TGFBR2 (FIG. 33).

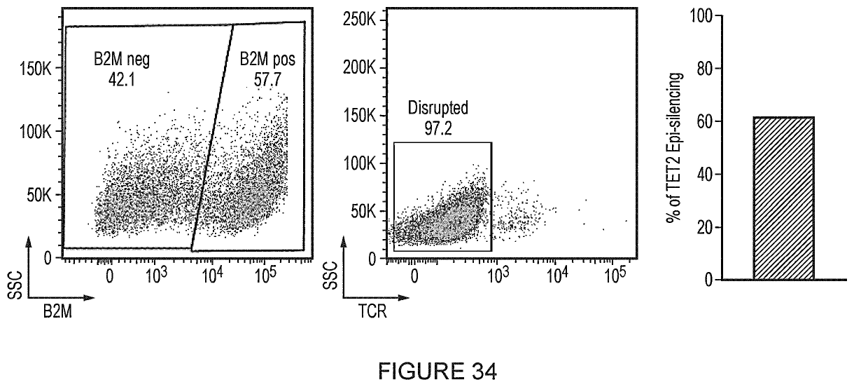

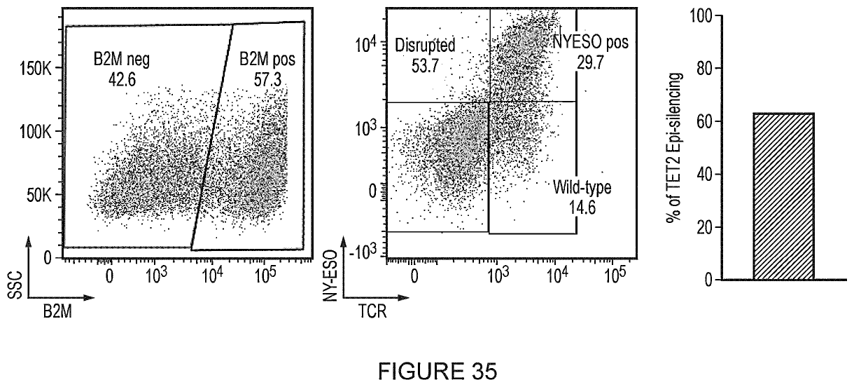

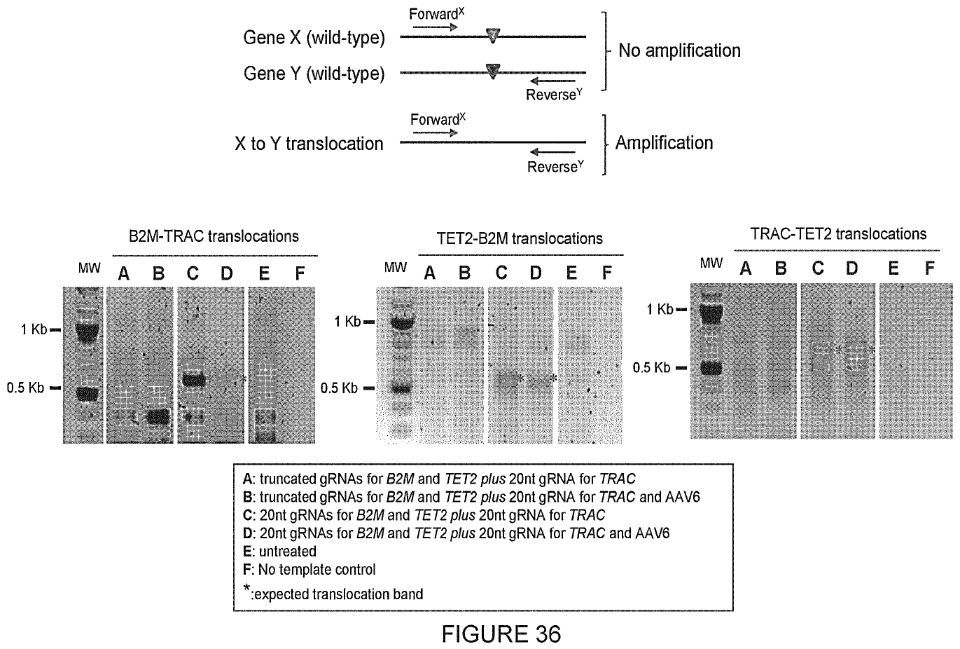

Concerning the experimental conditions of poly-functional editing of B2M, TRAC and TET2 without the AAV6 donor, the analyses showed that ZIM3:Cas9:DNMT3L was able to induce up to 46% and 99% of cells negative for B2M and endogenous TCR, respectively (FIG. 34). ddPCR analyses of bulk-treated cells showed that the expression levels of TET2 were markedly reduced in these samples, resulting up to 63% of epi-silencing (FIG. 34). Concerning the samples treated with the AAV6 donor, we found that up to 40% of treated T cells were negative for B2M, while 53% and 29% turned negative for the endogenous TCR and positive for NY-ESO, respectively (FIG. 35). In these cells, epi-silencing efficiency of TET2 was 60% (FIG. 35). Importantly, molecular analyses of treated T cells, either transduced or not with the AAV6 donor, did not show any sign of reciprocal chromosomal translocations among the three targeted genes (FIG. 36). At variance with this latter data, experiments performed with ZIM3:Cas9:DNMT3L and full-length gRNAs against the investigated genes showed clear evidence of numerous reciprocal chromosomal translocations among B2M, TRAC and TET2 (FIG. 36).

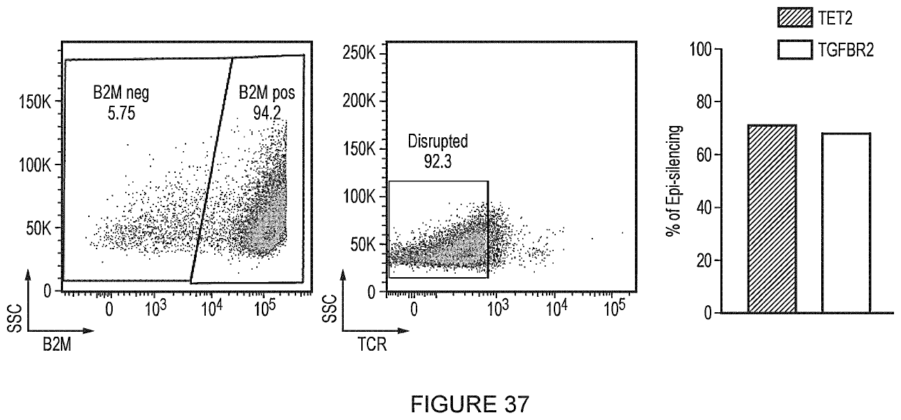

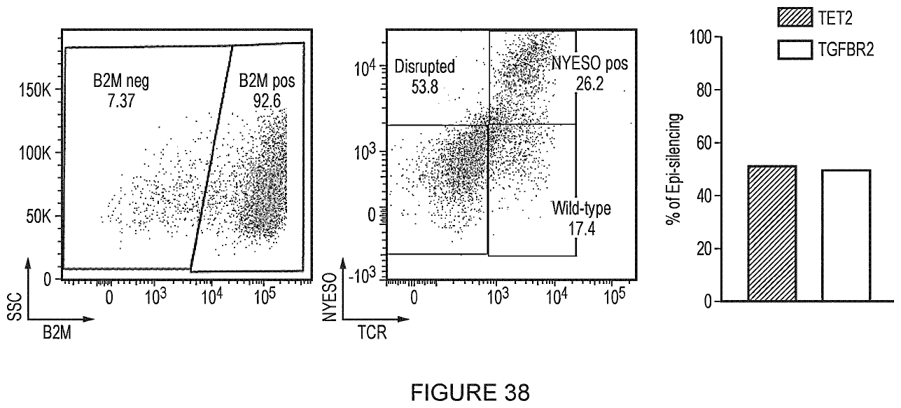

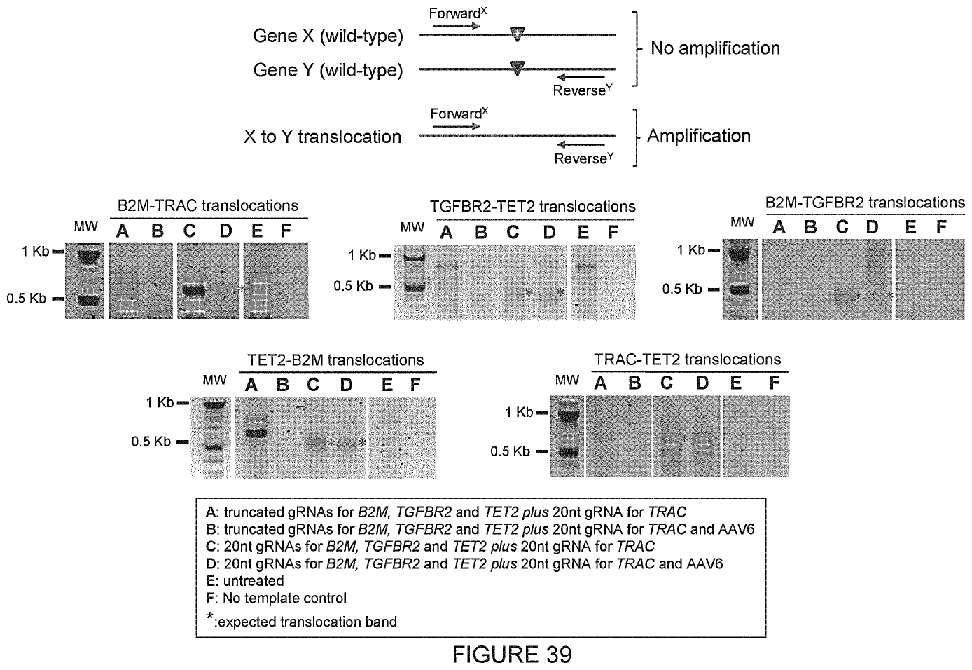

Finally, we tested quadruple poly-functional editing of B2M, TRAC, TGFBR2 and TET2 using ZIM3:Cas9:DNMT3L, with or without the AAV6 donor. In this experiment we used truncated gRNAs for B2M, TGFBR2 and TET2 and the full-length gRNA for TRAC. In the conditions without the AAV6 donor, up to 5.7% and 93% of treated cells proved negative for B2M and the endogenous TCR, respectively (FIG. 37). ddPCR analysis of these cells showed that the transcripts of TGFBR2 and TET2 were markedly reduced as compared to mock-treated samples, resulting in up to 70% and 71% of epi-silencing, respectively (FIG. 37). Concerning the samples treated with the AAV6 donor, we found that up to 7% of treated T cells were negative for B2M, while 54% and 26% turned negative for the endogenous TCR and positive for NY-ESO, respectively (FIG. 38). In these cells, the epi-silencing efficiencies of TGFBR2 and TET2 were 50% and 51%, respectively (FIG. 38). Importantly, molecular analyses of treated T cells, either transduced or not with the AAV6 donor, did not show any sign of reciprocal chromosomal translocations among the three targeted genes (FIG. 39). At variance with this latter data, experiments performed with ZIM3:Cas9:DNMT3L and full-length gRNAs against the investigated genes showed clear evidence of numerous reciprocal chromosomal translocations among B2M, TRAC and TET2 (FIG. 39).

Overall, these data show that Cas9-based ETRs (EMT) with truncated and full-length gRNAs can impose multiple orthogonal edits in T cells without inducing reciprocal chromosomal translocations.

Additional targets that may be silenced with epigenetic silencing include, for example: A2AR; CISH; PTPN11; PTPN6; PTPA; PTPN2; JUNB; TOX; TOX2; NR4A1; NR4A2; NR4A3; MAP4K1; REL; IRF4; DGKA; PIK3CD; HLA-A; USP16; DCK and FAS.

Epigenetic silencing of these targets may be coupled to gene editing of TRAC, PD-1 and CTLA4 genes that do not have CpG islands (CGIs).

### Cell Culture Conditions

Peripheral blood mononuclear cells (PBMCs) were freshly isolated from healthy donors using centrifugation on a Ficoll gradient (Lymphoprep™). CD3-positive lymphocytes were then purified by magnetic separation using Pan T cells isolation kit (Miltenyi Biotech), according to the manufacturer instructions. The purity of T lymphocytes was assessed by flow cytometry (FACSCanto™ II—BD Bioscience, Cytoflex—Beckman Coulter) using anti-CD3 (BD, 349201), CD4 (Biolegend, 317429) and -CD8 (Biolegend, 344708) antibodies. T lymphocytes were stimulated using anti-CD3/CD28 magnetic beads (Dynabeads human T-activator CD3/CD28, Thermo Fisher) in a 1:1 ratio and maintained in culture in RPMI (Corning) supplemented with penicillin (100 IU/ml), streptomycin (100 μg/ml), 2% glutamine, 10% FBS (Euroclone) and 5 ng/ml of each IL-7 and IL-15 (PeproTech). The K-562^{dTomato} reporter cell line was previously described (Amabile et al., supra) and maintained in culture in RPMI supplemented with penicillin (100 IU/ml), streptomycin (100 μg/ml), 2% glutamine and 10% FBS. All cells were cultured in a 5% CO₂ humidified atmosphere at 37° C.

mRNAs, gRNAs and Donor Templates

The gRNAs used in these studies were designed using CHOPCHOP (Labun et al., Nucleic Acids Res. (2019) 47(W1):W171-4). For T cell experiments, gRNAs were purchased highly chemically modified from IDT, including 2′-O-methyl residues and phosphorothioate modifications as previously described (Finn et al., Cell Rep (2018) 22(9):2227-35). mRNAs encoding for the ETRs, the Cas9-based ETRs and Cas9 were purchased from TriLink or produced in house using the MEGAscript™ T7 Transcription Kit (Invitrogen), according to the manufacturer instructions. In both cases, mRNAs were 5′ capped using CleanCap® Reagent (TriLink) and UTP was completely substituted by N1-Methylpseudouridine-5′-Triphosphate (TriLink). In house produced mRNAs were also concentrated using Amicon® Ultra-15 Centrifugal Filter Unit (Sigma-Aldrich). The construct IG4 NY-ESO TCR alpha/beta with homology arms for the TRAC locus was obtained by Addgene (plasmid #112021) and cloned inside an AAV transfer construct containing AAV2 inverted terminal repeats. AAV6 was produced by TIGEM Vector Core by triple-transfection method and purified by ultracentrifugation. For the K-562^{dTomato} experiments, full-length or truncated gRNAs were cloned downstream the human U6 promoter as fusion transcripts with the tracrRNA (Amabile et al., supra). ETRs, Cas9-based ETRs and Cas9 sequences were cloned inside expression plasmids under the control of CMV promoter (Amabile et al., supra).

### Gene Editing Procedures

T cells were edited two days after purification. Dynabeads were removed prior to electroporation. 5×10⁵ cells were electroporated with 1.5 μg (unless otherwise specified) of stabilized mRNA for each ETRs/Cas9-ETRs/Cas9 and 3 μg for each highly modified gRNA using the Lonza 4D-Nucleofector™ (P3 Primary Cell solution, EO-115 program). Immediately after nucleofection, 80 μl of RMPI were added directly to the cuvette and cells were incubated 15 minutes at 37° C. Cells were then moved in a 96-U bottom wells and 100 μl of complete 2× medium (RPMI with 20% FBS, 4 mM L-Glutamine, 2% P/S and 10 ng/ml of each IL-7 and IL-15) were added. In gene targeting experiments, AAV6 NY-ESO TCR was also added to the 2× medium at a dose of 10⁵ vg/cell. Percentage of B2M negative cells was assessed by flow cytometry using an anti-B2M antibody (Biolegend, 316312) while NY-ESO/TCR positive events were assessed by using an anti-Vβ13.1 antibody (Beckman Coulter) or an anti-human TCR alpha/beta antibody (Biolegend). Complete fresh medium was added to the culture every third day. For the K-562^{dTomato} experiments, 5×10⁵ cells were electroporated with 600 ng of each ETRs/Cas9-ETRs/Cas9 plasmid and 200 ng of the gRNA plasmid using the using the Lonza 4D-Nucleofector™ (SF Cell Line solution, FF-120 program). Immediately after nucleofection, cells were plated in 96-U bottom wells in complete RPMI. dTomato negative cells were analysed by flow cytometry. Cytofluorimetric analyses were performed using Flow Jo Software (FLOWJO, LLC).

### Molecular Analysis

Genomic DNA from the cell line was extracted using Maxwell 16 LEV Blood DNA kit (Promega) for samples consisting of less than 2×10⁶ cells. DNA from less than 5×10⁵ cells was extracted using the QuickExtract™ DNA Extraction Solution (Epicentre). Genetic indels were detected by using Surveyor nuclease assay (Surveyor Mutation Kit, IDT), according to the manufacturer instructions. The following primers were used to measure mutations at the B2M locus:

TABLE 5
B2M Primers for Measurement of Mutations

| | | SEQ |
|---|---|---|
| Description | Sequence | ID NO |
| B2M (F4, C8) | TACAGACAGCAAACTCACCCAGTC | 4527 |
| Forward | | |
| B2M (F4, C8) | AGAACTTGGAGAAGGGAAGTCACG | 4528 |
| Reverse | | |
| B2M (H8) Forward | ATCTTCTGGGTTTCCGTTTTCT | 4529 |
| B2M (H8) Reverse | TCTCGTGATGTTTAAGAAGGCA | 4530 |
| B2M (H10) Forward | CGTGAGTCTCTCCTACCCTCC | 4531 |
| B2M (H10) Reverse | TTATCGACGCCCTAAACTTTGT | 4532 |

The following primers were used to measure mutations on other loci of interest:

TABLE 6
Primers for Measurement of
Mutations in Other Loci

| | | SEQ |
|---|---|---|
| Description | Sequence | ID NO |
| TRAC Forward | CCGTATAAAGCATGAGACCGTG | 4533 |
| TRAC Reverse | ATTCCTGAAGCAAGGAAACAGC | 4534 |
| TGFBR2 Forward | TCGGTCTATGACGAGCAGC | 4535 |
| TGFBR2 Reverse | GAAACTTTCCTCGTTTCCGC | 4536 |
| TET2 Forward | AACAAGGCAGTGCTAATGCCT | 4537 |
| TET2 Reverse | GCTTTGGAGGCAGCTCAGAG | 4538 |

Translocation analyses were performed using GoTaq® DNA Polymerase (Promega) combining the forward and reverse primers listed above according to the gRNA employed in the experiment. Amplicons were run on a 1% agarose gel. The following primers were used to detect genomic translocations of interest:

TABLE 7
Primers for Detection of Genomic Translocations

| | | SEQ |
|---|---|---|
| Description | Sequence | ID NO |
| B2M Forward | TACAGACAGCAAACTCACCCAGTC | 4629 |
| B2M Reverse | ACAAAGTTTAGGGCGTCGATAA | 4630 |
| TRAC Forward | CCGTATAAAGCATGAGACCGTG | 4631 |
| TRAC Reverse | ATTCCTGAAGCAAGGAAACAGC | 4632 |
| TGFBR2 Forward | CACGTTCAGAAGTCGGGTGAGT | 4633 |
| TGFBR2 Reverse | TCCAGGAGCTAAGGACTGAGGA | 4634 |
| TET2 Forward | TAATTCCCTGGGAGCCGGGG | 4635 |
| TET2 Reverse | TTGCTCCCCAGTCCCTGGAA | 4636 |

For gene expression analysis, total RNA was extracted from 10⁶ cells using the RNeasy Mini kit (QIAGEN) and reverse-transcribed using random hexamers according to the SuperScript III First-Strand Synthesis System (Invitrogen) manufacturer's instructions. Transcripts levels were determined by digital droplet PCR using from 0.2-1 Ong of template cDNA. The PCR reaction was carried out by adding 1× of TaqMan Gene Expression assays (Applied Biosystems) following manufacturer's instructions (Biorad), read with QX200 reader and analysed with QuantaSoft software (Biorad). Data were normalized over HPRT and mock-treated samples. The reagents used are listed below:

| | |
|---|---|
| B2M | Hs00187842_m1 |
| TGFBR2 | Hs00234253_m1 |
| TET2 | Hs00325999_m1 |
| HPRT | Hs02800695_m1 |

### LIST OF SEQUENCES

Sequences disclosed in the present disclosure are listed below.

TABLE 8
Sequence Description

| SEQ ID NO | Description |
|---|---|
| 1 | ZNF10 KRAB domain |
| 2 | ZIM3 KRAB domain |
| 3 | ZNF350 KRAB domain |
| 4 | ZNF197 KRAB domain |
| 5 | RBAK KRAB domain |
| 6 | ZKSCAN1 KRAB domain |
| 7 | KRBOX4 KRAB domain |
| 8 | ZNF274 KRAB domain |
| 9 | DNMT3A catalytic domain |
| 10 | DNMT3B catalytic domain |
| 11 | DNMT3B |
| 12 | DNMT1 catalytic domain |
| 13 | DNMT3L |
| 14 | SETDB1 |
| 15 | SETDB1 catalytic domain |
| 16 | Cas9 (catalytically active) |
| 17 | dCas9 |
| 18 | exemplary ETM-KRAB |
| 19 | exemplary ETM-DNMT3A |
| 20 | exemplary ETM-ENMT3L |
| 21 | exemplary B2M target sequence, sense strand |
| 22 | exemplary B2M target sequence, antisense strand |
| 23-45 | B2M gRNA spacers |
| | 24: F4 |
| | 25-34: F4 truncated from 5′ end |
| | (19-10 nt sequences, respectively) |
| | 35: C8 |
| | 36-45: C8 truncated from 5′ end |
| | (19-10 nt sequences, respectively) |
| 46 | TRAC gRNA spacer |
| 47-96 | TRAC target sequences |
| 97-146 | TRBC1 target sequences |
| 147-196 | TRBC2 target sequences |
| 197-246 | PDCD1 target sequences |
| 247-296 | TIM-3/HAVCR2 target sequences |
| 297-346 | TIGIT target sequences |
| 347-396 | LAG3 target sequences |
| 397-446 | CTLA4 target sequences |
| 447-511 | AAVS1 target sequences |
| 512-561 | CCR5 target sequences |
| 562-611 | TRAC gRNA spacers |
| 612-661 | TRBC1 gRNA spacers |
| 662-711 | TRBC2 gRNA spacers |
| 712-761 | PDCD1 gRNA spacers |
| 762-811 | TIM-3/HAVCR2 gRNA spacers |
| 812-861 | TIGIT gRNA spacers |
| 862-911 | LAG3 gRNA spacers |
| 912-961 | CTLA4 gRNA spacers |
| 962-1026 | AAVS1 gRNA spacers |
| 1027-1076 | CCR5 gRNA spacers |
| 1077-1177 | B2M target sequences |
| 1178-1227 | HLA-A target sequences |
| 1228-1277 | TGFBR2 target sequences |
| 1278-1377 | A2AR target sequences |
| 1378-1427 | FAS target sequences |
| 1428-1477 | DCK target sequences |
| 1478-1527 | DGKA target sequences |
| 1528-1577 | USP16 target sequences |
| 1578-1627 | PTPN11 target sequences |
| 1628-1677 | PTPN6 target sequences |
| 1678-1727 | PTPA target sequences |
| 1728-1777 | PTPN2 target sequences |
| 1778-1827 | CISH target sequences |
| 1828-1927 | PI3KCD.1 target sequences |
| 1928-1977 | MAP4K1 target sequences |
| 1978-2027 | NR4A1 target sequences |
| 2028-2127 | NR4A2 target sequences |
| 2128-2277 | NR4A3 target sequences |
| 2278-2377 | JUNB target sequences |
| 2378-2427 | REL target sequences |
| 2428-2527 | TOX target sequences |
| 2528-2627 | TOX2 target sequences |
| 2628-2727 | IRF4 target sequences |
| 2728-2777 | TET2 target sequences |
| 2778-2878 | B2M gRNA spacers |
| | 2778: H11 |
| | 2780: H8 |
| | 2801: H12 |
| | 2813: C8 |
| | 2863: H10 |
| | 2878: F4 |
| 2879-2928 | HLA-A gRNA spacers |
| 2929-2978 | TGFBR2 gRNA spacers |
| 2979-3078 | A2AR gRNA spacers |
| 3079-3128 | FAS gRNA spacers |
| 3129-3178 | DCK gRNA spacers |
| 3179-3228 | DGKA gRNA spacers |
| 3229-3278 | USP16 gRNA spacers |
| 3279-3328 | PTPN11 gRNA spacers |
| 3329-3378 | PTPN6 gRNA spacers |
| 3379-3428 | PTPA gRNA spacers |
| 3429-3478 | PTPN2 gRNA spacers |
| 3479-3528 | CISH gRNA spacers |
| 3529-3628 | PI3KCD.1 gRNA spacers |
| 3629-3678 | MAP4K1 gRNA spacers |
| 3679-3728 | NR4A1 gRNA spacers |
| 3729-3828 | NR4A2 gRNA spacers |
| 3829-3978 | NR4A3 gRNA spacers |
| 3979-4078 | JUNB gRNA spacers |
| 4079-4128 | REL gRNA spacers |
| 4129-4228 | TOX gRNA spacers |
| 4229-4328 | TOX2 gRNA spacers |
| 4329-4428 | IRF4 gRNA spacers |
| 4429-4478 | TET2 gRNA spacers |
| 4479 | Example of B2M gRNA designed for epigenetic editing |
| | (comprises spacer of SEQ ID NO: 24) |
| 4480 | Example of B2M gRNA designed for epigenetic editing |
| | (comprises spacer of SEQ ID NO: 35) |
| 4481 | ZIM: dCas9: DNMT3L |
| 4482 | ZIM: Cas9: DNMT3L |
| 4483 | Example of B2M gRNA designed for epigenetic editing |
| | (comprises spacer of SEQ ID NO: 2780) |
| 4484-4493 | H8 truncated from 5′ end |
| | (19-10 nt sequences, respectively) |
| 4494 | Example of B2M gRNA designed for epigenetic editing |
| | (comprises spacer of SEQ ID NO: 2863) |
| 4495-4504 | H10 truncated from 5′ end |
| | (19-10 nt sequences, respectively) |
| 4505 | Example of B2M gRNA designed for epigenetic editing |
| | (comprises spacer of SEQ ID NO: 2778) |
| 4506-4515 | H11 truncated from 5′ end |
| | (19-10 nt sequences, respectively) |
| 4516 | Example of B2M gRNA designed for epigenetic editing |
| | (comprises spacer of SEQ ID NO: 2801) |
| 4517-4526 | H12 truncated from 5′ end |
| | (19-10 nt sequences, respectively) |
| 4527 | B2M (F4, C8) forward primer |
| 4528 | B2M (F4, C8) reverse primer |
| 4529 | B2M (H8) forward primer |
| 4530 | B2M (H8) reverse primer |
| 4531 | B2M (H10) forward primer |
| 4532 | B2M (H10) reverse primer |
| 4533 | TRAC forward primer |
| 4534 | TRAC reverse primer |
| 4535 | TGFBR2 forward primer |
| 4536 | TGFBR2 reverse primer |
| 4537 | TET2 forward primer |
| 4538 | TET2 reverse primer |
| 4539-4545 | TGFBR2 target sequences |
| 4546-4552 | TET2 target sequences |
| 4553-4559 | TGFBR2 gRNA spacers |
| 4560-4565 | TET2 gRNA spacers |
| 4566-4567 | Examples of tracr sequences |
| 4568-4573 | Examples of B2M gRNAs (comprise spacers of |
| | SEQ ID NOs: 24, 35, 2780, 2863, 2778, and |
| | 2801, respectively) |
| 4574-4575 | Exemplary gRNAs targeting TRAC |
| 4576-4579 | Exemplary full-length modified gRNAs targeting B2M |
| 4580-4583 | Exemplary truncated modified gRNAs targeting TET2 |
| 4584-4587 | Exemplary truncated modified gRNAs targeting TGFBR2 |
| 4588-4607 | Exemplary full-length modified gRNAs targeting TET2 |
| 4608-4627 | Exemplary full-length modified gRNAs targeting TGFBR2 |
| 4628 | Exemplary full-length modified gRNA targeting GFP |
| 4629 | B2M forward primer for translocation assessment |
| 4630 | B2M reverse primer for translocation assessment |
| 4631 | TRAC forward primer for translocation assessment |
| 4632 | TRAC reverse primer for translocation assessment |
| 4633 | TGFBR2 forward primer for translocation assessment |
| 4634 | TGFBR2 reverse primer for translocation assessment |
| 4635 | TET2 forward primer for translocation assessment |
| 4636 | TET2 reverse primer for translocation assessment |
| 4637 | Alternative ZIM3 KRAB domain |
| 4638 | Exemplary chemically modified gRNA |

1. A combination for modifying transcription, expression and/or activity of one or more gene in a cell, the combination comprising:
(A) one or more fusion proteins each comprising a catalytically active CRISPR/Cas endonuclease domain, wherein the one or more fusion proteins collectively comprise a transcriptional repressor domain and a DNMT3L domain, or polynucleotide(s) encoding the one or more fusion proteins;
(B) one or more guide RNAs (gRNAs) having a spacer sequence with a length that allows epigenetic editing and not gene editing of a first gene in the cell, wherein the first gene comprises a CpG island (CGI), or polynucleotide(s) coding for the one or more gRNAs; and
(C) one or more gRNAs having a spacer sequence with a length that allows gene editing of a second gene in the cell, or polynucleotide(s) coding for the one or more gRNAs.

2. The combination of claim 1, wherein:
(a) the one or more gRNAs in (B) has a spacer sequence of 11 to 17 nucleotides; and/or
(b) the one or more gRNAs in (C) has a spacer sequence of 18 to 21 nucleotides.

3. The combination of claim 1 or 2, wherein the combination comprises one to three fusion proteins.

4. The combination of any one of claims 1-3, further comprising a donor DNA comprising 5′ and 3′ arms that are homologous to sequences in the second gene.

5. The combination of any one of claims 1-4, wherein the endonuclease domain is derived from a Cas9 protein, optionally SpCas9.

6. The combination of any one of claims 1-5, wherein
the first gene is selected from B2M, TET2, TGFBR2, A2AR, CISH, PTPN11, PTPN6, PTPA, PTPN2, JUNB, TOX, TOX2, NR4A1, NR4A2, NR4A3, MAP4K1, REL, IRF4, DGKA, PIK3CD, HLA-A, USP16, DCK, and FAS; and/or
the second gene is selected from TRAC, TRBC, PDCD1, TIM-3, TIGIT, LAG3, CTLA4, AAVS1, and CCR5.

7. The combination of claim 6, wherein the second gene is a TRAC gene, optionally wherein the one or more gRNAs targeting the TRAC gene comprise a spacer having the sequence of one of SEQ ID NOs: 562-611.

8. The combination of any one of claims 1-7, wherein the first gene is a B2M gene, optionally wherein the one or more gRNAs targeting the B2M gene each comprise a spacer having the sequence of one of SEQ ID NOs: 28-33 and 39-44, or the sequence of one of SEQ ID NOs: 2778-2878 with a 3 to 9 nucleotide truncation at the 5′ end.

9. The combination of any one of claims 1-7, wherein the first gene is a TGFBR2 gene, optionally wherein the one or more gRNAs targeting the TGFBR2 gene each comprise a spacer having the sequence of one of SEQ ID NOs: 2929-2978 and 4553-4559 with a 3 to 9 nucleotide truncation at the 5′ end.

10. The combination of any one of claims 1-7, wherein the first gene is a TET2 gene, optionally wherein the one or more gRNAs targeting the TET2 gene each comprise a spacer having the sequence of one of SEQ ID NOs: 4429-4478 and 4560-4565 with a 3 to 9 nucleotide truncation at the 5′ end.

11. The combination of any one of claims 1-10, wherein the cell is a mammalian cell, optionally a human cell, further optionally wherein the cell is a human immune cell, or a human T cell.

12. A combination for regulating one or more gene in a human cell, optionally an immune cell or a T cell, the combination comprising:
one to three fusion proteins each comprising a catalytically inactive Cas9, optionally SpCas9, endonuclease domain, wherein the one to three fusion proteins collectively comprise a transcriptional repressor and a DNMT3L domain, or polynucleotide(s) encoding the one to three fusion proteins, wherein the gene comprises a CpG island (CGI) and is
(i) a B2M gene and the combination further comprises two or more gRNAs each comprising a spacer having the sequence of one of SEQ ID NOs: 2778-2878 optionally with a 1 to 9 nucleotide truncation at the 5′ end, or comprises polynucleotide(s) coding for the gRNAs;
(ii) a TGFBR2 gene and the combination further comprises a gRNA that comprises a spacer having the sequence of any one of SEQ ID NOs: 2929-2978 and 4553-4559 optionally with a 1 to 9 nucleotide truncation at the 5′ end, or comprises polynucleotide(s) coding for the gRNA; or
(iii) a TET2 gene and the combination further comprises a gRNA that comprises a spacer having the sequence of any one of SEQ ID NOs: 4429-4478 and 4560-4565 optionally with a 1 to 9 nucleotide truncation at the 5′ end, or comprises polynucleotide(s) coding for the gRNA.

13. The combination of any one of claims 1-12, wherein the gene comprising a CGI is a B2M gene and the gRNAs targeting it are two or three gRNAs each independently comprising a spacer having the sequence of
C8 (SEQ ID NO: 35),
F4 (SEQ ID NO: 24),
H8 (SEQ ID NO: 2780),
H10 (SEQ ID NO: 2863),
H11 (SEQ ID NO: 2778), or
H12 (SEQ ID NO: 2801),
optionally with a 1 to 9, optionally 3 to 9, nucleotide truncation at the 5′ end.

14. The combination of claim 13, wherein the B2M-targeting gRNAs comprise
(i) a gRNA comprising a spacer having the sequence of F4 optionally with a 1 to 9, optionally 3 to 9, nucleotide truncation at the 5′ end,
a gRNA comprising a spacer having the sequence of H8 optionally with a 1 to 9, optionally 3 to 9, nucleotide truncation at the 5′ end, and
a gRNA comprising a spacer having the sequence of H10 optionally with a 1 to 9, optionally 3 to 9, nucleotide truncation at the 5′ end;
(ii) a gRNA comprising a spacer having the sequence of C8 optionally with a 1 to 9, optionally 3 to 9, nucleotide truncation at the 5′ end,
a gRNA comprising a spacer having the sequence of H8 optionally with a 1 to 9, optionally 3 to 9, nucleotide truncation at the 5′ end, and
a gRNA comprising a spacer having the sequence of H10 optionally with a 1 to 9, optionally 3 to 9, nucleotide truncation at the 5′ end;
(iii) a gRNA comprising a spacer having the sequence of F4 optionally with a 1 to 9, optionally 3 to 9, nucleotide truncation at the 5′ end, and
a gRNA comprising a spacer having the sequence of H8 optionally with a 1 to 9, optionally 3 to 9, nucleotide truncation at the 5′ end;
(iv) a gRNA comprising a spacer having the sequence of F4 optionally with a 1 to 9, optionally 3 to 9, nucleotide truncation at the 5′ end, and
a gRNA comprising a spacer having the sequence of H10 optionally with a 1 to 9, optionally 3 to 9, nucleotide truncation at the 5′ end; or
(v) a gRNA comprising a spacer having the sequence of H8 optionally with a 1 to 9, optionally 3 to 9, nucleotide truncation at the 5′ end, and
a gRNA comprising a spacer having the sequence of H10 optionally with a 1 to 9, optionally 3 to 9, nucleotide truncation at the 5′ end.

15. The combination on of any one of claims 1-12, wherein the gene comprising a CGI is a TGFBR2 gene and the combination comprises one or more gRNAs targeting it, or coding sequences of the one or more gRNAs, the one or more gRNAs each independently comprising a spacer having the sequence of
TG1 (SEQ ID NO: 4553),
TG2 (SEQ ID NO: 4554),
TG3 (SEQ ID NO: 4555),
TG4 (SEQ ID NO: 4556),
TG5 (SEQ ID NO: 4557),
TG6 (SEQ ID NO: 2940),
TG7 (SEQ ID NO: 2937),
TG8 (SEQ ID NO: 2930),
TG9 (SEQ ID NO: 2955),
TG10 (SEQ ID NO: 4558),
TG11 (SEQ ID NO: 2957),
TG12 (SEQ ID NO: 2929),
TG13 (SEQ ID NO: 4559),
TG14 (SEQ ID NO: 2945),
TG15 (SEQ ID NO: 2931),
TG16 (SEQ ID NO: 2942),
TG17 (SEQ ID NO: 2939),
TG18 (SEQ ID NO: 2935),
TG19 (SEQ ID NO: 2938), or
TG20 (SEQ ID NO: 2932),
optionally with a 1 to 9, optionally 3 to 9, nucleotide truncation at the 5′ end.

16. The combination of claim 15, wherein the TGFBR2-targeting gRNAs comprise
(i) a gRNA comprising a spacer having the sequence of TG7 optionally with a 1 to 9, optionally 3 to 9, nucleotide truncation at the 5′ end, and
a gRNA comprising a spacer having the sequence of TG8 optionally with a 1 to 9, optionally 3 to 9, nucleotide truncation at the 5′ end; or
(ii) a gRNA comprising a spacer having the sequence of TG19 optionally with a 1 to 9, optionally 3 to 9, nucleotide truncation at the 5′ end, and
a gRNA comprising a spacer having the sequence of TG20 optionally with a 1 to 9, optionally 3 to 9, nucleotide truncation at the 5′ end.

17. The combination of any one of claims 1-12, wherein the gene comprising a CGI is a TET2 gene and the combination comprises one or more gRNAs targeting it, or coding sequences of the one or more gRNAs, the one or more gRNAs each independently comprising a spacer having the sequence of
TE1 (SEQ ID NO: 4560),
TE2 (SEQ ID NO: 4561),
TE3 (SEQ ID NO: 4562),
TE4 (SEQ ID NO: 4563),
TE5 (SEQ ID NO: 4443),
TE6 (SEQ ID NO: 4434),
TE7 (SEQ ID NO: 4466),
TE8 (SEQ ID NO: 4438),
TE9 (SEQ ID NO: 4429),
TE10 (SEQ ID NO: 4469),
TE11 (SEQ ID NO: 4564),
TE12 (SEQ ID NO: 4449),
TE13 (SEQ ID NO: 4433),
TE14 (SEQ ID NO: 4442),
TE15 (SEQ ID NO: 4430),
TE16 (SEQ ID NO: 4431),
TE17 (SEQ ID NO: 4474),
TE18 (SEQ ID NO: 4432),
TE19 (SEQ ID NO: 4565), or
TE20 (SEQ ID NO: 4478),
optionally with a 1 to 9, optionally 3 to 9, nucleotide truncation at the 5′ end.

18. The combination of claim 17, wherein the TET2-targeting gRNAs comprise
(i) a gRNA comprising a spacer having the sequence of TE13 optionally with a 1 to 9, optionally 3 to 9, nucleotide truncation at the 5′ end, and
a gRNA comprising a spacer having the sequence of TE14 optionally with a 1 to 9, optionally 3 to 9, nucleotide truncation at the 5′ end; or
(ii) a gRNA comprising a spacer having the sequence of TE19 optionally with a 1 to 9, optionally 3 to 9, nucleotide truncation at the 5′ end, and
a gRNA comprising a spacer having the sequence of TE20 optionally with a 1 to 9, optionally 3 to 9, nucleotide truncation at the 5′ end.

19. The combination of any one of claims 1-18, further comprising gRNAs for targeting one or more additional genes in the cell, optionally wherein the combination comprises gRNAs targeting the following genes, or comprises polynucleotides coding for the gRNAs:
(i) B2M and TRAC,
(ii) B2M, TRAC, and TGFBR2,
(iii) B2M, TRAC, and TET2,
(iv) B2M, TGFBR2, and TET2, or
(v) B2M, TGFBR2, TET2, and TRAC

20. The combination of any one of claims 1-19, wherein the one or more fusion proteins collectively further comprise a DNMT1, DNMT3A, DNMT3B, or SETDB1 domain, optionally DNMT3A.

21. The combination of any one of claims 1-20, wherein the combination comprises
(i) a first fusion protein comprising a transcriptional repressor domain and a Cas endonuclease domain, and a second fusion protein comprising a DNMT3L domain and a Cas endonuclease domain, or
(ii) a fusion protein comprising, optionally from N-terminus to C-terminus, a transcriptional repressor domain, a Cas endonuclease domain, and a DNMT3L domain.

22. The combination of any one of claims 1-20, wherein the combination comprises
(i) a first fusion protein comprising a transcriptional repressor domain and a Cas endonuclease domain, a second fusion protein comprising a DNMT3L domain and a Cas endonuclease domain, and a third fusion protein comprising a DNMT3A domain and a Cas endonuclease domain, or
(ii) a fusion protein comprising a transcriptional repressor domain, a Cas endonuclease domain, a DNMT3L domain, and a DNMT3A domain.

23. The combination of any one of claims 1-22, wherein the transcriptional repressor domain is a Kruppel-associated box (KRAB) domain, optionally derived from human Kox1 or ZIM3.

24. The combination of any one of claims 12-23, wherein the combination comprises a fusion protein comprising, optionally from N terminus to C terminus, a KRAB domain derived from ZIM3, a dCas9 domain, and a DNMT3L domain, optionally comprising an amino acid sequence of SEQ ID NO: 4481.

25. The combination of any one of claims 1-11 and 13-23, wherein the combination comprises a fusion protein comprising, optionally from N terminus to C terminus, a KRAB domain derived from ZIM3, a catalytically active Cas9 domain, and a DNMT3L domain, optionally comprising an amino acid sequence of SEQ ID NO: 4482.

26. The combination of any one of the claims 1-25, wherein the gRNA(s) are chemically modified, optionally wherein the chemically modified gRNA(s) comprise phosphorothioate internucleoside linkages at the 5′ and/or 3′ ends, and/or 2′-O-methyl nucleotides.

27. A pharmaceutical composition comprising the combination of any one of claims 1-26.

28. A cell comprising the combination of any one of claims 1-26, or a progeny of the cell.

29. A method of modifying transcription, expression and/or activity of one or more gene in a human T cell, comprising introducing the combination of any one of claim 1-26 into the cell in vitro or ex vivo.

30. A cell obtained by the method of claim 29, or a progeny of the cell.

31. The cell of claim 28 or 30, wherein the cell is a human T cell, optionally engineered to express a recombinant antigen receptor, optionally selected from a recombinant T cell receptor (TCR) or a chimeric antigen receptor (CAR).

32. The cell of claim 28, 30, or 31 for use in treating a human in need thereof.

33. Use of the cell of claim 28, 30, or 31 in the manufacture of medicament for treating a human in need thereof.

34. A method of treating a human in need thereof, comprising administering to the human the cell of claim 28, 30, or 31.

35. The cell for use according to claim 32, the use of claim 33, or the method of claim 34, wherein the cell is allogeneic or autologous to the human.
